SEQUENCE LISTING (217 sequences)

SEQ ID NO: 1 - protein, 7 aa, synthetic construct
TSGVGVG

SEQ ID NO: 2 - protein, 9 aa, synthetic construct
GFSLSTSGV

SEQ ID NO: 3 - protein, 12 aa, synthetic construct
GFSLSTSGVGVG

SEQ ID NO: 4 - protein, 16 aa, synthetic construct
QIYWNDEKRYSSSLKS

SEQ ID NO: 5 - protein, 5 aa, synthetic construct
YWNDE

SEQ ID NO: 6 - protein, 10 aa, synthetic construct
RRGIGNWFDP

SEQ ID NO: 7 - protein, 11 aa, synthetic construct
RASQGISSWLA

SEQ ID NO: 8 - protein, 7 aa, synthetic construct
AASGLQS

SEQ ID NO: 9 - protein, 9 aa, synthetic construct
QQANSFPFT

SEQ ID NO: 10 - protein, 120 aa, synthetic construct
QITLKESGPTLVKPTQTLTLTCTFSGFSLSTSGVGVGWIRQPPGKAPEWLAQIYWNDEKRYSSSLKSRLTITKDTSKNQVVLKMTNMDPVDTATYYCAHRRGIGNWFDPWGQGTLVTVSS

SEQ ID NO: 11 - protein, 107 aa, synthetic construct
DIQMTQSPSSVSASVGDRVTITCRASQGISSWLAWYQQKPGKAPNLLIYAASGLQSGVPSRFSGSGSGTDFTLTISSLQPEDFASYYCQQANSFPFTFGPGTKVDIK

SEQ ID NO: 12 - protein, 247 aa, synthetic construct
QITLKESGPTLVKPTQTLTLTCTFSGFSLSTSGVGVGWIRQPPGKAPEWLAQIYWNDEKRYSSSLKSRLTITKDTSKNQVVLKMTNMDPVDTATYYCAHRRGIGNWFDPWGQGTLVTVSSGGGGSGGGGSGGGGSGGGGSDIQMTQSPSSVSASVGDRVTITCRASQGISSWLAWYQQKPGKAPNLLIYAASGLQSGVPSRFSGSGSGTDFTLTISSLQPEDFASYYCQQANSFPFTFGPGTKVDIK

SEQ ID NO: 13 - protein, 491 aa, synthetic construct
MALPVTALLLPLALLLHAARPQITLKESGPTLVKPTQTLTLTCTFSGFSLSTSGVGVGWIRQPPGKAPEWLAQIYWNDEKRYSSSLKSRLTITKDTSKNQVVLKMTNMDPVDTATYYCAHRRGIGNWFDPWGQGTLVTVSSGGGGSGGGGSGGGGSGGGGSDIQMTQSPSSVSASVGDRVTITCRASQGISSWLAWYQQKPGKAPNLLIYAASGLQSGVPSRFSGSGSGTDFTLTISSLQPEDFASYYCQQANSFPFTFGPGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - DNA, 741 bp, synthetic construct
  source 1..741: mol_type = other DNA
cagattaccctgaaggaaagcgggcctacactggtgaagccaacccagacactgaccctgacatgcaccttcagcggcttttctctgagcacctccggagtgggagtgggatggatcaggcagccacctggcaaggcacctgagtggctggcccagatctactggaacgacgagaagcggtatagctcctctctgaagtctagactgacaatcaccaaggatacaagcaagaaccaggtggtgctgaagatgaccaatatggaccccgtggatacagccacctactattgtgcccaccggagaggcatcggcaattggttcgacccttggggccagggcacactggtgaccgtgagctccggaggaggaggatccggcggaggaggctctggcggcggcggctccggcggcggcggctccgacatccagatgacacagtccccatctagcgtgtctgccagcgtgggcgatagggtgacaatcacctgccgcgcctctcagggcatctcctcttggctggcctggtaccagcagaagccaggcaaggcccccaacctgctgatctatgcagcatccggactgcagtctggagtgccaagcagattctccggctctggcagcggcaccgactttacactgaccatcagctccctgcagcccgaggattttgcctcctactattgtcagcaggccaattcattcccattcaccttcggaccaggcacaaaagtggacatcaag

SEQ ID NO: 15 - DNA, 1473 bp, synthetic construct
  source 1..1473: mol_type = other DNA
atggctctgcctgtgactgccctgctgctgcccctggccctgctgctgcacgccgcacgacctcagattaccctgaaggaaagcgggcctacactggtgaagccaacccagacactgaccctgacatgcaccttcagcggcttttctctgagcacctccggagtgggagtgggatggatcaggcagccacctggcaaggcacctgagtggctggcccagatctactggaacgacgagaagcggtatagctcctctctgaagtctagactgacaatcaccaaggatacaagcaagaaccaggtggtgctgaagatgaccaatatggaccccgtggatacagccacctactattgtgcccaccggagaggcatcggcaattggttcgacccttggggccagggcacactggtgaccgtgagctccggaggaggaggatccggcggaggaggctctggcggcggcggctccggcggcggcggctccgacatccagatgacacagtccccatctagcgtgtctgccagcgtgggcgatagggtgacaatcacctgccgcgcctctcagggcatctcctcttggctggcctggtaccagcagaagccaggcaaggcccccaacctgctgatctatgcagcatccggactgcagtctggagtgccaagcagattctccggctctggcagcggcaccgactttacactgaccatcagctccctgcagcccgaggattttgcctcctactattgtcagcaggccaattcattcccattcaccttcggaccaggcacaaaagtggacatcaagacaactaccccagcacctaggccacctacacctgcaccaaccatcgccagccagcctctgtccctgagaccagaggcctgtaggccagcagcaggaggagcagtgcacacccggggcctggacttcgcctgcgatatctacatctgggcaccactggcaggaacatgtggcgtgctgctgctgtccctggtcatcaccctgtactgcaagagaggcaggaagaagctgctgtatatcttcaagcagcccttcatgagacccgtgcagacaacccaggaggaggacggctgcagctgtaggttcccagaggaggaggagggaggatgtgagctgcgcgtgaagttttcccggtctgccgatgcacctgcataccagcagggacagaaccagctgtataacgagctgaatctgggccggagagaggagtacgacgtgctggataagaggaggggaagggaccctgagatgggaggcaagcctcggagaaagaacccacaggagggcctgtacaatgagctgcagaaggacaagatggccgaggcctatagcgagatcggcatgaagggagagaggcgccggggcaagggacacgatggcctgtatcagggcctgtcaaccgctacaaaagatacctacgatgctctgcacatgcaggctctgccaccaaga

SEQ ID NO: 16 - protein, 7 aa, synthetic construct
TSGVGVG

SEQ ID NO: 17 - protein, 9 aa, synthetic construct
GFSLSTSGV

SEQ ID NO: 18 - protein, 12 aa, synthetic construct
GFSLSTSGVGVG

SEQ ID NO: 19 - protein, 16 aa, synthetic construct
QIYWNDEKRYSPSLRN

SEQ ID NO: 20 - protein, 5 aa, synthetic construct
YWNDE

SEQ ID NO: 21 - protein, 10 aa, synthetic construct
RRGVGNWFDP

SEQ ID NO: 22 - protein, 11 aa, synthetic construct
RASQGISSWLA

SEQ ID NO: 23 - protein, 7 aa, synthetic construct
AASSLQS

SEQ ID NO: 24 - protein, 9 aa, synthetic construct
QQANSFPFT

SEQ ID NO: 25 - protein, 120 aa, synthetic construct
QITLKESGPTLVKPTQTLTLTCTFSGFSLSTSGVGVGWIRQTPGKALEWLTQIYWNDEKRYSPSLRNRLTITKDTSKNQVVLTMTNMDPVDTATYYCAHRRGVGNWFDPWGQGILVTVSS

SEQ ID NO: 26 - protein, 107 aa, synthetic construct
DIQMTQSPSSVSASVGDRVTITCRASQGISSWLAWYQQKPGKAPKLLIYAASSLQSGVSSRFSGSESGTDFTLTISSLQPEDFATYYCQQANSFPFTFGPGTKVDIK

SEQ ID NO: 27 - protein, 247 aa, synthetic construct
QITLKESGPTLVKPTQTLTLTCTFSGFSLSTSGVGVGWIRQTPGKALEWLTQIYWNDEKRYSPSLRNRLTITKDTSKNQVVLTMTNMDPVDTATYYCAHRRGVGNWFDPWGQGILVTVSSGGGGSGGGGSGGGGSGGGGSDIQMTQSPSSVSASVGDRVTITCRASQGISSWLAWYQQKPGKAPKLLIYAASSLQSGVSSRFSGSESGTDFTLTISSLQPEDFATYYCQQANSFPFTFGPGTKVDIK

SEQ ID NO: 28 - protein, 491 aa, synthetic construct
MALPVTALLLPLALLLHAARPQITLKESGPTLVKPTQTLTLTCTFSGFSLSTSGVGVGWIRQTPGKALEWLTQIYWNDEKRYSPSLRNRLTITKDTSKNQVVLTMTNMDPVDTATYYCAHRRGVGNWFDPWGQGILVTVSSGGGGSGGGGSGGGGSGGGGSDIQMTQSPSSVSASVGDRVTITCRASQGISSWLAWYQQKPGKAPKLLIYAASSLQSGVSSRFSGSESGTDFTLTISSLQPEDFATYYCQQANSFPFTFGPGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - DNA, 741 bp, synthetic construct
  source 1..741: mol_type = other DNA
cagatcaccctgaaggaatccgggccaacactggtgaagcccacccagacactgaccctgacatgcaccttcagcggctttagcctgtccacctctggagtgggagtgggatggatcaggcagacacctggcaaggccctggagtggctgacccagatctactggaacgacgagaagcggtacagcccatccctgaggaatcgcctgacaatcaccaaggatacctccaagaaccaggtggtgctgacaatgaccaatatggaccccgtggatacagccacctactattgtgcccaccggagaggcgtgggcaactggttcgacccttggggccagggcatcctggtgacagtgagctccggaggaggaggatccggcggaggaggctctggcggcggcggctccggcggcggcggctccgacatccagatgacccagtctccatctagcgtgtctgccagcgtgggcgatcgggtgacaatcacctgcagagcctcccagggcatctcctcttggctggcctggtaccagcagaagcccggcaaggcccctaagctgctgatctatgcagcaagctccctgcagagcggcgtgtctagccggttctccggctctgagagcggcacagactttacactgaccatctcctctctgcagcccgaggattttgccacctactattgtcagcaggccaatagtttcccattcacttttggcccaggcactaaggtggacatcaag

SEQ ID NO: 30 - DNA, 1473 bp, synthetic construct
  source 1..1473: mol_type = other DNA
atggccctgcctgtcaccgcactgctgctgcccctggctctgctgctgcacgccgcaagacctcagatcaccctgaaggaatccgggccaacactggtgaagcccacccagacactgaccctgacatgcaccttcagcggctttagcctgtccacctctggagtgggagtgggatggatcaggcagacacctggcaaggccctggagtggctgacccagatctactggaacgacgagaagcggtacagcccatccctgaggaatcgcctgacaatcaccaaggatacctccaagaaccaggtggtgctgacaatgaccaatatggaccccgtggatacagccacctactattgtgcccaccggagaggcgtgggcaactggttcgacccttggggccagggcatcctggtgacagtgagctccggaggaggaggatccggcggaggaggctctggcggcggcggctccggcggcggcggctccgacatccagatgacccagtctccatctagcgtgtctgccagcgtgggcgatcgggtgacaatcacctgcagagcctcccagggcatctcctcttggctggcctggtaccagcagaagcccggcaaggcccctaagctgctgatctatgcagcaagctccctgcagagcggcgtgtctagccggttctccggctctgagagcggcacagactttacactgaccatctcctctctgcagcccgaggattttgccacctactattgtcagcaggccaatagtttcccattcacttttggcccaggcactaaggtggacatcaagactactactcccgctcctaggccacctacacctgcaccaaccatcgccagccagcctctgtccctgagaccagaggcctgtaggccagcagcaggaggagcagtgcacacccggggcctggacttcgcctgcgatatctacatctgggcaccactggcaggaacatgtggcgtgctgctgctgtccctggtcatcaccctgtactgcaagagaggcaggaagaagctgctgtatatcttcaagcagcccttcatgagacccgtgcagacaacccaggaggaggacggctgcagctgtaggttcccagaggaggaggagggaggatgtgagctgcgcgtgaagttttcccggtctgccgatgcacctgcataccagcagggacagaaccagctgtataacgagctgaatctgggccggagagaggagtacgacgtgctggataagaggaggggaagggaccctgagatgggaggcaagcctcggagaaagaacccacaggagggcctgtacaatgagctgcagaaggacaagatggccgaggcctatagcgagatcggcatgaagggagagaggcgccggggcaagggacacgatggcctgtatcagggcctgtcaaccgctacaaaagatacctacgatgctctgcacatgcaggctctgccaccaaga

SEQ ID NO: 31 - protein, 5 aa, synthetic construct
NYAMN

SEQ ID NO: 32 - protein, 7 aa, synthetic construct
GFTFSNY

SEQ ID NO: 33 - protein, 10 aa, synthetic construct
GFTFSNYAMN

SEQ ID NO: 34 - protein, 17 aa, synthetic construct
GISGSGGSTYDADSVKG

SEQ ID NO: 35 - protein, 6 aa, synthetic construct
SGSGGS

SEQ ID NO: 36 - protein, 10 aa, synthetic construct
QGYSFGYFES

SEQ ID NO: 37 - protein, 11 aa, synthetic construct
RASQNVNRYLA

SEQ ID NO: 38 - protein, 7 aa, synthetic construct
DAFNRAT

SEQ ID NO: 39 - protein, 9 aa, synthetic construct
QQRSDWPLT

SEQ ID NO: 40 - protein, 119 aa, synthetic construct
EVHLLESGGGLVQPWGSLTLSCAASGFTFSNYAMNWVRQAPGKGLEWVSGISGSGGSTYDADSVKGRFTISRDNSKNTLFLQMNSPRAEDTAVYYCATQGYSFGYFESWGQGTLVTVSS

SEQ ID NO: 41 - protein, 106 aa, synthetic construct
EIVLTQSPATLSLSPGERATLSCRASQNVNRYLAWYHQKPGQAPRLLIYDAFNRATGIPARFSGSGSGTDFTLTINSLEPEDFAVYYCQQRSDWPLTFGGGTKLEI

SEQ ID NO: 42 - protein, 246 aa, synthetic construct
EVHLLESGGGLVQPWGSLTLSCAASGFTFSNYAMNWVRQAPGKGLEWVSGISGSGGSTYDADSVKGRFTISRDNSKNTLFLQMNSPRAEDTAVYYCATQGYSFGYFESWGQGTLVTVSSGGGGSGGGGSGGGGSGGGGSEIVLTQSPATLSLSPGERATLSCRASQNVNRYLAWYHQKPGQAPRLLIYDAFNRATGIPARFSGSGSGTDFTLTINSLEPEDFAVYYCQQRSDWPLTFGGGTKLEIK

SEQ ID NO: 43 - protein, 490 aa, synthetic construct
MALPVTALLLPLALLLHAARPEVHLLESGGGLVQPWGSLTLSCAASGFTFSNYAMNWVRQAPGKGLEWVSGISGSGGSTYDADSVKGRFTISRDNSKNTLFLQMNSPRAEDTAVYYCATQGYSFGYFESWGQGTLVTVSSGGGGSGGGGSGGGGSGGGGSEIVLTQSPATLSLSPGERATLSCRASQNVNRYLAWYHQKPGQAPRLLIYDAFNRATGIPARFSGSGSGTDFTLTINSLEPEDFAVYYCQQRSDWPLTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - DNA, 738 bp, synthetic construct
  source 1..738: mol_type = other DNA
gaagtccacctgctggaatctgggggaggactggtgcagccatggggaagcctgaccctgtcctgcgccgcctctggcttcacattttctaactacgccatgaattgggtgcggcaggcacctggcaagggactggagtgggtgtccggaatctctggaagcggaggctctacctatgacgccgatagcgtgaagggccggttcaccatcagcagagacaactccaagaatacactgtttctgcagatgaacagccccagagccgaggataccgccgtgtactattgtgccacacagggctactccttcggctattttgagtcttggggccagggcaccctggtgacagtgagctccggaggaggaggatccggcggaggaggctctggcggcggcggctccggcggcggcggctccgagatcgtgctgacccagtccccagccacactgtccctgtctccaggagagagggccaccctgtcttgcagggccagccagaacgtgaataggtacctggcctggtatcaccagaagccaggacaggcacctcgcctgctgatctacgacgccttcaacagggcaaccggcatccctgccagattcagcggctccggctctggcacagactttaccctgacaatcaatagcctggagccagaggattttgccgtgtactattgtcagcagagatccgactggcccctgacttttggcggcggaactaaactggaaatcaag

SEQ ID NO: 45 - DNA, 1470 bp, synthetic construct
  source 1..1470: mol_type = other DNA
atggctctgcctgtcactgctctgctgctgcctctggctctgctgctgcacgctgctcgccctgaagtccacctgctggaatctgggggaggactggtgcagccatggggaagcctgaccctgtcctgcgccgcctctggcttcacattttctaactacgccatgaattgggtgcggcaggcacctggcaagggactggagtgggtgtccggaatctctggaagcggaggctctacctatgacgccgatagcgtgaagggccggttcaccatcagcagagacaactccaagaatacactgtttctgcagatgaacagccccagagccgaggataccgccgtgtactattgtgccacacagggctactccttcggctattttgagtcttggggccagggcaccctggtgacagtgagctccggaggaggaggatccggcggaggaggctctggcggcggcggctccggcggcggcggctccgagatcgtgctgacccagtccccagccacactgtccctgtctccaggagagagggccaccctgtcttgcagggccagccagaacgtgaataggtacctggcctggtatcaccagaagccaggacaggcacctcgcctgctgatctacgacgccttcaacagggcaaccggcatccctgccagattcagcggctccggctctggcacagactttaccctgacaatcaatagcctggagccagaggattttgccgtgtactattgtcagcagagatccgactggcccctgacttttggcggcggaactaaactggaaatcaagacaactacacctgctcctaggccacctacacctgcaccaaccatcgccagccagcctctgtccctgagaccagaggcctgtaggccagcagcaggaggagcagtgcacacccggggcctggacttcgcctgcgatatctacatctgggcaccactggcaggaacatgtggcgtgctgctgctgtccctggtcatcaccctgtactgcaagagaggcaggaagaagctgctgtatatcttcaagcagcccttcatgagacccgtgcagacaacccaggaggaggacggctgcagctgtaggttcccagaggaggaggagggaggatgtgagctgcgcgtgaagttttcccggtctgccgatgcacctgcataccagcagggacagaaccagctgtataacgagctgaatctgggccggagagaggagtacgacgtgctggataagaggaggggaagggaccctgagatgggaggcaagcctcggagaaagaacccacaggagggcctgtacaatgagctgcagaaggacaagatggccgaggcctatagcgagatcggcatgaagggagagaggcgccggggcaagggacacgatggcctgtatcagggcctgtcaaccgctacaaaagatacctacgatgctctgcacatgcaggctctgccaccaaga

SEQ ID NO: 46 - protein, 5 aa, synthetic construct
SYYWN

SEQ ID NO: 47 - protein, 7 aa, synthetic construct
AGSISSY

SEQ ID NO: 48 - protein, 10 aa, synthetic construct
AGSISSYYWN

SEQ ID NO: 49 - protein, 16 aa, synthetic construct
RIYTSGSTNYNPSLRS

SEQ ID NO: 50 - protein, 5 aa, synthetic construct
YTSGS

SEQ ID NO: 51 - protein, 11 aa, synthetic construct
ASYTYFDSFDI

SEQ ID NO: 52 - protein, 11 aa, synthetic construct
RASQDIRNFLA

SEQ ID NO: 53 - protein, 7 aa, synthetic construct
AASTLQS

SEQ ID NO: 54 - protein, 9 aa, synthetic construct
QQVNSYPRT

SEQ ID NO: 55 - protein, 119 aa, synthetic construct
QVQLQESGPGLVKPSETLSLTCTVSAGSISSYYWNWIRQPAGKGLEWIGRIYTSGSTNYNPSLRSRVTMSVDTSKNQFSLKLSSVTATDTAVYYCASASYTYFDSFDIWGQGTMVTVSS

SEQ ID NO: 56 - protein, 107 aa, synthetic construct
DIQLTQSPSFLSASVGDRVTITCRASQDIRNFLAWYQQKPGKAPKLLIYAASTLQSGVPSRFSGSGSGTEFALTVSSLQPEDFATYYCQQVNSYPRTFGQGTKVEIK

SEQ ID NO: 57 - protein, 246 aa, synthetic construct
QVQLQESGPGLVKPSETLSLTCTVSAGSISSYYWNWIRQPAGKGLEWIGRIYTSGSTNYNPSLRSRVTMSVDTSKNQFSLKLSSVTATDTAVYYCASASYTYFDSFDIWGQGTMVTVSSGGGGSGGGGSGGGGSGGGGSDIQLTQSPSFLSASVGDRVTITCRASQDIRNFLAWYQQKPGKAPKLLIYAASTLQSGVPSRFSGSGSGTEFALTVSSLQPEDFATYYCQQVNSYPRTFGQGTKVEIK

SEQ ID NO: 58 - protein, 490 aa, synthetic construct
MALPVTALLLPLALLLHAARPQVQLQESGPGLVKPSETLSLTCTVSAGSISSYYWNWIRQPAGKGLEWIGRIYTSGSTNYNPSLRSRVTMSVDTSKNQFSLKLSSVTATDTAVYYCASASYTYFDSFDIWGQGTMVTVSSGGGGSGGGGSGGGGSGGGGSDIQLTQSPSFLSASVGDRVTITCRASQDIRNFLAWYQQKPGKAPKLLIYAASTLQSGVPSRFSGSGSGTEFALTVSSLQPEDFATYYCQQVNSYPRTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - DNA, 738 bp, synthetic construct
  source 1..738: mol_type = other DNA
caggtccagctgcaggagtctgggccaggcctggtgaagccctctgagaccctgagcctgacctgcacagtgtccgccggctctatcagctcctactattggaactggatcagacagcctgcaggcaagggactggagtggatcggaaggatctacacatctggcagcaccaactataatccaagcctgcggtccagagtgacaatgtccgtggacacctctaagaatcagttcagcctgaagctgtctagcgtgaccgccacagataccgccgtgtactattgtgcctccgcctcttacacatatttcgactcctttgatatctggggccagggcacaatggtgaccgtgtcctctggaggaggaggaagcggaggaggaggaagcggcggcggcggctctggcggcggcggctccgacatccagctgacccagagcccatccttcctgtctgccagcgtgggcgacagggtgacaatcacctgccgcgccagccaggatatccggaactttctggcctggtaccagcagaagcccggcaaggcccctaagctgctgatctatgcagcaagcacactgcagtccggagtgccatctagattctccggctctggcagcggcacagagtttgccctgaccgtgagctccctgcagcctgaggattttgccacctactattgtcagcaggtgaattcatacccaagaacattcgggcaggggactaaagtggaaatcaag

SEQ ID NO: 60 - DNA, 1470 bp, synthetic construct
  source 1..1470: mol_type = other DNA
atggctctgcctgtcaccgctctgctgctgcctctggctctgctgctgcacgccgcccgccctcaggtccagctgcaggagtctgggccaggcctggtgaagccctctgagaccctgagcctgacctgcacagtgtccgccggctctatcagctcctactattggaactggatcagacagcctgcaggcaagggactggagtggatcggaaggatctacacatctggcagcaccaactataatccaagcctgcggtccagagtgacaatgtccgtggacacctctaagaatcagttcagcctgaagctgtctagcgtgaccgccacagataccgccgtgtactattgtgcctccgcctcttacacatatttcgactcctttgatatctggggccagggcacaatggtgaccgtgtcctctggaggaggaggaagcggaggaggaggaagcggcggcggcggctctggcggcggcggctccgacatccagctgacccagagcccatccttcctgtctgccagcgtgggcgacagggtgacaatcacctgccgcgccagccaggatatccggaactttctggcctggtaccagcagaagcccggcaaggcccctaagctgctgatctatgcagcaagcacactgcagtccggagtgccatctagattctccggctctggcagcggcacagagtttgccctgaccgtgagctccctgcagcctgaggattttgccacctactattgtcagcaggtgaattcatacccaagaacattcgggcaggggactaaagtggaaatcaagactactacaccagcccctaggccacctacacctgcaccaaccatcgccagccagcctctgtccctgagaccagaggcctgtaggccagcagcaggaggagcagtgcacacccggggcctggacttcgcctgcgatatctacatctgggcaccactggcaggaacatgtggcgtgctgctgctgtccctggtcatcaccctgtactgcaagagaggcaggaagaagctgctgtatatcttcaagcagcccttcatgagacccgtgcagacaacccaggaggaggacggctgcagctgtaggttcccagaggaggaggagggaggatgtgagctgcgcgtgaagttttcccggtctgccgatgcacctgcataccagcagggacagaaccagctgtataacgagctgaatctgggccggagagaggagtacgacgtgctggataagaggaggggaagggaccctgagatgggaggcaagcctcggagaaagaacccacaggagggcctgtacaatgagctgcagaaggacaagatggccgaggcctatagcgagatcggcatgaagggagagaggcgccggggcaagggacacgatggcctgtatcagggcctgtcaaccgctacaaaagatacctacgatgctctgcacatgcaggctctgccaccaaga

SEQ ID NO: 61 - protein, 5 aa, synthetic construct
RYWMS

SEQ ID NO: 62 - protein, 7 aa, synthetic construct
GFTFSRY

SEQ ID NO: 63 - protein, 10 aa, synthetic construct
GFTFSRYWMS

SEQ ID NO: 64 - protein, 17 aa, synthetic construct
NIKHDGSEKYYVDSVKG

SEQ ID NO: 65 - protein, 6 aa, synthetic construct
KHDGSE

SEQ ID NO: 66 - protein, 8 aa, synthetic construct
YYGGPFDY

SEQ ID NO: 67 - protein, 12 aa, synthetic construct
RASQSVRSSYLA

SEQ ID NO: 68 - protein, 7 aa, synthetic construct
GASSRAT

SEQ ID NO: 69 - protein, 8 aa, synthetic construct
QQFGSSLT

SEQ ID NO: 70 - protein, 117 aa, synthetic construct
EVQLVESGGGLVQPGGSLRLSCAASGFTFSRYWMSWVRQAPGKGLEWVANIKHDGSEKYYVDSVKGRFTFSRDNAKTSLYLQMNSLRVEDTALYYCARYYGGPFDYWGQGTLVTVSS

SEQ ID NO: 71 - protein, 107 aa, synthetic construct
EIVLTQSPGTLSLSPGERATLSCRASQSVRSSYLAWYQQKPGQAPRLLIFGASSRATGIPDRFSGSGSGTDFTLTISRLEPEDFAVYYCQQFGSSLTFGGGTKVEIK

SEQ ID NO: 72 - protein, 244 aa, synthetic construct
EVQLVESGGGLVQPGGSLRLSCAASGFTFSRYWMSWVRQAPGKGLEWVANIKHDGSEKYYVDSVKGRFTFSRDNAKTSLYLQMNSLRVEDTALYYCARYYGGPFDYWGQGTLVTVSSGGGGSGGGGSGGGGSGGGGSEIVLTQSPGTLSLSPGERATLSCRASQSVRSSYLAWYQQKPGQAPRLLIFGASSRATGIPDRFSGSGSGTDFTLTISRLEPEDFAVYYCQQFGSSLTFGGGTKVEIK

SEQ ID NO: 73 - protein, 488 aa, synthetic construct
MALPVTALLLPLALLLHAARPEVQLVESGGGLVQPGGSLRLSCAASGFTFSRYWMSWVRQAPGKGLEWVANIKHDGSEKYYVDSVKGRFTFSRDNAKTSLYLQMNSLRVEDTALYYCARYYGGPFDYWGQGTLVTVSSGGGGSGGGGSGGGGSGGGGSEIVLTQSPGTLSLSPGERATLSCRASQSVRSSYLAWYQQKPGQAPRLLIFGASSRATGIPDRFSGSGSGTDFTLTISRLEPEDFAVYYCQQFGSSLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - DNA, 732 bp, synthetic construct
  source 1..732: mol_type = other DNA
gaagtccagctggtcgaatctggcggaggactggtgcagccaggaggatccctgagactgtcttgcgccgccagcggcttcaccttttccagatactggatgtcttgggtgaggcaggcacctggcaagggactggagtgggtggccaacatcaagcacgacggctccgagaagtactatgtggattctgtgaagggccggttcacctttagcagagacaacgccaagacatccctgtacctgcagatgaacagcctgagagtggaggacacagccctgtactattgcgccaggtactatggcggccccttcgattattggggccagggcaccctggtgacagtgagctccggaggaggaggaagcggcggaggaggcagcggcggcggcggctctggcggcggcggcagcgagatcgtgctgacccagtccccaggcacactgagcctgtccccaggagagagggccaccctgagctgtcgcgcctctcagagcgtgcggtctagctacctggcctggtatcagcagaagccaggacaggcacctcgcctgctgatctttggagcatcctctagggcaaccggcatccctgaccggttctccggatctggaagcggcacagacttcaccctgacaatctcccggctggagccagaggatttcgccgtgtactattgtcagcagtttggctcatctctgaccttcggggggggcacaaaagtggaaatcaag

SEQ ID NO: 75 - DNA, 1464 bp, synthetic construct
  source 1..1464: mol_type = other DNA
atggctctgcctgtcaccgccctgctgctgcccctggccctgctgctgcacgccgctcgccctgaagtccagctggtcgaatctggcggaggactggtgcagccaggaggatccctgagactgtcttgcgccgccagcggcttcaccttttccagatactggatgtcttgggtgaggcaggcacctggcaagggactggagtgggtggccaacatcaagcacgacggctccgagaagtactatgtggattctgtgaagggccggttcacctttagcagagacaacgccaagacatccctgtacctgcagatgaacagcctgagagtggaggacacagccctgtactattgcgccaggtactatggcggccccttcgattattggggccagggcaccctggtgacagtgagctccggaggaggaggaagcggcggaggaggcagcggcggcggcggctctggcggcggcggcagcgagatcgtgctgacccagtccccaggcacactgagcctgtccccaggagagagggccaccctgagctgtcgcgcctctcagagcgtgcggtctagctacctggcctggtatcagcagaagccaggacaggcacctcgcctgctgatctttggagcatcctctagggcaaccggcatccctgaccggttctccggatctggaagcggcacagacttcaccctgacaatctcccggctggagccagaggatttcgccgtgtactattgtcagcagtttggctcatctctgaccttcggggggggcacaaaagtggaaatcaagacaacaactcctgctcctaggccacctacacctgcaccaaccatcgccagccagcctctgtccctgagaccagaggcctgtaggccagcagcaggaggagcagtgcacacccggggcctggacttcgcctgcgatatctacatctgggcaccactggcaggaacatgtggcgtgctgctgctgtccctggtcatcaccctgtactgcaagagaggcaggaagaagctgctgtatatcttcaagcagcccttcatgagacccgtgcagacaacccaggaggaggacggctgcagctgtaggttcccagaggaggaggagggaggatgtgagctgcgcgtgaagttttcccggtctgccgatgcacctgcataccagcagggacagaaccagctgtataacgagctgaatctgggccggagagaggagtacgacgtgctggataagaggaggggaagggaccctgagatgggaggcaagcctcggagaaagaacccacaggagggcctgtacaatgagctgcagaaggacaagatggccgaggcctatagcgagatcggcatgaagggagagaggcgccggggcaagggacacgatggcctgtatcagggcctgtcaaccgctacaaaagatacctacgatgctctgcacatgcaggctctgccaccaaga

SEQ ID NO: 76 - protein, 5 aa, synthetic construct
SYAMS

SEQ ID NO: 77 - protein, 7 aa, synthetic construct
GFTFSSY

SEQ ID NO: 78 - protein, 10 aa, synthetic construct
GFTFSSYAMS

SEQ ID NO: 79 - protein, 17 aa, synthetic construct
AISGSGGSTHYADSVKG

SEQ ID NO: 80 - protein, 6 aa, synthetic construct
SGSGGS

SEQ ID NO: 81 - protein, 13 aa, synthetic construct
EGYVGSWYAPFDY

SEQ ID NO: 82 - protein, 12 aa, synthetic construct
TLSSGHSSYAIA

SEQ ID NO: 83 - protein, 11 aa, synthetic construct
LNSGGSHSKGD

SEQ ID NO: 84 - protein, 9 aa, synthetic construct
QTWDTGIRV

SEQ ID NO: 85 - protein, 122 aa, synthetic construct
EVQLLESGGGLEQPGGSLRLSCAASGFTFSSYAMSWVRQAPGKGLEWVSAISGSGGSTHYADSVKGRFTISRDNARNTLYLQMNSLRAEDTAVYYCAKEGYVGSWYAPFDYWGQGTLVTVSS

SEQ ID NO: 86 - protein, 111 aa, synthetic construct
QLVLTQSPSASASLGASVKLTCTLSSGHSSYAIAWHQQQPEKGPRYLMKLNSGGSHSKGDGIPDRFSGSSSGAERYLTISSLQSEDEADYYCQTWDTGIRVFGGGTKLTVL

SEQ ID NO: 87 - protein, 497 aa, synthetic construct
MALPVTALLLPLALLLHAARPEVQLLESGGGLEQPGGSLRLSCAASGFTFSSYAMSWVRQAPGKGLEWVSAISGSGGSTHYADSVKGRFTISRDNARNTLYLQMNSLRAEDTAVYYCAKEGYVGSWYAPFDYWGQGTLVTVSSGGGGSGGGGSGGGGSGGGGSQLVLTQSPSASASLGASVKLTCTLSSGHSSYAIAWHQQQPEKGPRYLMKLNSGGSHSKGDGIPDRFSGSSSGAERYLTISSLQSEDEADYYCQTWDTGIRVFGGGTKLTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - DNA, 1491 bp, synthetic construct
  source 1..1491: mol_type = other DNA
atggctctgcctgtgactgccctgctgctgcccctggccctgctgctgcacgccgcacgacctgaagtccagctgctggagtcaggaggaggactggagcagccaggcggaagcctgaggctgtcctgcgcagcatctggcttcacctttagctcctatgcaatgagctgggtgagacaggcccccggcaagggactggagtgggtgtccgccatctccggatctggaggatccacacactatgccgactctgtgaagggcaggttcaccatctctcgggataacgccagaaatacactgtacctgcagatgaacagcctgagggcagaggacaccgccgtgtactattgcgccaaggagggctacgtgggcagctggtatgccccttttgattactggggccagggcaccctggtgacagtgtctagcggaggaggaggaagcggaggaggaggatctggcggcggcggctctggcggcggcggcagccagctggtgctgacacagagcccatccgcctctgccagcctgggcgcatccgtgaagctgacctgtacactgtcctctggccacagctcctatgcaatcgcatggcaccagcagcagccagagaagggacctcggtacctgatgaagctgaacagcggaggatcccactctaagggcgacggcatccccgataggttctctggatctagctccggagcagagcggtacctgaccatctctagcctgcagagcgaggacgaggccgattactattgtcagacatgggacactgggattcgggtcttcggcgggggaacaaaactgactgtcctgacaactaccccagcacctaggccacctacacctgcaccaaccatcgccagccagcctctgtccctgagaccagaggcctgtaggccagcagcaggaggagcagtgcacacccggggcctggacttcgcctgcgatatctacatctgggcaccactggcaggaacatgtggcgtgctgctgctgtccctggtcatcaccctgtactgcaagagaggcaggaagaagctgctgtatatcttcaagcagcccttcatgagacccgtgcagacaacccaggaggaggacggctgcagctgtaggttcccagaggaggaggagggaggatgtgagctgcgcgtgaagttttcccggtctgccgatgcacctgcataccagcagggacagaaccagctgtataacgagctgaatctgggccggagagaggagtacgacgtgctggataagaggaggggaagggaccctgagatgggaggcaagcctcggagaaagaacccacaggagggcctgtacaatgagctgcagaaggacaagatggccgaggcctatagcgagatcggcatgaagggagagaggcgccggggcaagggacacgatggcctgtatcagggcctgtcaaccgctacaaaagatacctacgatgctctgcacatgcaggctctgccaccaaga

SEQ ID NO: 89 - protein, 7 aa, synthetic construct
TSGMCVS

SEQ ID NO: 90 - protein, 9 aa, synthetic construct
GVSLSTSGM

SEQ ID NO: 91 - protein, 12 aa, synthetic construct
GVSLSTSGMCVS

SEQ ID NO: 92 - protein, 16 aa, synthetic construct
FIDWDDDKYYNTSLKT

SEQ ID NO: 93 - protein, 5 aa, synthetic construct
DWDDD

SEQ ID NO: 94 - protein, 13 aa, synthetic construct
IRGYSGSYDAFDI

SEQ ID NO: 95 - protein, 11 aa, synthetic construct
RASQGISNYLA

SEQ ID NO: 96 - protein, 7 aa, synthetic construct
AASTLQS

SEQ ID NO: 97 - protein, 9 aa, synthetic construct
QKYISAPFT

SEQ ID NO: 98 - protein, 123 aa, synthetic construct
QVTLRESGPALVKPTQTLTLTCTVSGVSLSTSGMCVSWIRQPLGKALEWLGFIDWDDDKYYNTSLKTRLTISKDTSKNQVVLTMTNMDPVDTATYYCARIRGYSGSYDAFDIWGQGTVVIVSS

SEQ ID NO: 99 - protein, 107 aa, synthetic construct
DIQMTQSPSSLSASVGDRVTITCRASQGISNYLAWYQQKPGRVPKLLIYAASTLQSGVPSRFSGSGSGTDFTLTISSLQPEDVATYYCQKYISAPFTFGPGTKVDIK

SEQ ID NO: 100 - protein, 494 aa, synthetic construct
MALPVTALLLPLALLLHAARPQVTLRESGPALVKPTQTLTLTCTVSGVSLSTSGMCVSWIRQPLGKALEWLGFIDWDDDKYYNTSLKTRLTISKDTSKNQVVLTMTNMDPVDTATYYCARIRGYSGSYDAFDIWGQGTVVIVSSGGGGSGGGGSGGGGSGGGGSDIQMTQSPSSLSASVGDRVTITCRASQGISNYLAWYQQKPGRVPKLLIYAASTLQSGVPSRFSGSGSGTDFTLTISSLQPEDVATYYCQKYISAPFTFGPGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - DNA, 1482 bp, synthetic construct
  source 1..1482: mol_type = other DNA
atggctctgcctgtgactgccctgctgctgcccctggccctgctgctgcacgccgcacgacctcaggtcactctgagggaatctggcccagccctggtgaagcccacccagacactgaccctgacatgcaccgtgtccggcgtgagcctgtccacctctggcatgtgcgtgagctggatcaggcagcccctgggcaaggccctggagtggctgggcttcatcgattgggacgatgacaagtactataacacatccctgaagacaagactgaccatctccaaggacacctctaagaaccaggtggtgctgacaatgaccaatatggatcctgtggacacagccacctactattgcgcccggatcagaggctacagcggctcctatgatgcctttgacatctggggccagggcaccgtggtcatcgtgagctccggcggcggcggctctggaggaggaggaagcggaggaggaggaagcgggggcggcggctctgatatccagatgacacagagcccatctagcctgtctgccagcgtgggcgacagggtgacaatcacctgccgcgccagccagggcatctccaattacctggcctggtatcagcagaagcccggccgggtgcctaagctgctgatctacgcagcatctacactgcagagcggagtgccttccagattctccggatctggaagcggaaccgacttcaccctgaccatctcctctctgcagccagaggacgtggccacatactattgtcagaagtatatctccgcaccattcacatttggacctggaactaaagtggacatcaagacaactaccccagcacctaggccacctacacctgcaccaaccatcgccagccagcctctgtccctgagaccagaggcctgtaggccagcagcaggaggagcagtgcacacccggggcctggacttcgcctgcgatatctacatctgggcaccactggcaggaacatgtggcgtgctgctgctgtccctggtcatcaccctgtactgcaagagaggcaggaagaagctgctgtatatcttcaagcagcccttcatgagacccgtgcagacaacccaggaggaggacggctgcagctgtaggttcccagaggaggaggagggaggatgtgagctgcgcgtgaagttttcccggtctgccgatgcacctgcataccagcagggacagaaccagctgtataacgagctgaatctgggccggagagaggagtacgacgtgctggataagaggaggggaagggaccctgagatgggaggcaagcctcggagaaagaacccacaggagggcctgtacaatgagctgcagaaggacaagatggccgaggcctatagcgagatcggcatgaagggagagaggcgccggggcaagggacacgatggcctgtatcagggcctgtcaaccgctacaaaagatacctacgatgctctgcacatgcaggctctgccaccaaga

SEQ ID NO: 102 - protein, 7 aa, synthetic construct
TSGVGVG

SEQ ID NO: 103 - protein, 9 aa, synthetic construct
GFSLSTSGV

SEQ ID NO: 104 - protein, 12 aa, synthetic construct
GFSLSTSGVGVG

SEQ ID NO: 105 - protein, 16 aa, synthetic construct
QIYWNDEKRYSPSLKN

SEQ ID NO: 106 - protein, 5 aa, synthetic construct
YWNDE

SEQ ID NO: 107 - protein, 10 aa, synthetic construct
RRGVGNWFDP

SEQ ID NO: 108 - protein, 11 aa, synthetic construct
RASQGISSWLA

SEQ ID NO: 109 - protein, 7 aa, synthetic construct
AASSLQS

SEQ ID NO: 110 - protein, 9 aa, synthetic construct
QQAFSFPFT

SEQ ID NO: 111 - protein, 120 aa, synthetic construct
QVTLKESGLTLMKPTQTHTLTCTFSGFSLSTSGVGVGWIRQTPGKALEWLTQIYWNDEKRYSPSLKNRLTITKDTSKNQVVLTMTNMDPVDTATYYCAHRRGVGNWFDPWGQGTLVTVSS

SEQ ID NO: 112 - protein, 107 aa, synthetic construct
DIQMTQSPSSVSASVGDRVTITCRASQGISSWLAWYQQKPGKAPKLLIYAASSLQSGVSSSFSGSASGTEFTLTISNLQPEDFAIYYCQQAFSFPFTFGPGTKVDIK

SEQ ID NO: 113 - protein, 491 aa, synthetic construct
MALPVTALLLPLALLLHAARPQVTLKESGLTLMKPTQTHTLTCTFSGFSLSTSGVGVGWIRQTPGKALEWLTQIYWNDEKRYSPSLKNRLTITKDTSKNQVVLTMTNMDPVDTATYYCAHRRGVGNWFDPWGQGTLVTVSSGGGGSGGGGSGGGGSGGGGSDIQMTQSPSSVSASVGDRVTITCRASQGISSWLAWYQQKPGKAPKLLIYAASSLQSGVSSSFSGSASGTEFTLTISNLQPEDFAIYYCQQAFSFPFTFGPGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 114 - DNA, 1473 bp, synthetic construct
  source 1..1473: mol_type = other DNA
atggctctgcctgtgactgccctgctgctgcccctggccctgctgctgcacgccgcacgacctcaggtgaccctgaaggagtccggcctgacactgatgaagcccacacagacccacacactgacctgcacattctctggcttttctctgagcacctccggagtgggagtgggatggatcagacagacccccggcaaggccctggagtggctgacacagatctactggaacgacgagaagcggtattctcctagcctgaagaatagactgaccatcacaaaggatacatccaagaaccaggtggtgctgaccatgacaaatatggacccagtggataccgccacatactattgtgcccaccggagaggagtgggaaactggttcgacccatggggacagggcaccctggtgacagtgagcagcggaggaggaggcagcggaggaggaggctccggcggcggcggctctggaggaggaggcagcgacatccagatgacccagtccccttctagcgtgtccgcctctgtgggcgatagggtgaccatcacatgcagggcaagccagggaatctcctcttggctggcctggtaccagcagaagccaggcaaggcccccaagctgctgatctatgcagcaagctccctgcagagcggcgtgtctagctccttcagcggctccgcctctggaaccgagtttaccctgacaatctctaatctgcagcctgaggactttgccatctactattgtcagcaggccttcagctttccattcacctttggccccggcacaaaggtggatatcaagacaactaccccagcacctaggccacctacacctgcaccaaccatcgccagccagcctctgtccctgagaccagaggcctgtaggccagcagcaggaggagcagtgcacacccggggcctggacttcgcctgcgatatctacatctgggcaccactggcaggaacatgtggcgtgctgctgctgtccctggtcatcaccctgtactgcaagagaggcaggaagaagctgctgtatatcttcaagcagcccttcatgagacccgtgcagacaacccaggaggaggacggctgcagctgtaggttcccagaggaggaggagggaggatgtgagctgcgcgtgaagttttcccggtctgccgatgcacctgcataccagcagggacagaaccagctgtataacgagctgaatctgggccggagagaggagtacgacgtgctggataagaggaggggaagggaccctgagatgggaggcaagcctcggagaaagaacccacaggagggcctgtacaatgagctgcagaaggacaagatggccgaggcctatagcgagatcggcatgaagggagagaggcgccggggcaagggacacgatggcctgtatcagggcctgtcaaccgctacaaaagatacctacgatgctctgcacatgcaggctctgccaccaaga

SEQ ID NO: 115 - protein, 5 aa, synthetic construct
SYAMS

SEQ ID NO: 116 - protein, 7 aa, synthetic construct
GFTFSSY

SEQ ID NO: 117 - protein, 10 aa, synthetic construct
GFTFSSYAMS

SEQ ID NO: 118 - protein, 17 aa, synthetic construct
AISGSGGSTYYADSVKG

SEQ ID NO: 119 - protein, 6 aa, synthetic construct
SGSGGS

SEQ ID NO: 120 - protein, 7 aa, synthetic construct
DLGATDY

SEQ ID NO: 121 - protein, 11 aa, synthetic construct
RASQGISSWLA

SEQ ID NO: 122 - protein, 7 aa, synthetic construct
AASSLQS

SEQ ID NO: 123 - protein, 9 aa, synthetic construct
QQANSFPLT

SEQ ID NO: 124 - protein, 116 aa, synthetic construct
QVTLKESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLGATDYWGQGTLVTVSS

SEQ ID NO: 125 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIK

SEQ ID NO: 126 - protein, 487 aa, synthetic construct
MALPVTALLLPLALLLHAARPQVTLKESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLGATDYWGQGTLVTVSSGGGGSGGGGSGGGGSGGGGSDIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - DNA, 1461 bp, synthetic construct
  source 1..1461: mol_type = other DNA
atggctctgcctgtgactgccctgctgctgcccctggccctgctgctgcacgccgcacgacctcaggtgaccctgaaggagagcggaggaggcctggtgcagcctggcggctccctgaggctgtcttgcgcagcaagcggcttcaccttcagctcctacgccatgtcctgggtgagacaggcccctggcaagggcctggagtgggtgtctgccatcagcggctccggaggctctacctactatgccgacagcgtgaagggccggttcacaatctccagagataactctaagaataccctgtacctgcagatgaactccctgcgcgccgaggacacagccgtgtactattgcgccaaggacctgggcgccaccgattattggggccagggcacactggtgaccgtgtctagcggcggcggcggctctggaggaggaggcagcggcggaggaggctccggcggcggcggctctgacatccagatgacccagagcccatccagcgtgagcgccagcgtgggcgatagggtgacaatcacctgtagggcatcccagggaatcagctcctggctggcctggtaccagcagaagccaggcaaggcccccaagctgctgatctatgcagcatctagcctgcagagcggagtgccatcccggttctccggctctggcagcggaacagactttacactgaccatctcctctctgcagcctgaggattttgccacctactattgtcagcaggccaatagcttcccactgacatttggcggcggcaccaaggtggagatcaagacaactaccccagcacctaggccacctacacctgcaccaaccatcgccagccagcctctgtccctgagaccagaggcctgtaggccagcagcaggaggagcagtgcacacccggggcctggacttcgcctgcgatatctacatctgggcaccactggcaggaacatgtggcgtgctgctgctgtccctggtcatcaccctgtactgcaagagaggcaggaagaagctgctgtatatcttcaagcagcccttcatgagacccgtgcagacaacccaggaggaggacggctgcagctgtaggttcccagaggaggaggagggaggatgtgagctgcgcgtgaagttttcccggtctgccgatgcacctgcataccagcagggacagaaccagctgtataacgagctgaatctgggccggagagaggagtacgacgtgctggataagaggaggggaagggaccctgagatgggaggcaagcctcggagaaagaacccacaggagggcctgtacaatgagctgcagaaggacaagatggccgaggcctatagcgagatcggcatgaagggagagaggcgccggggcaagggacacgatggcctgtatcagggcctgtcaaccgctacaaaagatacctacgatgctctgcacatgcaggctctgccaccaaga

SEQ ID NO: 128 - protein, 529 aa, synthetic construct
MALPVTALLLPLALLLHAARPGGGGSCPYSNPSLCGGGGSQITLKESGPTLVKPTQTLTLTCTFSGFSLSTSGVGVGWIRQPPGKAPEWLAQIYWNDEKRYSSSLKSRLTITKDTSKNQVVLKMTNMDPVDTATYYCAHRRGIGNWFDPWGQGTLVTVSSGGGGSGGGGSGGGGSGGGGSDIQMTQSPSSVSASVGDRVTITCRASQGISSWLAWYQQKPGKAPNLLIYAASGLQSGVPSRFSGSGSGTDFTLTISSLQPEDFASYYCQQANSFPFTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - DNA, 1587 bp, synthetic construct
  source 1..1587: mol_type = other DNA
atggccctgcccgtcactgctctgctgctgcctctggctctgctgctgcacgccgcccgccctggggggggaggatcttgtccatattccaacccatctctgtgcggaggaggaggatcccagatcacactgaaggagtctggccctaccctggtgaagccaacccagacactgaccctgacatgtacctttagcggcttctctctgagcacctccggagtgggagtgggatggatcaggcagccacctggcaaggcacctgagtggctggcccagatctactggaacgacgagaagcggtatagctcctctctgaagtctagactgacaatcaccaaggatacatccaagaaccaggtggtgctgaagatgaccaatatggacccagtggatacagccacctactattgcgcccaccggagaggcatcggcaattggtttgacccatggggacagggcacactggtgaccgtgagctccggaggaggaggaagcggcggaggaggcagcggcggcggcggctctggcggcggcggcagcgacatccagatgacacagagcccatctagcgtgtctgccagcgtgggcgatagggtgacaatcacctgcagggcatcccagggaatctcctcttggctggcctggtaccagcagaagccaggcaaggcccccaacctgctgatctatgcagcaagcggactgcagtccggagtgccttctagattttccggctctggcagcggcaccgacttcacactgaccatcagctccctgcagccagaggatttcgccagctactattgtcagcaggccaattccttcccctttacattcggccctggcaccaaggtggatatcaagggggggggcggaagttgtccatactcaaatccaagcctgtgcggcggaggcggctctactaccactccagcacctaggccacctacacctgcaccaaccatcgccagccagcctctgtccctgagaccagaggcctgtaggccagcagcaggaggagcagtgcacacccggggcctggacttcgcctgcgatatctacatctgggcaccactggcaggaacatgtggcgtgctgctgctgtccctggtcatcaccctgtactgcaagagaggcaggaagaagctgctgtatatcttcaagcagcccttcatgagacccgtgcagacaacccaggaggaggacggctgcagctgtaggttcccagaggaggaggagggaggatgtgagctgcgcgtgaagttttcccggtctgccgatgcacctgcataccagcagggacagaaccagctgtataacgagctgaatctgggccggagagaggagtacgacgtgctggataagaggaggggaagggaccctgagatgggaggcaagcctcggagaaagaacccacaggagggcctgtacaatgagctgcagaaggacaagatggccgaggcctatagcgagatcggcatgaagggagagaggcgccggggcaagggacacgatggcctgtatcagggcctgtcaaccgctacaaaagatacctacgatgctctgcacatgcaggctctgccaccaaga

SEQ ID NO: 130 - protein, 524 aa, synthetic construct
MALPVTALLLPLALLLHAARPGGGGSCPYSNPSLCGGGGSCPYSNPSLCGGGGSQITLKESGPTLVKPTQTLTLTCTFSGFSLSTSGVGVGWIRQTPGKALEWLTQIYWNDEKRYSPSLRNRLTITKDTSKNQVVLTMTNMDPVDTATYYCAHRRGVGNWFDPWGQGILVTVSSGGGGSGGGGSGGGGSGGGGSDIQMTQSPSSVSASVGDRVTITCRASQGISSWLAWYQQKPGKAPKLLIYAASSLQSGVSSRFSGSESGTDFTLTISSLQPEDFATYYCQQANSFPFTFGPGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - DNA, 1572 bp, synthetic construct
  source 1..1572: mol_type = other DNA
atggctctgcccgtcactgctctgctgctgcccctggctctgctgctgcacgccgcaagacccggaggaggaggaagctgtccctattccaacccatctctgtgcggcggcggaggaagctgtccctacagcaatccttccctgtgcggaggaggaggaagccagatcacactgaaggagtccggccctaccctggtgaagccaacccagacactgaccctgacatgtaccttctccggctttagcctgtccacctctggagtgggagtgggatggatcaggcagacaccaggcaaggccctggagtggctgacccagatctactggaacgacgagaagcggtacagcccttccctgaggaatcgcctgacaatcaccaaggataccagcaagaaccaggtggtgctgacaatgaccaatatggacccagtggatacagccacctactattgcgcacacaggagaggagtgggaaactggttcgacccatggggacagggcatcctggtgacagtgagctccggcggcggcggctctggaggaggaggaagcggaggaggaggaagcgggggcggcggctctgacatccagatgacccagtctccttctagcgtgtctgccagcgtgggcgatcgggtgacaatcacctgcagagcctcccagggcatctcctcttggctggcctggtaccagcagaagccaggcaaggcccccaagctgctgatctatgcagcaagctccctgcagagcggcgtgtctagccggttctccggctctgagagcggcacagactttacactgaccatctcctctctgcagccagaggattttgccacctactattgtcagcaggccaattccttccccttcacattcggacctggcacaaaagtggacatcaagactactacccccgcccctaggccacctacacctgcaccaaccatcgccagccagcctctgtccctgagaccagaggcctgtaggccagcagcaggaggagcagtgcacacccggggcctggacttcgcctgcgatatctacatctgggcaccactggcaggaacatgtggcgtgctgctgctgtccctggtcatcaccctgtactgcaagagaggcaggaagaagctgctgtatatcttcaagcagcccttcatgagacccgtgcagacaacccaggaggaggacggctgcagctgtaggttcccagaggaggaggagggaggatgtgagctgcgcgtgaagttttcccggtctgccgatgcacctgcataccagcagggacagaaccagctgtataacgagctgaatctgggccggagagaggagtacgacgtgctggataagaggaggggaagggaccctgagatgggaggcaagcctcggagaaagaacccacaggagggcctgtacaatgagctgcagaaggacaagatggccgaggcctatagcgagatcggcatgaagggagagaggcgccggggcaagggacacgatggcctgtatcagggcctgtcaaccgctacaaaagatacctacgatgctctgcacatgcaggctctgccaccaaga

SEQ ID NO: 132 - protein, 527 aa, synthetic construct
MALPVTALLLPLALLLHAARPQVQLQESGPGLVKPSETLSLTCTVSAGSISSYYWNWIRQPAGKGLEWIGRIYTSGSTNYNPSLRSRVTMSVDTSKNQFSLKLSSVTATDTAVYYCASASYTYFDSFDIWGQGTMVTVSSGGGGSGGGGSGGGGSGGGGSDIQLTQSPSFLSASVGDRVTITCRASQDIRNFLAWYQQKPGKAPKLLIYAASTLQSGVPSRFSGSGSGTEFALTVSSLQPEDFATYYCQQVNSYPRTFGQGTKVEIKGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - DNA, 1581 bp, synthetic construct
  source 1..1581: mol_type = other DNA
atggctctgcctgtcaccgctctgctgctgcctctggctctgctgctgcacgctgctcgcccccaggtccagctgcaggaatccgggcctggcctggtgaagccatctgagaccctgagcctgacctgcacagtgtccgccggctctatcagctcctactattggaactggatcagacagccagcaggcaagggactggagtggatcggaaggatctacacatctggcagcaccaactataatcccagcctgcggtccagagtgacaatgtccgtggacacctctaagaatcagttctccctgaagctgtctagcgtgaccgccacagataccgccgtgtactattgtgcctccgcctcttacacatatttcgacagctttgatatctggggccagggcacaatggtgaccgtgtcctctggaggaggaggaagcggaggaggaggaagcggcggcggcggctctggcggcggcggcagcgacatccagctgacccagagcccttccttcctgtctgccagcgtgggcgacagggtgacaatcacctgccgcgccagccaggatatccggaactttctggcctggtaccagcagaagcccggcaaggcccctaagctgctgatctatgcagcaagcacactgcagtccggagtgccatctcgcttctccggatctggaagcggcacagagtttgcactgaccgtgagctccctgcagccagaggatttcgccacctactattgtcagcaggtgaactcctaccctcggacatttggccagggcaccaaggtggagatcaagggatccggaggaggaggatcttgcccatattctaatcccagcctgtgctccggcggcggcggcagctgtccatactccaatccatcactgtgcagcggggggggggggtcaaccactacaccagcacctaggccacctacacctgcaccaaccatcgccagccagcctctgtccctgagaccagaggcctgtaggccagcagcaggaggagcagtgcacacccggggcctggacttcgcctgcgatatctacatctgggcaccactggcaggaacatgtggcgtgctgctgctgtccctggtcatcaccctgtactgcaagagaggcaggaagaagctgctgtatatcttcaagcagcccttcatgagacccgtgcagacaacccaggaggaggacggctgcagctgtaggttcccagaggaggaggagggaggatgtgagctgcgcgtgaagttttcccggtctgccgatgcacctgcataccagcagggacagaaccagctgtataacgagctgaatctgggccggagagaggagtacgacgtgctggataagaggaggggaagggaccctgagatgggaggcaagcctcggagaaagaacccacaggagggcctgtacaatgagctgcagaaggacaagatggccgaggcctatagcgagatcggcatgaagggagagaggcgccggggcaagggacacgatggcctgtatcagggcctgtcaaccgctacaaaagatacctacgatgctctgcacatgcaggctctgccaccaaga

SEQ ID NO: 134 - protein, 21 aa, synthetic construct
MALPVTALLLPLALLLHAARP

SEQ ID NO: 135 - protein, 20 aa, synthetic construct
GGGGSGGGGSGGGGSGGGGS

SEQ ID NO: 136 - protein, 66 aa, synthetic construct
TTTPAPRPPTPAPTIASQPLSLRPEACRPAAGGAVHTRGLDFACDIYIWAPLAGTCGVLLLSLVIT

SEQ ID NO: 137 - protein, 42 aa, synthetic construct
KRGRKKLLYIFKQPFMRPVQTTQEEDGCSCRFPEEEEGGCEL

SEQ ID NO: 138 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 113 aa, synthetic construct
LRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 9 aa, synthetic construct
CPYSNPSLC

SEQ ID NO: 141 - protein, 24 aa, synthetic construct
NSELLSLINDMPITNDQKKLMSNN

SEQ ID NO: 142 - protein, 12 aa, synthetic construct
CQFDLSTRRLKC

SEQ ID NO: 143 - protein, 12 aa, synthetic construct
CQYNLSSRALKC

SEQ ID NO: 144 - protein, 12 aa, synthetic construct
CVWQRWQKSYVC

SEQ ID NO: 145 - protein, 12 aa, synthetic construct
CMWDRFSRWYKC

SEQ ID NO: 146 - protein, 25 aa, synthetic construct
SFVLNWYRMSPSNQTDKLAAFPEDR

SEQ ID NO: 147 - protein, 19 aa, synthetic construct
SGTYLCGAISLAPKAQIKE

SEQ ID NO: 148 - protein, 24 aa, synthetic construct
ELPTQGTFSNVSTNVSPAKPTTTA

SEQ ID NO: 149 - protein, 16 aa, synthetic construct
ELPTQGTFSNVSTNVS

SEQ ID NO: 150 - protein, 12 aa, synthetic construct
GQNDTSQTSSPS

SEQ ID NO: 151 - DNA, 45 bp, synthetic construct
  source 1..45: mol_type = other DNA
ggcggtggaggctccggaggggggggctctggcggagggggctcc

SEQ ID NO: 152 - DNA, 60 bp, synthetic construct
  source 1..60: mol_type = other DNA
ggcggcggcggctctggaggaggaggcagcggcggaggaggctccggaggcggcggctct

SEQ ID NO: 153 - protein, 45 aa, synthetic construct
TTTPAPRPPTPAPTIASQPLSLRPEACRPAAGGAVHTRGLDFACD

SEQ ID NO: 154 - protein, 21 aa, synthetic construct
IYIWAPLAGTCGVLLLSLVIT

SEQ ID NO: 155 - protein, 24 aa, synthetic construct
IYIWAPLAGTCGVLLLSLVITLYC

SEQ ID NO: 156 - protein, 39 aa, synthetic construct
IEVMYPPPYLDNEKSNGTIIHVKGKHLCPSPLFPGPSKP

SEQ ID NO: 157 - protein, 27 aa, synthetic construct
FWVLVVVGGVLACYSLLVTVAFIIFWV

SEQ ID NO: 158 - protein, 41 aa, synthetic construct
RSKRSRGGHSDYMNMTPRRPGPTRKHYQPYAPPRDFAAYRS

SEQ ID NO: 159 - protein, 41 aa, synthetic construct
RSKRSRLLHSDYMFMTPRRPGPTRKHYQPYAPPRDFAAYRS

SEQ ID NO: 160 - protein, 15 aa, synthetic construct
GGGGSGGGGSGGGSG

SEQ ID NO: 161 - protein, 14 aa, synthetic construct
GGGSGGGGSGGGGS

SEQ ID NO: 162 - protein, 15 aa, synthetic construct
GGGGSGGGGSGGGGS

SEQ ID NO: 163 - protein, 5 aa, synthetic construct
GGGGS

SEQ ID NO: 164 - protein, 18 aa, synthetic construct
GSTSGSGKPGSGEGSTKG

SEQ ID NO: 165 - DNA, 54 bp, synthetic construct
  source 1..54: mol_type = other DNA
gggtctacatccggctccgggaagcccggaagtggcgaaggtagtacaaagggg

SEQ ID NO: 166 - DNA, 63 bp, synthetic construct
  source 1..63: mol_type = other DNA
atggcactccccgtaactgctctgctgctgccgttggcattgctcctgcacgccgcacgcccg

SEQ ID NO: 167 - protein, 136 aa, synthetic construct
CPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 168 - protein, 157 aa, synthetic construct
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 169 - protein, 21 aa, synthetic construct
MGTSLLCWMALCLLGADHADA

SEQ ID NO: 170 - protein, 16 aa, synthetic construct
GLAVSTISSFFPPGYQ

SEQ ID NO: 171 - protein, 231 aa, synthetic construct
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - DNA, 126 bp, synthetic construct
  source 1..126: mol_type = other DNA
aagcgcggcaggaagaagctcctctacatttttaagcagccttttatgaggcccgtacagacaacacaggaggaagatggctgtagctgcagatttcccgaggaggaggaaggtgggtgcgagctg

SEQ ID NO: 173 - DNA, 123 bp, synthetic construct
  source 1..123: mol_type = other DNA
agatccaaaagaagccgcctgctccatagcgattacatgaatatgactccacgccgccctggccccacaaggaaacactaccagccttacgcaccacctagagatttcgctgcctatcggagc

SEQ ID NO: 174 - protein, 41 aa, synthetic construct
RSKRSRLLHSDYMNMTPRRPGPTRKHYQPYAPPRDFAAYRS

SEQ ID NO: 175 - DNA, 336 bp, synthetic construct
  source 1..336: mol_type = other DNA
agggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 176 - protein, 37 aa, synthetic construct
GSGGGGSCPYSNPSLCSGGGGSCPYSNPSLCSGGGGS

SEQ ID NO: 177 - protein, 5 aa, synthetic construct
SGGGG

SEQ ID NO: 178 - protein, 6 aa, synthetic construct
SGGGGS

SEQ ID NO: 179 - protein, 470 aa, synthetic construct
QITLKESGPTLVKPTQTLTLTCTFSGFSLSTSGVGVGWIRQPPGKAPEWLAQIYWNDEKRYSSSLKSRLTITKDTSKNQVVLKMTNMDPVDTATYYCAHRRGIGNWFDPWGQGTLVTVSSGGGGSGGGGSGGGGSGGGGSDIQMTQSPSSVSASVGDRVTITCRASQGISSWLAWYQQKPGKAPNLLIYAASGLQSGVPSRFSGSGSGTDFTLTISSLQPEDFASYYCQQANSFPFTFGPGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 180 - protein, 470 aa, synthetic construct
QITLKESGPTLVKPTQTLTLTCTFSGFSLSTSGVGVGWIRQTPGKALEWLTQIYWNDEKRYSPSLRNRLTITKDTSKNQVVLTMTNMDPVDTATYYCAHRRGVGNWFDPWGQGILVTVSSGGGGSGGGGSGGGGSGGGGSDIQMTQSPSSVSASVGDRVTITCRASQGISSWLAWYQQKPGKAPKLLIYAASSLQSGVSSRFSGSESGTDFTLTISSLQPEDFATYYCQQANSFPFTFGPGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 181 - protein, 469 aa, synthetic construct
EVHLLESGGGLVQPWGSLTLSCAASGFTFSNYAMNWVRQAPGKGLEWVSGISGSGGSTYDADSVKGRFTISRDNSKNTLFLQMNSPRAEDTAVYYCATQGYSFGYFESWGQGTLVTVSSGGGGSGGGGSGGGGSGGGGSEIVLTQSPATLSLSPGERATLSCRASQNVNRYLAWYHQKPGQAPRLLIYDAFNRATGIPARFSGSGSGTDFTLTINSLEPEDFAVYYCQQRSDWPLTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 182 - protein, 469 aa, synthetic construct
QVQLQESGPGLVKPSETLSLTCTVSAGSISSYYWNWIRQPAGKGLEWIGRIYTSGSTNYNPSLRSRVTMSVDTSKNQFSLKLSSVTATDTAVYYCASASYTYFDSFDIWGQGTMVTVSSGGGGSGGGGSGGGGSGGGGSDIQLTQSPSFLSASVGDRVTITCRASQDIRNFLAWYQQKPGKAPKLLIYAASTLQSGVPSRFSGSGSGTEFALTVSSLQPEDFATYYCQQVNSYPRTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 183 - protein, 467 aa, synthetic construct
EVQLVESGGGLVQPGGSLRLSCAASGFTFSRYWMSWVRQAPGKGLEWVANIKHDGSEKYYVDSVKGRFTFSRDNAKTSLYLQMNSLRVEDTALYYCARYYGGPFDYWGQGTLVTVSSGGGGSGGGGSGGGGSGGGGSEIVLTQSPGTLSLSPGERATLSCRASQSVRSSYLAWYQQKPGQAPRLLIFGASSRATGIPDRFSGSGSGTDFTLTISRLEPEDFAVYYCQQFGSSLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 184 - protein, 508 aa, synthetic construct
GGGGSCPYSNPSLCGGGGSQITLKESGPTLVKPTQTLTLTCTFSGFSLSTSGVGVGWIRQPPGKAPEWLAQIYWNDEKRYSSSLKSRLTITKDTSKNQVVLKMTNMDPVDTATYYCAHRRGIGNWFDPWGQGTLVTVSSGGGGSGGGGSGGGGSGGGGSDIQMTQSPSSVSASVGDRVTITCRASQGISSWLAWYQQKPGKAPNLLIYAASGLQSGVPSRFSGSGSGTDFTLTISSLQPEDFASYYCQQANSFPFTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 185 - protein, 503 aa, synthetic construct
GGGGSCPYSNPSLCGGGGSCPYSNPSLCGGGGSQITLKESGPTLVKPTQTLTLTCTFSGFSLSTSGVGVGWIRQTPGKALEWLTQIYWNDEKRYSPSLRNRLTITKDTSKNQVVLTMTNMDPVDTATYYCAHRRGVGNWFDPWGQGILVTVSSGGGGSGGGGSGGGGSGGGGSDIQMTQSPSSVSASVGDRVTITCRASQGISSWLAWYQQKPGKAPKLLIYAASSLQSGVSSRFSGSESGTDFTLTISSLQPEDFATYYCQQANSFPFTFGPGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 186 - protein, 506 aa, synthetic construct
QVQLQESGPGLVKPSETLSLTCTVSAGSISSYYWNWIRQPAGKGLEWIGRIYTSGSTNYNPSLRSRVTMSVDTSKNQFSLKLSSVTATDTAVYYCASASYTYFDSFDIWGQGTMVTVSSGGGGSGGGGSGGGGSGGGGSDIQLTQSPSFLSASVGDRVTITCRASQDIRNFLAWYQQKPGKAPKLLIYAASTLQSGVPSRFSGSGSGTEFALTVSSLQPEDFATYYCQQVNSYPRTFGQGTKVEIKGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 187 - protein, 253 aa, synthetic construct
EVQLLESGGGLEQPGGSLRLSCAASGFTFSSYAMSWVRQAPGKGLEWVSAISGSGGSTHYADSVKGRFTISRDNARNTLYLQMNSLRAEDTAVYYCAKEGYVGSWYAPFDYWGQGTLVTVSSGGGGSGGGGSGGGGSGGGGSQLVLTQSPSASASLGASVKLTCTLSSGHSSYAIAWHQQQPEKGPRYLMKLNSGGSHSKGDGIPDRFSGSSSGAERYLTISSLQSEDEADYYCQTWDTGIRVFGGGTKLTVL

SEQ ID NO: 188 - DNA, 759 bp, synthetic construct
  source 1..759: mol_type = other DNA
gaagtccagctgctggagtcaggaggaggactggagcagccaggcggaagcctgaggctgtcctgcgcagcatctggcttcacctttagctcctatgcaatgagctgggtgagacaggcccccggcaagggactggagtgggtgtccgccatctccggatctggaggatccacacactatgccgactctgtgaagggcaggttcaccatctctcgggataacgccagaaatacactgtacctgcagatgaacagcctgagggcagaggacaccgccgtgtactattgcgccaaggagggctacgtgggcagctggtatgccccttttgattactggggccagggcaccctggtgacagtgtctagcggaggaggaggaagcggaggaggaggatctggcggcggcggctctggcggcggcggcagccagctggtgctgacacagagcccatccgcctctgccagcctgggcgcatccgtgaagctgacctgtacactgtcctctggccacagctcctatgcaatcgcatggcaccagcagcagccagagaagggacctcggtacctgatgaagctgaacagcggaggatcccactctaagggcgacggcatccccgataggttctctggatctagctccggagcagagcggtacctgaccatctctagcctgcagagcgaggacgaggccgattactattgtcagacatgggacactgggattcgggtcttcggcgggggaacaaaactgactgtcctg

SEQ ID NO: 189 - protein, 250 aa, synthetic construct
QVTLRESGPALVKPTQTLTLTCTVSGVSLSTSGMCVSWIRQPLGKALEWLGFIDWDDDKYYNTSLKTRLTISKDTSKNQVVLTMTNMDPVDTATYYCARIRGYSGSYDAFDIWGQGTVVIVSSGGGGSGGGGSGGGGSGGGGSDIQMTQSPSSLSASVGDRVTITCRASQGISNYLAWYQQKPGRVPKLLIYAASTLQSGVPSRFSGSGSGTDFTLTISSLQPEDVATYYCQKYISAPFTFGPGTKVDIK

SEQ ID NO: 190 - DNA, 750 bp, synthetic construct
  source 1..750: mol_type = other DNA
caggtcactctgagggaatctggcccagccctggtgaagcccacccagacactgaccctgacatgcaccgtgtccggcgtgagcctgtccacctctggcatgtgcgtgagctggatcaggcagcccctgggcaaggccctggagtggctgggcttcatcgattgggacgatgacaagtactataacacatccctgaagacaagactgaccatctccaaggacacctctaagaaccaggtggtgctgacaatgaccaatatggatcctgtggacacagccacctactattgcgcccggatcagaggctacagcggctcctatgatgcctttgacatctggggccagggcaccgtggtcatcgtgagctccggcggcggcggctctggaggaggaggaagcggaggaggaggaagcgggggcggcggctctgatatccagatgacacagagcccatctagcctgtctgccagcgtgggcgacagggtgacaatcacctgccgcgccagccagggcatctccaattacctggcctggtatcagcagaagcccggccgggtgcctaagctgctgatctacgcagcatctacactgcagagcggagtgccttccagattctccggatctggaagcggaaccgacttcaccctgaccatctcctctctgcagccagaggacgtggccacatactattgtcagaagtatatctccgcaccattcacatttggacctggaactaaagtggacatcaag

SEQ ID NO: 191 - protein, 247 aa, synthetic construct
QVTLKESGLTLMKPTQTHTLTCTFSGFSLSTSGVGVGWIRQTPGKALEWLTQIYWNDEKRYSPSLKNRLTITKDTSKNQVVLTMTNMDPVDTATYYCAHRRGVGNWFDPWGQGTLVTVSSGGGGSGGGGSGGGGSGGGGSDIQMTQSPSSVSASVGDRVTITCRASQGISSWLAWYQQKPGKAPKLLIYAASSLQSGVSSSFSGSASGTEFTLTISNLQPEDFAIYYCQQAFSFPFTFGPGTKVDIK

SEQ ID NO: 192 - DNA, 741 bp, synthetic construct
  source 1..741: mol_type = other DNA
caggtgaccctgaaggagtccggcctgacactgatgaagcccacacagacccacacactgacctgcacattctctggcttttctctgagcacctccggagtgggagtgggatggatcagacagacccccggcaaggccctggagtggctgacacagatctactggaacgacgagaagcggtattctcctagcctgaagaatagactgaccatcacaaaggatacatccaagaaccaggtggtgctgaccatgacaaatatggacccagtggataccgccacatactattgtgcccaccggagaggagtgggaaactggttcgacccatggggacagggcaccctggtgacagtgagcagcggaggaggaggcagcggaggaggaggctccggcggcggcggctctggaggaggaggcagcgacatccagatgacccagtccccttctagcgtgtccgcctctgtgggcgatagggtgaccatcacatgcagggcaagccagggaatctcctcttggctggcctggtaccagcagaagccaggcaaggcccccaagctgctgatctatgcagcaagctccctgcagagcggcgtgtctagctccttcagcggctccgcctctggaaccgagtttaccctgacaatctctaatctgcagcctgaggactttgccatctactattgtcagcaggccttcagctttccattcacctttggccccggcacaaaggtggatatcaag

SEQ ID NO: 193 - protein, 243 aa, synthetic construct
QVTLKESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLGATDYWGQGTLVTVSSGGGGSGGGGSGGGGSGGGGSDIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIK

SEQ ID NO: 194 - DNA, 729 bp, synthetic construct
  source 1..729: mol_type = other DNA
caggtgaccctgaaggagagcggaggaggcctggtgcagcctggcggctccctgaggctgtcttgcgcagcaagcggcttcaccttcagctcctacgccatgtcctgggtgagacaggcccctggcaagggcctggagtgggtgtctgccatcagcggctccggaggctctacctactatgccgacagcgtgaagggccggttcacaatctccagagataactctaagaataccctgtacctgcagatgaactccctgcgcgccgaggacacagccgtgtactattgcgccaaggacctgggcgccaccgattattggggccagggcacactggtgaccgtgtctagcggcggcggcggctctggaggaggaggcagcggcggaggaggctccggcggcggcggctctgacatccagatgacccagagcccatccagcgtgagcgccagcgtgggcgatagggtgacaatcacctgtagggcatcccagggaatcagctcctggctggcctggtaccagcagaagccaggcaaggcccccaagctgctgatctatgcagcatctagcctgcagagcggagtgccatcccggttctccggctctggcagcggaacagactttacactgaccatctcctctctgcagcctgaggattttgccacctactattgtcagcaggccaatagcttcccactgacatttggcggcggcaccaaggtggagatcaag

SEQ ID NO: 195 - protein, 476 aa, synthetic construct
EVQLLESGGGLEQPGGSLRLSCAASGFTFSSYAMSWVRQAPGKGLEWVSAISGSGGSTHYADSVKGRFTISRDNARNTLYLQMNSLRAEDTAVYYCAKEGYVGSWYAPFDYWGQGTLVTVSSGGGGSGGGGSGGGGSGGGGSQLVLTQSPSASASLGASVKLTCTLSSGHSSYAIAWHQQQPEKGPRYLMKLNSGGSHSKGDGIPDRFSGSSSGAERYLTISSLQSEDEADYYCQTWDTGIRVFGGGTKLTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 196 - protein, 473 aa, synthetic construct
QVTLRESGPALVKPTQTLTLTCTVSGVSLSTSGMCVSWIRQPLGKALEWLGFIDWDDDKYYNTSLKTRLTISKDTSKNQVVLTMTNMDPVDTATYYCARIRGYSGSYDAFDIWGQGTVVIVSSGGGGSGGGGSGGGGSGGGGSDIQMTQSPSSLSASVGDRVTITCRASQGISNYLAWYQQKPGRVPKLLIYAASTLQSGVPSRFSGSGSGTDFTLTISSLQPEDVATYYCQKYISAPFTFGPGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 197 - protein, 470 aa, synthetic construct
QVTLKESGLTLMKPTQTHTLTCTFSGFSLSTSGVGVGWIRQTPGKALEWLTQIYWNDEKRYSPSLKNRLTITKDTSKNQVVLTMTNMDPVDTATYYCAHRRGVGNWFDPWGQGTLVTVSSGGGGSGGGGSGGGGSGGGGSDIQMTQSPSSVSASVGDRVTITCRASQGISSWLAWYQQKPGKAPKLLIYAASSLQSGVSSSFSGSASGTEFTLTISNLQPEDFAIYYCQQAFSFPFTFGPGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 198 - protein, 466 aa, synthetic construct
QVTLKESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLGATDYWGQGTLVTVSSGGGGSGGGGSGGGGSGGGGSDIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 199 - protein, 40 aa, synthetic construct
KIEVMYPPPYLDNEKSNGTIIHVKGKHLCPSPLFPGPSKP

SEQ ID NO: 200 - protein, 467 aa, synthetic construct
QITLKESGPTLVKPTQTLTLTCTFSGFSLSTSGVGVGWIRQPPGKAPEWLAQIYWNDEKRYSSSLKSRLTITKDTSKNQVVLKMTNMDPVDTATYYCAHRRGIGNWFDPWGQGTLVTVSSGGGGSGGGGSGGGGSGGGGSDIQMTQSPSSVSASVGDRVTITCRASQGISSWLAWYQQKPGKAPNLLIYAASGLQSGVPSRFSGSGSGTDFTLTISSLQPEDFASYYCQQANSFPFTFGPGTKVDIKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 201 - protein, 467 aa, synthetic construct
QITLKESGPTLVKPTQTLTLTCTFSGFSLSTSGVGVGWIRQTPGKALEWLTQIYWNDEKRYSPSLRNRLTITKDTSKNQVVLTMTNMDPVDTATYYCAHRRGVGNWFDPWGQGILVTVSSGGGGSGGGGSGGGGSGGGGSDIQMTQSPSSVSASVGDRVTITCRASQGISSWLAWYQQKPGKAPKLLIYAASSLQSGVSSRFSGSESGTDFTLTISSLQPEDFATYYCQQANSFPFTFGPGTKVDIKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 202 - protein, 55 aa, synthetic construct
KNRKAKAKPVTRGAGAGGRQRGQNKERPPPVPNPDYEPIRKGQRDLYSGLNQRRI

SEQ ID NO: 203 - protein, 45 aa, synthetic construct
GQDGVRQSRASDKQTLLPNDQLYQPLKDREDDQYSHLQGNQLRRN

SEQ ID NO: 204 - protein, 245 aa, synthetic construct
QVTLKESGPVLVKPTETLTLTCTVSGFSLSNARMGVTWIRQPPGKALEWLAHIFSNDEKSYSTSLKSRLTISKDTSKTQVVLTMTNMDPVDTATYYCARIRDYYDISSYYDYWGQGTLVSVSSGGGGSGGGGSGGGGSDIQMTQSPSAMSASVGDRVTITCRASQDISNYLAWFQQKPGKVPKRLIYAASSLQSGVPSRFSGSGSGTEFTLTISSLLPEDFATYYCLQLNSFPFTFGGGTKVEIN

SEQ ID NO: 205 - protein, 107 aa, synthetic construct
DIQMTQSPSAMSASVGDRVTITCRASQDISNYLAWFQQKPGKVPKRLIYAASSLQSGVPSRFSGSGSGTEFTLTISSLLPEDFATYYCLQLNSFPFTFGGGTKVEIN

SEQ ID NO: 206 - protein, 123 aa, synthetic construct
QVTLKESGPVLVKPTETLTLTCTVSGFSLSNARMGVTWIRQPPGKALEWLAHIFSNDEKSYSTSLKSRLTISKDTSKTQVVLTMTNMDPVDTATYYCARIRDYYDISSYYDYWGQGTLVSVSS

SEQ ID NO: 207 - protein, 447 aa, synthetic construct
MALPVTALLLPLALLLHAARPQVTLKESGPVLVKPTETLTLTCTVSGFSLSNARMGVTWIRQPPGKALEWLAHIFSNDEKSYSTSLKSRLTISKDTSKTQVVLTMTNMDPVDTATYYCARIRDYYDISSYYDYWGQGTLVSVSSGGGGSGGGGSGGGGSDIQMTQSPSAMSASVGDRVTITCRASQDISNYLAWFQQKPGKVPKRLIYAASSLQSGVPSRFSGSGSGTEFTLTISSLLPEDFATYYCLQLNSFPFTFGGGTKVEINTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 208 - protein, 505 aa, synthetic construct
GGGGSCPYSNPSLCGGGGSQITLKESGPTLVKPTQTLTLTCTFSGFSLSTSGVGVGWIRQPPGKAPEWLAQIYWNDEKRYSSSLKSRLTITKDTSKNQVVLKMTNMDPVDTATYYCAHRRGIGNWFDPWGQGTLVTVSSGGGGSGGGGSGGGGSGGGGSDIQMTQSPSSVSASVGDRVTITCRASQGISSWLAWYQQKPGKAPNLLIYAASGLQSGVPSRFSGSGSGTDFTLTISSLQPEDFASYYCQQANSFPFTFGPGTKVDIKGGGGSCPYSNPSLCGGGGSIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 209 - protein, 500 aa, synthetic construct
GGGGSCPYSNPSLCGGGGSCPYSNPSLCGGGGSQITLKESGPTLVKPTQTLTLTCTFSGFSLSTSGVGVGWIRQTPGKALEWLTQIYWNDEKRYSPSLRNRLTITKDTSKNQVVLTMTNMDPVDTATYYCAHRRGVGNWFDPWGQGILVTVSSGGGGSGGGGSGGGGSGGGGSDIQMTQSPSSVSASVGDRVTITCRASQGISSWLAWYQQKPGKAPKLLIYAASSLQSGVSSRFSGSESGTDFTLTISSLQPEDFATYYCQQANSFPFTFGPGTKVDIKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 210 - protein, 778 aa, synthetic construct
GGGGSCPYSNPSLCGGGGSDIQMTQSPSAMSASVGDRVTITCRASQDISNYLAWFQQKPGKVPKRLIYAASSLQSGVPSRFSGSGSGTEFTLTISSLLPEDFATYYCLQLNSFPFTFGGGTKVEINGGGGSGGGGSGGGGSGGGGSQVTLKESGPVLVKPTETLTLTCTVSGFSLSNARMGVTWIRQPPGKALEWLAHIFSNDEKSYSTSLKSRLTISKDTSKTQVVLTMTNMDPVDTATYYCARIRDYYDISSYYDYWGQGTLVSVSSGGGGSGGGGSGGGGSGGGGSQITLKESGPTLVKPTQTLTLTCTFSGFSLSTSGVGVGWIRQPPGKAPEWLAQIYWNDEKRYSSSLKSRLTITKDTSKNQVVLKMTNMDPVDTATYYCAHRRGIGNWFDPWGQGTLVTVSSGGGGSGGGGSGGGGSGGGGSDIQMTQSPSSVSASVGDRVTITCRASQGISSWLAWYQQKPGKAPNLLIYAASGLQSGVPSRFSGSGSGTDFTLTISSLQPEDFASYYCQQANSFPFTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 211 - protein, 773 aa, synthetic construct
GGGGSCPYSNPSLCGGGGSCPYSNPSLCGGGGSDIQMTQSPSAMSASVGDRVTITCRASQDISNYLAWFQQKPGKVPKRLIYAASSLQSGVPSRFSGSGSGTEFTLTISSLLPEDFATYYCLQLNSFPFTFGGGTKVEINGGGGSGGGGSGGGGSGGGGSQVTLKESGPVLVKPTETLTLTCTVSGFSLSNARMGVTWIRQPPGKALEWLAHIFSNDEKSYSTSLKSRLTISKDTSKTQVVLTMTNMDPVDTATYYCARIRDYYDISSYYDYWGQGTLVSVSSGGGGSGGGGSGGGGSGGGGSQITLKESGPTLVKPTQTLTLTCTFSGFSLSTSGVGVGWIRQTPGKALEWLTQIYWNDEKRYSPSLRNRLTITKDTSKNQVVLTMTNMDPVDTATYYCAHRRGVGNWFDPWGQGILVTVSSGGGGSGGGGSGGGGSGGGGSDIQMTQSPSSVSASVGDRVTITCRASQGISSWLAWYQQKPGKAPKLLIYAASSLQSGVSSRFSGSESGTDFTLTISSLQPEDFATYYCQQANSFPFTFGPGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 212 - protein, 187 aa, synthetic construct
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQ

SEQ ID NO: 213 - protein, 187 aa, synthetic construct
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQ

SEQ ID NO: 214 - protein, 177 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 215 - protein, 25 aa, synthetic construct
  VARIANT 1..25: note = This sequence may encompass 1-5 GGGGS repeating units
GGGGSGGGGSGGGGSGGGGSGGGGS

SEQ ID NO: 216 - protein, 4 aa, synthetic construct
YMFM

SEQ ID NO: 217 - protein, 4 aa, synthetic construct
YMNM

(19) United States
(12) Patent Application Publication
(10) Pub. No.: US 2024/0216430 A1
(43) Pub. Date: Jul. 4, 2024

(54) CLAUDIN 18.2 TARGETING CHIMERIC ANTIGEN RECEPTORS AND BINDING AGENTS AND USES THEREOF

(71) Applicants: ALLOGENE THERAPEUTICS, INC., South San Francisco, CA (US); PFIZER INC., New York, NY (US)

(72) Inventors: Zhe LI, Burlingame, CA (US); Siler PANOWSKI, Berkeley, CA (US); Barbra Johnson SASU, San Francisco, CA (US); Bryan A. SMITH, San Francisco, CA (US); Thomas John VAN BLARCOM, Oakland, CA (US); Tao SAI, Foster City, CA (US); Guoyun ZHU, San Ramon, CA (US)

(21) Appl. No.: 18/521,196

(22) Filed: Nov. 28, 2023

Related U.S. Application Data
(60) Provisional application No. 63/590,719, filed on Oct. 16, 2023.
(60) Provisional application No. 63/428,308, filed on Nov. 28, 2022.

(51) Int. Cl. A61K 35/17; A61K 39/00; C07K 16/28

(52) U.S. Cl. CPC A61K 35/17; A61K 39/4611; A61K 39/4631; A61K 39/464438; C07K 16/2875; A61K 2039/505; A61K 2239/13; A61K 2239/17; A61K 2239/21; A61K 2239/22; A61K 2239/23; A61K 2239/29; A61K 2239/31; C07K 2317/31; C07K 2317/565; C07K 2317/622

(57) ABSTRACT

Provided herein are Claudin 18.2 binding agents and chimeric antigen receptors (CARs) comprising a Claudin 18.2 binding molecule that specifically binds to Claudin 18.2; and immune cells comprising these Claudin 18.2-specific CARs, e.g., CAR-T cells. Also provided are methods of making and using Claudin 18.2-specific CARs and Claudin 18.2 binding agents, and immune cells comprising Claudin 18.2-specific CARs.

### CROSS REFERENCE TO RELATED APPLICATIONS

The present application claims the benefit of priority to U.S. Provisional Application No. 63/428,308, filed on Nov. 28, 2022, and U.S. Provisional Application No. 63/590,719, filed on Oct. 16, 2023, the contents of both of which are hereby incorporated by reference in their entireties for all purposes.

### REFERENCE TO SEQUENCE LISTING

The instant application contains a Sequence Listing which has been submitted electronically in XML file format and is hereby incorporated by reference in its entirety. Said XML copy, created on Nov. 14, 2023, is named AT-057-03US_SL.xml and is 236,349 bytes in size.

### FIELD

This disclosure relates to Claudin 18.2 binding agents and chimeric antigen receptors (CARs) comprising an antigen binding molecule which binds to Claudin 18.2, polynucleotides encoding the same, and methods of treating a cancer in a patient using the same.

### BACKGROUND

A need exists to develop more targeted and potent therapies for proliferative disorders generally and specifically for gastric cancer, gastroesophageal junction (GEJ) cancer and pancreatic cancer.

Adoptive transfer of immune cells genetically modified to recognize malignancy-associated antigens shows promise as a new approach to treating cancer (see, e.g., Brenner et al., Current Opinion in Immunology, 22(2): 251-257 (2010); Rosenberg et al., Nature Reviews Cancer, 8(4): 299-308 (2008)). Immune cells can be genetically modified to express chimeric antigen receptors (CARs), fusion proteins comprised of a Claudin 18.2 antigen recognition moiety and T cell activation domains (see, e.g., Eshhar et al., Proc. Natd. Acad. Sci. USA, 90(2): 720-724 (1993), and Sadelain et al., Curr. Opin. Immunol, 21(2): 215-223 (2009)). Immune cells that contain CARs, e.g., CAR-T cells (“CAR-Ts”), are engineered to endow them with antigen specificity while retaining or enhancing their ability to recognize and kill a target cell.

Claudin 18.2, a splice variant of Claudin 18, is a tight junction molecule involved in the regulation of permeability, barrier function, and polarity of epithelial cells. The expression of Claudin 18.2 is strictly confined to the tight junctions of gastric mucosal cells, thereby making it inaccessible to targeted therapeutics. Besides the limited normal tissue expression, Claudin18.2 is highly expressed in different types of primary and metastatic cancers, including gastric, esophageal, pancreatic, lung, and ovarian cancers (Sahin et al., Clinical Cancer Research 14.23 (2008): 7624-7634). The malignant transformation in cancer cells leads to the exposure of claudin18.2 epitopes, making it an ideal target for targeted therapy. There is a need for treatments for cancer and in particular malignancies involving expression of Claudin 18.2, e.g. gastric cancer, gastroesophageal junction (GEJ) cancer and pancreatic cancer. Provided herein are methods and compositions addressing this need.

### SUMMARY

Provided herein are chimeric antigen receptors (CARs) comprising a Claudin 18.2 antigen binding domain that specifically binds to Claudin 18.2; and immune cells comprising these Claudin 18.2-specific CARs, e.g., CAR-T cells. Also provided are methods of making and using these Claudin 18.2-specific CARs, and immune cells comprising these Claudin 18.2-specific CARs. The Claudin 18.2 targeting CAR T cells described herein demonstrate good transduction efficiency, in vitro phenotype and potent in vitro and in vivo anti-tumor activity. Also provided herein are anti-Claudin 18.2 binding agents e.g. antibodies that bind to Claudin 18.2, as well as methods of making and methods of using the same. Anti-Claudin 18.2 binding agents provided herein bind human Claudin 18.2.

In one aspect, the present disclosure provides a chimeric antigen receptor (“CAR”) comprising an extracellular domain, a transmembrane domain, and an intracellular domain, wherein the extracellular domain comprises a Claudin 18.2 antigen binding domain (e.g. scFv) that specifically binds to Claudin 18.2, and wherein the antigen binding domain comprises at least one of: (a) a variable heavy chain CDR1 comprising an amino acid sequence selected from the group consisting of SEQ ID NOs 1-3, 16-18, 31-33, 46-48, 61-63, 76-78, 89-91, 102-104, 115, 116 and 117; (b) a variable heavy chain CDR2 comprising an amino acid sequence selected from the group consisting of SEQ ID NOs: 4-5, 19-20, 34-35, 49-50, 64-65, 79-80, 92-93, 105-106, 118 and 119; (c) a variable heavy chain CDR3 comprising an amino acid sequence selected from the group consisting of SEQ ID NOs 6, 21, 36, 51, 66, 81, 94, 107 and 120; (d) a variable light chain CDR1 comprising an amino acid sequence selected from the group consisting of SEQ ID NOs: 7, 22, 37, 52, 67, 82, 95, 108 and 121; (e) a variable light chain CDR2 comprising an amino acid sequence selected from the group consisting of SEQ ID NOs: 8, 23, 38, 53, 68, 83, 96, 109 and 122; and (f) a variable light chain CDR3 comprising an amino acid sequence selected from the group consisting of SEQ ID NOs: 9, 24, 39, 54, 69, 84, 97, 110 and 123.

In some embodiments of the CAR disclosed herein, the CAR comprises an extracellular domain, a transmembrane domain, and an intracellular domain, wherein the extracellular domain comprises a Claudin 18.2 antigen binding domain that specifically binds to Claudin 18.2, and wherein the antigen binding domain comprises: (a) a variable heavy chain CDR1 comprising an amino acid sequence selected from the group consisting of SEQ ID NOs: 1-3, 16-18, 31-33, 46-48, 61-63, 76-78, 89-91, 102-104, 115, 116 and 117; (b) a variable heavy chain CDR2 comprising an amino acid sequence selected from the group consisting of SEQ ID NOs: 4-5, 19-20, 34-35, 49-50, 64-65, 79-80, 92-93, 105-106, 118 and 119; and (c) a variable heavy chain CDR3 comprising an amino acid sequence selected from the group consisting of SEQ ID NOs: 6, 21, 36, 51, 66, 81, 94, 107 and 120.

In some embodiments of the CAR disclosed herein, the CAR comprises an extracellular domain, a transmembrane domain, and an intracellular domain, wherein the extracellular domain comprises a Claudin 18.2 antigen binding domain that specifically binds to Claudin 18.2, and wherein the antigen binding domain comprises: (a) a variable light chain CDR1 comprising an amino acid sequence selected from the group consisting of SEQ ID NOs: 7, 22, 37, 52, 67, 82, 95, 108 and 121; (b) a variable light chain CDR2 comprising an amino acid sequence selected from the group consisting of SEQ ID NOs: 8, 23, 38, 53, 68, 83, 96, 109 and 122; and (c) a variable light chain CDR3 comprising an amino acid sequence selected from the group consisting of SEQ ID NOs: 9, 24, 39, 54, 69, 84, 97, 110 and 123.

In some embodiments of the CAR disclosed herein, the CAR comprises an extracellular domain, a transmembrane domain, and an intracellular domain, wherein the extracellular domain comprises a Claudin 18.2 antigen binding domain that specifically binds to Claudin 18.2, and wherein the antigen binding domain comprises at least one of: (a) a variable heavy chain comprising an amino acid sequence selected from the group consisting of SEQ ID NOs: 10, 25, 40, 55, 70, 85, 98, 111, and 124; and (b) a variable light chain comprising an amino acid sequence selected from the group consisting of SEQ ID NOs: 11, 26, 41, 56, 71, 86, 99, 112, and 125, wherein the variable heavy chain and the variable light chain is linked by at least one linker.

In some embodiments of the CAR disclosed herein, the CAR comprises an extracellular domain, a transmembrane domain, and an intracellular domain, wherein the extracellular domain comprises a Claudin 18.2 antigen binding domain that specifically binds to Claudin 18.2, and wherein the antigen binding domain comprises: (a) a variable heavy chain comprising an amino acid sequence selected from the group consisting of SEQ ID NOs: 10, 25, 40, 55, 70, 85, 98, 111, and 124; and (b) a variable light chain comprising an amino acid sequence selected from the group consisting of SEQ ID NOs: 11, 26, 41, 56, 71, 86, 99, 112, and 125, wherein the variable heavy chain and the variable light chain is linked by at least one linker.

In some embodiments of the CAR disclosed herein, the VH region comprises a VH CDR1 comprising the amino acid sequence shown in SEQ ID NO: 1, 2 or 3; a VH CDR2 comprising the amino acid sequence shown in SEQ ID NO: 4 or 5; and a VH CDR3 comprising the amino acid sequence shown in SEQ ID NO: 6; and the VL region comprises a VL CDR1 comprising the amino acid sequence shown in SEQ ID NO: 7; a VL CDR2 comprising the amino acid sequence shown in SEQ ID NO: 8; and a VL CDR3 comprising the amino acid sequence shown in SEQ ID NO: 9.

In some embodiments of the CAR disclosed herein, the VH region comprises the amino acid sequence shown in SEQ ID NO: 10 and the VL region comprises the amino acid sequence shown in SEQ ID NO: 11.

In some embodiments of the CAR disclosed herein, the VH region comprises a VH CDR1 comprising the amino acid sequence shown in SEQ ID NO: 16, 17 or 18; a VH CDR2 comprising the amino acid sequence shown in SEQ ID NO: 19 or 20; and a VH CDR3 comprising the amino acid sequence shown in SEQ ID NO: 21; and the VL region comprises a VL CDR1 comprising the amino acid sequence shown in SEQ ID NO: 22; a VL CDR2 comprising the amino acid sequence shown in SEQ ID NO: 23; and a VL CDR3 comprising the amino acid sequence shown in SEQ ID NO: 24.

In some embodiments of the CAR disclosed herein, the VH region comprises the amino acid sequence shown in SEQ ID NO: 25 and the VL region comprises the amino acid sequence shown in SEQ ID NO: 26.

In some embodiments of the CAR disclosed herein, the VH region comprises a VH CDR1 comprising the amino acid sequence shown in SEQ ID NO: 31, 32 or 33; a VH CDR2 comprising the amino acid sequence shown in SEQ ID NO: 34 or 35; and a VH CDR3 comprising the amino acid sequence shown in SEQ ID NO: 36; and the VL region comprises a VL CDR1 comprising the amino acid sequence shown in SEQ ID NO: 37; a VL CDR2 comprising the amino acid sequence shown in SEQ ID NO: 38; and a VL CDR3 comprising the amino acid sequence shown in SEQ ID NO: 39.

In some embodiments of the CAR disclosed herein, the VH region comprises the amino acid sequence shown in SEQ ID NO: 40 and the VL region comprises the amino acid sequence shown in SEQ ID NO: 41.

In some embodiments of the CAR disclosed herein, the VH region comprises a VH CDR1 comprising the amino acid sequence shown in SEQ ID NO: 46, 47 or 48; a VH CDR2 comprising the amino acid sequence shown in SEQ ID NO: 49 or 50; and a VH CDR3 comprising the amino acid sequence shown in SEQ ID NO: 51; and the VL region comprises a VL CDR1 comprising the amino acid sequence shown in SEQ ID NO: 52; a VL CDR2 comprising the amino acid sequence shown in SEQ ID NO: 53; and a VL CDR3 comprising the amino acid sequence shown in SEQ ID NO: 54.

In some embodiments of the CAR disclosed herein, the VH region comprises the amino acid sequence shown in SEQ ID NO: 55 and the VL region comprises the amino acid sequence shown in SEQ ID NO: 56.

In some embodiments of the CAR disclosed herein, the VH region comprises a VH CDR1 comprising the amino acid sequence shown in SEQ ID NO: 61, 62 or 63; a VH CDR2 comprising the amino acid sequence shown in SEQ ID NO: 64 or 65; and a VH CDR3 comprising the amino acid sequence shown in SEQ ID NO: 66; and the VL region comprises a VL CDR1 comprising the amino acid sequence shown in SEQ ID NO: 67; a VL CDR2 comprising the amino acid sequence shown in SEQ ID NO: 68; and a VL CDR3 comprising the amino acid sequence shown in SEQ ID NO: 69.

In some embodiments of the CAR disclosed herein, the VH region comprises the amino acid sequence shown in SEQ ID NO: 70 and the VL region comprises the amino acid sequence shown in SEQ ID NO: 71.

In some embodiments of the CAR disclosed herein, the VH region comprises a VH CDR1 comprising the amino acid sequence shown in SEQ ID NO: 76, 77 or 78; a VH CDR2 comprising the amino acid sequence shown in SEQ ID NO: 79 or 80; and a VH CDR3 comprising the amino acid sequence shown in SEQ ID NO: 81; and the VL region comprises a VL CDR1 comprising the amino acid sequence shown in SEQ ID NO: 82; a VL CDR2 comprising the amino acid sequence shown in SEQ ID NO: 83; and a VL CDR3 comprising the amino acid sequence shown in SEQ ID NO: 84.

In some embodiments of the CAR disclosed herein, the VH region comprises the amino acid sequence shown in SEQ ID NO: 85 and the VL region comprises the amino acid sequence shown in SEQ ID NO: 86.

In some embodiments of the CAR disclosed herein, the VH region comprises a VH CDR1 comprising the amino acid sequence shown in SEQ ID NO: 89, 90 or 91; a VH CDR2 comprising the amino acid sequence shown in SEQ ID NO: 92 or 93; and a VH CDR3 comprising the amino acid sequence shown in SEQ ID NO: 94; and the VL region comprises a VL CDR1 comprising the amino acid sequence shown in SEQ ID NO: 95; a VL CDR2 comprising the amino acid sequence shown in SEQ ID NO: 96; and a VL CDR3 comprising the amino acid sequence shown in SEQ ID NO: 97.

In some embodiments of the CAR disclosed herein, the VH region comprises the amino acid sequence shown in SEQ ID NO: 98 and the VL region comprises the amino acid sequence shown in SEQ ID NO: 99.

In some embodiments of the CAR disclosed herein, the VH region comprises a VH CDR1 comprising the amino acid sequence shown in SEQ ID NO: 102, 103 or 104; a VH CDR2 comprising the amino acid sequence shown in SEQ ID NO: 105 or 106; and a VH CDR3 comprising the amino acid sequence shown in SEQ ID NO: 107; and the VL region comprises a VL CDR1 comprising the amino acid sequence shown in SEQ ID NO: 108; a VL CDR2 comprising the amino acid sequence shown in SEQ ID NO: 109; and a VL CDR3 comprising the amino acid sequence shown in SEQ ID NO: 110.

In some embodiments of the CAR disclosed herein, the VH region comprises the amino acid sequence shown in SEQ ID NO: 111 and the VL region comprises the amino acid sequence shown in SEQ ID NO: 112.

In some embodiments of the CAR disclosed herein, the VH region comprises a VH CDR1 comprising the amino acid sequence shown in SEQ ID NO: 115, 116 or 117; a VH CDR2 comprising the amino acid sequence shown in SEQ ID NO: 118 or 119; and a VH CDR3 comprising the amino acid sequence shown in SEQ ID NO: 120; and the VL region comprises a VL CDR1 comprising the amino acid sequence shown in SEQ ID NO: 121; a VL CDR2 comprising the amino acid sequence shown in SEQ ID NO: 122; and a VL CDR3 comprising the amino acid sequence shown in SEQ ID NO: 123.

In some embodiments of the CAR disclosed herein, the VH region comprises the amino acid sequence shown in SEQ ID NO: 124 and the VL region comprises the amino acid sequence shown in SEQ ID NO: 125.

In some embodiments of the CAR disclosed herein, the CAR comprises an extracellular domain, a transmembrane domain, and an intracellular domain, wherein the extracellular domain comprises a Claudin 18.2 antigen binding domain that specifically binds to Claudin 18.2, and wherein the antigen binding domain comprises a sequence selected from the group consisting of those scFvs presented in Table 1c. In some embodiments, the extracellular domain of the CAR comprises an amino acid sequence that is at least about 80%, 85%, 90%, 95%, 96%, 98%, 99% or 100% identical to any one of the amino acid sequences of SEQ ID NOs: 12, 27, 42, 57, 72, 187, 189, 191, and 193.

In some embodiments, the disclosure provides a Claudin 18.2-specific CAR comprising an extracellular ligand-binding domain, a transmembrane domain, and an intracellular signaling domain, wherein the extracellular domain comprises a single chain Fv fragment (scFv) binding to the extracellular domain of Claudin 18.2, wherein the scFv comprises a heavy chain variable (VH) region and a light chain variable (VL) region; wherein the VH region comprises an amino acid sequence that shares at least about 94%, 95%, 96%, 97%, 98%, 99%, to 100% with SEQ ID NO: 10 and the VL region comprises an amino acid sequence that shares at least about 94%, 95%, 96%, 97%, 98%, 99%, to 100% with SEQ ID NO: 11; or the VH region comprises an amino acid sequence that shares at least about 94%, 95%, 96%, 97%, 98%, 99%, to 100% with SEQ ID NO: 25 and the VL region comprises an amino acid sequence that shares at least about 94%, 95%, 96%, 97%, 98%, 99%, to 100% with SEQ ID NO: 26; or the VH region comprises an amino acid sequence that shares at least about 94%, 95%, 96%, 97%, 98%, 99%, to 100% with SEQ ID NO: 40 and the VL region comprises an amino acid sequence that shares at least about 94%, 95%, 96%, 97%, 98%, 99%, to 100% with SEQ ID NO: 41; or the VH region comprises an amino acid sequence that shares at least about 94%, 95%, 96%, 97%, 98%, 99%, to 100% with SEQ ID NO: 55 and the VL region comprises an amino acid sequence that shares at least about 94%, 95%, 96%, 97%, 98%, 99%, to 100% with SEQ ID NO: 56; or the VH region comprises an amino acid sequence that shares at least about 94%, 95%, 96%, 97%, 98%, 99%, to 100% with SEQ ID NO: 70 and the VL region comprises an amino acid sequence that shares at least about 94%, 95%, 96%, 97%, 98%, 99%, to 100% with SEQ ID NO: 71; or the VH region comprises an amino acid sequence that shares at least about 94%, 95%, 96%, 97%, 98%, 99%, to 100% with SEQ ID NO: 85 and the VL region comprises an amino acid sequence that shares at least about 94%, 95%, 96%, 97%, 98%, 99%, to 100% with SEQ ID NO: 86; or the VH region comprises an amino acid sequence that shares at least about 94%, 95%, 96%, 97%, 98%, 99%, to 100% with SEQ ID NO: 98 and the VL region comprises an amino acid sequence that shares at least about 94%, 95%, 96%, 97%, 98%, 99%, to 100% with SEQ ID NO: 99; or the VH region comprises an amino acid sequence that shares at least about 94%, 95%, 96%, 97%, 98%, 99%, to 100% with SEQ ID NO: 111 and the VL region comprises an amino acid sequence that shares at least about 94%, 95%, 96%, 97%, 98%, 99%, to 100% with SEQ ID NO: 112; or the VH region comprises an amino acid sequence that shares at least about 94%, 95%, 96%, 97%, 98%, 99%, to 100% with SEQ ID NO: 124 and the VL region comprises an amino acid sequence that shares at least about 94%, 95%, 96%, 97%, 98%, 99%, to 100% with SEQ ID NO: 125.

In some embodiments of the CAR disclosed herein, the CAR comprises an amino acid sequence that is at least about 80%, 85%, 90%, 95%, 96%, 98%, 99% or 100% identical to any one of SEQ ID NOs: 13, 28, 43, 58, 73, 87, 100, 113, 126, 128, 130,132, 179-183, 184-186, and 195-198, with or without a signal sequence. In some embodiments, the chimeric antigen receptor comprises an amino acid sequence of any one of SEQ ID NOs: 13, 28, 43, 58, 73, 87, 100, 113, 126, 128, 130, 132, 179-183, 184-186, 195-198, 200-201 and 208-211, with or without a signal sequence. The present disclosure provides amino acid sequences of the CARs disclosed herein with a signal sequence and without a signal sequence.

In some embodiments, the chimeric antigen receptor described herein further comprises a hinge domain. In some embodiments, the hinge and transmembrane domains comprise the hinge and transmembrane domains of human CD8α. In some embodiments, the hinge and transmembrane domains comprise the hinge and transmembrane domains of human CD28.

In some embodiments, the intracellular domain of the chimeric antigen receptor comprises at least one costimulatory domain. In some embodiments, the CAR disclosed herein comprises one costimulatory domain. In some embodiments, the CAR disclosed herein comprises two costimulatory domains.

In some embodiments, the costimulatory domain of the chimeric antigen receptor is a signaling region of CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1 (CD1 1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), activating NK cell receptors, BTLA, a Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, CD49D, ITGA6, VLA-6, CD49f, ITGAD, CD1 1d, ITGAE, CD103, ITGAL, CD1 1a, LFA-1, ITGAM, CD1 1b, ITGAX, CD1 1c, ITGB1, CD29, ITGB2, CD18, LFA-1, ITGB7, NKG2D, TNFR2, TRANCE/RANKL, DNAMI (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

In some embodiments, the costimulatory domain comprises a signaling region of CD28, or a variant thereof. In some embodiments, the CD28 costimulatory domain comprises the amino acid sequence of SEQ ID NO: 158 or 174.

In some embodiments, the costimulatory domain comprises a CD28.YMFM intracellular domain (SEQ ID NO: 216). In some embodiments, the CD28.YMFM intracellular domain (SEQ ID NO: 216) comprises the amino acid sequence of SEQ ID NO: 159.

In some embodiments, the costimulatory domain comprises a signaling region of 4-1BB/CD137. In some embodiments, the 4-1BB/CD137 costimulatory domain comprises SEQ ID NO: 137.

In some embodiments, the intracellular domain comprises at least one activating domain. In some embodiments, the activating domain comprises CD3. In some embodiments, the activating domain comprises the CD3 activating domain CD3 zeta. In some embodiments, the CD3 zeta comprises the amino acid sequence of SEQ ID NO: 138. In some embodiments, the CD3 zeta comprises the amino acid sequence of SEQ ID NO: 139.

In some embodiments, the chimeric antigen receptor is encoded by the polynucleotide sequence of any one of SEQ ID NOs: 15, 30, 45, 60, 75, 88, 101, 114 and 127.

In some embodiments, the disclosure provides a polynucleotide encoding a Claudin 18.2-specific CAR, wherein the polynucleotide comprises a nucleic acid sequence that shares at least 95%, 96%, 97%, 98%, 99%, or 100% with any one of SEQ ID NOs: 14, 15, 29, 30, 44, 45, 59, 60, 74, 75, 88, 101, 114 and 127.

For all polynucleotide sequences disclosed herein, alternative versions of the disclosed sequences can be substituted in order to optimize (wholly or partially) the sequence according to the codon preferences of the organism in which the sequence will be expressed or according to any other known methods of codon optimization, to avoid recombination of polynucleotide sequences that encode similar molecules (e.g. two different versions of a CD3 zeta domain), and/or for any other practical reason. The person of ordinary skill in the art is aware of the degeneracy of the genetic code and of codon preferences of various organisms e.g. laboratory model organisms and cell lines used for small (e.g. laboratory) scale and commercial scale production of CARs and antibodies like those disclosed herein. See. e.g., C. H. Kim et al., Codon optimization for high-level expression of human erythropoietin (EPO) in mammalian cells. Gene. 1997 Oct. 15; 199 (1-2):293-301. doi: 10.1016/s0378-1119(97)00384-3. PMID: 9358069: S. Guedan et al., Engineering and Design of Chimeric Antigen Receptors. Mol Ther Methods Clin Dev. 2018 Dec. 31; 12:145-156. doi: 10.1016/j.omtm.2018.12.009. PMID: 30666307; PMCID: PMC6330382.

In some embodiments, the chimeric antigen receptor further comprises a safety switch.

In some embodiments, the safety switch comprises a CD20 mimotope or a QBEND-10 epitope.

In some embodiments, the safety switch comprises one or more CD20 mimotopes or one or more QBEND-10 epitopes, or combinations thereof.

In some embodiments, the chimeric antigen receptor comprises one or more safety switch in the format of QR3, SR2, RSR, or R2S.

In some embodiments, the chimeric antigen receptor comprises the amino acid sequence that is at least about 80%, 85%, 90%, 95%, 96%, 98%, 99% or 100% identical to any one of SEQ ID NOs: 128, 130, 132, and 184-186.

In some embodiments, the chimeric antigen receptor comprises the amino acid sequence that is at least about 80%, 85%, 90%, 95%, 96%, 98%, 99% or 100% identical to any one of SEQ ID NOs: 128, 130, and 132, with or without a signal sequence.

In some aspects, the present disclosure provides an isolated polynucleotide encoding any one of the chimeric antigen receptors described herein.

In another aspect, the present disclosure provides a vector comprising the polynucleotide encoding any one of the chimeric antigen receptors described herein.

In some embodiments, the vector is a retroviral vector, a DNA vector, a plasmid, an RNA vector, an adenoviral vector, an adeno-associated virus vector, a lentiviral vector, or any combination thereof.

In some embodiments, the extracellular domain of the chimeric antigen receptor described herein further comprises an anti-CD70 scFv that specifically binds to CD70. In some embodiments, the anti-CD70 scFv comprises the amino acid sequence of SEQ ID NO: 204, 205, and/or 206.

In another aspect, the present disclosure provides an engineered immune cell comprising or expressing (e.g. expressing at its cell surface membrane) a Claudin 18.2-specific chimeric antigen receptor described herein. In some embodiments, the engineered immune cell comprises another CAR which is not specific for Claudin 18.2. In some embodiments, the engineered immune cell can comprise a polynucleotide encoding a suicide polypeptide. In some embodiments, the suicide polypeptide is RQR8.

In some aspects, the present disclosure provides an engineered immune cell comprising or expressing the polynucleotide or vector encoding any one of the chimeric antigen receptors described herein.

In some embodiments, the engineered immune cell further comprises or expresses a CD70 binding protein. In some embodiments, the CD70 binding protein comprises an anti-CD70 antibody or an antigen binding fragment thereof, and a transmembrane domain, and optionally a hinge domain. In some embodiments, the anti-CD70 antibody comprises the amino acid sequence of SEQ ID NO: 204, 205 and/or 206. In some embodiments, the CD70 binding protein further comprises a CD3z signaling domain and does not comprise a costimulatory domain. In some embodiments, the CD70 binding protein comprises the amino acid sequence of SEQ ID NO: 207.

In some embodiments, the engineered immune cell further comprises or expresses a dominant negative receptor. In some embodiments, the dominant negative receptor can mitigate the immunoinhibitory signals present in the tumor microenvironment. In some embodiments, the dominant negative receptor is a PD1 or TGFβ receptor (TGFβR) dominant negative receptor. In some embodiments, the dominant negative receptor comprises the extracellular domain of PD1 or TGFβR, a transmembrane domain, and without a functional intracellular signaling domain. In some embodiments, the PD1 or TGFβR extracellular domain comprises the extracellular domain from WT PD1 or WT TGFβR, or a variant thereof. In some embodiments, the dominant negative receptor comprises a CD8 or CD28 transmembrane domain or the transmembrane domain from PD1 or TGFβR. In some embodiments, exemplary PD1 and TGFβR dominant negative receptor can comprise the amino acid sequence of SEQ ID NO: 212, 213, or 214.

In some embodiments, the engineered immune cell is or is derived from a T cell, tumor infiltrating lymphocyte (TIL), NK cell, TCR-expressing cell, dendritic cell, or NK-T cell. In some embodiments, the engineered immune is or is derived from an inflammatory T-lymphocyte, a cytotoxic T-lymphocyte, a regulatory T-lymphocyte, or a helper T-lymphocyte.

In some embodiments, the engineered immune cell is an autologous T cell. In some embodiments, the engineered immune cell is an allogeneic T cell. In some embodiments, the engineered immune cell is obtained from a healthy donor. In some embodiments, the engineered immune cell is obtained from a patient.

In some embodiments, the engineered immune cell comprises a disruption (e.g. knock-out) at one or more endogenous genes, wherein the endogenous gene encodes TCRα, TCRβ, CD52, glucocorticoid receptor (GR), deoxycytidine kinase (dCK), CD70 or an immune checkpoint protein such as for example programmed death-1 (PD-1).

In another aspect, the disclosure provides a method of engineering an immune cell comprising: providing an immune cell; and expressing at the surface of the cell at least one Claudin 18.2-specific CAR as described herein. In some embodiments, the method comprises: providing an immune cell; introducing into the cell at least one polynucleotide encoding a Claudin 18.2-specific CAR as described herein; and expressing said polynucleotide in the cell or causing said polynucleotide to be expressed in the cell, e.g. by providing in the cell appropriate elements (e.g. one or more transcription promoters and/or enhancers) that direct the expression of the polynucleotide encoding the CAR.

In some embodiments, the method comprises: providing an immune cell; introducing into the cell at least one polynucleotide encoding a Claudin 18.2-specific CAR as described herein; and introducing at least one other polynucleotide encoding a second polypeptide e.g. a CAR that is not specific for Claudin 18.2; and expressing said polynucleotides in the cell or causing said polynucleotides to be expressed in the cell, e.g. by providing in the cell appropriate elements (e.g. one or more transcription promoters and/or enhancers) that direct the expression of the polynucleotides.

In one aspect, the present disclosure provides a pharmaceutical composition. In some embodiments, the pharmaceutical composition comprises an engineered immune cell disclosed herein and at least one pharmaceutically acceptable excipient. In some embodiments, the pharmaceutical composition comprises an anti-Claudin 18.2 binding agent disclosed herein and at least one pharmaceutically acceptable excipient. In some embodiments, the pharmaceutical composition comprises an engineered immune cell expressing an anti-Claudin 18.2 chimeric antigen receptor disclosed herein and at least one pharmaceutically acceptable excipient.

In some aspects, the present disclosure provides a method of treating a disease or disorder in a subject in need thereof comprising administering to the subject an engineered immune cell disclosed herein or a pharmaceutical composition disclosed herein, e.g. a pharmaceutical composition comprising an engineered immune cell expressing an anti-Claudin 18.2 chimeric antigen receptor disclosed herein and at least one pharmaceutically acceptable excipient. In some embodiments, the engineered immune cell expresses an anti-Claudin 18.2 chimeric antigen receptor disclosed herein and expresses a second polypeptide e.g. a second CAR

In some embodiments, the disease or disorder is cancer.

In some embodiments, the disease or disorder is gastric cancer, gastroesophageal junction (GEJ) cancer or pancreatic cancer.

In some embodiments, the disease or disorder is an autoimmune disease.

In some aspects, the present disclosure provides a method of treating a disease or disorder in a subject in need thereof comprising administering to the subject an anti-Claudin 18.2 binding agent, or a pharmaceutical composition comprising the anti-Claudin 18.2 binding agent, as disclosed herein. In some embodiments, the disease or disorder is cancer. In some embodiments, the disease or disorder is gastric cancer, gastroesophageal junction (GEJ) cancer or pancreatic cancer.

In another aspect, the disclosure provides a method of inhibiting tumor growth or progression in a subject who has malignant cells expressing Claudin 18.2, the method comprising administering to the subject in need thereof an effective amount of a pharmaceutical composition disclosed herein, e.g. a pharmaceutical composition comprising an engineered immune cell as described herein and at least one pharmaceutically acceptable excipient or a pharmaceutical composition comprising an anti-Claudin 18.2 binding agent as described herein and at least one pharmaceutically acceptable excipient.

In another aspect, the disclosure provides a method of inhibiting metastasis of malignant cells expressing Claudin 18.2 in a subject, comprising administering to the subject in need thereof an effective amount of the pharmaceutical composition disclosed herein, e.g. a pharmaceutical composition comprising an engineered immune cell as described herein and at least one pharmaceutically acceptable excipient or a pharmaceutical composition comprising an anti-Claudin 18.2 binding agent as described herein and at least one pharmaceutically acceptable excipient.

In another aspect, the disclosure provides a method of inducing tumor regression in a subject who has malignant cells expressing Claudin 18.2, comprising administering to the subject in need thereof an effective amount of the pharmaceutical composition disclosed herein, e.g. a pharmaceutical composition comprising an engineered immune cell as described herein and at least one pharmaceutically acceptable excipient or a pharmaceutical composition comprising an anti-Claudin 18.2 binding agent as described herein and at least one pharmaceutically acceptable excipient.

In some embodiments, the engineered immune cell or the pharmaceutical composition is administered to the subject intravenously, subcutaneously, or intraperitoneally, or is administered to the subject by intravenous injection, subcutaneous injection of intraperitoneal injection.

In some embodiments, any of the above methods further comprises administering one or more additional therapies, such as, for example, a monoclonal antibody and/or a chemotherapeutic. In some embodiments, the monoclonal antibody can be, for example, an antibody that binds to a checkpoint inhibitor such as, for example, an anti-PD-1 antibody or an anti-PD-L1 antibody. In some embodiments, any of the above methods further comprises administering a Receptor Tyrosine Kinase inhibitor such as sunitinib or axitinib.

In another aspect, the disclosure provides an engineered immune cell expressing at its cell-surface membrane a Claudin 18.2-specific CAR as described herein for use as a medicament. In some embodiments, the medicament is for use in treatment of a cancer. In some embodiments, the medicament is for treatment of gastric cancer, gastroesophageal junction (GEJ) cancer and pancreatic cancer. In some embodiments, the medicament is for use in treatment of an autoimmune disease.

In another aspect, the disclosure provides an anti-Claudin 18.2 binding agent as described herein for use as a medicament. In some embodiments, the medicament is for use in treatment of a cancer. In some embodiments, the medicament is for treatment of gastric cancer, gastroesophageal junction (GEJ) cancer and pancreatic cancer. In some embodiments, the anti-Claudin 18.2 binding agent is an antibody, an antibody conjugate, or an antigen-binding fragment thereof, optionally, a F(ab′)₂ fragment, a Fab′ fragment, a Fab fragment, a Fv fragment, a scFv fragment, a dsFv fragment, or a domain antibody (dAb) fragment, or a monoclonal antibody comprising an IgG constant region.

In some aspects, the present disclosure provides an article of manufacture comprising the engineered immune cell disclosed herein or the pharmaceutical composition disclosed herein, e.g. the engineered immune cell or the pharmaceutical composition comprising the engineered immune cell expressing a chimeric antigen receptor disclosed herein.

In some aspects, the present disclosure provides an anti-Claudin 18.2 binding agent. In some embodiments, the anti-Claudin 18.2 binding agent comprises (a) a variable heavy chain CDR1 comprising an amino acid sequence selected from the group consisting of SEQ ID NOs: 1-3, 16-18, 31-33, 46-48, 61-63, 76-78, 89-91, 102-104, and 115-117; (b) a variable heavy chain CDR2 comprising an amino acid sequence selected from the group consisting of SEQ ID NOs: 4-5, 19-20, 34-35, 49-50, 64-65, 79-80, 92-93, 105-106, 118-119; (c) a variable heavy chain CDR3 comprising an amino acid sequence selected from the group consisting of SEQ ID NOs: 6, 21, 36, 51, 66, 81, 94, 107, 120; (d) a variable light chain CDR1 comprising an amino acid sequence selected from the group consisting of SEQ ID NOs: 7, 22, 37, 52, 67, 82, 95, 108, 121; (e) a variable light chain CDR2 comprising an amino acid sequence selected from the group consisting of SEQ ID NOs: 8, 23, 38, 53, 68, 83, 96, 109, 122; and (f) a variable light chain CDR3 comprising an amino acid sequence selected from the group consisting of SEQ ID NOs: 9, 24, 39, 54, 69, 84, 97, 110, 123.

In some embodiments, the anti-Claudin 18.2 binding agent is an antibody, an antibody conjugate, or an antigen-binding fragment thereof, optionally, a F(ab′)₂ fragment, a Fab′ fragment, a Fab fragment, a Fv fragment, a scFv fragment, a dsFv fragment, or a domain antibody (dAb) fragment.

In some embodiments, the anti-Claudin 18.2 binding agent is a monoclonal antibody comprising an IgG constant region.

In some embodiments, the anti-Claudin 18.2 binding agent comprises a variable heavy (VH) chain sequence that is at least about 80%, 85%, 90%, 95%, %%, 98%, 99% or 100% identical to an amino acid sequence selected from the group consisting of SEQ ID NOs: 10, 25, 40, 55, 70, 85, 98, 111, and 124.

In some embodiments, the anti-Claudin 18.2 binding agent comprises a variable light (VL) chain sequence that is at least about 80%, 85%, 90%, 95%, 96%, 98%, 99% or 100% identical to an amino acid sequence selected from the group consisting of SEQ ID NOs: 11, 26, 41, 56, 71, 86, 99, 112, and 125.

In some embodiments, the anti-Claudin 18.2 binding agent comprises a sequence that is at least about 80%, 85%, 90%, 95%, 96%, 98%, 99% or 100% identical to an amino acid sequence selected from the group consisting of SEQ ID NOS: 12, 27, 42, 57, 72, 187, 189, 191, and 193.

In some embodiments, the anti-Claudin 18.2 binding agent is a fusion protein comprising a scFv fragment fused to an Fc constant region.

In some embodiments, the anti-Claudin 18.2 binding agent comprises a VH CDR1 comprising the amino acid sequence shown in SEQ ID NO: 1, 2 or 3; a VH CDR2 comprising the amino acid sequence shown in SEQ ID NO: 4 or 5; a VH CDR3 comprising the amino acid sequence shown in SEQ ID NO: 6; a VL CDR1 comprising the amino acid sequence shown in SEQ ID NO: 7; a VL CDR2 comprising the amino acid sequence shown in SEQ ID NO: 8; and a VL CDR3 comprising the amino acid sequence shown in SEQ ID NO: 9.

In some embodiments, the anti-Claudin 18.2 binding agent comprises the amino acid sequence shown in SEQ ID NO: 10 and the VL region comprises the amino acid sequence shown in SEQ ID NO: 11.

In some embodiments, the anti-Claudin 18.2 binding agent comprises a VH CDR1 comprising the amino acid sequence shown in SEQ ID NO: 16, 17 or 18; a VH CDR2 comprising the amino acid sequence shown in SEQ ID NO: 19 or 20; a VH CDR3 comprising the amino acid sequence shown in SEQ ID NO: 21; a VL CDR1 comprising the amino acid sequence shown in SEQ ID NO: 22; a VL CDR2 comprising the amino acid sequence shown in SEQ ID NO: 23; and a VL CDR3 comprising the amino acid sequence shown in SEQ ID NO: 24.

In some embodiments, the anti-Claudin 18.2 binding agent comprises the amino acid sequence shown in SEQ ID NO: 25 and the VL region comprises the amino acid sequence shown in SEQ ID NO: 26.

In some embodiments, the anti-Claudin 18.2 binding agent comprises a VH CDR1 comprising the amino acid sequence shown in SEQ ID NO: 31, 32 or 33; a VH CDR2 comprising the amino acid sequence shown in SEQ ID NO: 34 or 35; a VH CDR3 comprising the amino acid sequence shown in SEQ ID NO: 36; a VL CDR1 comprising the amino acid sequence shown in SEQ ID NO: 37; a VL CDR2 comprising the amino acid sequence shown in SEQ ID NO: 38; and a VL CDR3 comprising the amino acid sequence shown in SEQ ID NO: 39.

In some embodiments, the anti-Claudin 18.2 binding agent comprises the amino acid sequence shown in SEQ ID NO: 40 and the VL region comprises the amino acid sequence shown in SEQ ID NO: 41.

In some embodiments, the anti-Claudin 18.2 binding agent comprises a VH CDR1 comprising the amino acid sequence shown in SEQ ID NO: 46, 47 or 48; a VH CDR2 comprising the amino acid sequence shown in SEQ ID NO: 49 or 50; a VH CDR3 comprising the amino acid sequence shown in SEQ ID NO: 51; a VL CDR1 comprising the amino acid sequence shown in SEQ ID NO: 52; a VL CDR2 comprising the amino acid sequence shown in SEQ ID NO: 53; and a VL CDR3 comprising the amino acid sequence shown in SEQ ID NO: 54.

In some embodiments, the anti-Claudin 18.2 binding agent comprises the amino acid sequence shown in SEQ ID NO: 55 and the VL region comprises the amino acid sequence shown in SEQ ID NO: 56.

In some embodiments, the anti-Claudin 18.2 binding agent comprises a VH CDR1 comprising the amino acid sequence shown in SEQ ID NO: 61, 62 or 63; a VH CDR2 comprising the amino acid sequence shown in SEQ ID NO: 64 or 65; a VH CDR3 comprising the amino acid sequence shown in SEQ ID NO: 66; a VL CDR1 comprising the amino acid sequence shown in SEQ ID NO: 67; a VL CDR2 comprising the amino acid sequence shown in SEQ ID NO: 68; and a VL CDR3 comprising the amino acid sequence shown in SEQ ID NO: 69.

In some embodiments, the anti-Claudin 18.2 binding agent comprises the amino acid sequence shown in SEQ ID NO: 70 and the VL region comprises the amino acid sequence shown in SEQ ID NO: 71.

In some embodiments, the anti-Claudin 18.2 binding agent comprises a VH CDR1 comprising the amino acid sequence shown in SEQ ID NO: 76, 77 or 78; a VH CDR2 comprising the amino acid sequence shown in SEQ ID NO: 79 or 80; a VH CDR3 comprising the amino acid sequence shown in SEQ ID NO: 81; a VL CDR1 comprising the amino acid sequence shown in SEQ ID NO: 82; a VL CDR2 comprising the amino acid sequence shown in SEQ ID NO: 83; and a VL CDR3 comprising the amino acid sequence shown in SEQ ID NO: 84.

In some embodiments, the anti-Claudin 18.2 binding agent comprises the amino acid sequence shown in SEQ ID NO: 85 and the VL region comprises the amino acid sequence shown in SEQ ID NO: 86.

In some embodiments, the anti-Claudin 18.2 binding agent comprises a VH CDR1 comprising the amino acid sequence shown in SEQ ID NO: 89, 90 or 91; a VH CDR2 comprising the amino acid sequence shown in SEQ ID NO: 92 or 93; a VH CDR3 comprising the amino acid sequence shown in SEQ ID NO: 94; a VL CDR1 comprising the amino acid sequence shown in SEQ ID NO: 95; a VL CDR2 comprising the amino acid sequence shown in SEQ ID NO: 96; and a VL CDR3 comprising the amino acid sequence shown in SEQ ID NO: 97.

In some embodiments, the anti-Claudin 18.2 binding agent comprises the amino acid sequence shown in SEQ ID NO: 98 and the VL region comprises the amino acid sequence shown in SEQ ID NO: 99.

In some embodiments, the anti-Claudin 18.2 binding agent comprises a VH CDR1 comprising the amino acid sequence shown in SEQ ID NO: 102, 103 or 104; a VH CDR2 comprising the amino acid sequence shown in SEQ ID NO: 105 or 106; a VH CDR3 comprising the amino acid sequence shown in SEQ ID NO: 107; a VL CDR1 comprising the amino acid sequence shown in SEQ ID NO: 108; a VL CDR2 comprising the amino acid sequence shown in SEQ ID NO: 109; and a VL CDR3 comprising the amino acid sequence shown in SEQ ID NO: 110.

In some embodiments, the anti-Claudin 18.2 binding agent comprises the amino acid sequence shown in SEQ ID NO: 111 and the VL region comprises the amino acid sequence shown in SEQ ID NO: 112.

In some embodiments, the anti-Claudin 18.2 binding agent comprises a VH CDR1 comprising the amino acid sequence shown in SEQ ID NO: 115, 116 or 117; a VH CDR2 comprising the amino acid sequence shown in SEQ ID NO: 118 or 119; a VH CDR3 comprising the amino acid sequence shown in SEQ ID NO: 120; a VL CDR1 comprising the amino acid sequence shown in SEQ ID NO: 121; a VL CDR2 comprising the amino acid sequence shown in SEQ ID NO: 122; and a VL CDR3 comprising the amino acid sequence shown in SEQ ID NO: 123.

In some embodiments, the anti-Claudin 18.2 binding agent comprises the amino acid sequence shown in SEQ ID NO: 124 and the VL region comprises the amino acid sequence shown in SEQ ID NO: 125.

In some embodiments, the anti-Claudin 18.2 binding agent comprises a variable heavy (VH) chain sequence that is at least about 80%, 85%, 90%, 95%, %%, 98%, 99% or 100% identical to the amino acid sequence of SEQ ID NO: 10 and a variable light (VL) chain sequence that is at least about 80%, 85%, 90%, 95%, 96%, 98%, 99% or 100% identical to the amino acid sequence of SEQ ID NO: 11.

In some embodiments, the anti-Claudin 18.2 binding agent comprises a variable heavy (VH) chain sequence that is at least about 80%, 85%, 90%, 95%, %%, 98%, 99% or 100% identical to the amino acid sequence of SEQ ID NO: 25 and a variable light (VL) chain sequence that is at least about 80%, 85%, 90%, 95%, 96%, 98%, 99% or 100% identical to the amino acid sequence of SEQ ID NO: 26.

In some embodiments, the anti-Claudin 18.2 binding agent comprises a variable heavy (VH) chain sequence that is at least about 80%, 85%, 90%, 95%, %%, 98%, 99% or 100% identical to the amino acid sequence of SEQ ID NO: 40 and a variable light (VL) chain sequence that is at least about 80%, 85%, 90%, 95%, 96%, 98%, 99% or 100% identical to the amino acid sequence of SEQ ID NO: 41.

In some embodiments, the anti-Claudin 18.2 binding agent comprises a variable heavy (VH) chain sequence that is at least about 80%, 85%, 90%, 95%, 96%, 98%, 99% or 100% identical to the amino acid sequence of SEQ ID NO: 55 and a variable light (VL) chain sequence that is at least about 80%, 85%, 90%, 95%, 96%, 98%, 99% or 100% identical to the amino acid sequence of SEQ ID NO: 56.

In some embodiments, the anti-Claudin 18.2 binding agent comprises a variable heavy (VH) chain sequence that is at least about 80%, 85%, 90%, 95%, 96%, 98%, 99% or 100% identical to the amino acid sequence of SEQ ID NO: 70 and a variable light (VL) chain sequence that is at least about 80%, 85%, 90%, 95%, 96%, 98%, 99% or 100% identical to the amino acid sequence of SEQ ID NO: 71.

In some embodiments, the anti-Claudin 18.2 binding agent comprises a variable heavy (VH) chain sequence that is at least about 80%, 85%, 90%, 95%, 96%, 98%, 99% or 100% identical to the amino acid sequence of SEQ ID NO: 85 and a variable light (VL) chain sequence that is at least about 80%, 85%, 90%, 95%, 96%, 98%, 99% or 100% identical to the amino acid sequence of SEQ ID NO: 86.

In some embodiments, the anti-Claudin 18.2 binding agent comprises a variable heavy (VH) chain sequence that is at least about 80%, 85%, 90%, 95%, 96%, 98%, 99% or 100% identical to the amino acid sequence of SEQ ID NO: 98 and a variable light (VL) chain sequence that is at least about 80%, 85%, 90%, 95%, 96%, 98%, 99% or 100% identical to the amino acid sequence of SEQ ID NO: 99.

In some embodiments, the anti-Claudin 18.2 binding agent comprises a variable heavy (VH) chain sequence that is at least about 80%, 85%, 90%, 95%, 96%, 98%, 99% or 100% identical to the amino acid sequence of SEQ ID NO: 111 and a variable light (VL) chain sequence that is at least about 80%, 85%, 90%, 95%, 96%, 98%, 99% or 100% identical to the amino acid sequence of SEQ ID NO: 112.

In some embodiments, the anti-Claudin 18.2 binding agent comprises a variable heavy (VH) chain sequence that is at least about 80%, 85%, 90%, 95%, 96%, 98%, 99% or 100% identical to the amino acid sequence of SEQ ID NO: 124 and a variable light (VL) chain sequence that is at least about 80%, 85%, 90%, 95%, 96%, 98%, 99% or 100% identical to the amino acid sequence of SEQ ID NO: 125.

In some embodiments, the anti-Claudin 18.2 binding agent is a monospecific antibody. In some embodiments, the anti-Claudin 18.2 binding agent is a bispecific antibody. In some embodiments, the bispecific antibody also binds to CD3.

In some aspects, the present disclosure provides a pharmaceutical composition comprising the anti-Claudin 18.2 binding agent disclosed herein and a pharmaceutically acceptable excipient. In some aspects, the present disclosure provides a method of treating a disease or disorder in a subject in need thereof comprising administering to the subject an anti-Claudin 18.2 binding agent, or a pharmaceutical composition comprising the anti-Claudin 18.2 binding agent, as disclosed herein. In some embodiments, the disease or disorder is cancer. In some embodiments, the disease or disorder is gastric cancer, gastroesophageal junction (GEJ) cancer or pancreatic cancer. In some embodiments, the disease or disorder is an autoimmune disease.

### BRIEF DESCRIPTION OF THE DRAWINGS

FIG. 1 is a series of plots showing that purified anti-Claudin 18.2 antibodies described herein bind to HEK-293T cells expressing human or mouse Claudin 18.2 but not parental HEK-293T cells. The solid line and dashed line represent staining with anti-Claudin 18.2 antibodies or isotype control, respectively.

FIG. 2 is a series of plots showing the antigen-specific killing of target cells using anti-Claudin 18.2 CAR T cells in a 3-Day cytotoxicity assay. Non-transduced (NTD) T cells were used as negative control.

FIGS. 3A-3C are a series of plots and tables showing the transduction efficiency and phenotype of anti-Claudin 18.2 CARs with safety switches. FIG. 3A shows representative FACS plots demonstrating efficient transduction of anti-Claudin 18.2 CARs in different rituximab off-switch formats. FIG. 3B summarizes transduction efficiency in two different donors. FIG. 3C shows memory phenotype of CAR T cells at the end of the production, as determined by FACS analysis of the expression of CD62L and CD45RO markers.

FIGS. 4A-4B are a series of plots showing serial killing of target cells using anti-Claudin 18.2 CAR T cells with and without (FIG. 4A only) safety switches. FIG. 4A shows the long-term cytotoxicity against one gastric cancer cell line overexpressing Claudin 18.2 (MKN45/hClaudin 18.2) and pancreatic cancer cell lines expressing endogenous Claudin 18.2 (PATU8988s, Panc05.04). FIG. 4B shows the long-term cytotoxicity against gastric cancer cell lines expressing endogenous Claudin 18.2 (SNU-601, SNU-620, NUGC-4, GSU).

FIG. 5 is a series of bar graphs showing that anti-Claudin 18.2 CAR T cells released cytokines after coculture with Claudin 18.2 positive gastric (SNU-601) and pancreatic (PATU8988s) cell lines at a 1:1 effector: target ratio for 24 hours. Supematant was collected and IFN-γ, IL-2 and TNF-α levels were measured using human ProInflammatory 9-Plex Kit from MSD. Dotted line indicates limit of detection for individual cytokines.

FIG. 6A and FIG. 6B are plots showing tumor volumes (FIG. 6A) and body weights (FIG. 6B) of mice treated with different anti-Claudin 18.2 CARs at 1×10⁶ cell dose in a subcutaneous xenograft model. (N=5 per group)

FIG. 6C and FIG. 6D are plots showing tumor volumes (FIG. 6C) and body weights (FIG. 6D) of mice treated with different anti-Claudin 18.2 CARs at 3×10⁶ cell dose and 10×10⁶ cell dose in the same subcutaneous xenograft model (N=8 per group). Individual mouse body weight changes were plotted against days post-CAR T treatment in FIGS. 6E-61.

FIGS. 7A-7B are plots showing tumor volumes (FIG. 7A) and body weight (FIG. 7B) from in vivo experiments using the SNU-601 intraperitoneal xenograft model. Representative bioluminescence images of same mice as in FIG. 7C.

FIGS. 8A-8D are plots showing tumor volumes and body weights of mice treated with different anti-Claudin 18.2 CARs at the 3×10⁶ cell dose (FIGS. 8A and 8C) and 1×10⁶ cell dose (FIGS. 8B and 8D) in the NUGC-4 subcutaneous model (N=5 per group). Individual mouse body weight changes were plotted against days post-CAR T treatment in FIGS. 8E-8I. FIG. 8J shows CAR T cells expansion in the blood collected from mice treated with 3×10⁶ CAR+ cells. Results represent mean f SEM.

FIG. 9 shows results of the off-target or on-target risk analysis of the anti-Claudin 18.2 CAR Ts.

FIGS. 10A-C show results of the analysis of the Claudin 18.2 clone 2A4 CAR T cells or the Claudin 18.2 clone 2A4/CD70 tandem or dual CAR T cells.

FIGS. 11A-11B show data of the MLR assays. Claudin 18.2 CAR T cells expressing a CD70 CAR depleted alloreactive T cells (right panels) and resisted T cell-mediated rejection (left panels). Alloreactive T cell MLRs were performed using TRAC^{KO} graft donor T cells co-expressing a Claudin 18.2 CAR and a CD70 CAR. Data are representative of two graft-host donor pairs (FIG. 11A and FIG. 11B). Data represent mean±SEM.

FIGS. 12A-B show results comparing the activities of Claudin 18.2 CAR with the CD8 hinge and transmembrane domains with the CD28 hinge and transmembrane domains.

### DETAILED DESCRIPTION

Provided herein are Claudin 18.2-specific antibodies and chimeric antigen receptors (CARs). The Claudin 18.2 specific CARs described herein comprise an extracellular domain, a transmembrane domain, and an intracellular domain, wherein the extracellular domain comprises a Claudin 18.2 antigen binding domain that specifically binds to Claudin 18.2, and polynucleotides encoding these CARs. Also provided are immune cells comprising these Claudin 18.2-specific CARs, e.g., CAR-T cells, and pharmaceutical compositions comprising these immune cells. Methods of making and using these Claudin 18.2-specific CARs and immune cells comprising these Claudin 18.2-specific CARs are also disclosed, e.g., for the treatment of cancer.

### I. Claudin 18.2 Binding Agents

The present disclosure provides Claudin 18.2 binding agents (e.g., molecules comprising a Claudin 18.2 antigen binding domain, Claudin 18.2 antibodies or fragments thereof), that specifically bind to Claudin 18.2. As used herein, the term “antibody” refers to a polypeptide that includes canonical immunoglobulin sequence elements sufficient to confer specific binding to a particular target antigen (e.g., Claudin 18.2). As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CHI, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Those skilled in the art are well familiar with antibody structure and sequence elements, recognize “variable” and “constant” regions in provided sequences, and understand that there may be some flexibility in definition of a “boundary” between such domains such that different presentations of the same antibody chain sequence may, for example, indicate such a boundary at a location that is shifted one or a few residues relative to a different presentation of the same antibody chain sequence.

The assignment of amino acids to each of the framework, CDR, and variable domains is typically in accordance with numbering schemes of Kabat numbering (see, e.g., Kabat et al. in Sequences of Proteins of Immunological Interest, 5th Ed., NIH Publication 91-3242, Bethesda Md. 1991), Chothia numbering (see, e.g., Chothia & Lesk, (1987), J Mol Biol 196: 901-917; Al-Lazikani et al., (1997) J Mol Biol 273: 927-948; Chothia et al., (1992) J Mol Biol 227: 799-817; Tramontano et al., (1990) J Mol Biol 215(1): 175-82; and U.S. Pat. No. 7,709,226), contact numbering, or the AbM scheme (Antibody Modeling program, Oxford Molecular).

Accordingly, in some embodiments, the CDRs of the Claudin 18.2 binding agents presented herein are numbered according to the Kabat numbering scheme. In other embodiments, the CDRs of the Claudin 18.2 binding agents presented herein are numbered according to the Chothia numbering scheme. In other embodiments, the CDRs of the Claudin 18.2 binding agents presented herein are numbered according to the contact numbering scheme. In other embodiments, the CDRs of the Claudin 18.2 binding agents presented herein are numbered according to the AbM numbering scheme.

Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation.

For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art.

Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc), or other pendant group (e.g., poly-ethylene glycol, etc).

Antibodies include antibody fragments. Antibodies also include, but are not limited to, polyclonal, monoclonal, chimeric dAb (domain antibody), single chain, F_{ab}, Fₐ, F<)₂ fragments, scFvs, and F_{ab} expression libraries. An antibody may be a whole antibody, or immunoglobulin, or an antibody fragment.

As detailed above, whole antibodies consist of two pairs of a “light chain” (LC) and a “heavy chain” (HC) (such light chain (LC)/heavy chain pairs are abbreviated herein as LC/HC). The light chains and heavy chains of such antibodies are polypeptides consisting of several domains. In a whole antibody, each heavy chain comprises a heavy chain variable region (abbreviated herein as HCVR or VH) and a heavy chain constant region. The heavy chain constant region comprises the heavy chain constant domains CH1, CH2 and CH3 (antibody classes IgA, IgD, and IgG) and optionally the heavy chain constant domain CH4 (antibody classes IgE and IgM). Each light chain comprises a light chain variable domain VL and a light chain constant domain CL. The variable domains VH and VL can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (Janeway, C. A., Jr, et al, (2001). Immunobiology, 5th ed., Garland Publishing; and Woof, J., Burton, D., Nat Rev Immunol 4 (2004) 89-99). The two pairs of heavy chain and light chain (HC/LC) are capable of specifically binding to the same antigen. Thus said whole antibody is a bivalent, monospecific antibody. Such “antibodies” include e.g., mouse antibodies, human antibodies, chimeric antibodies, humanized antibodies and genetically engineered antibodies (variant or mutant antibodies) as long as their characteristic properties are retained. In some embodiments, antibodies or binding agents are humanized antibodies, especially as recombinant human or humanized antibodies.

In some embodiments, the antibody or binding agent can be “symmetrical.” By “symmetrical” is meant that the antibody or binding agent has the same kind of Fv regions (e.g., the antibody has two Fab regions). In some embodiments, the antibody or binding agent can be “asymmetrical.” By “asymmetrical” is meant that the antibody or binding agent has at least two different kinds of Fv regions (e.g., the antibody has: Fab and scFv regions, Fab and scFv2 regions, or Fab-VHH regions). Various asymmetrical antibody or binding agent architectures are known in the art (Brinkman and Kontermann et al. 2017 Mabs (9)(2): 182-212).

As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc, as is known in the art. In many embodiments, the term “antibody agent” is used to refer to one or more of the art-known or developed constructs or formats for utilizing antibody structural and functional features in alternative presentation. For example, an antibody agent utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™); single chain or Tandem diabodies (TandAb®); VHHs; Anticalins®; Nanobodies® minibodies; BiTE® s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s.

In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments, an antibody agent is or comprises a polypeptide whose amino acid sequence includes at least one CDR (e.g., at least one heavy chain CDR and/or at least one light chain CDR) that is substantially identical to one found in a reference antibody In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

An antibody or antigen binding molecule encoded of the present invention can be single chained or double chained. In some embodiments, the antibody or antigen binding molecule is single chained. In certain embodiments, the antigen binding molecule is selected from the group consisting of an scFv, a Fab, a Fab′, a Fv, a F(ab′)₂, a dAb, and any combination thereof.

In some embodiments, an anti-Claudin 18.2 antibody agent is isolated. In some embodiments, an antibody agent can be purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) (See, e.g., Flatman et al., J. Chromatogr., B 848:79-87 (2007)). In some aspects, the present disclosure provides a composition comprising a Claudin 18.2 binding agent (e.g., a Claudin 18.2 specific antibody) and a pharmaceutically acceptable carrier or excipient.

In some embodiments, an anti-Claudin 18.2 antibody agent comprises an Fc. Fc domains can interact with cell surface receptors which can allow antibodies to activate the immune system. In IgG, IgA and IgD antibody isotypes, a Fc region is composed of two identical protein fragments, derived from the second and third constant domains of the antibody's two heavy chains; IgM and IgE Fc regions contain three heavy chain constant domains (C_{H} domains 2-4) in each polypeptide chain. The Fc regions of IgG may bear a highly conserved N-glycosylation site (N297). Glycosylation of the Fc fragment may be essential for Fc receptor-mediated activity. The N-glycans attached to this site can predominantly be core-fucosylated diantennary structures of the complex type.

While the constant regions of the light and heavy chains may not be directly involved in binding of the antibody to an antigen, the constant regions can influence the orientation of the variable regions. The constant regions can also exhibit various effector functions, such as participation in antibody-dependent complement-mediated lysis or antibody-dependent cellular toxicity via interactions with effector molecules and cells.

The disclosed anti-Claudin 18.2 antibody agents can be antibodies of any isotype, including isotype IgA, isotype IgD, isotype IgE, isotype IgG, or isotype IgM. In some embodiments, an anti-Claudin 18.2 antibody contains a IgG1, IgG2, IgG3, or IgG4 constant domain.

Provided herein are Claudin 18.2 binding agents (e.g., antibodies) that can bind to various regions or domains of the Claudin 18.2 target. The epitope can be, for example, contiguous amino acids of the Claudin 18.2 target (linear or contiguous epitope) or come together from two or more non-contiguous regions of the Claudin 18.2 target (conformational, non-linear, discontinuous, or non-contiguous epitope). The epitope to which the Claudin 18.2 antigen binding domain binds can be determined by various assays, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, flow cytometry, and/or mutagenesis mapping (e.g., site-directed mutagenesis mapping).

In some embodiments, the Claudin 18.2 binding agent comprises a variable heavy chain (VH), wherein the amino acid sequence of the VH is selected from the VH sequences presented in Table 1a. In some embodiments, an anti-Claudin 18.2 binding agent comprises an immunoglobulin variable heavy chain having at least 60%, 65%, 70%, 75%, 80%, 85%, 90%, 91%, 92%, 93%, 94%, 95%, 96%, 97%, 98%, 99%, or 100% sequence identity to an amino acid sequence presented in Table 1a. Kabat CDR definitions are in bold and Chothia CDR definitions are underlined.

TABLE 1a
Amino Acid Sequences of Exemplary anti-Claudin 18.2 Heavy Chain Variable
Regions (VH)

| Clone | VH Sequence | SEQ ID NO: |
|---|---|---|
| 1E7 | QITLKESGPTLVKPTQTLTLTCTFSGFSLSTSGVGVGWIRQPPGKAPE | 10 |
| | WLAQIYWNDEKRYSSSLKSRLTITKDTSKNQVVLKMTNMDPVDTA | |
| | TYYCAHRRGIGNWFDPWGQGTLVTVSS | |
| 2A4 | QITLKESGPTLVKPTQTLTLTCTFSGFSLSTSGVGVGWIRQTPGKALE | 25 |
| | WLTQIYWNDEKRYSPSLRNRLTITKDTSKNQVVLTMTNMDPVDTA | |
| | TYYCAHRRGVGNWEDPWGQGILVTVSS | |
| 9G2 | EVHLLESGGGLVQPWGSLTLSCAASGFTFSNYAMNWVRQAPGKGLE | 40 |
| | WVSGISGSGGSTYDADSVKGRFTISRDNSKNTLFLQMNSPRAEDTA | |
| | VYYCATQGYSFGYFESWGQGTLVTVSS | |
| 2A10 | QVQLQESGPGLVKPSETLSLTCTVSAGSISSYYWNWIRQPAGKGLEW | 55 |
| | IGRIYTSGSTNYNPSLRSRVTMSVDTSKNQFSLKLSSVTATDTAVYY | |
| | CASASYTYFDSFDIWGQGTMVTVSS | |
| 12H6 | EVQLVESGGGLVQPGGSLRLSCAASGFTFSRYWMSWVRQAPGKGL | 70 |
| | EWVANIKHDGSEKYYVDSVKGRFTFSRDNAKTSLYLQMNSLRVED | |
| | TALYYCARYYGGPFDYWGQGTLVTVSS | |
| 10D11 | EVQLLESGGGLEQPGGSLRLSCAASGFTFSSYAMSWVRQAPGKGLE | 85 |
| | WVSAISGSGGSTHYADSVKGRFTISRDNARNTLYLQMNSLRAEDTA | |
| | VYYCAKEGYVGSWYAPFDYWGQGTLVTVSS | |
| 17F11 | QVTLRESGPALVKPTQTLTLTCTVSGVSLSTSGMCVSWIRQPLGKAL | 98 |
| | EWLGFIDWDDDKYYNTSLKTRLTISKDTSKNQVVLTMTNMDPVDT | |
| | ATYYCARIRGYSGSYDAFDIWGQGTVVIVSS | |
| 6B2 | QVTLKESGLTLMKPTQTHTLTCTFSGFSLSTSGVGVGWIRQTPGKAL | 111 |
| | EWLTQIYWNDEKRYSPSLKNRLTITKDTSKNQVVLTMTNMDPVDT | |
| | ATYYCAHRRGVGNWFDPWGQGTLVTVSS | |
| 4A5 | QVTLKESGGGLVQPGGSLRLSCAASGFTFSSYAMSWVRQAPGKGLE | 124 |
| | WVSAISGSGGSTYYADSVKGRFTISRDNSKNTLYLQMNSLRAEDTA | |
| | VYYCAKDLGATDYWGQGTLVTVSS | |

In some embodiments, the Claudin 18.2 binding agent comprises a variable light chain (VL), wherein the amino acid sequence of the VL is selected from the VL sequences presented in Table 1b. In some embodiments, an anti-Claudin 18.2 binding agent comprises an immunoglobulin light chain variable region having at least 60%, 65%, 70%, 75%, 80%, 85%, 90%, 91%, 92%, 93%, 94%, 95%, 96%, 97%, 98%, 99%, or 100% sequence identity to an amino acid sequence presented in Table 1b.

TABLE 1b
Amino Acid Sequences of Exemplary Light Chain Variable Regions (VL)

| Clone | VL Sequence | SEQ ID NO: |
|---|---|---|
| 1E7 | DIQMTQSPSSVSASVGDRVTITCRASQGISSWLAWYQQKPGKA | 11 |
| | PNLLIYAASGLQSGVPSRFSGSGSGTDFTLTISSLQPEDFASYYC | |
| | QQANSFPFTFGPGTKVDIK | |
| 2A4 | DIQMTQSPSSVSASVGDRVTITCRASQGISSWLAWYQQKPGKA | 26 |
| | PKLLIYAASSLQSGVSSRFSGSESGTDFTLTISSLQPEDFATYYCQ | |
| | QANSFPFTFGPGTKVDIK | |
| 9G2 | EIVLTQSPATLSLSPGERATLSCRASQNVNRYLAWYHQKPGQAP | 41 |
| | RLLIYDAFNRATGIPARFSGSGSGTDFTLTINSLEPEDFAVYYCQ | |
| | QRSDWPLTFGGGTKLEI | |
| 2A10 | DIQLTQSPSFLSASVGDRVTITCRASQDIRNFLAWYQQKPGKAP | 56 |
| | KLLIYAASTLQSGVPSRFSGSGSGTEFALTVSSLQPEDFATYYCQ | |
| | QVNSYPRTFGQGTKVEIK | |
| 12H6 | EIVLTQSPGTLSLSPGERATLSCRASQSVRSSYLAWYQQKPGQA | 71 |
| | PRLLIFGASSRATGIPDRFSGSGSGTDFTLTISRLEPEDFAVYYCQ | |
| | QFGSSLTFGGGTKVEIK | |
| 10D11 | QLVLTQSPSASASLGASVKLTCTLSSGHSSYAIAWHQQQPEKGP | 86 |
| | RYLMKLNSGGSHSKGDGIPDRFSGSSSGAERYLTISSLQSEDEA | |
| | DYYCQTWDTGIRVFGGGTKLTVL | |
| 17F11 | DIQMTQSPSSLSASVGDRVTITCRASQGISNYLAWYQQKPGRVP | 99 |
| | KLLIYAASTLQSGVPSRFSGSGSGTDFTLTISSLQPEDVATYYCQ | |
| | KYISAPFTFGPGTKVDIK | |
| 6B2 | DIQMTQSPSSVSASVGDRVTITCRASQGISSWLAWYQQKPGKA | 112 |
| | PKLLIYAASSLQSGVSSSFSGSASGTEFTLTISNLQPEDFAIYYCQ | |
| | QAFSFPFTFGPGTKVDIK | |
| 4A5 | DIQMTQSPSSVSASVGDRVTITCRASQGISSWLAWYQQKPGKA | 125 |
| | PKLLIYAASSLQSGVPSRFSGSGSGTDFTLTISSLQPEDFATYYCQ | |
| | QANSFPLTFGGGTKVEIK | |

Provided herein are Claudin 18.2 binding agents (e.g., antibodies), wherein the Claudin 18.2 antigen binding domain comprises a variable heavy chain (VH) and a variable light chain, wherein the amino acid sequence of the VH is selected from the VH sequences presented in Table 1a; and the amino acid sequence of the VL is selected from the VL sequences presented in Table 1b.

In some embodiments, the Claudin 18.2 binding agent comprises a heavy chain CDR1, CDR2, and CDR3. In some embodiments, the heavy chain CDR1, CDR2, and CDR3 sequences are selected from the heavy chain CDRs presented in Table 1c. In Table 1d, Kabat CDR definitions are in bold and Chothia CDR definitions are underlined.

TABLE 1c
Amino Acid Sequences of Heavy Chain CDRs

| Clone | CDRH1 | CDRH2 | CDRH3 |
|---|---|---|---|
| 1E7 | TSGVGVG (SEQ ID NO: 1) | QIYWNDEKRYSSSLKS | RRGIGNWFDP |
| | (Kabat) | (SEQ ID NO: 4) (Kabat) | (SEQ ID NO: 6) |
| | GFSLSTSGV (SEQ ID NO: 2) | YWNDE (SEQ ID NO: 5) | |
| | (Chothia) | (Chothia) | |
| | GFSLSTSGVGVG (SEQ ID | | |
| | NO: 3) (Extended) | | |
| 2A4 | TSGVGVG (SEQ ID NO: 16) | QIYWNDEKRYSPSLRN | RRGVGNWFDP |
| | (Kabat) | (SEQ ID NO: 19) (Kabat) | (SEQ ID NO: 21) |
| | GFSLSTSGV (SEQ ID NO: | YWNDE (SEQ ID NO: 20) | |
| | 17) (Chothia) | (Chothia) | |
| | GFSLSTSGVGVG (SEQ ID | | |
| | NO: 18) (Extended) | | |
| 9G2 | NYAMN (SEQ ID NO: 31) | GISGSGGSTYDADSVKG | QGYSFGYFES |
| | (Kabat) | (SEQ ID NO: 34) (Kabat) | (SEQ ID NO: 36) |
| | GFTFSNY (SEQ ID NO: 32) | SGSGGS (SEQ ID NO: 35) | |
| | (Chothia) | (Chothia) | |
| | GFTFSNYAMN (SEQ ID NO: | | |
| | 33) (Extended) | | |
| 2A10 | SYYWN (SEQ ID NO: 46) | RIYTSGSTNYNPSLRS | ASYTYFDSFDI |
| | (Kabat) | (SEQ ID NO: 49) (Kabat) | (SEQ ID NO: 51) |
| | AGSISSY (SEQ ID NO: 47) | YTSGS (SEQ ID NO: 50) | |
| | (Chothia) | (Chothia) | |
| | AGSISSYYWN (SEQ ID NO: | | |
| | 48) (Extended) | | |
| 12H6 | RYWMS (SEQ ID NO: 61) | NIKHDGSEKYYVDSVKG | YYGGPFDY (SEQ |
| | (Kabat) | (SEQ ID NO: 64) KHDGSE | ID NO: 66) |
| | GFTFSRY (SEQ ID NO: 62) | (Kabat) | |
| | (Chothia) | (SEQ ID NO: 65) (Chothia) | |
| | GFTFSRYWMS (SEQ ID NO: | | |
| | 63) (Extended) | | |
| 10D11 | SYAMS (SEQ ID NO: 76) | AISGSGGSTHYADSVKG | EGYVGSWYAPFD |
| | (Kabat) | (SEQ ID NO: 79) SGSGGS | Y (SEQ ID NO: 81) |
| | GFTFSSY (SEQ ID NO: 77) | (Kabat) | |
| | (Chothia) | (SEQ ID NO: 80) (Chothia) | |
| | GFTFSSYAMS (SEQ ID NO: | | |
| | 78) (Extended) | | |
| 17F11 | TSGMCVS (SEQ ID NO: 89) | FIDWDDDKYYNTSLKT | IRGYSGSYDAFDI |
| | (Kabat) | (SEQ ID NO: 92) (Kabat) | (SEQ ID NO: 94) |
| | GVSLSTSGM (SEQ ID NO: | DWDDD (SEQ ID NO: 93) | |
| | 90) (Chothia) | (Chothia) | |
| | GVSLSTSGMCVS (SEQ ID | | |
| | NO: 91) (Extended) | | |
| 6B2 | TSGVGVG (SEQ ID NO: 102) | QIYWNDEKRYSPSLKN | RRGVGNWFDP |
| | (Kabat) | (SEQ ID NO: 105) (Kabat) | (SEQ ID NO: 107) |
| | GFSLSTSGV (SEQ ID NO: | YWNDE (SEQ ID NO: 106) | |
| | 103) (Chothia) | (Chothia) | |
| | GFSLSTSGVGVG (SEQ ID | | |
| | NO: 104) (Extended) | | |
| 4A5 | SYAMS (SEQ ID NO: 115) | AISGSGGSTYYADSVKG | DLGATDY (SEQ ID |
| | (Kabat) | (SEQ ID NO: 118) (Kabat) | NO: 120) |
| | GFTFSSY (SEQ ID NO: 116) | SGSGGS (SEQ ID NO: 119) | |
| | (Chothia) | (Chothia) | |
| | GFTFSSYAMS (SEQ ID NO: | | |
| | 117) (Extended) | | |

In some embodiments, the Claudin 18.2 binding agent comprises a light chain CDR1, CDR2, and CDR3. In some embodiments, the light chain CDR1, CDR2, and CDR3 sequences are selected from the light chain CDRs presented in Table 1d. In Table 1e, Kabat CDR definitions are in bold and Chothia CDR definitions are underlined.

TABLE 1d
Amino Acid Sequences of Light Chain CDRs

| CAR | CDRL1 | CDRL2 | CDRL3 |
|---|---|---|---|
| 1E7 | RASQGISSWLA (SEQ ID NO: | AASGLQS (SEQ ID NO: | QQANSFPFT (SEQ |
| | 7) | 8) | ID NO: 9) |
| 2A4 | RASQGISSWLA (SEQ ID NO: | AASSLQS (SEQ ID NO: | QQANSFPFT (SEQ |
| | 22) | 23) | ID NO: 24) |
| 9G2 | RASQNVNRYLA (SEQ ID | DAFNRAT (SEQ ID NO: | QQRSDWPLT |
| | NO: 37) | 38) | (SEQ ID NO: 39) |
| 2A10 | RASQDIRNFLA (SEQ ID NO: | AASTLQS (SEQ ID NO: | QQVNSYPRT (SEQ |
| | 52) | 53) | ID NO: 54) |
| 12H6 | RASQSVRSSYLA (SEQ ID | GASSRAT (SEQ ID NO: | QQFGSSLT (SEQ |
| | NO: 67) | 68) | ID NO: 69) |
| 10D11 | TLSSGHSSYAIA (SEQ ID | LNSGGSHSKGD (SEQ | QTWDTGIRV |
| | NO: 82) | ID NO: 83) | (SEQ ID NO: 84) |
| 17F11 | RASQGISNYLA (SEQ ID NO: | AASTLQS (SEQ ID NO: | QKYISAPFT (SEQ |
| | 95) | 96) | ID NO: 97) |
| 6B2 | RASQGISSWLA (SEQ ID NO: | AASSLQS (SEQ ID NO: | QQAFSFPFT (SEQ |
| | 108) | 109) | ID NO: 110) |
| 4A5 | RASQGISSWLA (SEQ ID NO: | AASSLQS (SEQ ID NO: | QQANSFPLT (SEQ |
| | 121) | 122) | ID NO: 123) |

The disclosure encompasses modifications to the Claudin 18.2 antibody agents comprising the sequences shown in Tables 1a, 1b, 1c, 1d and 1e, including functionally equivalent Claudin 18.2 antibody agents having modifications which do not significantly affect their properties and variants which have enhanced or decreased activity and/or affinity. For example, the amino acid sequence may be mutated to obtain a Claudin 18.2 antigen binding agent with a desired binding affinity to Claudin 18.2. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the half-life of the antibody in the blood circulation.

Substitution variants have at least one amino acid residue in the antigen binding domain removed and a different residue inserted in its place. In some embodiments, sites of interest for substitutional mutagenesis include the hypervariable regions/CDRs, but FR alterations are also contemplated. Conservative substitutions are shown in Table 2 under the heading of “conservative substitutions.” If such substitutions result in a change in biological activity, then more substantial changes, denominated “exemplary substitutions” in Table 2, or as further described below in reference to amino acid classes, may be introduced and the products screened.

TABLE 2
Amino Acid Substitutions

| Original Residue | | |
|---|---|---|
| (naturally occurring | Conservative | Exemplary |
| amino acid) | Substitutions | Substitutions |
| Ala (A) | Val | Val; Leu; Ile |
| Arg (R) | Lys | Lys; Gln; Asn; Ala |
| Asn (N) | Gln | Gln; His; Asp, Lys; Arg; Ala |
| Asp (D) | Glu | Glu; Asn; Ala |
| Cys (C) | Ser | Ser; Ala |
| Gln (Q) | Asn | Asn; Glu; Ala |
| Glu (E) | Asp | Asp; Gln; Ala |
| Gly (G) | Ala | Ala |
| His (H) | Arg | Asn; Gln; Lys; Arg; Ala |
| Ile (I) | Leu | Leu; Val; Met; Ala; Phe; |
| | | Norleucine; Ala |
| Leu (L) | Ile | Norleucine; Ile; Val; Met; |
| | | Ala; Phe |
| Lys (K) | Arg | Arg; Gln; Asn; Ala |
| Met (M) | Leu | Leu; Phe; Ile; Ala |
| Phe (F) | Tyr | Leu; Val; Ile; Ala; Tyr |
| Pro (P) | Ala | Ala |
| Ser (S) | Thr | Thr; Ala |
| Thr (T) | Ser | Ser; Ala |
| Trp (W) | Tyr | Tyr; Phe; Ala |
| Tyr (Y) | Phe | Trp; Phe; Thr; Ser; Ala |
| Val (V) | Leu | Ile; Leu; Met; Phe; Ala; |
| | | Norleucine |

i. Antibody Fragments

In one aspect, an anti-Claudin 18.2 antibody agent according to any of the above embodiments can be an antibody fragment. An antibody fragment comprises a portion of an intact antibody, such as the antigen binding or variable region of the intact antibody. Antibody fragments include, but are not limited to, Fab, Fab′, Fab′-SH, F(ab′)₂, Fv, diabody, linear antibodies, multispecific formed from antibody fragments antibodies and scFv fragments, and other fragments described below. In some embodiments, the antibody is a full-length antibody, e.g., an intact IgG1 antibody or other antibody class or isotype as described herein. (See, e.g., Hudson et al., Nat. Med., 9: 129-134 (2003); Pluckthun, The Pharmacology of Monoclonal Antibodies, vol. 113, pp. 269-315 (1994); Hollinger et al., Proc. Natd. Acad. Sci. USA, 90: 6444-6448 (1993); WO93/01161; and U.S. Pat. Nos. 5,571,894, 5,869,046, 6,248,516, and 5,587,458). A full-length antibody, intact antibody, or whole antibody is an antibody having a structure substantially similar to a native antibody structure or having heavy chains that contain an Fc region as defined herein. Antibody fragments can be made by various techniques, including but not limited to proteolytic digestion of an intact antibody as well as production by recombinant host cells (e.g., E. coli or phage), as known in the art.

An Fv antibody fragment comprises a complete antigen-recognition and antigen-binding site. This fragment may comprise a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable region (or half of an Fv comprising only three CDRs specific for an antigen) has the ability to recognize and bind antigen, although at a lower affinity than the entire binding site.

A diabody is a small antibody fragment prepared by constructing an sFv fragment with a short linker (e.g., about 5-10 residues) between the V_{H} and V_{L} domains such that interchain but not intra-chain pairing of the V domains is achieved, resulting in a bivalent fragment. Bispecific diabodies are heterodimers of two crossover sFv fragments in which the V_{H} and V_{L} domains of the two antibodies are present on different polypeptide chains (See, e.g., EP 404,097; WO 93/11161; and Hollinger et al, Proc. Natl. Acad. Sci. USA, 90:6444-6448 (1993)).

Domain antibodies (dAbs), which can be produced in fully human form, are the smallest known antigen-binding fragments of antibodies, ranging from about 11 kDa to about 15 kDa. DAbs are the robust variable regions of the heavy and light chains of immunoglobulins (V_{H} and V_{L}, respectively). They are highly expressed in microbial cell culture, show favorable biophysical properties including, for example, but not limited to, solubility and temperature stability, and are well suited to selection and affinity maturation by in vitro selection systems such as, for example, phage display. dAbs are bioactive as monomers and, owing to their small size and inherent stability can be formatted into larger molecules to create drugs with prolonged serum half-lives or other pharmacological activities. (See, e.g., WO 94/25591 and US20030130496).

Fv and scFv are species that have intact combining sites that are devoid of constant regions. Thus, they may be suitable for reduced nonspecific binding during in vivo use. A single-chain Fv (sFv or scFv) is an antibody fragment that comprises the V_{H} and V_{L} antibody domains connected into a single polypeptide chain. The sFv polypeptide can further comprise a polypeptide linker between the V_{H} and V_{L} domains that enable the sFv to form the desired structure for antigen binding (See, e.g., Pluckthun, The Pharmacology of Monoclonal Antibodies, vol. 113, Rosenburg and Moore eds., Springer-Verlag, New York, pp. 269-315 (1994); Borrebaeck 1995, infra). scFv fusion proteins can be constructed to yield fusion of an effector protein at either the amino or the carboxy terminus of an sFv. The antibody fragment also can be a “linear antibody” (See, e.g., U.S. Pat. No. 5,641,870). Such linear antibody fragments can be monospecific or bispecific. Amino acid sequences of exemplary Claudin 18.2-specific scFvs are provided in Table 1e, in which Kabat CDR definitions are in bold and Chothia CDR definitions are underlined.

TABLE 1e
Amino Acid Sequences of Exemplary Claudin 18.2 specific scFvs

| | | SEQ ID |
|---|---|---|
| Clone | scFv Sequence | NO: |
| 1E7 | QITLKESGPTLVKPTQTLTLTCTFSGFSLSTSGVGVGWIRQPPGKAP | 12 |
| | EWLAQIYWNDEKRYSSSLKSRLTITKDTSKNQVVLKMTNMDPVD | |
| | TATYYCAHRRGIGNWFDPWGQGTLVTVSSGGGGSGGGGSGGGGS | |
| | GGGGSDIQMTQSPSSVSASVGDRVTITCRASQGISSWLAWYQQKP | |
| | GKAPNLLIYAASGLQSGVPSRFSGSGSGTDFTLTISSLQPEDFASYY | |
| | CQQANSFPFTFGPGTKVDIK | |
| 2A4 | QITLKESGPTLVKPTQTLTLTCTFSGFSLSTSGVGVGWIRQTPGKAL | 27 |
| | EWLTQIYWNDEKRYSPSLRNRLTITKDTSKNQVVLTMTNMDPVD | |
| | TATYYCAHRRGVGNWEDPWGQGILVTVSSGGGGSGGGGSGGGG | |
| | SGGGGSDIQMTQSPSSVSASVGDRVTITCRASQGISSWLAWYQQK | |
| | PGKAPKLLIYAASSLQSGVSSRFSGSESGTDFTLTISSLQPEDFATYY | |
| | CQQANSFPFTFGPGTKVDIK | |
| 9G2 | EVHLLESGGGLVQPWGSLTLSCAASGFTFSNYAMNWVRQAPGKG | 42 |
| | LEWVSGISGSGGSTYDADSVKGRFTISRDNSKNTLFLQMNSPRAE | |
| | DTAVYYCATQGYSFGYFESWGQGTLVTVSSGGGGSGGGGSGGGG | |
| | SGGGGSEIVLTQSPATLSLSPGERATLSCRASQNVNRYLAWYHQKP | |
| | GQAPRLLIYDAFNRATGIPARFSGSGSGTDFTLTINSLEPEDFAVYY | |
| | CQQRSDWPLTFGGGTKLEIK | |
| 2A10 | QVQLQESGPGLVKPSETLSLTCTVSAGSISSYYWNWIRQPAGKGLE | 57 |
| | WIGRIYTSGSTNYNPSLRSRVTMSVDTSKNQFSLKLSSVTATDTAV | |
| | YYCASASYTYFDSFDIWGQGTMVTVSSGGGGSGGGGSGGGGSGG | |
| | GGSDIQLTQSPSFLSASVGDRVTITCRASQDIRNFLAWYQQKPGKA | |
| | PKLLIYAASTLQSGVPSRFSGSGSGTEFALTVSSLQPEDFATYYCQQ | |
| | VNSYPRTFGQGTKVEIK | |
| 12H6 | EVQLVESGGGLVQPGGSLRLSCAASGFTFSRYWMSWVRQAPGKG | 72 |
| | LEWVANIKHDGSEKYYVDSVKGRFTFSRDNAKTSLYLQMNSLRV | |
| | EDTALYYCARYYGGPFDYWGQGTLVTVSSGGGGSGGGGSGGGGS | |
| | GGGGSEIVLTQSPGTLSLSPGERATLSCRASQSVRSSYLAWYQQKP | |
| | GQAPRLLIFGASSRATGIPDRFSGSGSGTDFTLTISRLEPEDFAVYYC | |
| | QQFGSSLTFGGGTKVEIK | |
| 10D11 | EVQLLESGGGLEQPGGSLRLSCAASGFTFSSYAMSWVRQAPGKGL | 187 |
| | EWVSAISGSGGSTHYADSVKGRFTISRDNARNTLYLQMNSLRAED | |
| | TAVYYCAKEGYVGSWYAPFDYWGQGTLVTVSSGGGGSGGGGSG | |
| | GGGSGGGGSQLVLTQSPSASASLGASVKLTCTLSSGHSSYAIAWH | |
| | QQQPEKGPRYLMKLNSGGSHSKGDGIPDRFSGSSSGAERYLTISSL | |
| | QSEDEADYYCQTWDTGIRVFGGGTKLTVL | |
| 17F11 | QVTLRESGPALVKPTQTLTLTCTVSGVSLSTSGMCVSWIRQPLGKA | 189 |
| | LEWLGFIDWDDDKYYNTSLKTRLTISKDTSKNQVVLTMTNMDPV | |
| | DTATYYCARIRGYSGSYDAFDIWGQGTVVIVSSGGGGSGGGGSGG | |
| | GGSGGGGSDIQMTQSPSSLSASVGDRVTITCRASQGISNYLAWYQQ | |
| | KPGRVPKLLIYAASTLQSGVPSRFSGSGSGTDFTLTISSLQPEDVAT | |
| | YYCQKYISAPFTFGPGTKVDIK | |
| 6B2 | QVTLKESGLTLMKPTQTHTLTCTFSGFSLSTSGVGVGWIRQTPGKA | 191 |
| | LEWLTQIYWNDEKRYSPSLKNRLTITKDTSKNQVVLTMTNMDPV | |
| | DTATYYCAHRRGVGNWFDPWGQGTLVTVSSGGGGSGGGGSGGG | |
| | GSGGGGSDIQMTQSPSSVSASVGDRVTITCRASQGISSWLAWYQQ | |
| | KPGKAPKLLIYAASSLQSGVSSSFSGSASGTEFTLTISNLQPEDFAIY | |
| | YCQQAFSFPFTFGPGTKVDIK | |
| 4A5 | QVTLKESGGGLVQPGGSLRLSCAASGFTFSSYAMSWVRQAPGKGL | 193 |
| | EWVSAISgSGGSTYYADSVKGRFTISRDNSKNTLYLQMNSLRAEDT | |
| | AVYYCAKDLGATDYWGQGTLVTVSSGGGGSGGGGSGGGGSGGG | |
| | GSDIQMTQSPSSVSASVGDRVTITCRASQGISSWLAWYQQKPGKA | |
| | PKLLIYAASSLQSGVPSRFSGSGSGTDFTLTISSLQPEDFATYYCQQ | |
| | ANSFPLTFGGGTKVEIK | |

TABLE 3
Exemplary Nucleic Acid Coding Sequences of Anti-Claudin 18.2 specific scFvs

| | | SEQ ID |
|---|---|---|
| Clone | scFv Nucleic Acid (DNA) Sequence | NO: |
| 1E7 | CAGATTACCCTGAAGGAAAGCGGGCCTACACTGGTGAAGCC | 14 |
| Germlines | AACCCAGACACTGACCCTGACATGCACCTTCAGCGGCTTTTC | |
| IGHV2-5*01 | TCTGAGCACCTCCGGAGTGGGAGTGGGATGGATCAGGCAGC | |
| (94% ID) | CACCTGGCAAGGCACCTGAGTGGCTGGCCCAGATCTACTGGA | |
| IGKV1-12*01 | ACGACGAGAAGCGGTATAGCTCCTCTCTGAAGTCTAGACTGA | |
| (97% ID) | CAATCACCAAGGATACAAGCAAGAACCAGGTGGTGCTGAAG | |
| | ATGACCAATATGGACCCCGTGGATACAGCCACCTACTATTGT | |
| | GCCCACCGGAGAGGCATCGGCAATTGGTTCGACCCTTGGGGC | |
| | CAGGGCACACTGGTGACCGTGAGCTCCGGAGGAGGAGGATC | |
| | CGGCGGAGGAGGCTCTGGCGGCGGCGGCTCCGGCGGCGGCG | |
| | GCTCCGACATCCAGATGACACAGTCCCCATCTAGCGTGTCTG | |
| | CCAGCGTGGGCGATAGGGTGACAATCACCTGCCGCGCCTCTC | |
| | AGGGCATCTCCTCTTGGCTGGCCTGGTACCAGCAGAAGCCAG | |
| | GCAAGGCCCCCAACCTGCTGATCTATGCAGCATCCGGACTGC | |
| | AGTCTGGAGTGCCAAGCAGATTCTCCGGCTCTGGCAGCGGCA | |
| | CCGACTTTACACTGACCATCAGCTCCCTGCAGCCCGAGGATT | |
| | TTGCCTCCTACTATTGTCAGCAGGCCAATTCATTCCCATTCAC | |
| | CTTCGGACCAGGCACAAAAGTGGACATCAAG | |
| 2A4 | CAGATCACCCTGAAGGAATCCGGGCCAACACTGGTGAAGCC | 29 |
| Germlines | CACCCAGACACTGACCCTGACATGCACCTTCAGCGGCTTTAG | |
| IGHV2-5*01 | CCTGTCCACCTCTGGAGTGGGAGTGGGATGGATCAGGCAGAC | |
| (93% ID) | ACCTGGCAAGGCCCTGGAGTGGCTGACCCAGATCTACTGGAA | |
| IGKV1-12*01 | CGACGAGAAGCGGTACAGCCCATCCCTGAGGAATCGCCTGA | |
| (98% ID) | CAATCACCAAGGATACCTCCAAGAACCAGGTGGTGCTGACA | |
| | ATGACCAATATGGACCCCGTGGATACAGCCACCTACTATTGT | |
| | GCCCACCGGAGAGGCGTGGGCAACTGGTTCGACCCTTGGGGC | |
| | CAGGGCATCCTGGTGACAGTGAGCTCCGGAGGAGGAGGATC | |
| | CGGCGGAGGAGGCTCTGGCGGCGGCGGCTCCGGCGGCGGCG | |
| | GCTCCGACATCCAGATGACCCAGTCTCCATCTAGCGTGTCTG | |
| | CCAGCGTGGGCGATCGGGTGACAATCACCTGCAGAGCCTCCC | |
| | AGGGCATCTCCTCTTGGCTGGCCTGGTACCAGCAGAAGCCCG | |
| | GCAAGGCCCCTAAGCTGCTGATCTATGCAGCAAGCTCCCTGC | |
| | AGAGCGGCGTGTCTAGCCGGTTCTCCGGCTCTGAGAGCGGCA | |
| | CAGACTTTACACTGACCATCTCCTCTCTGCAGCCCGAGGATTT | |
| | TGCCACCTACTATTGTCAGCAGGCCAATAGTTTCCCATTCACT | |
| | TTTGGCCCAGGCACTAAGGTGGACATCAAG | |
| 9G2 | GAAGTCCACCTGCTGGAATCTGGGGGAGGACTGGTGCAGCC | 44 |
| Germlines | ATGGGGAAGCCTGACCCTGTCCTGCGCCGCCTCTGGCTTCAC | |
| IGHV3-23*01 | ATTTTCTAACTACGCCATGAATTGGGTGCGGCAGGCACCTGG | |
| (90% ID) | CAAGGGACTGGAGTGGGTGTCCGGAATCTCTGGAAGCGGAG | |
| IGKV3-11*01 | GCTCTACCTATGACGCCGATAGCGTGAAGGGCCGGTTCACCA | |
| (93% ID) | TCAGCAGAGACAACTCCAAGAATACACTGTTTCTGCAGATGA | |
| | ACAGCCCCAGAGCCGAGGATACCGCCGTGTACTATTGTGCCA | |
| | CACAGGGCTACTCCTTCGGCTATTTTGAGTCTTGGGGCCAGG | |
| | GCACCCTGGTGACAGTGAGCTCCGGAGGAGGAGGATCCGGC | |
| | GGAGGAGGCTCTGGCGGCGGCGGCTCCGGCGGCGGCGGCTC | |
| | CGAGATCGTGCTGACCCAGTCCCCAGCCACACTGTCCCTGTC | |
| | TCCAGGAGAGAGGGCCACCCTGTCTTGCAGGGCCAGCCAGA | |
| | ACGTGAATAGGTACCTGGCCTGGTATCACCAGAAGCCAGGAC | |
| | AGGCACCTCGCCTGCTGATCTACGACGCCTTCAACAGGGCAA | |
| | CCGGCATCCCTGCCAGATTCAGCGGCTCCGGCTCTGGCACAG | |
| | ACTTTACCCTGACAATCAATAGCCTGGAGCCAGAGGATTTTG | |
| | CCGTGTACTATTGTCAGCAGAGATCCGACTGGCCCCTGACTT | |
| | TTGGCGGCGGAACTAAACTGGAAATCAAG | |
| 2A10 | CAGGTCCAGCTGCAGGAGTCTGGGCCAGGCCTGGTGAAGCCC | 59 |
| Germlines | TCTGAGACCCTGAGCCTGACCTGCACAGTGTCCGCCGGCTCT | |
| IGHV4-4*07 | ATCAGCTCCTACTATTGGAACTGGATCAGACAGCCTGCAGGC | |
| (95% ID) | AAGGGACTGGAGTGGATCGGAAGGATCTACACATCTGGCAG | |
| IGKV1-9*01 | CACCAACTATAATCCAAGCCTGCGGTCCAGAGTGACAATGTC | |
| (93% ID) | CGTGGACACCTCTAAGAATCAGTTCAGCCTGAAGCTGTCTAG | |
| | CGTGACCGCCACAGATACCGCCGTGTACTATTGTGCCTCCGC | |
| | CTCTTACACATATTTCGACTCCTTTGATATCTGGGGCCAGGGC | |
| | ACAATGGTGACCGTGTCCTCTGGAGGAGGAGGAAGCGGAGG | |
| | AGGAGGAAGCGGCGGCGGCGGCTCTGGCGGCGGCGGCTCCG | |
| | ACATCCAGCTGACCCAGAGCCCATCCTTCCTGTCTGCCAGCG | |
| | TGGGCGACAGGGTGACAATCACCTGCCGCGCCAGCCAGGAT | |
| | ATCCGGAACTTTCTGGCCTGGTACCAGCAGAAGCCCGGCAAG | |
| | GCCCCTAAGCTGCTGATCTATGCAGCAAGCACACTGCAGTCC | |
| | GGAGTGCCATCTAGATTCTCCGGCTCTGGCAGCGGCACAGAG | |
| | TTTGCCCTGACCGTGAGCTCCCTGCAGCCTGAGGATTTTGCCA | |
| | CCTACTATTGTCAGCAGGTGAATTCATACCCAAGAACATTCG | |
| | GGCAGGGGACTAAAGTGGAAATCAAG | |
| 12H6 | GAAGTCCAGCTGGTCGAATCTGGCGGAGGACTGGTGCAGCC | 74 |
| Germlines | AGGAGGATCCCTGAGACTGTCTTGCGCCGCCAGCGGCTTCAC | |
| IGHV3-7*01 | CTTTTCCAGATACTGGATGTCTTGGGTGAGGCAGGCACCTGG | |
| (94% ID) | CAAGGGACTGGAGTGGGTGGCCAACATCAAGCACGACGGCT | |
| IGKV3-20*01 | CCGAGAAGTACTATGTGGATTCTGTGAAGGGCCGGTTCACCT | |
| (96% ID) | TTAGCAGAGACAACGCCAAGACATCCCTGTACCTGCAGATGA | |
| | ACAGCCTGAGAGTGGAGGACACAGCCCTGTACTATTGCGCCA | |
| | GGTACTATGGCGGCCCCTTCGATTATTGGGGCCAGGGCACCC | |
| | TGGTGACAGTGAGCTCCGGAGGAGGAGGAAGCGGCGGAGGA | |
| | GGCAGCGGCGGCGGCGGCTCTGGCGGCGGCGGCAGCGAGAT | |
| | CGTGCTGACCCAGTCCCCAGGCACACTGAGCCTGTCCCCAGG | |
| | AGAGAGGGCCACCCTGAGCTGTCGCGCCTCTCAGAGCGTGCG | |
| | GTCTAGCTACCTGGCCTGGTATCAGCAGAAGCCAGGACAGGC | |
| | ACCTCGCCTGCTGATCTTTGGAGCATCCTCTAGGGCAACCGG | |
| | CATCCCTGACCGGTTCTCCGGATCTGGAAGCGGCACAGACTT | |
| | CACCCTGACAATCTCCCGGCTGGAGCCAGAGGATTTCGCCGT | |
| | GTACTATTGTCAGCAGTTTGGCTCATCTCTGACCTTCGGGGGG | |
| | GGCACAAAAGTGGAAATCAAG | |
| 10D11 | GAAGTCCAGCTGCTGGAGTCAGGAGGAGGACTGGAGCAGCC | 188 |
| | AGGCGGAAGCCTGAGGCTGTCCTGCGCAGCATCTGGCTTCAC | |
| | CTTTAGCTCCTATGCAATGAGCTGGGTGAGACAGGCCCCCGG | |
| | CAAGGGACTGGAGTGGGTGTCCGCCATCTCCGGATCTGGAGG | |
| | ATCCACACACTATGCCGACTCTGTGAAGGGCAGGTTCACCAT | |
| | CTCTCGGGATAACGCCAGAAATACACTGTACCTGCAGATGAA | |
| | CAGCCTGAGGGCAGAGGACACCGCCGTGTACTATTGCGCCAA | |
| | GGAGGGCTACGTGGGCAGCTGGTATGCCCCTTTTGATTACTG | |
| | GGGCCAGGGCACCCTGGTGACAGTGTCTAGCGGAGGAGGAG | |
| | GAAGCGGAGGAGGAGGATCTGGCGGCGGCGGCTCTGGCGGC | |
| | GGCGGCAGCCAGCTGGTGCTGACACAGAGCCCATCCGCCTCT | |
| | GCCAGCCTGGGCGCATCCGTGAAGCTGACCTGTACACTGTCC | |
| | TCTGGCCACAGCTCCTATGCAATCGCATGGCACCAGCAGCAG | |
| | CCAGAGAAGGGACCTCGGTACCTGATGAAGCTGAACAGCGG | |
| | AGGATCCCACTCTAAGGGCGACGGCATCCCCGATAGGTTCTC | |
| | TGGATCTAGCTCCGGAGCAGAGCGGTACCTGACCATCTCTAG | |
| | CCTGCAGAGCGAGGACGAGGCCGATTACTATTGTCAGACATG | |
| | GGACACTGGGATTCGGGTCTTCGGCGGGGGAACAAAACTGA | |
| | CTGTCCTG | |
| 17F11 | CAGGTCACTCTGAGGGAATCTGGCCCAGCCCTGGTGAAGCCC | 190 |
| | ACCCAGACACTGACCCTGACATGCACCGTGTCCGGCGTGAGC | |
| | CTGTCCACCTCTGGCATGTGCGTGAGCTGGATCAGGCAGCCC | |
| | CTGGGCAAGGCCCTGGAGTGGCTGGGCTTCATCGATTGGGAC | |
| | GATGACAAGTACTATAACACATCCCTGAAGACAAGACTGACC | |
| | ATCTCCAAGGACACCTCTAAGAACCAGGTGGTGCTGACAATG | |
| | ACCAATATGGATCCTGTGGACACAGCCACCTACTATTGCGCC | |
| | CGGATCAGAGGCTACAGCGGCTCCTATGATGCCTTTGACATC | |
| | TGGGGCCAGGGCACCGTGGTCATCGTGAGCTCCGGCGGCGGC | |
| | GGCTCTGGAGGAGGAGGAAGCGGAGGAGGAGGAAGCGGGG | |
| | GCGGCGGCTCTGATATCCAGATGACACAGAGCCCATCTAGCC | |
| | TGTCTGCCAGCGTGGGCGACAGGGTGACAATCACCTGCCGCG | |
| | CCAGCCAGGGCATCTCCAATTACCTGGCCTGGTATCAGCAGA | |
| | AGCCCGGCCGGGTGCCTAAGCTGCTGATCTACGCAGCATCTA | |
| | CACTGCAGAGCGGAGTGCCTTCCAGATTCTCCGGATCTGGAA | |
| | GCGGAACCGACTTCACCCTGACCATCTCCTCTCTGCAGCCAG | |
| | AGGACGTGGCCACATACTATTGTCAGAAGTATATCTCCGCAC | |
| | CATTCACATTTGGACCTGGAACTAAAGTGGACATCAAG | |
| 6B2 | CAGGTGACCCTGAAGGAGTCCGGCCTGACACTGATGAAGCCC | 192 |
| | ACACAGACCCACACACTGACCTGCACATTCTCTGGCTTTTCTC | |
| | TGAGCACCTCCGGAGTGGGAGTGGGATGGATCAGACAGACC | |
| | CCCGGCAAGGCCCTGGAGTGGCTGACACAGATCTACTGGAAC | |
| | GACGAGAAGCGGTATTCTCCTAGCCTGAAGAATAGACTGACC | |
| | ATCACAAAGGATACATCCAAGAACCAGGTGGTGCTGACCAT | |
| | GACAAATATGGACCCAGTGGATACCGCCACATACTATTGTGC | |
| | CCACCGGAGAGGAGTGGGAAACTGGTTCGACCCATGGGGAC | |
| | AGGGCACCCTGGTGACAGTGAGCAGCGGAGGAGGAGGCAGC | |
| | GGAGGAGGAGGCTCCGGCGGCGGCGGCTCTGGAGGAGGAGG | |
| | CAGCGACATCCAGATGACCCAGTCCCCTTCTAGCGTGTCCGC | |
| | CTCTGTGGGCGATAGGGTGACCATCACATGCAGGGCAAGCCA | |
| | GGGAATCTCCTCTTGGCTGGCCTGGTACCAGCAGAAGCCAGG | |
| | CAAGGCCCCCAAGCTGCTGATCTATGCAGCAAGCTCCCTGCA | |
| | GAGCGGCGTGTCTAGCTCCTTCAGCGGCTCCGCCTCTGGAAC | |
| | CGAGTTTACCCTGACAATCTCTAATCTGCAGCCTGAGGACTTT | |
| | GCCATCTACTATTGTCAGCAGGCCTTCAGCTTTCCATTCACCT | |
| | TTGGCCCCGGCACAAAGGTGGATATCAAG | |
| 4A5 | CAGGTGACCCTGAAGGAGAGCGGAGGAGGCCTGGTGCAGCC | 194 |
| | TGGCGGCTCCCTGAGGCTGTCTTGCGCAGCAAGCGGCTTCAC | |
| | CTTCAGCTCCTACGCCATGTCCTGGGTGAGACAGGCCCCTGG | |
| | CAAGGGCCTGGAGTGGGTGTCTGCCATCAGCGGCTCCGGAGG | |
| | CTCTACCTACTATGCCGACAGCGTGAAGGGCCGGTTCACAAT | |
| | CTCCAGAGATAACTCTAAGAATACCCTGTACCTGCAGATGAA | |
| | CTCCCTGCGCGCCGAGGACACAGCCGTGTACTATTGCGCCAA | |
| | GGACCTGGGCGCCACCGATTATTGGGGCCAGGGCACACTGGT | |
| | GACCGTGTCTAGCGGCGGCGGCGGCTCTGGAGGAGGAGGCA | |
| | GCGGCGGAGGAGGCTCCGGCGGCGGCGGCTCTGACATCCAG | |
| | ATGACCCAGAGCCCATCCAGCGTGAGCGCCAGCGTGGGCGA | |
| | TAGGGTGACAATCACCTGTAGGGCATCCCAGGGAATCAGCTC | |
| | CTGGCTGGCCTGGTACCAGCAGAAGCCAGGCAAGGCCCCCA | |
| | AGCTGCTGATCTATGCAGCATCTAGCCTGCAGAGCGGAGTGC | |
| | CATCCCGGTTCTCCGGCTCTGGCAGCGGAACAGACTTTACAC | |
| | TGACCATCTCCTCTCTGCAGCCTGAGGATTTTGCCACCTACTA | |
| | TTGTCAGCAGGCCAATAGCTTCCCACTGACATTTGGCGGCGG | |
| | CACCAAGGTGGAGATCAAG | |

In some embodiments, the Claudin 18.2 antigen binding domain comprises a scFv comprising a light chain variable (VL) region and a heavy chain variable (VH) region of a Claudin 18.2-specific monoclonal antibody joined by a flexible linker. Single chain variable region fragments may be made by linking light and/or heavy chain variable regions by using a linking peptide An example of a linking peptide is the GS linker having the amino acid sequence (GGGGS)ₓ wherein x is 1, 2, 3, 4, or 5 (SEQ ID NO: 215) (GGGGS (GS sequence (1)) is SEQ ID NO: 163). In some embodiments, x is 6, 7, 8, 9, 10, 11, 12, 13, 14, 15, or any integer less than about 20. In some embodiments, the linker is (GGGGS)₄ (SEQ ID NO: 135). In general, linkers can be short, flexible polypeptides, which in some embodiments are comprised of about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

In exemplary embodiments, provided herein are Claudin 18.2 antigen binding domains comprising: a VH region comprising a VH CDR1, VH CDR2, and VH CDR3 of the VH sequence shown in Table 1a and/or a VL region comprising VL CDR1, VL CDR2, and VL CDR3 of the VL sequence shown in Table 1b. In some embodiments, the VH and VL are linked together by a linker, e.g. a linker listed in Table 7a, e.g. a “GS” linker comprising only G (glycine) and S (serine) residues. In some embodiments the linker comprises the amino acid sequence GGGGSGGGGSGGGGSGGGGS (SEQ ID NO: 135) (“(GGGGS)₄”). In some embodiments the linker may be encoded by a DNA sequence comprising

(SEQ ID NO: 151)
GGCGGTGGAGGCTCCGGAGGGGGGGGCTCTGGCGGAGGGGGCTCC.

In some embodiments, the linker may be encoded by a DNA sequence comprising GGCGGCGGCGGCTCTGGAGGAGGAGGCAGCGGCGGAGGAGGCTCCGGAGGCGGCGGC TCT (SEQ ID NO: 152). In some embodiments the linker comprises the amino acid sequence GGGGSGGGGSGGGGS (SEQ ID NO: 162). In some embodiments the linker is a scFv Whitlow linker, which may comprise the amino acid sequence GSTSGSGKPGSGEGSTKG (SEQ ID NO: 164). The scFv Whitlow linker may be encoded by a DNA sequence comprising GGGTCTACATCCGGCTCCGGGAAGCCCGGAAGTGGCGAAGGTAGTACAAAGGGG (SEQ ID NO: 165). In some embodiments, the VH and VL sequences of the scFv's disclosed can be oriented with the VH sequence being located at the N-terminus of the scFv and followed by a linker and then the VL sequence, while in other embodiments the scFv can be oriented with the VL sequence at the N-Terminus and followed by a linker and then the VH sequence.

ii. Chimeric and Humanized Antibodies

In some embodiments, an anti-Claudin 18.2 antibody agent is or comprises a monoclonal antibody, including a chimeric, humanized or human antibody.

In some embodiments, an anti-Claudin 18.2 antibody agent provided herein can be a chimeric antibody (See, e.g., U.S. Pat. No. 4,816,567; and Morrison et al., Proc. Natl. Acad. Sci. USA, 81: 6851-6855 (1984)). A chimeric antibody can be an antibody in which a portion of the heavy and/or light chain is derived from a particular source or species, while the remainder of the heavy and/or light chain is derived from a different source or species. In one example, a chimeric antibody can comprise a non-human variable region (e.g., a variable region derived from a mouse, rat, hamster, rabbit, or non-human primate, such as a monkey) and a human constant region. In a further example, a chimeric antibody can be a “class switched” antibody in which the class or subclass has been changed from that of the parent antibody. Chimeric antibodies include antigen-binding fragments thereof.

In some embodiments, a chimeric antibody can be a humanized antibody (See, e.g., Almagro and Fransson, Front. Biosci., 13: 1619-1633 (2008); Riechmann et al., Nature, 332:323-329 (1988); Queen et al., Proc. Natl Acad. Sci. USA 86: 10029-10033 (1989); U.S. Pat. Nos. 5,821,337, 7,527,791, 6,982,321, and 7,087,409; Kashmiri et al., Methods 36:25-34 (2005); Padlan, Mol. Immunol, 28:489-498 (1991); Dall'Acqua et al., Methods, 36:43-60 (2005); Osboum et al., Methods, 36:61-68 (2005); and Klimka et al., Br. J. Cancer, 83: 252-260 (2000)). A humanized antibody is a chimeric antibody comprising amino acid residues from non-human hypervariable regions and amino acid residues from human FRs. In certain embodiments, a humanized antibody will comprise substantially all of at least one, and typically two, variable domains, in which all or substantially all of the hypervariable regions (e.g., CDRs) correspond to those of a non-human antibody, and all or substantially all of the FRs correspond to those of a human antibody. A humanized antibody optionally may comprise at least a portion of an antibody constant region derived from a human antibody.

A non-human antibody can be humanized to reduce immunogenicity to humans, while retaining the specificity and affinity of the parental non-human antibody. A humanized antibody can comprise one or more variable domains comprising one or more CDRs, or portions thereof, derived from a non-human antibody. A humanized antibody can comprise one or more variable domains comprising one or more FRs, or portions thereof, derived from human antibody sequences. A humanized antibody can optionally comprise at least a portion of a human constant region. In some embodiments, one or more FR residues in a humanized antibody are substituted with corresponding residues from a non-human antibody (e.g., the antibody from which the CDR residues are derived), to restore or improve antibody specificity or affinity.

Human framework regions that may be used for humanization include but are not limited to: framework regions selected using a “best-fit” method; framework regions derived from the consensus sequence of human antibodies of a particular subgroup of light or heavy chain variable regions; human mature (somatically mutated) framework regions or human germline framework regions; and framework regions derived from screening FR libraries (See, e.g., Sims et al., J. Immunol, 151:2296 (1993); Carter et al., Proc. Natl. Acad. Sci. USA, 89:4285 (1992); Presta et al., J. Immunol, 151:2623 (1993); Baca et al., J. Biol. Chem., 272: 10678-10684 (1997); and Rosok et al., J. Biol. Chem., 271:22611-22618 (1996)).

iii. Human Antibodies

In some embodiments, an anti-Claudin 18.2 antibody agent provided herein is a human antibody. Human antibodies can be produced using various techniques known in the art (See, e.g., van Dijk and van de Winkel, Curr. Opin. Pharmacol, 5: 368-74 (2001); and Lonberg, Curr. Opin. Immunol, 20:450-459 (2008)). A human antibody can be one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies may be prepared by administering an immunogen (e.g., a Claudin 18.2 protein) to a transgenic animal that has been modified to produce intact human antibodies or intact antibodies with human variable regions in response to antigenic challenge (See, e.g., Lonberg, Nat. Biotech., 23: 1117-1125 (2005); U.S. Pat. Nos. 6,075,181, 6,150,584, 5,770,429, and 7,041,870; and U.S. Pat. App. Pub. No. US 2007/0061900). Human variable regions from intact antibodies generated by such animals may be further modified, e.g., by combining with a different human constant region.

Human antibodies can also be made by hybridoma-based methods. For example, human antibodies can be produced from human myeloma and mouse-human heteromyeloma cell lines, using human B-cell hybridoma technology, and other methods (See, e.g., Kozbor, J. Immunol, 133: 3001 (1984); Brodeur et al., Monoclonal Antibody Production Techniques and Applications, pp. 51-63 (1987); Boerner et al., J. Immunol, 147: 86 (1991); Li et al., Proc. Natl. Acad. Sci. USA, 103:3557-3562 (2006); U.S. Pat. No. 7,189,826; Ni, Xiandai Mianyixue, 26(4): 265-268 (2006); Vollmers and Brandlein, Histology and Histopathology, 20(3): 927-937 (2005); and Vollmers and Brandlein, Methods and Findings in Experimental and Clinical Pharmacology, 27(3): 185-91 (2005)). Human antibodies may also be generated by isolating Fv clone variable domain sequences selected from human-derived phage display libraries. Such variable domain sequences may then be combined with a desired human constant region.

Modifications of the oligosaccharide in an antibody can be made, for example, to create antibody variants with certain improved properties. For example, antibody glycosylation variants can have improved CDC function. In some embodiments, the present disclosure can contemplate an antibody variant that possesses some but not all effector functions, which make it a desirable candidate for applications in which the half-life of the antibody in vivo is important yet certain effector functions (such as complement) are unnecessary or deleterious. In vitro and/or in vivo cytotoxicity assays can be conducted to confirm the reduction/depletion of CDC activities.

iv. Antibody Derivatives

In some embodiments, an antibody agent provided herein may be further modified to contain additional nonproteinaceous moieties that are known in the art and readily available. The moieties suitable for derivatization of the antibody can include but are not limited to water soluble polymers. Non-limiting examples of water soluble polymers can include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethyl ene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water.

The polymer may be of any molecular weight, and may be branched or unbranched. The number of polymers attached to the antibody may vary, and if two or more polymers are attached, they can be the same or different molecules.

In some embodiments, conjugates of an antibody and nonproteinaceous moiety that may be selectively heated by exposure to radiation are provided. In some embodiments, the nonproteinaceous moiety can be a carbon nanotube (See, e.g., Kam et al., Proc. Natd. Acad. Sci. USA, 102: 11600-11605 (2005)). The radiation may be of any wavelength, and can include, but is not limited to, wavelengths that do not harm ordinary cells, but which heat the nonproteinaceous moiety to a temperature at which cells proximal to the antibody-nonproteinaceous moiety are killed.

A Claudin 18.2 binding agent (e.g., a molecule comprising an antigen binding domain) is said to “specifically bind” its target antigen (e.g., human, cyno or mouse Claudin 18.2) when the dissociation constant (Kd) is ˜1 nM. The antigen binding domain specifically binds antigen with “high affinity” when the Kd is 1-5 nM, and with “very high affinity” when the Kd is 0.1-0.5 nM. In one embodiment, the antigen binding domain has a Kd of ˜1 nM. In one embodiment, the off-rate is <1×10⁻⁵. In other embodiments, the antigen binding domains will bind to human Claudin 18.2 with a Kd of between about 1×10⁻⁷ M and 1×10⁻¹² M, and in yet another embodiment the antigen binding domains will bind with a Kd between about 1×10⁻⁵ M and 1×10⁻¹² M.

As provided herein, the antigen binding domains of the present disclosure specifically bind mammalian Claudin 18.2 (e.g., human Claudin 18.2, cyno Claudin 18.2 or mouse Claudin 18.2). In certain embodiments, a Claudin 18.2 antigen binding domain of the present disclosure binds mammalian Claudin 18.2 with a Kd of less than 1×10⁻⁶ M, less than 1×10⁻⁷ M, less than 1×10⁻⁸ M, or less than 1×10⁻⁹ M. In one particular embodiment, the Claudin 18.2 antigen binding domains binds mammalian Claudin 18.2 (e.g., human Claudin 18.2, cyno Claudin 18.2 or mouse Claudin 18.2) with a Kd of less than 1×10⁻⁷ M. In another embodiment, the Claudin 18.2 antigen binding domains binds mammalian Claudin 18.2 (e.g., human Claudin 18.2, cyno Claudin 18.2 or mouse Claudin 18.2) with a Kd of less than 1×10⁻⁸ M. In some embodiments, the Claudin 18.2 antigen binding domains binds mammalian Claudin 18.2 (e.g., human Claudin 18.2, cyno Claudin 18.2) with a Kd of about 1×10⁻⁷ M, about 2×10⁻⁷ M, about 3×10⁻⁷ M, about 4×10⁻⁷ M, about 5×10⁻⁷ M, about 6×10⁻⁷ M, about 7×10⁻⁷ M, about 8×10⁻⁷ M, about 9×10⁻⁷ M, about 1×10⁻⁸ M, about 2×10⁻⁸ M, about 3×10⁻⁸ M, about 4×10⁻⁸ M, about 5×10⁻⁸ M, about 6×10⁻⁸ M, about 7×10⁻⁸ M, about 8×10⁻⁸ M, about 9×10⁻⁸ M, about 1×10⁻⁸ M, about 2×10⁻⁹ M, about 3×10⁻⁹ M, about 4×10⁻⁹ M, about 5×10⁻⁹ M, about 6×10⁻⁹ M, about 7×10⁻⁹ M, about 8×10⁻⁹ M, about 9×10⁻⁹ M, about 1×10⁻¹⁰ M, or about 5×10⁻¹⁰ M. In certain embodiments, the Kd is calculated as the quotient of K_{off}/Kₒₙ, and the Kₒₙ and K_{off} are determined using a monovalent antibody, such as a Fab fragment, as measured by, e.g., BIAcore® surface plasmon resonance technology. In other embodiments, the Kd is calculated as the quotient of K_{off}/Kₒₙ, and the Kₒₙ and K_{off} are determined using a bivalent antibody, such as a Fab fragment, as measured by, e.g., BIAcore® surface plasmon resonance technology.

In some embodiments, the Claudin 18.2 antigen binding domain binds mammalian Claudin 18.2 (e.g., human Claudin 18.2, cyno Claudin 18.2 or mouse Claudin 18.2) with an association rate (kₒₙ) of less than 1×10⁻⁴ M⁻¹ s⁻¹, less than 2×10⁴ M⁻¹ s⁻¹, less than 3×10⁴ M⁻¹ s⁻¹, less than 4×10⁻⁴ M⁻¹ s⁻¹, less than 5×10⁻⁴ M⁻¹ s⁻¹, less than 7×10⁻⁴ M⁻¹ s⁻¹, less than 8×10⁻⁴ M⁻¹ s⁻¹, less than 9×10⁻⁴ M⁻¹ s⁻¹, less than 1×10⁻⁵ M⁻¹ s⁻¹, less than 2×10⁻⁵ M⁻¹ s⁻¹, less than 3×10⁻⁵ M⁻¹ s⁻¹, less than 4×10⁻⁵ M⁻¹ s⁻¹, less than 5×10⁻⁵ M⁻¹ s⁻¹, less than 6×10⁻⁵ M⁻¹ s⁻¹, less than 7×10⁻⁵ M⁻¹ s⁻¹, less than 8×10⁻⁵ M⁻¹ s⁻¹, less than 9×10⁻⁵ M⁻¹ s⁻¹, less than 1×10⁻⁶ M⁻¹ s⁻¹, less than 2×10⁻⁶ M⁻¹ s⁻¹, less than 3×10⁻⁶ M⁻¹ s⁻¹, less than 4×10⁻⁶ M⁻¹ s⁻¹, less than 5×10⁻⁶ M⁻¹ s⁻¹, less than 6×10⁻⁶ M⁻¹ s⁻¹, less than 7×10⁻⁶ M⁻¹ s⁻¹, less than 8×10⁻⁶ M⁻¹ s⁻¹, less than 9×10⁻⁶ M⁻¹ s⁻¹, or less than 1×10⁻⁷ M⁻¹ s⁻¹. In certain embodiments, the kₒₙ is determined using a monovalent antibody, such as a Fab fragment, as measured by, e.g., BIAcore® surface plasmon resonance technology. In other embodiments, the kₒₙ is determined using a bivalent antibody as measured by, e.g., BIAcore® surface plasmon resonance technology.

In some embodiments, the Claudin 18.2 antigen binding domain binds mammalian Claudin 18.2 (e.g., human Claudin 18.2, cyno Claudin 18.2 or mouse Claudin 18.2) with an dissociation rate (k_{off}) of less than 1×10⁻² s⁻¹, less than 2×10⁻² s⁻¹, less than 3×10⁻² s⁻¹, less than 4×10⁻² s⁻¹, less than 5×10⁻² s⁻¹, less than 6×10⁻² s⁻¹, less than 7×10⁻² s⁻¹, less than 8×10⁻² s⁻¹, less than 9×10⁻² s⁻¹, less than 1×10⁻³ s⁻¹, less than 2×10⁻³ s⁻¹, less than 3×10⁻³ s⁻¹, less than 4×10⁻³ s⁻¹, less than 5×10⁻³ s⁻¹, less than 6×10⁻³ s⁻¹, less than 7×10⁻³ s⁻¹, less than 8×10⁻³ s⁻¹, less than 9×10⁻³ s⁻¹, less than 1×10⁻⁴ s⁻¹, less than 2×10⁻⁴ s⁻¹, less than 3×10⁻⁴ s⁻¹, less than 4×10⁻⁴ s⁻¹, less than 5×10⁻⁴ s⁻¹, less than 6×10⁻⁴ s⁻¹ less than 7×10⁻⁴ s⁻¹, less than 8×10⁻⁴ s⁻¹, less than 9×10⁻⁴ s⁻¹, less than 1×10⁻⁵ s⁻¹, or less than 5×10⁻⁴ s⁻¹. In certain embodiments, the k_{off} is determined using a monovalent antibody, such as a Fab fragment, as measured by, e.g., BIAcore® surface plasmon resonance technology. In other embodiments, the k_{off} is determined using a bivalent antibody as measured by, e.g., BIAcore® surface plasmon resonance technology.

### II. Chimeric Antigen Receptors

As used herein, chimeric antigen receptors (CARs) are proteins that specifically recognize target antigens (e.g., target antigens on cancer cells). When bound to the target antigen, the CAR may activate the immune cell to attack and destroy the cell bearing that antigen (e.g., the cancer cell). CARs may also incorporate costimulatory or signaling domains to increase their potency. See Krause et al., J. Exp. Med., Volume 188, No. 4, 1998 (619-626); Finney et al., Journal of Immunology, 1998, 161: 2791-2797, Song et al., Blood 119:696-706 (2012); Kalos et al., Sci. Transl. Med. 3:95 (2011); Porter et al., N. Engl. J. Med. 365:725-33 (2011), and Gross et al., Annu. Rev. Pharmacol. Toxicol. 56:59-83 (2016); U.S. Pat. Nos. 7,741,465, and 6,319,494.

Chimeric antigen receptors described herein comprise an extracellular domain, a transmembrane domain, and an intracellular domain, wherein the extracellular domain comprises a Claudin 18.2 antigen binding domain that specifically binds to Claudin 18.2. In some embodiments, the Claudin 18.2 specific CAR comprises the following elements from 5′ to 3′: a signal sequence, a Claudin 18.2 antigen binding domain (e.g., an anti-Claudin 18.2 scFv), a hinge and transmembrane region, and one or more successive signaling domains. In certain embodiments, the Claudin 18.2 specific CAR disclosed herein comprises the following elements from 5′ to 3′: a CD8α signal sequence, a Claudin 18.2 scFv comprising a Claudin 18.2 variable heavy chain and/or variable light chain described herein, a CD8α hinge and transmembrane region, a 41BB cytoplasmic signaling domain, and a CD3ζ cytoplasmic signaling domain. In some embodiments, the Claudin 18.2 specific CAR comprises the following elements from 5′ to 3′: a Claudin 18.2 antigen binding domain (e.g., an anti-Claudin 18.2 scFv), a hinge and transmembrane region, and one or more successive signaling domains. In certain embodiments, the Claudin 18.2 specific CAR disclosed herein comprises the following elements from 5′ to 3′: a Claudin 18.2 scFv comprising a Claudin 18.2 variable heavy chain and/or variable light chain described herein, a CD8α hinge and transmembrane region, a 41BB cytoplasmic signaling domain, and a CD3ζ cytoplasmic signaling domain. Table 7a lists exemplary CAR component amino acid sequences.

In some embodiments, the Claudin 18.2 specific CARs further comprise one or more safety switches and/or monoclonal antibody specific-epitopes.

a. Antigen Binding Domain

As discussed above, the Claudin 18.2 CARs described herein comprise an antigen binding domain. An “antigen binding domain” as used herein means any polypeptide that binds a specified target antigen, for example the specified target antigen can be the Claudin 18.2 (Claudin 18.2) protein or fragment thereof (referred to interchangeably herein as a “Claudin 18.2 antigen”, “Claudin 18.2 target antigen”, or “Claudin 18.2 target”). In some embodiments, the antigen binding domain binds to a Claudin 18.2 antigen on a tumor cell. In some embodiments, the antigen binding domain binds to a Claudin 18.2 antigen on a cell involved in a hyperproliferative disease.

In some embodiments, the antigen binding domain comprises a variable heavy chain, variable light chain, and/or one or more CDRs described herein. In some embodiments, the antigen binding domain is a single chain variable fragment (scFv), comprising light chain CDRs CDR1, CDR2 and CDR3, and heavy chain CDRs CDR1, CDR2 and CDR3.

In some embodiments, Claudin 18.2 specific CARs comprise a VH amino acid sequence shown in Table 1a. In some embodiments, Claudin 18.2 specific CARs comprise a VL amino acid sequence shown in Table 1b. In some embodiments, Claudin 18.2 specific CARs comprise a heavy chain CDR1, CDR2, CDR3 amino acid sequence shown in Table 1d. In some embodiments, Claudin 18.2 specific CARs comprise a light chain CDR1, CDR2, CDR3 amino acid sequence shown in Table 1e.

Variants of the antigen binding domains (e.g., variants of the CDRs, VH and/or VL) are also within the scope of the disclosure, e.g., variable light and/or variable heavy chains that each have at least 70-80%, 80-85%, 85-90%, 90-95%, 95-97%, 97-99%, or above 99% identity to the amino acid sequences of the antigen binding domain sequences described herein. In some instances, such molecules include at least one heavy chain and one light chain, whereas in other instances the variant forms contain two variable light chains and two variable heavy chains (or subparts thereof). A skilled artisan will be able to determine suitable variants of the antigen binding domains as set forth herein using well-known techniques. In certain embodiments, one skilled in the art can identify suitable areas of the molecule that may be changed without destroying activity by targeting regions not believed to be important for activity.

In certain embodiments, the polypeptide structure of the antigen binding domains is based on antibodies, including, but not limited to, monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the antigen binding domain comprises or consists of avimers.

A Claudin 18.2 antigen binding domain is said to be “selective” when it binds to one target more tightly than it binds to a second target.

In some embodiments, the Claudin 18.2 antigen binding domain is a scFv. In some embodiments, the Claudin 18.2 specific CAR comprises an scFv provided in Table 1c.

In some embodiments, the Claudin 18.2 specific CAR comprises a leader or signal peptide; in some embodiments the leader peptide comprises an amino acid sequence that is at least about 75%, at least about 80%, at least about 85%, at least about 90%, at least about 95%, at least about 96%, at least about 97%, at least about 98%, at least about 99%, or 100% identical to the amino acid sequence MALPVTALLLPLALLLHAARP (SEQ ID NO: 134). In some embodiments, the leader (signal) peptide comprises the amino acid sequence of SEQ ID NO: 134. In some embodiments, the leader (signal) peptide is encoded by a nucleic acid sequence comprising:

(SEQ ID NO: 166)
ATGGCACTCCCCGTAACTGCTCTGCTGCTGCCGTTGGCATTGCTCCTGC
ACGCCGCACGCCCG.

In other embodiments, the disclosure relates to isolated polynucleotides encoding any one of the Claudin 18.2 antigen binding domains described herein. In some embodiments, the disclosure relates to isolated polynucleotides encoding a Claudin 18.2 CAR described in Table 10. Also provided herein are vectors comprising the polynucleotides, and methods of making the same.

TABLE 10
Polynucleotide Sequences encoding exemplary Claudin 18.2 targeting CARs

| SEQ | | |
|---|---|---|
| ID | CAR | |
| NO | Structure | Nucleotide (DNA) Sequence |
| 15 | CD8α signal | ATGGCTCTGCCTGTGACTGCCCTGCTGCTGCCCCTGGCC |
| | sequence, 1E7 | CTGCTGCTGCACGCCGCACGACCTCAGATTACCCTGAAG |
| | scFv, CD8α | GAAAGCGGGCCTACACTGGTGAAGCCAACCCAGACACT |
| | hinge and | GACCCTGACATGCACCTTCAGCGGCTTTTCTCTGAGCAC |
| | transmembrane | CTCCGGAGTGGGAGTGGGATGGATCAGGCAGCCACCTG |
| | regions, | GCAAGGCACCTGAGTGGCTGGCCCAGATCTACTGGAAC |
| | 41BB | GACGAGAAGCGGTATAGCTCCTCTCTGAAGTCTAGACTG |
| | cytoplasmic | ACAATCACCAAGGATACAAGCAAGAACCAGGTGGTGCT |
| | signaling | GAAGATGACCAATATGGACCCCGTGGATACAGCCACCT |
| | domain, CD3ζ | ACTATTGTGCCCACCGGAGAGGCATCGGCAATTGGTTCG |
| | cytoplasmic | ACCCTTGGGGCCAGGGCACACTGGTGACCGTGAGCTCCG |
| | signaling | GAGGAGGAGGATCCGGCGGAGGAGGCTCTGGCGGCGGC |
| | domain | GGCTCCGGCGGCGGCGGCTCCGACATCCAGATGACACA |
| | | GTCCCCATCTAGCGTGTCTGCCAGCGTGGGCGATAGGGT |
| | | GACAATCACCTGCCGCGCCTCTCAGGGCATCTCCTCTTG |
| | | GCTGGCCTGGTACCAGCAGAAGCCAGGCAAGGCCCCCA |
| | | ACCTGCTGATCTATGCAGCATCCGGACTGCAGTCTGGAG |
| | | TGCCAAGCAGATTCTCCGGCTCTGGCAGCGGCACCGACT |
| | | TTACACTGACCATCAGCTCCCTGCAGCCCGAGGATTTTG |
| | | CCTCCTACTATTGTCAGCAGGCCAATTCATTCCCATTCAC |
| | | CTTCGGACCAGGCACAAAAGTGGACATCAAGACAACTA |
| | | CCCCAGCACCTAGGCCACCTACACCTGCACCAACCATCG |
| | | CCAGCCAGCCTCTGTCCCTGAGACCAGAGGCCTGTAGGC |
| | | CAGCAGCAGGAGGAGCAGTGCACACCCGGGGCCTGGAC |
| | | TTCGCCTGCGATATCTACATCTGGGCACCACTGGCAGGA |
| | | ACATGTGGCGTGCTGCTGCTGTCCCTGGTCATCACCCTG |
| | | TACTGCAAGAGAGGCAGGAAGAAGCTGCTGTATATCTTC |
| | | AAGCAGCCCTTCATGAGACCCGTGCAGACAACCCAGGA |
| | | GGAGGACGGCTGCAGCTGTAGGTTCCCAGAGGAGGAGG |
| | | AGGGAGGATGTGAGCTGCGCGTGAAGTTTTCCCGGTCTG |
| | | CCGATGCACCTGCATACCAGCAGGGACAGAACCAGCTG |
| | | TATAACGAGCTGAATCTGGGCCGGAGAGAGGAGTACGA |
| | | CGTGCTGGATAAGAGGAGGGGAAGGGACCCTGAGATGG |
| | | GAGGCAAGCCTCGGAGAAAGAACCCACAGGAGGGCCTG |
| | | TACAATGAGCTGCAGAAGGACAAGATGGCCGAGGCCTA |
| | | TAGCGAGATCGGCATGAAGGGAGAGAGGCGCCGGGGCA |
| | | AGGGACACGATGGCCTGTATCAGGGCCTGTCAACCGCTA |
| | | CAAAAGATACCTACGATGCTCTGCACATGCAGGCTCTGC |
| | | CACCAAGA |
| 30 | CD8α signal | ATGGCCCTGCCTGTCACCGCACTGCTGCTGCCCCTGGCT |
| | sequence, 2A4 | CTGCTGCTGCACGCCGCAAGACCTCAGATCACCCTGAAG |
| | scFv, CD8α | GAATCCGGGCCAACACTGGTGAAGCCCACCCAGACACT |
| | hinge and | GACCCTGACATGCACCTTCAGCGGCTTTAGCCTGTCCAC |
| | transmembrane | CTCTGGAGTGGGAGTGGGATGGATCAGGCAGACACCTG |
| | regions, | GCAAGGCCCTGGAGTGGCTGACCCAGATCTACTGGAAC |
| | 41BB | GACGAGAAGCGGTACAGCCCATCCCTGAGGAATCGCCT |
| | cytoplasmic | GACAATCACCAAGGATACCTCCAAGAACCAGGTGGTGC |
| | signaling | TGACAATGACCAATATGGACCCCGTGGATACAGCCACCT |
| | domain, CD3ζ | ACTATTGTGCCCACCGGAGAGGCGTGGGCAACTGGTTCG |
| | cytoplasmic | ACCCTTGGGGCCAGGGCATCCTGGTGACAGTGAGCTCCG |
| | signaling | GAGGAGGAGGATCCGGCGGAGGAGGCTCTGGCGGCGGC |
| | domain | GGCTCCGGCGGCGGCGGCTCCGACATCCAGATGACCCA |
| | | GTCTCCATCTAGCGTGTCTGCCAGCGTGGGCGATCGGGT |
| | | GACAATCACCTGCAGAGCCTCCCAGGGCATCTCCTCTTG |
| | | GCTGGCCTGGTACCAGCAGAAGCCCGGCAAGGCCCCTA |
| | | AGCTGCTGATCTATGCAGCAAGCTCCCTGCAGAGCGGCG |
| | | TGTCTAGCCGGTTCTCCGGCTCTGAGAGCGGCACAGACT |
| | | TTACACTGACCATCTCCTCTCTGCAGCCCGAGGATTTTGC |
| | | CACCTACTATTGTCAGCAGGCCAATAGTTTCCCATTCAC |
| | | TTTTGGCCCAGGCACTAAGGTGGACATCAAGACTACTAC |
| | | TCCCGCTCCTAGGCCACCTACACCTGCACCAACCATCGC |
| | | CAGCCAGCCTCTGTCCCTGAGACCAGAGGCCTGTAGGCC |
| | | AGCAGCAGGAGGAGCAGTGCACACCCGGGGCCTGGACT |
| | | TCGCCTGCGATATCTACATCTGGGCACCACTGGCAGGAA |
| | | CATGTGGCGTGCTGCTGCTGTCCCTGGTCATCACCCTGT |
| | | ACTGCAAGAGAGGCAGGAAGAAGCTGCTGTATATCTTC |
| | | AAGCAGCCCTTCATGAGACCCGTGCAGACAACCCAGGA |
| | | GGAGGACGGCTGCAGCTGTAGGTTCCCAGAGGAGGAGG |
| | | AGGGAGGATGTGAGCTGCGCGTGAAGTTTTCCCGGTCTG |
| | | CCGATGCACCTGCATACCAGCAGGGACAGAACCAGCTG |
| | | TATAACGAGCTGAATCTGGGCCGGAGAGAGGAGTACGA |
| | | CGTGCTGGATAAGAGGAGGGGAAGGGACCCTGAGATGG |
| | | GAGGCAAGCCTCGGAGAAAGAACCCACAGGAGGGCCTG |
| | | TACAATGAGCTGCAGAAGGACAAGATGGCCGAGGCCTA |
| | | TAGCGAGATCGGCATGAAGGGAGAGAGGCGCCGGGGCA |
| | | AGGGACACGATGGCCTGTATCAGGGCCTGTCAACCGCTA |
| | | CAAAAGATACCTACGATGCTCTGCACATGCAGGCTCTGC |
| | | CACCAAGA |
| 45 | CD8α signal | ATGGCTCTGCCTGTCACTGCTCTGCTGCTGCCTCTGGCTC |
| | sequence, 9G2 | TGCTGCTGCACGCTGCTCGCCCTGAAGTCCACCTGCTGG |
| | scFv, CD8α | AATCTGGGGGAGGACTGGTGCAGCCATGGGGAAGCCTG |
| | hinge and | ACCCTGTCCTGCGCCGCCTCTGGCTTCACATTTTCTAACT |
| | transmembrane | ACGCCATGAATTGGGTGCGGCAGGCACCTGGCAAGGGA |
| | regions, | CTGGAGTGGGTGTCCGGAATCTCTGGAAGCGGAGGCTCT |
| | 41BB | ACCTATGACGCCGATAGCGTGAAGGGCCGGTTCACCATC |
| | cytoplasmic | AGCAGAGACAACTCCAAGAATACACTGTTTCTGCAGATG |
| | signaling | AACAGCCCCAGAGCCGAGGATACCGCCGTGTACTATTGT |
| | domain, CD3ζ | GCCACACAGGGCTACTCCTTCGGCTATTTTGAGTCTTGG |
| | cytoplasmic | GGCCAGGGCACCCTGGTGACAGTGAGCTCCGGAGGAGG |
| | signaling | AGGATCCGGCGGAGGAGGCTCTGGCGGCGGCGGCTCCG |
| | domain | GCGGCGGCGGCTCCGAGATCGTGCTGACCCAGTCCCCAG |
| | | CCACACTGTCCCTGTCTCCAGGAGAGAGGGCCACCCTGT |
| | | CTTGCAGGGCCAGCCAGAACGTGAATAGGTACCTGGCCT |
| | | GGTATCACCAGAAGCCAGGACAGGCACCTCGCCTGCTG |
| | | ATCTACGACGCCTTCAACAGGGCAACCGGCATCCCTGCC |
| | | AGATTCAGCGGCTCCGGCTCTGGCACAGACTTTACCCTG |
| | | ACAATCAATAGCCTGGAGCCAGAGGATTTTGCCGTGTAC |
| | | TATTGTCAGCAGAGATCCGACTGGCCCCTGACTTTTGGC |
| | | GGCGGAACTAAACTGGAAATCAAGACAACTACACCTGC |
| | | TCCTAGGCCACCTACACCTGCACCAACCATCGCCAGCCA |
| | | GCCTCTGTCCCTGAGACCAGAGGCCTGTAGGCCAGCAGC |
| | | AGGAGGAGCAGTGCACACCCGGGGCCTGGACTTCGCCT |
| | | GCGATATCTACATCTGGGCACCACTGGCAGGAACATGTG |
| | | GCGTGCTGCTGCTGTCCCTGGTCATCACCCTGTACTGCA |
| | | AGAGAGGCAGGAAGAAGCTGCTGTATATCTTCAAGCAG |
| | | CCCTTCATGAGACCCGTGCAGACAACCCAGGAGGAGGA |
| | | CGGCTGCAGCTGTAGGTTCCCAGAGGAGGAGGAGGGAG |
| | | GATGTGAGCTGCGCGTGAAGTTTTCCCGGTCTGCCGATG |
| | | CACCTGCATACCAGCAGGGACAGAACCAGCTGTATAAC |
| | | GAGCTGAATCTGGGCCGGAGAGAGGAGTACGACGTGCT |
| | | GGATAAGAGGAGGGGAAGGGACCCTGAGATGGGAGGC |
| | | AAGCCTCGGAGAAAGAACCCACAGGAGGGCCTGTACAA |
| | | TGAGCTGCAGAAGGACAAGATGGCCGAGGCCTATAGCG |
| | | AGATCGGCATGAAGGGAGAGAGGCGCCGGGGCAAGGG |
| | | ACACGATGGCCTGTATCAGGGCCTGTCAACCGCTACAAA |
| | | AGATACCTACGATGCTCTGCACATGCAGGCTCTGCCACC |
| | | AAGA |
| 60 | CD8α signal | ATGGCTCTGCCTGTCACCGCTCTGCTGCTGCCTCTGGCTC |
| | sequence, | TGCTGCTGCACGCCGCCCGCCCTCAGGTCCAGCTGCAGG |
| | 2A10 scFv, | AGTCTGGGCCAGGCCTGGTGAAGCCCTCTGAGACCCTGA |
| | CD8α hinge | GCCTGACCTGCACAGTGTCCGCCGGCTCTATCAGCTCCT |
| | and | ACTATTGGAACTGGATCAGACAGCCTGCAGGCAAGGGA |
| | transmembrane | CTGGAGTGGATCGGAAGGATCTACACATCTGGCAGCAC |
| | regions, | CAACTATAATCCAAGCCTGCGGTCCAGAGTGACAATGTC |
| | 41BB | CGTGGACACCTCTAAGAATCAGTTCAGCCTGAAGCTGTC |
| | cytoplasmic | TAGCGTGACCGCCACAGATACCGCCGTGTACTATTGTGC |
| | signaling | CTCCGCCTCTTACACATATTTCGACTCCTTTGATATCTGG |
| | domain, CD3ζ | GGCCAGGGCACAATGGTGACCGTGTCCTCTGGAGGAGG |
| | cytoplasmic | AGGAAGCGGAGGAGGAGGAAGCGGCGGCGGCGGCTCT |
| | signaling | GGCGGCGGCGGCTCCGACATCCAGCTGACCCAGAGCCC |
| | domain | ATCCTTCCTGTCTGCCAGCGTGGGCGACAGGGTGACAAT |
| | | CACCTGCCGCGCCAGCCAGGATATCCGGAACTTTCTGGC |
| | | CTGGTACCAGCAGAAGCCCGGCAAGGCCCCTAAGCTGC |
| | | TGATCTATGCAGCAAGCACACTGCAGTCCGGAGTGCCAT |
| | | CTAGATTCTCCGGCTCTGGCAGCGGCACAGAGTTTGCCC |
| | | TGACCGTGAGCTCCCTGCAGCCTGAGGATTTTGCCACCT |
| | | ACTATTGTCAGCAGGTGAATTCATACCCAAGAACATTCG |
| | | GGCAGGGGACTAAAGTGGAAATCAAGACTACTACACCA |
| | | GCCCCTAGGCCACCTACACCTGCACCAACCATCGCCAGC |
| | | CAGCCTCTGTCCCTGAGACCAGAGGCCTGTAGGCCAGCA |
| | | GCAGGAGGAGCAGTGCACACCCGGGGCCTGGACTTCGC |
| | | CTGCGATATCTACATCTGGGCACCACTGGCAGGAACATG |
| | | TGGCGTGCTGCTGCTGTCCCTGGTCATCACCCTGTACTGC |
| | | AAGAGAGGCAGGAAGAAGCTGCTGTATATCTTCAAGCA |
| | | GCCCTTCATGAGACCCGTGCAGACAACCCAGGAGGAGG |
| | | ACGGCTGCAGCTGTAGGTTCCCAGAGGAGGAGGAGGGA |
| | | GGATGTGAGCTGCGCGTGAAGTTTTCCCGGTCTGCCGAT |
| | | GCACCTGCATACCAGCAGGGACAGAACCAGCTGTATAA |
| | | CGAGCTGAATCTGGGCCGGAGAGAGGAGTACGACGTGC |
| | | TGGATAAGAGGAGGGGAAGGGACCCTGAGATGGGAGGC |
| | | AAGCCTCGGAGAAAGAACCCACAGGAGGGCCTGTACAA |
| | | TGAGCTGCAGAAGGACAAGATGGCCGAGGCCTATAGCG |
| | | AGATCGGCATGAAGGGAGAGAGGCGCCGGGGCAAGGG |
| | | ACACGATGGCCTGTATCAGGGCCTGTCAACCGCTACAAA |
| | | AGATACCTACGATGCTCTGCACATGCAGGCTCTGCCACC |
| | | AAGA |
| 75 | CD8α signal | ATGGCTCTGCCTGTCACCGCCCTGCTGCTGCCCCTGGCC |
| | sequence, | CTGCTGCTGCACGCCGCTCGCCCTGAAGTCCAGCTGGTC |
| | 12H6 scFv, | GAATCTGGCGGAGGACTGGTGCAGCCAGGAGGATCCCT |
| | CD8α hinge | GAGACTGTCTTGCGCCGCCAGCGGCTTCACCTTTTCCAG |
| | and | ATACTGGATGTCTTGGGTGAGGCAGGCACCTGGCAAGG |
| | transmembrane | GACTGGAGTGGGTGGCCAACATCAAGCACGACGGCTCC |
| | regions, | GAGAAGTACTATGTGGATTCTGTGAAGGGCCGGTTCACC |
| | 41BB | TTTAGCAGAGACAACGCCAAGACATCCCTGTACCTGCAG |
| | cytoplasmic | ATGAACAGCCTGAGAGTGGAGGACACAGCCCTGTACTA |
| | signaling | TTGCGCCAGGTACTATGGCGGCCCCTTCGATTATTGGGG |
| | domain, CD3ζ | CCAGGGCACCCTGGTGACAGTGAGCTCCGGAGGAGGAG |
| | cytoplasmic | GAAGCGGCGGAGGAGGCAGCGGCGGCGGCGGCTCTGGC |
| | signaling | GGCGGCGGCAGCGAGATCGTGCTGACCCAGTCCCCAGG |
| | domain | CACACTGAGCCTGTCCCCAGGAGAGAGGGCCACCCTGA |
| | | GCTGTCGCGCCTCTCAGAGCGTGCGGTCTAGCTACCTGG |
| | | CCTGGTATCAGCAGAAGCCAGGACAGGCACCTCGCCTG |
| | | CTGATCTTTGGAGCATCCTCTAGGGCAACCGGCATCCCT |
| | | GACCGGTTCTCCGGATCTGGAAGCGGCACAGACTTCACC |
| | | CTGACAATCTCCCGGCTGGAGCCAGAGGATTTCGCCGTG |
| | | TACTATTGTCAGCAGTTTGGCTCATCTCTGACCTTCGGGG |
| | | GGGGCACAAAAGTGGAAATCAAGACAACAACTCCTGCT |
| | | CCTAGGCCACCTACACCTGCACCAACCATCGCCAGCCAG |
| | | CCTCTGTCCCTGAGACCAGAGGCCTGTAGGCCAGCAGCA |
| | | GGAGGAGCAGTGCACACCCGGGGCCTGGACTTCGCCTG |
| | | CGATATCTACATCTGGGCACCACTGGCAGGAACATGTGG |
| | | CGTGCTGCTGCTGTCCCTGGTCATCACCCTGTACTGCAA |
| | | GAGAGGCAGGAAGAAGCTGCTGTATATCTTCAAGCAGC |
| | | CCTTCATGAGACCCGTGCAGACAACCCAGGAGGAGGAC |
| | | GGCTGCAGCTGTAGGTTCCCAGAGGAGGAGGAGGGAGG |
| | | ATGTGAGCTGCGCGTGAAGTTTTCCCGGTCTGCCGATGC |
| | | ACCTGCATACCAGCAGGGACAGAACCAGCTGTATAACG |
| | | AGCTGAATCTGGGCCGGAGAGAGGAGTACGACGTGCTG |
| | | GATAAGAGGAGGGGAAGGGACCCTGAGATGGGAGGCA |
| | | AGCCTCGGAGAAAGAACCCACAGGAGGGCCTGTACAAT |
| | | GAGCTGCAGAAGGACAAGATGGCCGAGGCCTATAGCGA |
| | | GATCGGCATGAAGGGAGAGAGGCGCCGGGGCAAGGGA |
| | | CACGATGGCCTGTATCAGGGCCTGTCAACCGCTACAAAA |
| | | GATACCTACGATGCTCTGCACATGCAGGCTCTGCCACCA |
| | | AGA |
| 88 | 10D11 | ATGGCTCTGCCTGTGACTGCCCTGCTGCTGCCCCTGGCC |
| | | CTGCTGCTGCACGCCGCACGACCTGAAGTCCAGCTGCTG |
| | | GAGTCAGGAGGAGGACTGGAGCAGCCAGGCGGAAGCCT |
| | | GAGGCTGTCCTGCGCAGCATCTGGCTTCACCTTTAGCTC |
| | | CTATGCAATGAGCTGGGTGAGACAGGCCCCCGGCAAGG |
| | | GACTGGAGTGGGTGTCCGCCATCTCCGGATCTGGAGGAT |
| | | CCACACACTATGCCGACTCTGTGAAGGGCAGGTTCACCA |
| | | TCTCTCGGGATAACGCCAGAAATACACTGTACCTGCAGA |
| | | TGAACAGCCTGAGGGCAGAGGACACCGCCGTGTACTAT |
| | | TGCGCCAAGGAGGGCTACGTGGGCAGCTGGTATGCCCCT |
| | | TTTGATTACTGGGGCCAGGGCACCCTGGTGACAGTGTCT |
| | | AGCGGAGGAGGAGGAAGCGGAGGAGGAGGATCTGGCG |
| | | GCGGCGGCTCTGGCGGCGGCGGCAGCCAGCTGGTGCTG |
| | | ACACAGAGCCCATCCGCCTCTGCCAGCCTGGGCGCATCC |
| | | GTGAAGCTGACCTGTACACTGTCCTCTGGCCACAGCTCC |
| | | TATGCAATCGCATGGCACCAGCAGCAGCCAGAGAAGGG |
| | | ACCTCGGTACCTGATGAAGCTGAACAGCGGAGGATCCC |
| | | ACTCTAAGGGCGACGGCATCCCCGATAGGTTCTCTGGAT |
| | | CTAGCTCCGGAGCAGAGCGGTACCTGACCATCTCTAGCC |
| | | TGCAGAGCGAGGACGAGGCCGATTACTATTGTCAGACA |
| | | TGGGACACTGGGATTCGGGTCTTCGGCGGGGGAACAAA |
| | | ACTGACTGTCCTGACAACTACCCCAGCACCTAGGCCACC |
| | | TACACCTGCACCAACCATCGCCAGCCAGCCTCTGTCCCT |
| | | GAGACCAGAGGCCTGTAGGCCAGCAGCAGGAGGAGCAG |
| | | TGCACACCCGGGGCCTGGACTTCGCCTGCGATATCTACA |
| | | TCTGGGCACCACTGGCAGGAACATGTGGCGTGCTGCTGC |
| | | TGTCCCTGGTCATCACCCTGTACTGCAAGAGAGGCAGGA |
| | | AGAAGCTGCTGTATATCTTCAAGCAGCCCTTCATGAGAC |
| | | CCGTGCAGACAACCCAGGAGGAGGACGGCTGCAGCTGT |
| | | AGGTTCCCAGAGGAGGAGGAGGGAGGATGTGAGCTGCG |
| | | CGTGAAGTTTTCCCGGTCTGCCGATGCACCTGCATACCA |
| | | GCAGGGACAGAACCAGCTGTATAACGAGCTGAATCTGG |
| | | GCCGGAGAGAGGAGTACGACGTGCTGGATAAGAGGAGG |
| | | GGAAGGGACCCTGAGATGGGAGGCAAGCCTCGGAGAAA |
| | | GAACCCACAGGAGGGCCTGTACAATGAGCTGCAGAAGG |
| | | ACAAGATGGCCGAGGCCTATAGCGAGATCGGCATGAAG |
| | | GGAGAGAGGCGCCGGGGCAAGGGACACGATGGCCTGTA |
| | | TCAGGGCCTGTCAACCGCTACAAAAGATACCTACGATGC |
| | | TCTGCACATGCAGGCTCTGCCACCAAGA |
| 101 | 17F11 | ATGGCTCTGCCTGTGACTGCCCTGCTGCTGCCCCTGGCC |
| | | CTGCTGCTGCACGCCGCACGACCTCAGGTCACTCTGAGG |
| | | GAATCTGGCCCAGCCCTGGTGAAGCCCACCCAGACACTG |
| | | ACCCTGACATGCACCGTGTCCGGCGTGAGCCTGTCCACC |
| | | TCTGGCATGTGCGTGAGCTGGATCAGGCAGCCCCTGGGC |
| | | AAGGCCCTGGAGTGGCTGGGCTTCATCGATTGGGACGAT |
| | | GACAAGTACTATAACACATCCCTGAAGACAAGACTGAC |
| | | CATCTCCAAGGACACCTCTAAGAACCAGGTGGTGCTGAC |
| | | AATGACCAATATGGATCCTGTGGACACAGCCACCTACTA |
| | | TTGCGCCCGGATCAGAGGCTACAGCGGCTCCTATGATGC |
| | | CTTTGACATCTGGGGCCAGGGCACCGTGGTCATCGTGAG |
| | | CTCCGGCGGCGGCGGCTCTGGAGGAGGAGGAAGCGGAG |
| | | GAGGAGGAAGCGGGGGCGGCGGCTCTGATATCCAGATG |
| | | ACACAGAGCCCATCTAGCCTGTCTGCCAGCGTGGGCGAC |
| | | AGGGTGACAATCACCTGCCGCGCCAGCCAGGGCATCTCC |
| | | AATTACCTGGCCTGGTATCAGCAGAAGCCCGGCCGGGTG |
| | | CCTAAGCTGCTGATCTACGCAGCATCTACACTGCAGAGC |
| | | GGAGTGCCTTCCAGATTCTCCGGATCTGGAAGCGGAACC |
| | | GACTTCACCCTGACCATCTCCTCTCTGCAGCCAGAGGAC |
| | | GTGGCCACATACTATTGTCAGAAGTATATCTCCGCACCA |
| | | TTCACATTTGGACCTGGAACTAAAGTGGACATCAAGACA |
| | | ACTACCCCAGCACCTAGGCCACCTACACCTGCACCAACC |
| | | ATCGCCAGCCAGCCTCTGTCCCTGAGACCAGAGGCCTGT |
| | | AGGCCAGCAGCAGGAGGAGCAGTGCACACCCGGGGCCT |
| | | GGACTTCGCCTGCGATATCTACATCTGGGCACCACTGGC |
| | | AGGAACATGTGGCGTGCTGCTGCTGTCCCTGGTCATCAC |
| | | CCTGTACTGCAAGAGAGGCAGGAAGAAGCTGCTGTATA |
| | | TCTTCAAGCAGCCCTTCATGAGACCCGTGCAGACAACCC |
| | | AGGAGGAGGACGGCTGCAGCTGTAGGTTCCCAGAGGAG |
| | | GAGGAGGGAGGATGTGAGCTGCGCGTGAAGTTTTCCCG |
| | | GTCTGCCGATGCACCTGCATACCAGCAGGGACAGAACC |
| | | AGCTGTATAACGAGCTGAATCTGGGCCGGAGAGAGGAG |
| | | TACGACGTGCTGGATAAGAGGAGGGGAAGGGACCCTGA |
| | | GATGGGAGGCAAGCCTCGGAGAAAGAACCCACAGGAGG |
| | | GCCTGTACAATGAGCTGCAGAAGGACAAGATGGCCGAG |
| | | GCCTATAGCGAGATCGGCATGAAGGGAGAGAGGCGCCG |
| | | GGGCAAGGGACACGATGGCCTGTATCAGGGCCTGTCAA |
| | | CCGCTACAAAAGATACCTACGATGCTCTGCACATGCAGG |
| | | CTCTGCCACCAAGA |
| 114 | 6B2 | ATGGCTCTGCCTGTGACTGCCCTGCTGCTGCCCCTGGCC |
| | | CTGCTGCTGCACGCCGCACGACCTCAGGTGACCCTGAAG |
| | | GAGTCCGGCCTGACACTGATGAAGCCCACACAGACCCA |
| | | CACACTGACCTGCACATTCTCTGGCTTTTCTCTGAGCACC |
| | | TCCGGAGTGGGAGTGGGATGGATCAGACAGACCCCCGG |
| | | CAAGGCCCTGGAGTGGCTGACACAGATCTACTGGAACG |
| | | ACGAGAAGCGGTATTCTCCTAGCCTGAAGAATAGACTG |
| | | ACCATCACAAAGGATACATCCAAGAACCAGGTGGTGCT |
| | | GACCATGACAAATATGGACCCAGTGGATACCGCCACAT |
| | | ACTATTGTGCCCACCGGAGAGGAGTGGGAAACTGGTTC |
| | | GACCCATGGGGACAGGGCACCCTGGTGACAGTGAGCAG |
| | | CGGAGGAGGAGGCAGCGGAGGAGGAGGCTCCGGCGGC |
| | | GGCGGCTCTGGAGGAGGAGGCAGCGACATCCAGATGAC |
| | | CCAGTCCCCTTCTAGCGTGTCCGCCTCTGTGGGCGATAG |
| | | GGTGACCATCACATGCAGGGCAAGCCAGGGAATCTCCT |
| | | CTTGGCTGGCCTGGTACCAGCAGAAGCCAGGCAAGGCC |
| | | CCCAAGCTGCTGATCTATGCAGCAAGCTCCCTGCAGAGC |
| | | GGCGTGTCTAGCTCCTTCAGCGGCTCCGCCTCTGGAACC |
| | | GAGTTTACCCTGACAATCTCTAATCTGCAGCCTGAGGAC |
| | | TTTGCCATCTACTATTGTCAGCAGGCCTTCAGCTTTCCAT |
| | | TCACCTTTGGCCCCGGCACAAAGGTGGATATCAAGACAA |
| | | CTACCCCAGCACCTAGGCCACCTACACCTGCACCAACCA |
| | | TCGCCAGCCAGCCTCTGTCCCTGAGACCAGAGGCCTGTA |
| | | GGCCAGCAGCAGGAGGAGCAGTGCACACCCGGGGCCTG |
| | | GACTTCGCCTGCGATATCTACATCTGGGCACCACTGGCA |
| | | GGAACATGTGGCGTGCTGCTGCTGTCCCTGGTCATCACC |
| | | CTGTACTGCAAGAGAGGCAGGAAGAAGCTGCTGTATAT |
| | | CTTCAAGCAGCCCTTCATGAGACCCGTGCAGACAACCCA |
| | | GGAGGAGGACGGCTGCAGCTGTAGGTTCCCAGAGGAGG |
| | | AGGAGGGAGGATGTGAGCTGCGCGTGAAGTTTTCCCGG |
| | | TCTGCCGATGCACCTGCATACCAGCAGGGACAGAACCA |
| | | GCTGTATAACGAGCTGAATCTGGGCCGGAGAGAGGAGT |
| | | ACGACGTGCTGGATAAGAGGAGGGGAAGGGACCCTGAG |
| | | ATGGGAGGCAAGCCTCGGAGAAAGAACCCACAGGAGGG |
| | | CCTGTACAATGAGCTGCAGAAGGACAAGATGGCCGAGG |
| | | CCTATAGCGAGATCGGCATGAAGGGAGAGAGGCGCCGG |
| | | GGCAAGGGACACGATGGCCTGTATCAGGGCCTGTCAAC |
| | | CGCTACAAAAGATACCTACGATGCTCTGCACATGCAGGC |
| | | TCTGCCACCAAGA |
| 127 | 4A5 | ATGGCTCTGCCTGTGACTGCCCTGCTGCTGCCCCTGGCC |
| | | CTGCTGCTGCACGCCGCACGACCTCAGGTGACCCTGAAG |
| | | GAGAGCGGAGGAGGCCTGGTGCAGCCTGGCGGCTCCCT |
| | | GAGGCTGTCTTGCGCAGCAAGCGGCTTCACCTTCAGCTC |
| | | CTACGCCATGTCCTGGGTGAGACAGGCCCCTGGCAAGG |
| | | GCCTGGAGTGGGTGTCTGCCATCAGCGGCTCCGGAGGCT |
| | | CTACCTACTATGCCGACAGCGTGAAGGGCCGGTTCACAA |
| | | TCTCCAGAGATAACTCTAAGAATACCCTGTACCTGCAGA |
| | | TGAACTCCCTGCGCGCCGAGGACACAGCCGTGTACTATT |
| | | GCGCCAAGGACCTGGGCGCCACCGATTATTGGGGCCAG |
| | | GGCACACTGGTGACCGTGTCTAGCGGCGGCGGCGGCTCT |
| | | GGAGGAGGAGGCAGCGGCGGAGGAGGCTCCGGCGGCG |
| | | GCGGCTCTGACATCCAGATGACCCAGAGCCCATCCAGCG |
| | | TGAGCGCCAGCGTGGGCGATAGGGTGACAATCACCTGT |
| | | AGGGCATCCCAGGGAATCAGCTCCTGGCTGGCCTGGTAC |
| | | CAGCAGAAGCCAGGCAAGGCCCCCAAGCTGCTGATCTA |
| | | TGCAGCATCTAGCCTGCAGAGCGGAGTGCCATCCCGGTT |
| | | CTCCGGCTCTGGCAGCGGAACAGACTTTACACTGACCAT |
| | | CTCCTCTCTGCAGCCTGAGGATTTTGCCACCTACTATTGT |
| | | CAGCAGGCCAATAGCTTCCCACTGACATTTGGCGGCGGC |
| | | ACCAAGGTGGAGATCAAGACAACTACCCCAGCACCTAG |
| | | GCCACCTACACCTGCACCAACCATCGCCAGCCAGCCTCT |
| | | GTCCCTGAGACCAGAGGCCTGTAGGCCAGCAGCAGGAG |
| | | GAGCAGTGCACACCCGGGGCCTGGACTTCGCCTGCGATA |
| | | TCTACATCTGGGCACCACTGGCAGGAACATGTGGCGTGC |
| | | TGCTGCTGTCCCTGGTCATCACCCTGTACTGCAAGAGAG |
| | | GCAGGAAGAAGCTGCTGTATATCTTCAAGCAGCCCTTCA |
| | | TGAGACCCGTGCAGACAACCCAGGAGGAGGACGGCTGC |
| | | AGCTGTAGGTTCCCAGAGGAGGAGGAGGGAGGATGTGA |
| | | GCTGCGCGTGAAGTTTTCCCGGTCTGCCGATGCACCTGC |
| | | ATACCAGCAGGGACAGAACCAGCTGTATAACGAGCTGA |
| | | ATCTGGGCCGGAGAGAGGAGTACGACGTGCTGGATAAG |
| | | AGGAGGGGAAGGGACCCTGAGATGGGAGGCAAGCCTCG |
| | | GAGAAAGAACCCACAGGAGGGCCTGTACAATGAGCTGC |
| | | AGAAGGACAAGATGGCCGAGGCCTATAGCGAGATCGGC |
| | | ATGAAGGGAGAGAGGCGCCGGGGCAAGGGACACGATG |
| | | GCCTGTATCAGGGCCTGTCAACCGCTACAAAAGATACCT |
| | | ACGATGCTCTGCACATGCAGGCTCTGCCACCAAGA |

b. Safety Switches and Monoclonal Antibody Specific-Epitopes Safety Switches

It will be appreciated that adverse events may be minimized by transducing the immune cells (containing one or more CARs) with a suicide gene. It may also be desired to incorporate an inducible “on” or “accelerator” switch into the immune cells. Suitable techniques include use of inducible caspase-9 (U.S. Appl. 2011/0286980) or a thymidine kinase, before, after or at the same time, as the cells are transduced with the CAR construct of the present disclosure. Additional methods for introducing suicide genes and/or “on” switches include TALENS, zinc fingers, RNAi, siRNA, shRNA, antisense technology, and other techniques known in the art.

In accordance with the disclosure, additional on-off or other types of control switch techniques may be incorporated herein. These techniques may employ the use of dimerization domains and optional activators of such domain dimerization. These techniques include, e.g., those described by Wu et al., Science 2014 350 (6258) utilizing FKBP/Rapalog dimerization systems in certain cells, the contents of which are incorporated by reference herein in their entirety. Additional dimerization technology is described in, e.g., Fegan et al. Chem. Rev. 2010, 110, 3315-3336 as well as U.S. Pat. Nos. 5,830,462; 5,834,266; 5,869,337; and 6,165,787, the contents of which are also incorporated by reference herein in their entirety. Additional dimerization pairs may include cyclosporine-A/cyclophilin, receptor, estrogen/estrogen receptor (optionally using tamoxifen), glucocorticoids/glucocorticoid receptor, tetracycline/tetracycline receptor, vitamin D/vitamin D receptor. Further examples of dimerization technology can be found in e.g., WO 2014/127261, WO 2015/090229, US 2014/0286987, US2015/0266973, US2016/0046700, U.S. Pat. No. 8,486,693, US 2014/0171649, and US 2012/0130076, the contents of which are further incorporated by reference herein in their entirety.

In some embodiments, the CAR-immune cell (e.g., CAR-T cell) of the disclosure comprises a polynucleotide encoding a suicide polypeptide or a safety switch, such as for example RQR8. See, e.g., WO2013153391A, which is hereby incorporated by reference in its entirety. In CAR-immune cells (e.g., CAR-T cells) comprising the polynucleotide, the suicide polypeptide is expressed at the surface of the CAR-immune cell (e.g., CAR-T cell). In some embodiments, the suicide polypeptide comprises the amino acid sequence shown in SEQ ID NO: 167:

(SEQ ID NO: 167)
CPYSNPSLCSGGGGSELPTQGTFSNVSTNVSPAKPTTTACPYSNPSL
CSGGGGSPAPRPPTPAPTIASQPLSLRPEACRPAAGGAVHTRGLDFA
CDIYIWAPLAGTCGVLLLSLVITLYCNHRNRRRVCKCPRPVV.

The suicide polypeptide may also comprise a signal peptide at the amino terminus—for example, MGTSLLCWMALCLLGADHADA (SEQ ID NO: 169). In some embodiments, the suicide polypeptide comprises the amino acid sequence shown in SEQ ID NO: 168, which includes the signal sequence of SEQ ID NO: 169:

(SEQ ID NO: 168)
MGTSLLCWMALCLLGADHADACPYSNPSLCSGGGGSELPTQGTFSNV
STNVSPAKPTTTACPYSNPSLCSGGGGSPAPRPPTPAPTIASQPLSL
RPEACRPAAGGAVHTRGLDFACDIYIWAPLAGTCGVLLLSLVITLYC
NHRNRRRVCKCPRPVV.

When the suicide polypeptide is expressed at the surface of a CAR-immune cell (e.g., CAR-T cell), binding of rituximab to the R epitopes of the polypeptide causes lysis of the cell. More than one molecule of rituximab may bind per polypeptide expressed at the cell surface. Each R epitope of the polypeptide may bind a separate molecule of rituximab. Deletion of Claudin 18.2-specific CAR-immune cell (e.g., CAR-T cell) may occur in vivo, for example by administering rituximab to a patient. The decision to delete the transferred cells may arise from undesirable effects being detected in the patient which are attributable to the transferred cells, such as for example, when unacceptable levels of toxicity are detected.

In some embodiments, a suicide polypeptide is expressed on the surface of the cell. In some embodiments, a suicide polypeptide is included in the CAR construct. In some embodiments, a suicide polypeptide is not part of the Claudin 18.2 CAR construct.

In some embodiments, the extracellular domain of any one of the Claudin 18.2-specific CARs disclosed herein may comprise one or more epitopes specific for (i.e., specifically recognized by) a monoclonal antibody. These epitopes are also referred to herein as mAb-specific epitopes. Exemplary mAb-specific epitopes are disclosed in International Patent Publication No. WO 2016/120216, which is incorporated herein in its entirety. In these embodiments, the extracellular domain of the CARs comprises antigen binding domains that specifically bind to Claudin 18.2 and one or more epitopes that bind to one or more monoclonal antibodies (mAbs). CARs comprising the mAb-specific epitopes can be single-chain or multi-chain.

The inclusion of epitopes specific for monoclonal antibodies in the extracellular domain of the CARs described herein allows sorting and depletion of engineered immune cells expressing the CARs. In some embodiments, this feature also promotes recovery of endogenous Claudin 18.2-expressing cells that were depleted by administration of engineered immune cells expressing the CARs. In some embodiments, allowing for depletion provides a safety switch in case of deleterious effects, e.g., upon administration to a subject.

Accordingly, in some embodiments, the present disclosure relates to a method for sorting and/or depleting the engineered immune cells endowed with the CARs comprising mAb-specific epitopes and a method for promoting recovery of endogenous Claudin 18.2-expressing cells. 1019%1 Several epitope-monoclonal antibody couples can be used to generate CARs comprising monoclonal antibody specific epitopes; in particular, those already approved for medical use, such as CD20 epitope/rituximab as anon-limiting example.

The disclosure also encompasses methods for sorting the engineered immune cells endowed with the Claudin 18.2-specific CARs expressing the mAb-specific epitope(s) and therapeutic methods where the activation of the engineered immune cells endowed with these CARs is modulated by depleting the cells using an antibody that targets the external ligand binding domain of said CARs. Table 4 provides exemplary mimotope sequences that can be inserted into the extracellular domains of any one of the CARs of the disclosure.

TABLE 4
Exemplary mimotope amino acid sequences

| Description | SEQ ID NO: | Amino Acid Sequence |
|---|---|---|
| Rituximab Mimotope | SEQ ID NO: 140 | CPYSNPSLC |
| Palivizumab Epitope | SEQ ID NO: 141 | NSELLSLINDMPITNDQKKLMSNN |
| Cetuximab Mimotope 1 | SEQ ID NO: 142 | CQFDLSTRRLKC |
| Cetuximab Mimotope 2 | SEQ ID NO: 143 | CQYNLSSRALKC |
| Cetuximab Mimotope 3 | SEQ ID NO: 144 | CVWQRWQKSYVC |
| Cetuximab Mimotope 4 | SEQ ID NO: 145 | CMWDRFSRWYKC |
| Nivolumab Epitope 1 | SEQ ID NO: 146 | SFVLNWYRMSPSNQTDKLAAFPEDR |
| Nivolumab Epitope 2 | SEQ ID NO: 147 | SGTYLCGAISLAPKAQIKE |
| QBEND-10 Epitope 1 | SEQ ID NO: 148 | ELPTQGTFSNVSTNVSPAKPTTTA |
| QBEND-10 Epitope 2 | SEQ ID NO: 149 | ELPTQGTFSNVSTNVS |
| Alemtuzumab Epitope | SEQ ID NO: 150 | GQNDTSQTSSPS |
| 2xRituximab mimotope | SEQ ID NO: 176 | GSGGGGSCPYSNPSLCSGGGGSCPYS |
| | | NPSLCSGGGGS |

In some embodiments, the extracellular binding domain of the CAR comprises the following sequence:
V₁-L₁-V₂-(L)ₓ-Epitope1-(L)ₓ-;
V₁-L₁-V₂-(L)ₓ-Epitope1-(L)ₓ-Epitope2-(L)ₓ-;
V₁-L₁-V₂-(L)ₓ-Epitope1-(L)ₓ-Epitope2-(L)ₓ-Epitope3-(L)ₓ-;
(L)ₓ-Epitope1-(L)ₓ-V₁-L₁-V₂;
(L)ₓ-Epitope1-(L)ₓ-Epitope2-(L)ₓ-V₁-L₁-V₂;
Epitope1-(L)ₓ-Epitope2-(L)ₓ-Epitope3-(L)ₓ-V-L-V₂;
(L)ₓ-Epitope1-(L)ₓ-V₁-L₁-V₂-(L)ₓ-Epitope2-(L)ₓ-;
(L)ₓ-Epitope1-(L)ₓ-V₁-L₁-V₂-(L)ₓ-Epitope2-(L)ₓ-Epitope3-(L)ₓ-;
(L)ₓ-Epitope1-(L)ₓ-V₁-L₁-V₂-(L)ₓ-Epitope2-(L)ₓ-Epitope3-(L)ₓ-Epitope4-(L)ₓ-;
(L)ₓ-Epitope1-(L)ₓ-Epitope2-(L)ₓ-V₁-L₁-V₂-(L)ₓ-Epitope3-(L)ₓ-;
(L)ₓ-Epitope1-(L)ₓ-Epitope2-(L)ₓ-V-L-V-(L)ₓ-Epitope3-(L)ₓ-Epitope4-(L)ₓ-;
V₁(L)ₓ-Epitope1-(L)ₓ-V
V₁-(L)ₓ-Epitope1-(L)ₓ-V₂-(L)ₓ-Epitope2-(L)ₓ;
V₁-(L)ₓ-Epitope1-(L)ₓ-V₂-(L)ₓ-Epitope2-(L)ₓ-Epitope3-(L)ₓ;
V₁-(L)ₓ-Epitope1-(L)ₓ-V₂-(L)ₓ-Epitope2-(L)ₓ-Epitope3-(L)ₓ-Epitope4-(L)ₓ;
(L)ₓ-Epitope1-(L)ₓ-V₁-(L)ₓ-Epitope2-(L)ₓ-V₂; or,
(L)ₓ-Epitope1-(L)ₓ-V₁-(L)ₓ-Epitope2-(L)ₓ-V₁-(L)ₓ-Epitope3-(L)ₓ;
wherein,
V₁ is V_{L} and V₂ is V_{H} or V₁ is V_{H} and V₂ is V_{L};
L₁ is a linker suitable to link the V_{H} chain to the V₁ chain;
L is a linker comprising glycine and serine residues, and each occurrence of L in the extracellular binding domain can be identical or different to other occurrence of L in the same extracellular binding domain, which in embodiments comprises or is SGGGG (SEQ ID NO: 177). GGGGS (SEQ ID NO: 163) or SGGGGS (SEQ ID NO. 178), and.
x is 0 or 1 or 2 and each occurrence of x is selected independently from the others; and,
Epitope 1, Epitope 2, Epitope 3 and Epitope 4 are mAb-specific epitopes and can be identical or different and wherein V_{H} is a heavy chain variable fragment and V_{L} is a light chain variable fragment. In some embodiments, Epitope 1, Epitope 2, Epitope 3 and Epitope 4 can each comprise the amino acid sequence of SEQ ID NO: 140. In some embodiments, Epitope 1, Epitope 2, Epitope 3 and Epitope 4 can each comprise the amino acid sequence of SEQ ID NO: 148 or 149. In some embodiments. Epitope 1. Epitope 2 and Epitope 4 are a mAb-specific epitope having an amino acid sequence of SEQ ID NO:140 and Epitope 3 is a mAb-specific epitope having an amino acid sequence of SEQ ID NO: 148. In some embodiments, Epitope 1, Epitope 2 and Epitope 4 are a mAb-specific epitope having an amino acid sequence of SEQ ID NO: 140 and Epitope 3 is a mAb-specific epitope having an amino acid sequence of SEQ ID NO: 149.
   c. Hinge Domain

The extracellular domain of the CARs of the disclosure may comprise a “hinge” domain (or hinge region). The term generally refers to any polypeptide that functions to link the transmembrane domain in a CAR to the extracellular antigen binding domain in a CAR. In particular, hinge domains can be used to provide more flexibility and accessibility for the extracellular antigen binding domain.

A hinge domain may comprise up to 300 amino acids—in some embodiments 10 to 100 amino acids or in some embodiments 25 to 50 amino acids. The hinge domain may be derived from all or part of naturally occurring molecules, such as from all or part of the extracellular region of CD8, CD4, CD28, 4-1BB, or IgG (in particular, the hinge region of an IgG; it will be appreciated that the hinge region may contain some or all of a member of the immunoglobulin family such as IgG1, IgG2, IgG3, IgG4, IgA, IgD, IgE, IgM, or fragment thereof), or from all or part of an antibody heavy-chain constant region. Alternatively, the A domain may be a synthetic sequence that corresponds to a naturally occurring A sequence or may be an entirely synthetic A sequence. In some embodiments said A domain is a part of human CD8α chain (e.g., NP_001139345.1). In another particular embodiment, said hinge and transmembrane domains comprise a part of human CD8α chain. In some embodiments, the hinge domain of CARs described herein comprises a subsequence of CD8α, CD28, an IgG1, IgG4, PD-1 or an FcγRIIIα, in particular the hinge region of any of an CD8α, CD28, an IgG1, IgG4, PD-1 or an FcγRIIIα. In some embodiments, the hinge domain comprises a human CD8α hinge, a human CD28 hinge domain, a human IgG1 hinge, a human IgG4, a human PD-1 or a human FcγRIIIα hinge. In some embodiments the CARs disclosed herein comprise a scFv, a human CD8α hinge and transmembrane domains, the CD3ζ signaling domain, and 4-1BB signaling domain. In some embodiments the CARs disclosed herein comprise a scFv, a human CD28 hinge and transmembrane domains, the CD3ζ signaling domain, and 4-1BB signaling domain. Table 5 provides amino acid sequences for exemplary hinges provided herein.

TABLE 5
Amino Acid Sequences of Exemplary Hinges

| | | SEQ ID |
|---|---|---|
| Domain | Amino Acid Sequence | NO: |
| FcγRIIIα | GLAVSTISSFFPPGYQ | 170 |
| hinge | | |
| CD8α | TTTPAPRPPTPAPTIASQPLSLRPEACRPAAGGAVHTRGL | 153 |
| hinge | DFACD | |
| IgG1 | EPKSPDKTHTCPPCPAPPVAGPSVFLFPPKPKDTLMIARTPEVT | 171 |
| hinge | CVVVDVSHEDPEVKFNWYVDGVEVHNAKTKPREEQYNSTYR | |
| | VVSVLTVLHQDWLNGKEYKCKVSNKALPAPIEKTISKAKGQP | |
| | REPQVYTLPPSRDELTKNQVSLTCLVKGFYPSDIAVEWESNGQ | |
| | PENNYKTTPPVLDSDGSFFLYSKLTVDKSRWQQGNVFSCSVM | |
| | HEALHNHYTQKSLSLSPGK | |

In certain embodiments, the hinge region comprises an amino acid sequence that is at least about 75%, at least about 80%, at least about 85%, at least about 90%, at least about 95%, at least about 96%, at least about 97%, at least about 98%, at least about 99%, or 100% identical to the extracellular domain amino acid sequences set forth herein in Table 5.

d. Transmembrane Domain

The CARs of the disclosure are designed with a transmembrane domain that is fused to the extracellular domain of the CAR. It can similarly be fused to the intracellular domain of the CAR. In some instances, the transmembrane domain can be selected or modified by amino acid substitution to avoid binding of such domains to the transmembrane domains of the same or different surface membrane proteins to minimize interactions with other members of the receptor complex. In some embodiments, short linkers may form linkages between any or some of the extracellular, transmembrane, and intracellular domains of the CAR.

Suitable transmembrane domains for a CAR disclosed herein have the ability to (a) be expressed at the surface an immune cell such as, for example without limitation, a lymphocyte cell, such as a T helper (Tₕ) cell, cytotoxic T (T_{c}) cell, T regulatory (T_{reg}) cell, or Natural killer (NK) cells, and/or (b) interact with the extracellular antigen binding domain and intracellular signaling domain for directing the cellular response of an immune cell against a target cell.

The transmembrane domain may be derived either from a natural or from a synthetic source. Where the source is natural, the domain may be derived from any membrane-bound or transmembrane protein.

Transmembrane regions of particular use in this disclosure may be derived from (comprise, or correspond to) CD8α, CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1 1d, ITGAE, CD103, ITGAL, CD1 1a, LFA-1, ITGAM, CD1 1b, ITGAX, CD1 1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

As non-limiting examples, the transmembrane region can be a derived from, or be a portion of a T cell receptor such as α, β, γ or δ, polypeptide constituting CD3 complex, IL-2 receptor p55 (a chain), p75 (β chain) or γ chain, subunit chain of Fc receptors, in particular Fcγ receptor III or CD proteins. Alternatively, the transmembrane domain can be synthetic and can comprise predominantly hydrophobic residues such as leucine and valine. In some embodiments said transmembrane domain is derived from the human CD8α chain (e.g., NP_001139345.1).

In some embodiments, the transmembrane domain in the CAR of the disclosure is a CD8α transmembrane domain. In some embodiments, the transmembrane domain in the CAR of the disclosure is a CD8α transmembrane domain comprising the amino acid sequence IYIWAPLAGTCGVLLLSLVIT (SEQ ID NO: 154). In some embodiments, the hinge and transmembrane domain in the CAR of the disclosure is a CD8α hinge and transmembrane domain comprising the amino acid sequence of SEQ ID NO: 136.

In some embodiments, the transmembrane domain in the CAR of the disclosure is a CD28 transmembrane domain. In some embodiments, the transmembrane domain in the CAR of the disclosure is a CD28 transmembrane domain comprising the amino acid sequence of

(SEQ ID NO: 157)
FWVLVVVGGVLACYSLLVTVAFIIFWV.

e. Intracellular Domain

The intracellular (cytoplasmic) domain of the CARs of the disclosure can provide activation of at least one of the normal effector functions of the immune cell comprising the CAR, e.g., Signal 1/activation and/or Signal 2/costimulation. Effector function of a T cell, for example, may refer to cytolytic activity or helper activity, including the secretion of cytokines. In some embodiments, an activating intracellular signaling domain for use in a CAR can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability.

It will be appreciated that suitable (e.g., activating) intracellular domains include, but are not limited to signaling domains derived from (or corresponding to) CD3 zeta, CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1 1d, ITGAE, CD103, ITGAL, CD1 1a, LFA-1, ITGAM, CD1 1b, ITGAX, CD1 1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

The intracellular domains of the CARs of the disclosure may incorporate, in addition to the activating domains described above, costimulatory signaling domains (interchangeably referred to herein as costimulatory molecules) to increase their potency. Costimulatory domains can provide a signal in addition to the primary signal provided by an activating molecule as described herein.

It will be appreciated that suitable costimulatory domains within the scope of the disclosure can be derived from (or correspond to) for example, CD28, OX40, 4-1BB/CD137, CD2, CD3 (alpha, beta, delta, epsilon, gamma, zeta), CD4, CD5, CD7, CD9, CD16, CD22, CD27, CD30, CD 33, CD37, CD40, CD 45, CD64, CD80, CD86, CD134, CD137, CD154, PD-1, ICOS, lymphocyte function-associated antigen-1 (LFA-1 (CD1 1a/CD18), CD247, CD276 (B7-H3), LIGHT (tumor necrosis factor superfamily member 14; TNFSF14), NKG2C, Ig alpha (CD79a), DAP-10, Fc gamma receptor, MHC class I molecule, TNFR, integrin, signaling lymphocytic activation molecule, BTLA, Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1-1d, ITGAE, CD103, ITGAL, CD1-1a, LFA-1, ITGAM, CD1-1b, ITGAX, CD1-1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, or fragments or combinations thereof. It will be appreciated that additional costimulatory molecules, or fragments thereof, not listed above are within the scope of the disclosure.

In some embodiments, the intracellular/cytoplasmic domain of the CAR can be designed to comprise the 41BB/CD137 domain by itself or combined with any other desired intracellular domain(s) useful in the context of the CAR of the disclosure. The complete native amino acid sequence of 41BB/CD137 is described in NCBI Reference Sequence: NP_001552.2. The complete native 41BB/CD137 nucleic acid sequence is described in NCBI Reference Sequence: NM_001561.5.

In some embodiments, the intracellular/cytoplasmic domain of the CAR can be designed to comprise the CD28 domain by itself or combined with any other desired intracellular domain(s) useful in the context of the CAR of the disclosure. The complete native amino acid sequence of CD28 is described in NCBI Reference Sequence: NP_006130.1. The complete native CD28 nucleic acid sequence is described in NCBI Reference Sequence: NM_006139.1.

In some embodiments, the intracellular/cytoplasmic domain of the CAR can be designed to comprise the CD3 zeta domain by itself or combined with any other desired intracellular domain(s) useful in the context of the CAR of the disclosure. In some embodiments, the intracellular signaling domain of the CAR can comprise the CD3ζ signaling domain which has amino acid sequence with at least about 70%, at least 80%, at least 90%, 95%, 97%, or 99% sequence identity with an amino acid sequence shown in SEQ ID NO: 138 or in SEQ ID NO: 139 (see Table 7a). For example, the intracellular domain of the CAR can comprise a CD3 zeta chain portion and a portion of a costimulatory signaling molecule. The intracellular signaling sequences within the intracellular signaling portion of the CAR of the disclosure may be linked to each other in a random or specified order. In some embodiments, the intracellular domain is designed to comprise the activating domain of CD3 zeta and a signaling domain of CD28. In some embodiments, the intracellular domain is designed to comprise the activating domain of CD3 zeta and a costimulatory/signaling domain of 4-1BB.

In some embodiments, the 4-1BB (intracellular domain) comprises the amino acid sequence KRGRKKLLYIFKQPFMRPVQTTQEEDGCSCRFPEEEEGGCEL (SEQ ID NO: 137). In some embodiments, the 4-1BB (intracellular domain) is encoded by the nucleic acid sequence:

(SEQ ID NO: 172)
AAGCGCGGCAGGAAGAAGCTCCTCTACATTTTTAAGCAGCCTTTTATGAG
GCCCGTACAGACAACACAGGAGGAAGATGGCTGTAGCTGCAGATTTCCCG
AGGAGGAGGAAGGTGGGTGCGAGCTG.

In some embodiments, the intracellular domain in the CAR is designed to comprise a portion of CD28 and CD3 zeta, wherein the intracellular CD28 is encoded by the nucleic acid sequence set forth in SEQ ID NO: 173.

(SEQ ID NO: 173)
AGATCCAAAAGAAGCCGCCTGCTCCATAGCGATTACATGAATATGACTCC
ACGCCGCCCTGGCCCCACAAGGAAACACTACCAGCCTTACGCACCACCTA
GAGATTTCGCTGCCTATCGGAGC.

In some embodiments, the intracellular domain in the CAR is designed to comprise the amino acid sequence RSKRSRLLHSDYMNMTPRRPGPTRKHYQPYAPPRDFAAYRS (CD28-YMNM intracellular domain (“YMNM” is disclosed as SEQ ID NO: 217), SEQ ID NO: 174). The CD3 zeta amino acid sequence may comprise SEQ ID NO: 138 or 139 and the nucleic acid sequence that encodes the CD3 zeta amino acid sequence may comprise SEQ ID NO: 175:

(SEQ ID NO: 175)
AGGGTGAAGTTTTCCAGATCTGCAGATGCACCAGCGTATCAGCAGGGCCA
GAACCAACTGTATAACGAGCTCAACCTGGGACGCAGGGAAGAGTATGACG
TTTTGGACAAGCGCAGAGGACGGGACCCTGAGATGGGTGGCAAACCAAGA
CGAAAAAACCCCCAGGAGGGTCTCTATAATGAGCTGCAGAAGGATAAGAT
GGCTGAAGCCTATTCTGAAATAGGCATGAAAGGAGAGCGGAGAAGGGGAA
AAGGGCACGACGGTTTGTACCAGGGACTCAGCACTGCTACGAAGGATACT
TATGACGCTCTCCACATGCAAGCCCTGCCACCTAGG.

In some embodiments the intracellular signaling domain of the CAR of the disclosure comprises a domain of a co-stimulatory molecule. In some embodiments, the intracellular signaling domain of a CAR of the disclosure comprises a part of co-stimulatory molecule selected from the group consisting of fragment of 4-1BB (GenBank: AAA53133) and CD28 (NP_006130.1). In some embodiments, the intracellular signaling domain of the CAR of the present disclosure comprises an amino acid sequence which comprises at least 70%, at least 80%, at least 90%, 95%, 97%, or 99% sequence identity with an amino acid sequence shown in SEQ ID NO: 137 and SEQ ID NO: 174. In some embodiments, the intracellular signaling domain of the CAR of the disclosure comprises amino acid sequence which comprises at least 70%, at least 80%, at least 90%, 95%, 97%, or 99% sequence identity with an amino acid sequence shown in SEQ ID NO: 137 and/or at least 70%, at least 80%, at least 90%, 95%, 97%, or 99% sequence identity with an amino acid sequence shown in SEQ ID NO: 174.

In exemplary embodiments, a CAR of the disclosure comprises, from N-terminus to C-terminus: a (cleavable) CD8α signal sequence, a Claudin 18.2 scFv, a CD8α hinge and transmembrane region, a 4-1BB cytoplasmic (costimulatory) signaling domain, and a CD3ζ cytoplasmic (stimulatory) signaling domain.

### III. Immune Cells Comprising CARs

a. Immune Cells

Provided herein are engineered immune cells expressing the CARs of the present disclosure (e.g., CAR-T cells).

In some embodiments, an engineered immune cell comprises a population of CARs, each CAR comprising different extracellular antigen-binding domains. In some embodiments, an immune cell comprises a population of CARs, each CAR comprising the same extracellular antigen-binding domains.

The engineered immune cells can be allogeneic or autologous.

In some embodiments, the engineered immune cell is a T cell (e.g., inflammatory T lymphocyte, cytotoxic T lymphocyte, regulatory T lymphocyte (Treg), helper T lymphocyte, tumor infiltrating lymphocyte (TIL)), natural killer T cell (NKT), TCR-expressing cell, dendritic cell, killer dendritic cell, a mast cell, or a B-cell. In some embodiments, the cell can be derived from the group consisting of CD4+T-lymphocytes and CD8+T-lymphocytes. In some exemplary embodiments, the engineered immune cell is a T cell. In some exemplary embodiments, the engineered immune cell is a gamma delta T cell. In some exemplary embodiments, the engineered immune cell is a macrophage. In some exemplary embodiments, the engineered immune cell is a natural killer (NK) cell.

In some embodiments, the engineered immune cell can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells.

In some embodiments, the cell is obtained or prepared from peripheral blood. In some embodiments, the cell is obtained or prepared from peripheral blood mononuclear cells (PBMCs). In some embodiments, the cell is obtained or prepared from bone marrow. In some embodiments, the cell is obtained or prepared from umbilical cord blood. In some embodiments, the cell is a human cell.

In some embodiments, the cell is transfected or transduced by the nucleic acid vector using a method selected from the group consisting of electroporation, sonoporation, biolistics (e.g., Gene Gun), lipid transfection, polymer transfection, nanoparticles, viral transfection (e.g., retrovirus, lentivirus, AAV) or polyplexes.

In some embodiments, the engineered immune cells expressing at their cell surface membrane a Claudin 18.2-specific CAR of the disclosure comprise a percentage of stem cell memory and central memory cells greater than 10%, 20%, 30%, 40%, 50%, 60%, 70%, 80%, 90%, or 100%. In some embodiments, the engineered immune cells expressing at their cell surface membrane a Claudin 18.2-specific CAR of the disclosure comprise a percentage of stem cell memory and central memory cells of about 10% to about 100%, about 10% to about 90%, about 10% to about 80%, about 10% to about 70%, about 10% to about 60%, about 10% to about 50%, about 10% to about 40%, about 10% to about 30%, about 10% to about 20%, about 15% to about 100%, about 15% to about 90%, about 15% to about 80%, about 15% to about 70%, about 15% to about 60%, about 15% to about 50%, about 15% to about 40%, about 15% to about 30%, about 20% to about 100%, about 20% to about 90%, about 20% to about 80%, about 20% to about 70%, about 20% to about 60%, about 20% to about 50%, about 20% to about 40%, about 20% to about 30%, about 30% to about 100%, about 30% to about 90%, about 30% to about 80%, about 30% to about 70%, about 30% to about 60%, about 30% to about 50%, about 30% to about 40%, about 40% to about 100%, about 40% to about 90%, about 40% to about 80%, about 40% to about 70%, about 40% to about 60%, about 40% to about 50%, about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 90%, about 70% to about 80%, about 80% to about 100%, about 80% to about 90%, about 90% to about 100%, about 25% to about 50%, about 75% to about 100%, or about 50% to about 75%.

In some embodiments, the immune cell is an inflammatory T-lymphocyte that expresses any one of the CARs described herein. In some embodiments, the immune cell is a cytotoxic T-lymphocyte that expresses any one of the CARs described herein. In some embodiments, the immune cell is a regulatory T-lymphocyte that expresses any one of the CARs described herein. In some embodiments, the immune cell is a helper T-lymphocyte that expresses any one of the CARs described herein.

Prior to expansion and genetic modification, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available and known to those skilled in the art, may be used. In some embodiments, cells can be derived from a healthy donor or from a patient e.g. a patient diagnosed with cancer or from a patient diagnosed with an infection. In some embodiments, cells can be part of a mixed population of cells which present different phenotypic characteristics.

Also provided herein are cell lines obtained from a transformed immune cell (e.g., T-cell) according to any of the above-described methods. Also provided herein are modified cells resistant to an immunosuppressive treatment. In some embodiments, an isolated cell according to the disclosure comprises a polynucleotide encoding a CAR

The immune cells of the disclosure can be activated and expanded, either prior to or after genetic modification of the immune cells, using methods as generally known. Generally, the engineered immune cells of the disclosure can be expanded, for example, by contacting with an agent that stimulates a CD3 TCR complex and a costimulatory molecule on the surface of the T-cells to create an activation signal for the T cell. For example, chemicals such as calcium ionophore A23187, phorbol 12-myristate 13-acetate (PMA), or mitogenic lectins like phytohemagglutinin (PHA) can be used to create an activation signal for the T cell.

In some embodiments, T cell populations may be stimulated in vitro by contact with, for example, an anti-CD3 antibody such as an OKT3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody (e.g., an OKT3 antibody) and an anti-CD28 antibody, under conditions appropriate for stimulating proliferation of the T cells. The anti-CD3 antibody and an anti-CD28 antibody can be disposed on a bead, such as a plastic or magnetic bead, or plate or other substrate. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-2, IL-15, TGFbeta, and TNF, or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, A1M-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells (e.g., IL-7 and/or IL-15). Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). T cells that have been exposed to varied stimulation times may exhibit different characteristics. In some embodiments, the cells of the disclosure can be expanded by co-culturing with tissue or cells. The cells can also be expanded in vivo, for example in the subject's blood after administering the cell into the subject.

In some embodiments, an engineered immune cell according to the present disclosure may comprise one or more disrupted or inactivated genes. In some embodiments, an engineered immune cell according to the present disclosure comprises one disrupted or inactivated gene selected from the group consisting of CD52, Claudin 18.2, GR, PD-1, CTLA-4, LAG3, TIM3, BTLA, BY55, TIGIT, B7H5, LAIR1, SIGLEC10, 2B4, HLA, TCRα and TCRβ and/or expresses a CAR, a multi-chain CAR and/or a pTα transgene. In some embodiments, an isolated cell comprises polynucleotides encoding polypeptides comprising a multi-chain CAR. In some embodiments, the isolated cell according to the present disclosure comprises two disrupted or inactivated genes selected from the group consisting of: CD52 and GR, CD52 and TCRα, CDR52 and TCRβ, Claudin 18.2 and CD52, Claudin 18.2 and TCRα, Claudin 18.2 and TCRβ, GR and TCRα, GR and TCRβ, TCRα and TCRβ, PD-1 and TCRα, PD-1 and TCRβ, CTLA-4 and TCRα, CTLA-4 and TCRβ, LAG3 and TCRα, LAG3 and TCRβ, TIM3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ and/or expresses a CAR, a multi-chain CAR and a pTα transgene. In some embodiments the method comprises disrupting or inactivating one or more genes by introducing into the cells an endonuclease able to selectively inactivate a gene by selective DNA cleavage. In some embodiments the endonuclease can be, for example, a zinc finger nuclease (ZFN), megaTAL nuclease, meganuclease, transcription activator-like effector nuclease (TALE-nuclease/TALEN), or CRISPR (e.g., Cas9 or Cas12) endonuclease.

In some embodiments, TCR is rendered not functional in the cells according to the disclosure by disrupting or inactivating TCRα gene and/or TCRβ gene(s). In some embodiments, a method to obtain modified cells derived from an individual is provided, wherein the cells can proliferate independently of the major histocompatibility complex (MHC) signaling pathway. Modified cells, which can proliferate independently of the MHC signaling pathway, susceptible to be obtained by this method are encompassed in the scope of the present disclosure. Modified cells disclosed herein can be used in for treating patients in need thereof against Host versus Graft (HvG) rejection and Graft versus Host Disease (GvHD); therefore in the scope of the present disclosure is a method of treating patients in need thereof against Host versus Graft (HvG) rejection and Graft versus Host Disease (GvHD) comprising treating said patient by administering to said patient an effective amount of modified cells comprising disrupted or inactivated TCRα and/or TCRβ genes.

In some embodiments, the immune cells are engineered to be resistant to one or more chemotherapy drugs. The chemotherapy drug can be, for example, a purine nucleotide analogue (PNA), thus making the immune cell suitable for cancer treatment combining adoptive immunotherapy and chemotherapy. Exemplary PNAs include, for example, clofarabine, fludarabine, cyclophosphamide, and cytarabine, alone or in combination. PNAs are metabolized by deoxycytidine kinase (dCK) into mono-, di-, and tri-phosphate PNA. Their tri-phosphate forms compete with ATP for DNA synthesis, act as pro-apoptotic agents, and are potent inhibitors of ribonucleotide reductase (RNR), which is involved in trinucleotide production. Provided herein are Claudin 18.2-specific CAR-T cells comprising a disrupted or inactivated dCK gene. In some embodiments, the dCK knockout cells are made by transfection of T cells using polynucleotides encoding specific TAL-nuclease directed against dCK genes by, for example, electroporation of mRNA. The dCK knockout Claudin 18.2-specific CAR-T cells are resistant to PNAs, including for example clofarabine and/or fludarabine, and maintain T cell cytotoxic activity toward Claudin 18.2-expressing cells.

In some embodiments, isolated cells or cell lines of the disclosure can comprise a pTα or a functional variant thereof. In some embodiments, an isolated cell or cell line can be further genetically modified by disrupting or inactivating the TCRα gene.

The disclosure also provides engineered immune cells comprising any of the CAR polynucleotides described herein. In some embodiments, a CAR can be introduced into an immune cell as a transgene via a plasmid vector. In some embodiments, the plasmid vector can also contain, for example, a selection marker which provides for identification and/or selection of cells which received the vector.

CAR polypeptides may be synthesized in situ in the cell after introduction of polynucleotides encoding the CAR polypeptides into the cell. Alternatively, CAR polypeptides may be produced outside of cells, and then introduced into cells. Methods for introducing a polynucleotide construct into cells are known in the art. In some embodiments, stable transformation methods (e.g., using a lentiviral vector) can be used to integrate the polynucleotide construct into the genome of the cell. The polynucleotide construct can be integrated into the genome of the cell by, e.g., a lentiviral vector-mediated random integration, or by, e.g., homologous recombination-mediated site-specific integration via an adeno-associated viral vector. The polynucleotide construct can be integrated into a genomic locus of interest by homologous recombination, for example at one or more genomic loci where a disruption(s) (e.g. knock-out) at one or more endogenous genes occurs. Exemplary endogenous gene includes, without limitation, TCRα, TCRβ, CD52, glucocorticoid receptor (GR), deoxycytidine kinase (dCK), CD70 or an immune checkpoint protein such as for example programmed death-1 (PD-1).

In other embodiments, transient transformation methods can be used to transiently express the polynucleotide construct, and the polynucleotide construct not integrated into the genome of the cell. In other embodiments, virus-mediated methods can be used. The polynucleotides may be introduced into a cell by any suitable means such as for example, recombinant viral vectors (e.g., retroviruses, adenoviruses), liposomes, and the like. Transient transformation methods include, for example without limitation, microinjection, electroporation or particle bombardment. Polynucleotides may be included in vectors, such as for example plasmid vectors or viral vectors.

In some embodiments, isolated nucleic acids are provided comprising a promoter operably linked to a first polynucleotide encoding a Claudin 18.2 antigen binding domain, at least one costimulatory molecule, and an activating domain. In some embodiments, the nucleic acid construct is contained within a viral vector. In some embodiments, the viral vector is selected from the group consisting of retroviral vectors, murine leukemia virus vectors, SFG vectors, adenoviral vectors, lentiviral vectors, adeno-associated virus (AAV) vectors, Herpes virus vectors, and vaccinia virus vectors. In some embodiments, the nucleic acid is contained within a plasmid.

b. Immune Cells with Enhanced Resistance to Immune Rejection

In certain aspects, the disclosure provides engineered immune cell or a population of engineered immune cells comprising or expressing a Claudin 18.2-specific CAR and (1) further comprising or expressing an immune rejection avoidance protein, and/or (2) further comprising one or more genomic modifications that functionally impair or reduce expression of one or more CD58, NLRC5, RFX5, ICAM-1, TAP2, p2M, CIITA, RFXAP, RFXANK, and CD48. In some embodiments, the engineered immune cell or the population of engineered immune cells are allogeneic engineered immune cells. In some embodiments, the engineered immune cell or the population of engineered immune cells exhibit enhanced or increased resistance to host alloreactive immune cell rejection. In some embodiments, the increased resistance against alloreactive immune cell rejection is determinable and/or determined by a mixed lymphocyte reaction (MLR) assay, e.g., an MLR assay as described herein.

In some embodiments, the engineered immune cell or the population of engineered immune cells comprise one or more polynucleotides that encode a Claudin 18.2 CAR and an immune rejection avoidance protein. In some embodiments, the polynucleotide that encodes the Claudin 18.2 CAR and the polynucleotide that encodes the immune rejection avoidance protein can be part of the same polynucleotide or can be different polynucleotides. In some embodiments, the immune rejection avoidance protein comprises a CD70 binding protein. In some embodiments, the CD70 binding protein comprises or is a CD70 chimeric antigen receptor (CAR). In some embodiments, the CD70 binding protein comprises a CD70 binding domain and a transmembrane domain. In some embodiments, the CD70 binding domain comprises a CD70 antibody or an antigen binding fragment thereof, or a receptor for CD70 or a CD70 binding fragment thereof. In certain embodiments, the CD70 antibody comprises the amino acid sequence of SEQ ID NO: 204, 205, 206 and/or 207. In other embodiments, the CD70 binding domain comprises an anti-CD70 antibody, optionally the anti-CD70 antibody is a scFv.

In some embodiments, the CD70 binding protein comprises a CD8α transmembrane domain or a CD28 transmembrane domain. In further embodiments, the CD70 binding protein further comprises a hinge domain, optionally the hinge domain comprises a CD8α hinge domain or a CD28 hinge domain. In other embodiments, the CD70 binding protein further comprises one or more intracellular signaling domains selected from the group consisting of a CD3z signaling domain, a CD3d signaling domain, a CD3g signaling domain, a CD3e signaling domain, a CD28 signaling domain, a CD2 signaling domain, an OX40 signaling domain, and a 4-1BB signaling domain, or a variant thereof. In further embodiments, the CD70 binding protein comprises a CD3z or a CD3g signaling domain and does not comprise a costimulatory domain, such as a CD28 signaling domain or a 4-1BB signaling domain, or a variant thereof. In other embodiments, the CD70 binding protein comprises a 4-1BB signaling domain and does not comprise a CD3z signaling domain. In another embodiment, the CD70 binding protein comprises a 4-1BB signaling domain and a CD3z signaling domain. In other embodiments, the one or more intracellular domain comprises the amino acid sequence of one or more of SEQ ID NOs: 137, 138, 139, 158, 159, and 174. In some embodiments, the CD70 binding protein comprises In another embodiment, the CD70 binding protein does not comprise an intracellular signaling domain. In some embodiments, the engineered immune cell further comprises one or more genomic modifications that functionally impair or reduce the expression of CD70.

In additional embodiments, the genomic modification can be introduced by the zinc finger nuclease (ZFN), megaTAL nuclease, meganuclease, transcription activator-like effector nuclease (TALE-nuclease/TALEN), or CRISPR (e.g., Cas9 or Cas12) endonuclease.

c. Methods of Making

Provided herein are methods of making the CARs and the CAR-containing immune cells of the disclosure.

A variety of known techniques can be utilized in making the polynucleotides, polypeptides, vectors, antigen binding domains, immune cells, compositions, and the like according to the disclosure.

Prior to the in vitro manipulation or genetic modification of the immune cells described herein, the cells may be obtained from a subject. The cells expressing a Claudin 18.2 CAR may be derived from an allogenic or autologous process.

i. Source Material

In some embodiments, the immune cells comprise T cells. T cells can be obtained from a number of sources, including peripheral blood mononuclear cells (PBMCs), bone marrow, lymph nodes tissue, cord blood, thymus tissue, tissue from a site of infection, ascites, pleural effusion, spleen tissue, and tumors. In certain embodiments, T cells can be obtained from a unit of blood collected from the subject using any number of techniques known to the skilled person, such as FICOLLTH separation.

Cells may be obtained from the circulating blood of an individual by apheresis. The apheresis product typically contains lymphocytes, including T cells, monocytes, granulocytes, B cells, other nucleated white blood cells, red blood cells, and platelets. In certain embodiments, the cells collected by apheresis may be washed to remove the plasma fraction, and placed in an appropriate buffer or media for subsequent processing.

In certain embodiments, T cells are isolated from PBMCs by lysing the red blood cells and depleting the monocytes, for example, using centrifugation through a PERCOLLT™ gradient. A specific subpopulation of T cells, (e.g., CD28+, CD4+, CDS+, CD45RA−, CD45RO+, CDS+, CD62−, CD95−, CD95+, IL2β+, IL2β−, CCR7+, CCR7−, CDL−, CD62L+ and combinations thereof) can be further isolated by positive or negative selection techniques known in the art. In one example the subpopulation of T cells is CD45RA+, CD95−, IL-2β−, CCR7+, CD62L+. In one example the subpopulation of T cells is CD45RA+, CD95+, IL-2β+, CCR7+, CD62L+. In one example the subpopulation of T cells is CD45RO+, CD95+, IL-2β+, CCR7+, CD62L+. In one example the subpopulation of T cells is CD45RO+, CD95+, IL-2β+, CCR7−, CD62L−. In one example the subpopulation of T cells is CD45RA+, CD95+, IL-2β+, CCR7−, CD62L−. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD1 1b, CD16, HLA-DR, and CD8. Flow cytometry and cell sorting may also be used to isolate cell populations of interest for use in the present disclosure.

PBMCs may be used directly for genetic modification with the immune cells (such as CARs or TCRs) using methods as described herein. In certain embodiments, after isolating the PBMCs, T lymphocytes can be further isolated and both cytotoxic and helper T lymphocytes can be sorted into naive, memory, and effector T cell subpopulations either before or after genetic modification and/or expansion.

In some embodiments, CD8+ cells are further sorted into naive, stem cell memory, central memory, and effector cells by identifying characteristic cell surface antigens that are associated with each of these types of CD8+ cells. In some embodiments, the expression of phenotypic markers of central memory T cells include CD45RO, CD62L, CCR7, CD28, CD3, and CD127 and are negative for granzyme B. In some embodiments, stem cell memory T cells are CD45RO−, CD62L+, CD8+ T cells. In some embodiments, central memory T cells are CD45RO+, CD62L+, CD8+ T cells. In some embodiments, effector T cells are negative for CD62L, CCR7, CD28, and CD127, and positive for granzyme B and perforin.

In certain embodiments, CD4+ T cells are further sorted into subpopulations. For example, CD4+T helper cells can be sorted into naive, central memory, and effector cells by identifying cell populations that have characteristic cell surface antigens.

ii. Stem Cell-Derived Immune Cells

In some embodiments, the immune cells may be derived from stem cells, such as a progenitor cell, a bone barrow stem cell, an inducible pluripotent stem cell, an iPSC, a hematopoietic stem cell, and a mesenchymal stem cell. iPS cells and other types of stem cells may be cultivated immortal cell lines or isolated directly from a patient. Various methods for isolating, developing, and/or cultivating stem cells are known in the art and may be used to practice the present invention.

In some embodiments, the immune cell is an induced pluripotent stem cell (iPSC) derived from a reprogrammed T-cell. In some embodiments, the source material may be an induced pluripotent stem cell (iPSC) derived from a T cell or non-T cell. The source material may alternatively be a B cell, or any other cell from peripheral blood mononuclear cell isolates, hematopoietic progenitor, hematopoietic stem cell, mesenchymal stem cell, adipose stem cell, or any other somatic cell type.

iii. Genetic Modification of Isolated Cells

The immune cells, such as T cells, can be genetically modified following isolation using known methods, or the immune cells can be activated and expanded (or differentiated in the case of progenitors) in vitro prior to being genetically modified. In some embodiments, the isolated immune cells are genetically modified to reduce or eliminate expression of endogenous TCRα and/or CD52. In some embodiments, the cells are genetically modified using gene editing technology (e.g., CRISPR/Cas9, CRISPR/CAS12, a zinc finger nuclease (ZFN), a TALEN, a MegaTAL, a meganuclease) to reduce or eliminate expression of endogenous proteins (e.g., TCRα and/or CD52). In another embodiment, the immune cells, such as T cells, are optionally further genetically modified with the chimeric antigen receptors described herein (e.g., transduced with a viral vector comprising one or more nucleotide sequences encoding a CAR) and then are activated and/or expanded in vitro.

Methods for activating and expanding T cells are known in the art and are described, for example, in U.S. Pat. Nos. 6,905,874; 6,867,041; 6,797,514; and PCT WO2012/079000, the contents of which are hereby incorporated by reference in their entirety. Generally, such methods include contacting PBMC or isolated T cells with a stimulatory molecule and a costimulatory molecule, such as anti-CD3 and anti-CD28 antibodies, generally attached to a plastic or magnetic bead or other surface, in a culture medium with appropriate cytokines, such as IL-2. Anti-CD3 and anti-CD28 antibodies attached to the same bead serve as a “surrogate” antigen presenting cell (APC). One example is the Dynabeads® system, which is a CD3/CD28 activator/stimulator system for physiological activation of human T cells. In other embodiments, the T cells may be activated and stimulated to proliferate with feeder cells and appropriate antibodies and cytokines using methods such as those described in U.S. Pat. Nos. 6,040,177; 5,827,642; and WO2012129514, the contents of which are hereby incorporated by reference in their entirety.

Certain methods for making the constructs and engineered immune cells of the disclosure are described in PCT application PCT/US15/14520, the contents of which are hereby incorporated by reference in their entirety.

It will be appreciated that PBMCs can further include other cytotoxic lymphocytes such as NK cells or NKT cells. An expression vector carrying the coding sequence of a chimeric receptor as disclosed herein can be introduced into a population of human donor T cells, NK cells or NKT cells. Successfully transduced T cells that carry the expression vector can be sorted using flow cytometry to isolate CD3 positive T cells and then further propagated to increase the number of these CAR expressing T cells in addition to cell activation using anti-CD3 antibodies and IL-2 or other methods known in the art as described elsewhere herein. Standard procedures are used for cryopreservation of T cells expressing the CAR for storage and/or preparation for use in a human subject. In one embodiment, the in vitro transduction, culture and/or expansion of T cells are performed in the absence of non-human animal derived products such as fetal calf serum and fetal bovine serum. In an embodiment, cryopreservation can comprise freezing in a suitable medium, such as CryoStor® CS10, CryoStor® CS2 or CryoStor® CS5 (BioLife Solutions).

For cloning of polynucleotides, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vector provided herein. The host cells containing the vector may be useful in expression or cloning of the polynucleotide contained in the vector. Suitable host cells can include, without limitation, prokaryotic cells, fungal cells, yeast cells, or higher eukaryotic cells such as mammalian cells, and more specifically human cells.

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for viral transfection and transformation of cells for expression of a vector of interest are well known in the art. In a further embodiment, a mixture of different expression vectors can be used in genetically modifying a donor population of immune effector cells wherein each vector encodes a different CAR as disclosed herein. The resulting transduced immune effector cells form a mixed population of engineered cells, with a proportion of the engineered cells expressing more than one different CARs.

In one embodiment, the disclosure provides a method of storing genetically engineered cells expressing CARs which target a Claudin 18.2 protein. In an embodiment this involves cryopreserving the immune cells such that the cells remain viable upon thawing. In an embodiment, cryopreservation can comprise freezing in a suitable medium, such as CryoStor® CS10, CryoStor® CS2 or CryoStor® CS5 (BioLife Solutions). A fraction of the immune cells expressing the CARs can be cryopreserved by methods known in the art to provide a permanent source of such cells for the future treatment of patients afflicted with a malignancy. When needed, the cryopreserved transformed immune cells can be thawed, grown and expanded for more such cells.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion media can be any isotonic medium formulation, typically normal saline, Normosol™ R (Abbott) or Plasma-Lyte™ A (Baxter), but also 5% dextrose in water or Ringer's lactate can be utilized. The infusion medium can be supplemented with human serum albumin.

iv. Allogeneic CAR T cells

In brief, the process for manufacturing allogeneic CAR T therapy involves harvesting healthy, selected, screened and tested PBMCs or T cells from healthy donors. Allogeneic T cells are gene editing to reduce the risk of graft versus host disease (GvHD) and to prevent allogeneic rejection. A selected T cell receptor gene (e.g., TCRα, TCRβ) is knocked out to avoid GvHD. The CD52 gene can also be knocked out to render the CAR T product resistant to anti-CD52 antibody treatment. Anti-CD52 antibody treatment can therefore be used to lymphodeplete the host immune system and allow the CAR T cells to stay engrafted to achieve full therapeutic impact. Next, the T cells are engineered to express CARs, which recognize certain cell surface proteins (e.g., Claudin 18.2) that are expressed in hematologic or solid tumors. The engineered T cells then undergo a purification step and are ultimately cryopreserved in vials for delivery to patients.

v. Autologous CAR T Cells

Autologous chimeric antigen receptor (CAR) T cell therapy involves collecting a patient's own cells (e.g., white blood cells, including T cells) and genetically engineering the T cells to express CARs that recognize a target antigen expressed on the cell surface of one or more specific cancer cells and kill cancer cells. The engineered cells are then cryopreserved and subsequently administered to the patient from which the cells were removed for engineering.

### IV. Methods of Treatment

The disclosure comprises methods for treating or preventing a condition associated with Claudin 18.2, or undesired and/or elevated Claudin 18.2 levels in a patient, comprising administering to a patient in need thereof an effective amount of at least one CAR, or immune-cell comprising a CAR disclosed herein.

Methods are provided for treating diseases or disorders, including cancer. In some embodiments, the disclosure relates to creating a T cell-mediated immune response in a subject, comprising administering an effective amount of the engineered immune cells of the present application to the subject. In some embodiments, the T cell-mediated immune response is directed against a target cell or cells. In some embodiments, the engineered immune cell comprises a chimeric antigen receptor (CAR). In some embodiments, the target cell is a tumor cell. In some aspects the disclosure comprises a method for treating or preventing a malignancy, said method comprising administering to a subject in need thereof an effective amount of at least one isolated antigen binding domain described herein. In some aspects, the disclosure comprises a method for treating or preventing a malignancy, said method comprising administering to a subject in need thereof an effective amount of at least one immune cell, wherein the immune cell comprises at least one chimeric antigen receptor, and/or isolated antigen binding domain as described herein. The CAR containing immune cells of the disclosure can be used to treat malignancies involving aberrant expression of Claudin 18.2. In some embodiments, CAR containing immune cells of the disclosure can be used to treat such malignancies as gastric cancer, gastroesophageal junction (GEJ) cancer, pancreatic cancer, small cell lung cancer, melanoma, low grade gliomas, glioblastoma, medullary thyroid cancer, carcinoids, dispersed neuroendocrine tumors in the pancreas, bladder and prostate, testicular cancer, and lung adenocarcinomas with neuroendocrine features. In exemplary embodiments, the CAR-containing immune cells, e.g., the anti-Claudin 18.2 CAR-T cells of the disclosure, are used to treat small cell lung cancer.

Also provided are methods for reducing the size of a tumor in a subject, comprising administering to the subject an engineered cell of the present disclosure to the subject, wherein the cell comprises a chimeric antigen receptor comprising a Claudin 18.2 antigen binding domain and binds to a Claudin 18.2 antigen on the tumor.

In some embodiments, the subject has a solid tumor, or a blood malignancy such as lymphoma or leukemia. In some embodiments, the engineered cell is delivered to a tumor bed, such as a tumor bed found in small cell lung cancer. In some embodiments, the cancer is present in the bone marrow of the subject. In some embodiments, the engineered cells are autologous immune cells, e.g., autologous T cells. In some embodiments, the engineered cells are allogeneic immune cells, e.g., allogeneic T cells. In some embodiments, the engineered cells are heterologous immune cells, e.g., heterologous T cells. In some embodiments, the engineered cells are transfected or transduced ex vivo. As used herein, the term “in vitro cell” refers to any cell that is cultured ex vivo.

An “effective amount” is any amount that, when used alone or with another agent, provides desired or beneficial results. A “therapeutically effective amount,” “effective dose,” or “therapeutically effective dosage” of a therapeutic agent, e.g., engineered CAR T cells, is any amount that, when used alone or in combination with another therapeutic agent, protects a subject against the onset of a disease or promotes disease regression evidenced by a decrease in severity of disease symptoms, an increase in frequency and duration of disease symptom-ftee periods, or a prevention of impairment or disability due to the disease affliction. The ability of a therapeutic agent to promote disease regression can be evaluated using a variety of methods known to the skilled practitioner (e.g., a physician or clinician), such as in human subjects during clinical trials, in animal model systems predictive of efficacy in humans, or by assaying the activity of the agent in in vitro assays.

The terms “patient” and “subject” are used interchangeably and include human and non-human animal subjects as well as those with formally diagnosed disorders, those without formally recognized disorders, those receiving medical attention, those at risk of developing the disorders, etc.

The term “treat” and “treatment” includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. The term “prevent” does not require the 100% elimination of the possibility of an event. Rather, it denotes that the likelihood of the occurrence of the event has been reduced in the presence of the compound or method.

Desired treatment total amounts of cells in the composition comprise at least 2 cells (for example, at least one CD8+ T cell and at least one CD4+ T cell, or two CD8+ T cells, or two CD4+ T cells) or is more typically greater than 10² cells, and up to 10⁶, up to and including 10⁸ or 10⁹ cells and can be 10¹⁰ or 10¹² or more cells. The number of cells will depend upon the desired use for which the composition is intended, and the type of cells included therein. The density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10³, 10¹¹, 10¹⁰, 10¹¹, or 10¹² cells. In some aspects of the present disclosure, particularly since all the infused cells will be redirected to a particular target antigen (e.g., Claudin 18.2), lower numbers of cells, in the range of 10⁶/kilogram (10⁶-10¹¹ per patient) may be administered. CAR treatments may be administered multiple times at dosages within these ranges. The cells may be autologous, allogeneic, or heterologous to the patient undergoing therapy.

In some embodiments, the therapeutically effective amount of the CAR T cells is about 1×10⁵ cells/kg, about 2×10⁵ cells/kg, about 3×10⁵ cells/kg, about 4×10⁵ cells/kg, about 5×10⁵ cells/kg, about 6×10⁵ cells/kg, about 7×10⁵ cells/kg, about 8×10⁵ cells/kg, about 9×10⁵ cells/kg, 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

In some embodiments, target doses for CAR+/CAR-T+ cells range from about 1×10⁶ to about 1×10¹⁰ cells/kg, for example about 1×10⁶ cells/kg, about 1×10⁷ cells/kg, about 1×10⁸ cells/kg, about 1×10⁹ cells/kg or about 1×10¹⁰ cells/kg. It will be appreciated that doses above and below this range may be appropriate for certain subjects, and appropriate dose levels can be determined by the healthcare provider as needed. Additionally, multiple doses of cells can be provided in accordance with the disclosure.

In some aspects the disclosure comprises a pharmaceutical composition comprising at least one antigen binding domain as described herein and a pharmaceutically acceptable excipient. In some embodiments, the pharmaceutical composition further comprises an additional active agent.

The CAR expressing cell populations of the present disclosure may be administered either alone, or as a pharmaceutical composition in combination with diluents and/or with other components such as IL-2 or other cytokines or cell populations. Pharmaceutical compositions of the present disclosure may comprise a CAR expressing cell population, such as T cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for intravenous administration.

The pharmaceutical compositions (solutions, suspensions or the like), may include one or more of the following: sterile diluents such as water for injection, saline solution, preferably physiological saline, Ringer's solution, isotonic sodium chloride, fixed oils such as synthetic mono- or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. For therapeutic applications, an injectable pharmaceutical composition is preferably sterile.

In some embodiments, upon administration to a patient, engineered immune cells expressing at their cell surface any one of the Claudin 18.2-specific CARs described herein may reduce, kill or lyse endogenous Claudin 18.2-expressing cells of the patient. In one embodiment, a percentage reduction or lysis of Claudin 18.2-expressing endogenous cells or cells of a cell line expressing Claudin 18.2 by engineered immune cells expressing any one of the Claudin 18.2-specific CARs described herein is at least about or greater than 10%, 15%, 20%, 25%, 30%, 35%, 40%, 45%, 50%, 55%, 60%, 65%, 70%, 75%, 80%, 85%, 90%, or 95%. In one embodiment, a percentage reduction or lysis of Claudin 18.2-expressing endogenous cells or cells of a cell line expressing Claudin 18.2 by engineered immune cells expressing any one of the Claudin 18.2-specific CARs described herein is about 5% to about 95%, about 10% to about 95%, about 10% to about 90%, about 10% to about 80%, about 10% to about 70%, about 10% to about 60%, about 10% to about 50%, about 10% to about 40%, about 20% to about 90%, about 20% to about 80%, about 20% to about 70%, about 20% to about 60%, about 20% to about 50%, about 25% to about 75%, or about 25% to about 60%.

In one embodiment, the percent reduction or lysis of target cells, e.g., a cell line expressing Claudin 18.2, by engineered immune cells expressing at their cell surface membrane a Claudin 18.2-specific CAR of the disclosure can be measured using the assay disclosed herein.

In certain embodiments, compositions comprising CAR-expressing immune effector cells disclosed herein may be administered to a patient systemically, e.g., by intravenous injection, or locally close to the disease site, e.g., by intraperitoneal injection. In some embodiments, the compositions comprising anti-Claudin 18.2 CAR T cells may be administered intraperitoneally. In some embodiments, local, non-systemic delivery of the anti-Claudin 18.2 CAR T cells described herein can achieve efficacy with lower doses and/or reduce adverse safety events, as compared to systemic delivery and, as a result can improve the overall therapeutic index of the CAR T treatment.

The methods can further comprise administering one or more chemotherapeutic agents to a patient prior to administering the engineered cells provided herein. In certain embodiments, the chemotherapeutic agent is a lymphodepleting (preconditioning) chemotherapeutic. For example, methods of conditioning a patient in need of a T cell therapy comprising administering to the patient specified beneficial doses of cyclophosphamide (between 200 mg/m²/day and 2000 mg/m²/day, about 100 mg/m²/day and about 2000 mg/m²/day; e.g., about 100 mg/m²/day, about 200 mg/m²/day, about 300 mg/m²/day, about 400 mg/m²/day, about 500 mg/m²/day, about 600 mg/m²/day, about 700 mg/m²/day, about 800 mg/m²/day, about 900 mg/m²/day, about 1000 mg/m²/day, about 1500 mg/m²/day or about 2000 mg/m²/day) and specified doses of fludarabine (between 20 mg/m²/day and 900 mg/m²/day, between about 10 mg/m²/day and about 900 mg/m²/day; e.g., about 10 mg/m²/day, about 20 mg/m²/day, about 30 mg/m²/day, about 40 mg/m²/day, about 40 mg/m²/day, about 50 mg/m²/day, about 60 mg/m²/day, about 70 mg/m²/day, about 80 mg/m²/day, about 90 mg/m²/day, about 100 mg/m²/day, about 500 mg/m²/day or about 900 mg/m²/day). An exemplary dosing regimen involves treating a patient comprising administering daily to the patient about 300 mg/m²/day of cyclophosphamide in combination or before or after administering about 30 mg/m²/day of fludarabine for three days prior to administration of a therapeutically effective amount of engineered T cells to the patient.

In some embodiments, notably in the case when the engineered cells provided herein have been gene edited to eliminate or minimize surface expression of CD52, lymphodepletion further comprises administration of an anti-CD52 antibody, such as alemtuzumab. In some embodiments, the CD52 antibody is administered at a dose of about 1-20 mg/day IV, e.g., about 13 mg/day IV, e.g., about 20 mg/day IV, e.g., about 30 mg/day IV, for 1, 2, 3 or more days. The antibody can be administered in combination with, before, or after administration of other elements of a lymphodepletion regime (e.g., cyclophosphamide and/or fludarabine).

In other embodiments, the antigen binding domain, transduced (or otherwise engineered) cells and the chemotherapeutic agent are administered each in an amount effective to treat the disease or condition in the subject.

In certain embodiments, compositions comprising CAR-expressing immune effector cells disclosed herein may be administered in conjunction with any number of chemotherapeutic agents. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idanibicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′, 2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RF S2000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®), Doxorubicin (hydroxydoxorubicin), Vincristine (Oncovin®), and Prednisone.

In some embodiments, the chemotherapeutic agent is administered at the same time or within one week after the administration of the engineered cell, polypeptide, or nucleic acid. In other embodiments, the chemotherapeutic agent is administered from about 1-7 days, about 1 to about 4 weeks or from about 1 week to about 1 month, about 1 week to about 2 months, about 1 week to about 3 months, about 1 week to about 6 months, about 1 week to about 9 months, or about 1 week to about 12 months after the administration of the engineered cell, polypeptide, or nucleic acid. In other embodiments, the chemotherapeutic agent is administered at least 1 month before administering the cell, polypeptide, or nucleic acid. In some embodiments, the methods further comprise administering two or more chemotherapeutic agents.

A variety of additional therapeutic agents may be used in conjunction with the compositions described herein. For example, potentially useful additional therapeutic agents include PD-1 inhibitors such as nivolumab (Opdivo®), pembrolizumab (Keytruda®), pembrolizumab, pidilizumab, and atezolizumab.

Additional therapeutic agents suitable for use in combination with the disclosure include, but are not limited to, ibrutinib (Imbruvica®), ofatumumab (Arzerra®, rituximab (Rituxan®), bevacizumab (Avastin®), trastuzumab (Herceptin®), trastuzumab emtansine (KADCYLA®, imatinib (Gleevec®), cetuximab (Erbitux®, panitumumab) (Vectibix®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib).

In some embodiments, the composition comprising CAR-containing immune cells may be administered with a therapeutic regimen to prevent or reduce cytokine release syndrome (CRS) or neurotoxicity. The therapeutic regimen to prevent cytokine release syndrome (CRS) or neurotoxicity may include lenzilumab, tocilizumab, atrial natriuretic peptide (ANP), anakinra, iNOS inhibitors (e.g., L-NIL or 1400W). In additional embodiments, the composition comprising CAR-containing immune cells can be administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of propoxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

In certain embodiments, the compositions described herein are administered in conjunction with a cytokine. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, IL-21 a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

### V. Methods of Sorting and Depletion

In some embodiments, provided are methods for in vitro sorting of a population of immune cells, wherein a subset of the population of immune cells comprises engineered immune cells expressing any one of the Claudin 18.2-specific CARs comprising epitopes specific for monoclonal antibodies (e.g., exemplary mimotope sequences). The method comprises contacting the population of immune cells with a monoclonal antibody specific for the epitopes and selecting the immune cells that bind to the monoclonal antibody to obtain a population of cells enriched in engineered immune cells expressing the Claudin 18.2-specific CAR.

In some embodiments, said monoclonal antibody specific for said epitope is optionally conjugated to a fluorophore. In this embodiment, the step of selecting the cells that bind to the monoclonal antibody can be done by Fluorescence Activated Cell Sorting (FACS).

In some embodiments, said monoclonal antibody specific for said epitope is optionally conjugated to a magnetic particle. In this embodiment, the step of selecting the cells that bind to the monoclonal antibody can be done by Magnetic Activated Cell Sorting (MACS).

In some embodiments, the mAb used in the method for sorting immune cells expressing the CAR is chosen from alemtuzumab, ibritumomab tiuxetan, muromonab-CD3, tositumomab, abciximab, basiliximab, brentuximab vedotin, cetuximab, infliximab, rituximab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and/or ustekinumab. In some embodiments, said mAb is rituximab. In another embodiment, said mAb is QBEND-10.

In some embodiments, the population CAR-expressing immune cells obtained when using the method for in vitro sorting CAR-expressing immune cells described above, comprises at least 70%, 75%, 80%, 85%, 90%, 95% of CAR-expressing immune cells. In some embodiments, the population of CAR-expressing immune cells obtained when using the method for in vitro sorting CAR-expressing immune cells, comprises at least 85% CAR-expressing immune cells.

In some embodiments, the population of CAR-expressing immune cells obtained when using the method for in vitro sorting CAR-expressing immune cells described above shows increased cytotoxic activity in vitro compared with the initial (non-sorted) cell population. In some embodiments, said cytotoxic activity in vitro is increased by 10%, 20%, 30% or 50%. In some embodiments, the immune cells are T-cells.

In some embodiments, the mAbs are previously bound onto a support or surface. Non-limiting examples of solid support may include a bead, agarose bead, a plastic bead a magnetic bead, a plastic welled plate, a glass welled plate, a ceramic welled plate, a column, or a cell culture bag.

The CAR-expressing immune cells to be administered to the recipient may be enriched in vitro from the source population. Methods of expanding source populations may include selecting cells that express an antigen such as CD34 antigen, using combinations of density centrifugation, immuno-magnetic bead purification, affinity chromatography, and fluorescent activated cell sorting.

Flow cytometry may be used to quantify specific cell types within a population of cells. In general, flow cytometry is a method for quantitating components or structural features of cells primarily by optical means. Since different cell types can be distinguished by quantitating structural features, flow cytometry and cell sorting can be used to count and sort cells of different phenotypes in a mixture.

A flow cytometry analysis involves two primary steps: 1) labeling selected cell types with one or more labeled markers, and T) determining the number of labeled cells relative to the total number of cells in the population. In some embodiments, the method of labeling cell types includes binding labeled antibodies to markers expressed by the specific cell type. The antibodies may be either directly labeled with a fluorescent compound or indirectly labeled using, for example, a fluorescent-labeled second antibody which recognizes the first antibody.

In some embodiments, the method used for sorting T cells expressing CAR is the Magnetic-Activated Cell Sorting (MACS). Magnetic-activated cell sorting (MACS) is a method for separation of various cell populations depending on their surface antigens (CD molecules) by using superparamagnetic nanoparticles and columns. MACS may be used to obtain a pure cell population. Cells in a single-cell suspension may be magnetically labeled with microbeads. The sample is applied to a column composed of ferromagnetic spheres, which are covered with a cell-friendly coating allowing fast and gentle separation of cells. The unlabeled cells pass through while the magnetically labeled cells are retained within the column. The flow-through can be collected as the unlabeled cell fraction. After a washing step, the column is removed from the separator, and the magnetically labeled cells are eluted from the column.

Detailed protocol for the purification of specific cell population such as T-cell can be found in Basu S et al. (2010). (Basu S, Campbell H M, Dittel B N, Ray A. Purification of specific cell population by fluorescence activated cell sorting (FACS). J Vis Exp. (41): 1546).

In some aspects the present disclosure provides a method for depleting Claudin 18.2 specific CAR-expressing immune cells by in vivo depletion. in vivo depletion may include the administration of a treatment (e.g., a molecule that binds an epitope on the CAR) to a mammalian organism aiming to stop the proliferation of the CAR-expressing immune cells by inhibition or elimination.

One aspect of the invention is related to a method for in vivo depleting an engineered immune cell expressing a Claudin 18.2 CAR comprising a mAb specific epitope, comprising contacting said engineered immune cell or said CAR-expressing immune cell with at least one epitope-specific mAb. Another aspect of the invention relates to a method for in vivo depleting CAR-expressing immune cell which comprises a chimeric scFv (e.g., formed by insertion of a mAb-specific epitope) by contacting said engineered immune cell with epitope-specific antibodies. In some embodiments, the immune cells are T-cells and/or the antibodies are monoclonal.

According to one embodiment, the in vivo depletion of the immune engineered cells is performed on engineered immune cells which has been previously sorted using the in vitro method of the present invention. In this case, the same infused mAb may be used. In some embodiments, the mAb-specific antigen is CD20 antigen and the epitope-specific mAb is rituximab. In some embodiments, the invention relates to a method for in vivo depleting an engineered immune cell expressing a CAR comprising an mAb-specific epitope (CAR-expressing immune cell) in a patient comprising contacting said CAR-expressing immune cell with at least one epitope-specific mAb.

In some embodiments, the step of contacting said engineered immune cell or said CAR-expressing immune cell with at least one epitope-specific mAb comprises infusing the patient with epitope-specific mAb (e.g., rituximab). In some embodiments, the amount of epitope-specific mAb administered to the patient is sufficient to eliminate at least 20%, 30%, 40%, 50%, 60%, 70%, 80% or 90% of the CAR-expressing immune cell in the patient.

In some embodiments, the step of contacting said engineered immune cell or said CAR-expressing immune cell with at least one epitope-specific mAb comprises infusing the patient with about 375 mg/m² of rituximab, once or several times. In some embodiments, the mAb (e.g., rituximab) is administered once weekly.

In some embodiments, when immune cells expressing a CAR comprising an mAb-specific epitope (CAR-expressing immune cells) are depleted in a complement dependent cytotoxicity (CDC) assay using epitope-specific mAb, the amount of viable CAR-expressing immune cells decreases. In some embodiments, the amount of viable CAR-expressing immune cells decreases by at least 10%, 20%, 30%, 40%, 50%, 60%, 70%, 80% or 90%. In some embodiments, said mAb-specific epitope is a CD20 epitope or mimotope and/or the epitope-specific mAb is rituximab.

In certain embodiments, the in vivo depletion of CAR-engineered immune cells is performed by infusing bi-specific antibodies. By definition, a bispecific monoclonal antibody (BsAb) is an artificial protein that is composed of fragments of two different monoclonal antibodies and consequently binds to two different types of antigen. These BsAbs and their use in immunotherapy have been reviewed in Muller D and Kontermann R. E. (2010) Bispecific Antibodies for Cancer Immunotherapy, BioDrugs 24 (2): 89-98.

According to another particular embodiment, the infused bi-specific mAb is able to bind both the mAb-specific epitope bome on engineered immune cells expressing the chimeric scFv and to a surface antigen on an effector and cytotoxic cell (e.g., immune cells such as lymphocytes, macrophages, dendritic cells, natural killer cells (NK Cell), cytotoxic T lymphocytes (CTL)). By doing so, the depletion of engineered immune cells triggered by the BsAb may occur through antibody-dependent cellular cytotoxicity (ADCC). (Deo Y M, Sundarapandiyan K, Keler T, Wallace P K, and Graziano R F, (2000), Journal of Immunology, 165 (10):5954-5961]).

In some embodiments, a cytotoxic drug is coupled to the epitope-specific mAbs which may be used to deplete CAR-expressing immune cells. By combining targeting capabilities of monoclonal antibodies with the cancer-killing ability of cytotoxic drugs, antibody-drug conjugate (ADC) allows a sensitive discrimination between healthy and diseased tissue when compared to the use of the drug alone. Market approvals were received for several ADCs; the technology for making them -particularly on linkers-are described in (Payne, G. (2003) Cancer Cell 3:207-212; Trail et al (2003) Cancer Immunol. Immunother. 52:328-337; Syrigos and Epenetos (1999) Anticancer Research 19:605-614; Niculescu-Duvaz and Springer (1997) Adv. Drug Del. Rev. 26:151-172; U.S. Pat. No. 4,975,278).

In some embodiments, the epitope-specific mAb to be infused is conjugated beforehand with a molecule able to promote complement dependent cytotoxicity (CDC). Therefore, the complement system helps or complements the ability of antibodies to clear pathogens from the organism. When stimulated an activation cascade is triggered as a massive amplification of the response and activation of the cell-killing membrane attack complex. Different molecule may be used to conjugate the mAb, such as glycans (Courtois, A, Gac-Breton, S., Berthou, C, Guezennec, J., Bordron, A. and Boisset, C. (2012), Complement dependent cytotoxicity activity of therapeutic antibody fragments may be acquired by immunogenic glycan coupling, Electronic Journal of Biotechnology ISSN: 0717-3458; http://www.ejbiotechnology.info DOI: 10.2225/vol15-issue5).

### VI. Kits and Articles of Manufacture

The present application provides kits comprising any one of the Claudin 18.2 containing CARs or Claudin 18.2 CAR containing immune cells described herein, and pharmaceutical compositions of the same. In an embodiment of a kit the engineered CAR cells are frozen in a suitable medium, such as CryoStor® CS10, CryoStor® CS2 or CryoStor® CS5 (BioLife Solutions).

In some exemplary embodiments, a kit of the disclosure comprises allogeneic Claudin 18.2 CAR-containing T-cells and a CD52 antibody for administering to the subject for use in a lymphodepletion regiment and a CAR-T regimen.

The present application also provides articles of manufacture comprising any one of the therapeutic compositions or kits described herein. Examples of an article of manufacture include vials (e.g., sealed vials).

### EXAMPLES

### Example 1. Characterization of Claudin 18.2 Targeting Antibodies

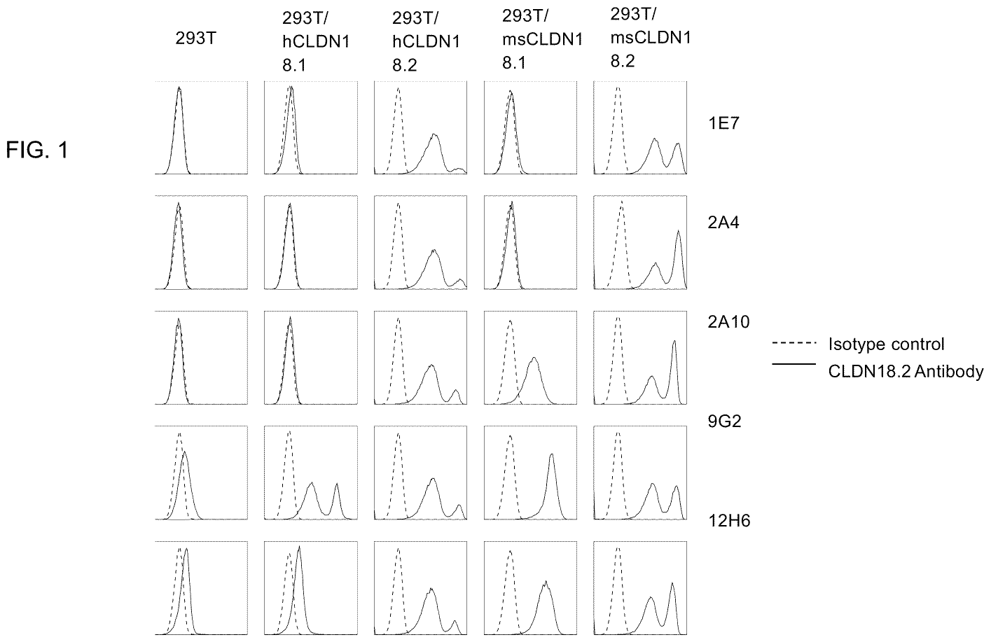

To test the binding capacity and specificity of Claudin 18.2 antibodies, HEK-293T, a Claudin 18 negative cell line, was engineered to overexpress human or mouse Claudin 18.1 or 18.2. Cells were stained with 5 μg/mL of purified anti-Claudin 18.2 scFv-hIgG2Fc or control scFv-hIgG Fc in PBS for 30 minutes at 4° C. Bound Claudin 18.2 antibodies were then detected with PE labeled anti-human IgG Fcγ antibody (Jackson ImmunoResearch, Cat #109-0116-098) at 1:200 dilution. The stained samples were analyzed by flow cytometry. Representative images showing binding of Claudin 18.2 antibodies to 293T parental and engineered cells are included in FIG. 1. The solid line and dashed line represent staining with anti-Claudin 18.2 antibodies or isotype control, respectively. Due to the high sequence homology between human and mouse Claudin 18.2, all anti-human Claudin 18.2 antibodies cross-react and bind to mouse Claudin 18.2. Clones that specifically bind to human Claudin 18.2 but not human Claudin 18.1 are considered optimal clones for reformatting to CARs due to greater specificity.

### Example 2. Generation of Claudin 18.2 CAR T Cells

The Claudin 18.2 CAR T cells were prepared using lentiviral transduction. To make lentivirus encoding Claudin 18.2 CARs, HEK-293T cells were plated at 0.8×10⁶ cells/mL in 2 mL of DMEM (Gibco) supplemented with 10% FBS, 1× Non-Essential Amino Acids, 1 mM Sodium Pyruvate and 25 mM HEPES per well of a 6-well plate. The next day, lentiviral packaging vectors, including 1.5 μg psPAX2 and 0.5 μg pMD2.G, were mixed with 0.5 μg plasmids expressing CAR constructs in 250 μL Opti-MEM. 10 μL Lipofectamine 2000 in 250 μL Opti-MEM was incubated at room temperature for 5 minutes and then added to the DNA mix. The total 500 μL DNA/lipofectamine mixture were incubated at room temperature for 20 minutes before adding to the wells seeded with HEK-293T cells. The cells were then returned to a 37° C. incubator with 5% CO₂ for overnight. One day post transfection, the media from each well of the 6-well plate was replaced with 2 mL X-VIVO™ 15 supplemented with 10% FBS. The supernatants were collected after 24 hours and passed through a 0.45 μm filter (EMD Millipore) followed by lentivirus concentration using Lenti-X (Takara). The concentrated viruses were resuspended in 200 μL X-VIVO™ 15 with 10% FBS and used directly for T cell transduction.

To generate T cells expressing Claudin 18.2 CARs, primary human T cells were directly purified from LeukoPak (StemCell Technologies) using EasySep™ Human T Cell Isolation Kit (StemCell Technologies, Cat #17951) and cryopreserved. T cells were activated immediately after recovery from cryopreservation with human T Cell TransAct (Miltenyi Biotec, Cat #130-111-160, 1:100 dilution) in X-VIVO™ 15 (Lonza) supplemented with 10% FBS and 100 IU/mL human IL-2 (Miltenyi Biotec). Two days after, activated T cells were harvested and resuspended at 1×10⁶ cells/mL in 1 mL fresh medium containing IL-2. The prepared viruses were then added for CAR transduction. On Day 5 post activation, cells were fed by replacing the spent media with T cell expansion media, i.e., X-VIVO™ 15 supplemented with 5% human AB serum (Gemini Bio), along with 100 IU/mL human IL-2. On Day 6, the TCRα constant (TRAC) and CD52 genes were knocked out by Transcription Activator-Like Effector Nucleases (TALEN)-mediated gene editing. Cells were expanded into larger flasks or G-Rex vessels (Wilson Wolf) as needed using T cell expansion media with IL-2. On Day 14, TCRα/β negative cells were purified using the EasySep Human TCRα/b depletion kit (StemCell Technologies) and rested overnight in T cell expansion media containing 100 IU/mL IL-2 before cryopreservation on Day 15. Percentage of CAR+ cells across all samples were normalized to the one with the lowest transduction efficiency by the addition of non-transduced (NTD) T cells right before cryopreservation. On Day 9 and Day 14, CAR transduction efficiency and phenotype were assessed by flow cytometry. To determine the percentage of CAR transduction, T cells were first stained with 50 μg/mL biotinylated recombinant Protein L (Thermo Scientific, Cat #29997) in PBS, for 30 minutes at 4° C. and followed by PE Streptavidin (Biolegend, Cat #405204) at 1:200 dilution for 30 minutes at 4° C. The memory phenotype was analyzed according to CD62L and CD45RO expression within the CAR+ cell population (gated based on PE-conjugated Rituximab detection): stem cell memory (TSCM, CD45RO−/CD62L+), central memory (TCM, CD45RO+/CD62L+), effector memory (TEM, CD45RO+/CD62L−), effector cells (TEFF, CD45RO−/CD62L−).

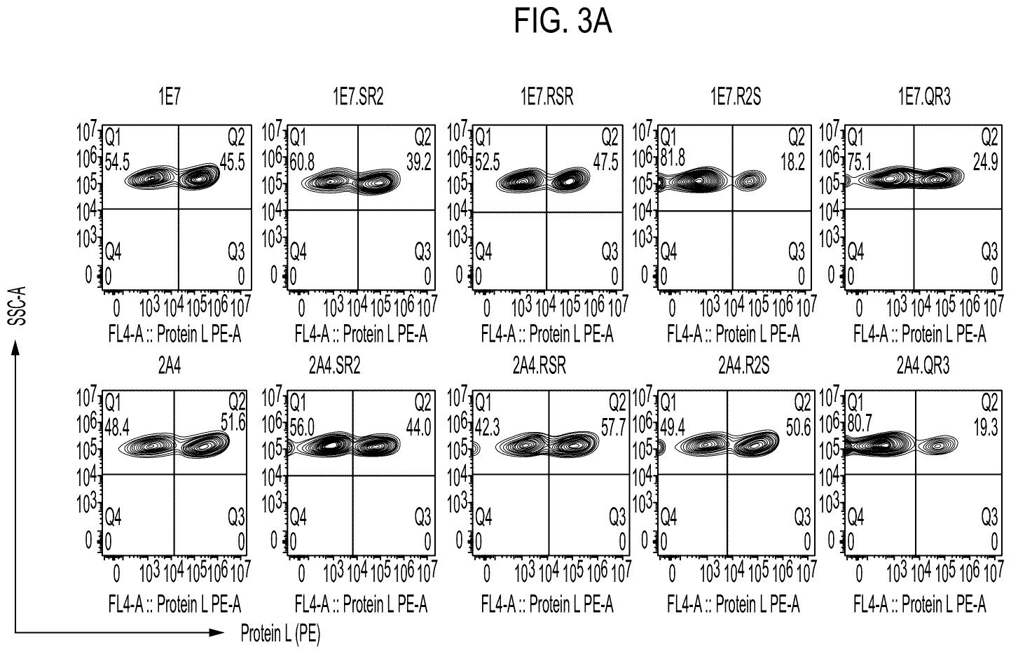

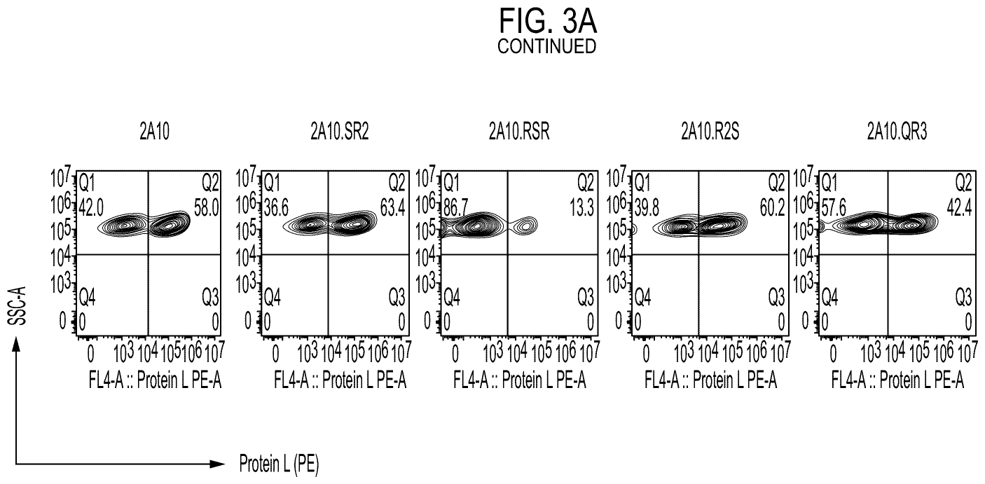

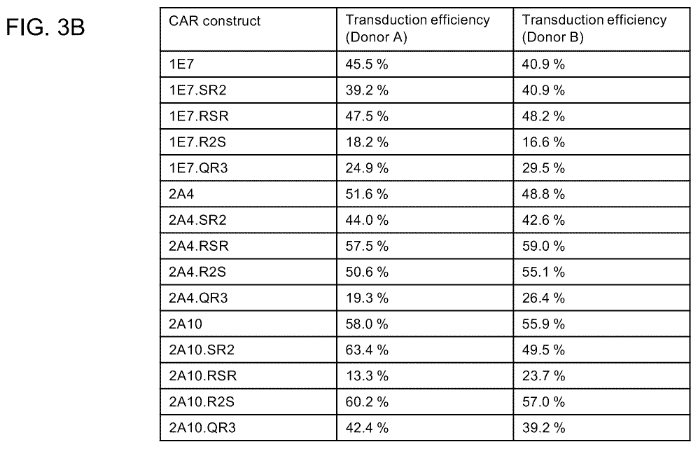

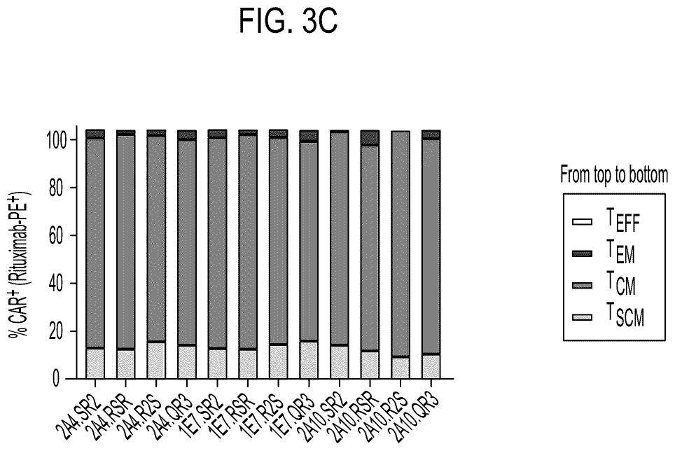

Examples of Claudin 18.2 CAR T cells with safety switches are shown in FIGS. 3A-3C. FIG. 3A shows representative FACS plots demonstrating varying levels of transduction of anti-Claudin 18.2 CARs in different rituximab off-switch formats. Constructs that resulted in low transduction efficiencies were considered less desirable for large scale manufacture. FIG. 3B summarizes transduction efficiency in two different donors. FIG. 3C shows the comparable distribution of T cell differentiation subsets among different Claudin 18.2 CAR T cells.

### Example 3. In Vitro Cytotoxicity of Claudin 18.2 CAR T Cells

To test Claudin 18.2-specific killing, multiple human cancer cell lines were used, including Claudin 18.2 positive cells (PATU8988s, MKN45/hClaudin 18.2), Claudin 18.1 positive cells (MKN45/hClaudin 18.1) and cells that are negative for both Claudin 18.1 and 18.2 (22rv1). All cell lines were engineered to express firefly luciferase. On Day 0 of the assay, 1×10⁴ target cells were seeded in 100 μL RPMI supplemented with 10% FBS per well in a white flat-bottomed 96-well tissue culture plate. After target cells attached to the bottom of the plates, Claudin 18.2 CAR T cells were thawed and added to plated target cells at different effector:target (E:T) ratios ranging from 1:9 to 3:1 in 100 μL RPMI supplemented with 10% FBS. Cell viability was measured after 72 hours using one-glo luciferase assay kit (Promega). Each condition was assayed in duplicate and percentage lysis of target cells was calculated by normalizing luciferase activity for each CAR treatment to target alone control.

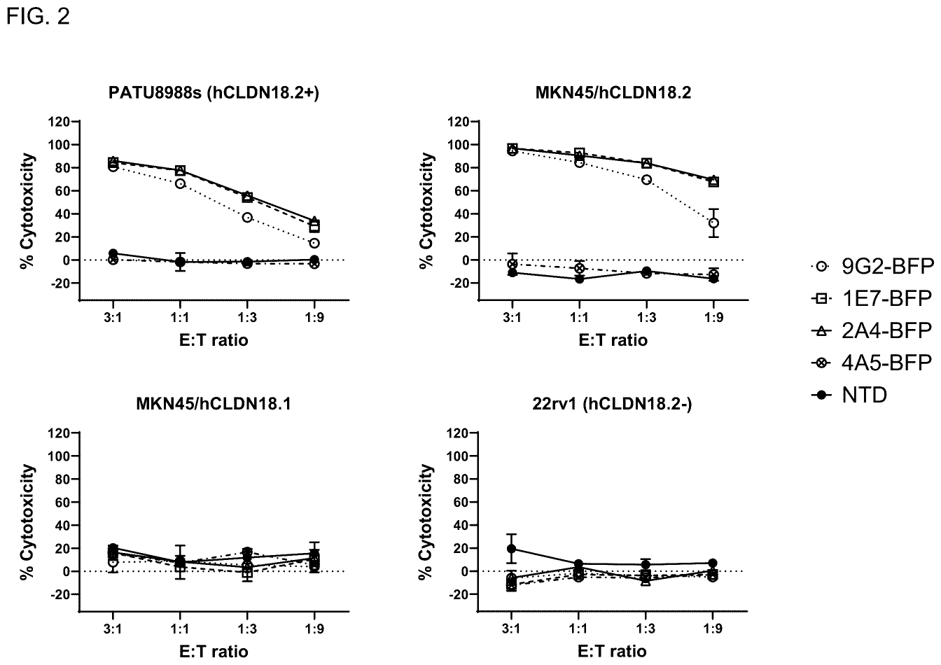

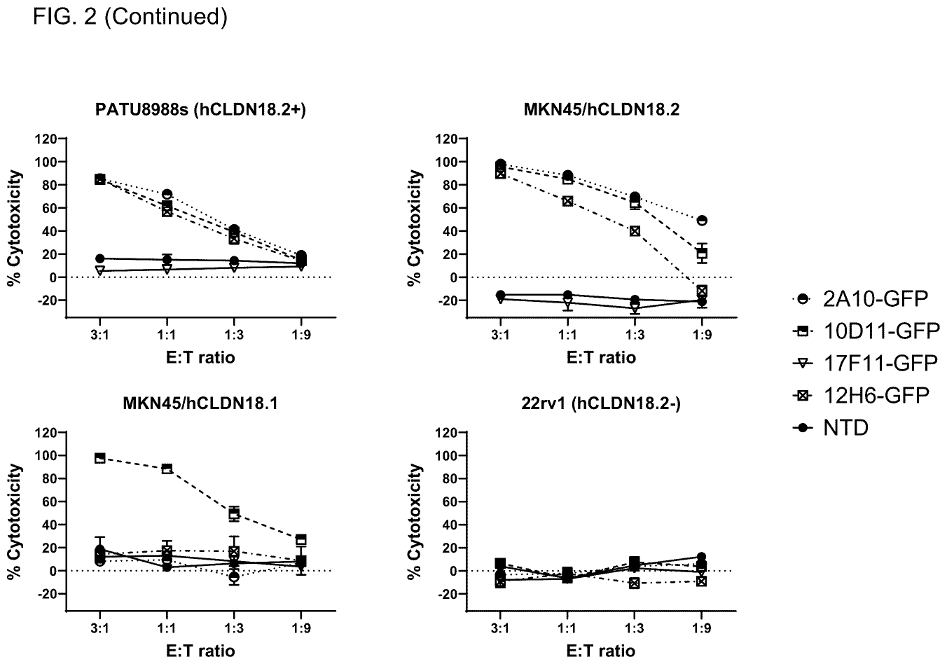

FIG. 2 demonstrates that most of the Claudin 18.2 CAR T cells specially killed target cells expressing Claudin 18.2. Clones that show no cytotoxicity against Claudin 18.2 (4A5, 17F11) or show cross-reactivity with Claudin 18.1 (10D11) were excluded from the following screening.

In addition to the short-term killing assay, a serial killing assay involving repeated antigen exposure was also used to further evaluate Claudin 18.2 CARs with different safety switches. Briefly, on day 0 of the assay, 1×10⁴ target cells were seeded in 100 μL RPMI supplemented with 10% FBS per well in a white flat-bottomed 96-well tissue culture plate. After target cells attached to the bottom of the plates, Claudin 18.2 CAR T cells were thawed and added to plated target cells at an effector:target (E:T) ratio of 3:1 in 100 μL RPMI supplemented with 10% FBS. Every 2 to 3 days thereafter, 100 μL medium containing T cells was transferred to freshly plated target cells and the percentage lysis of target cells was determined at each time point using the one-glo luciferase assay kit (Promega). Each condition was assayed in 3 replicates.

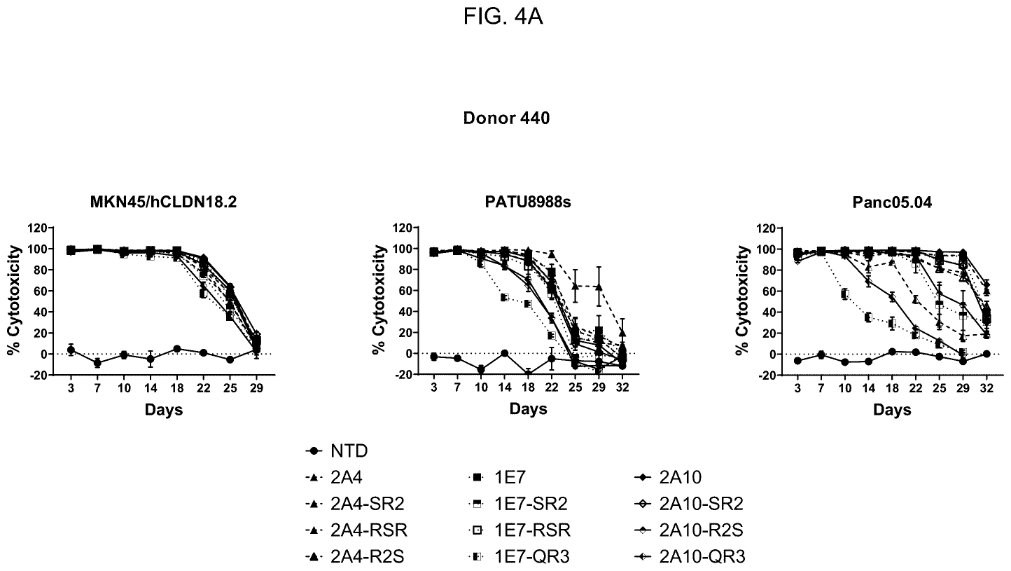

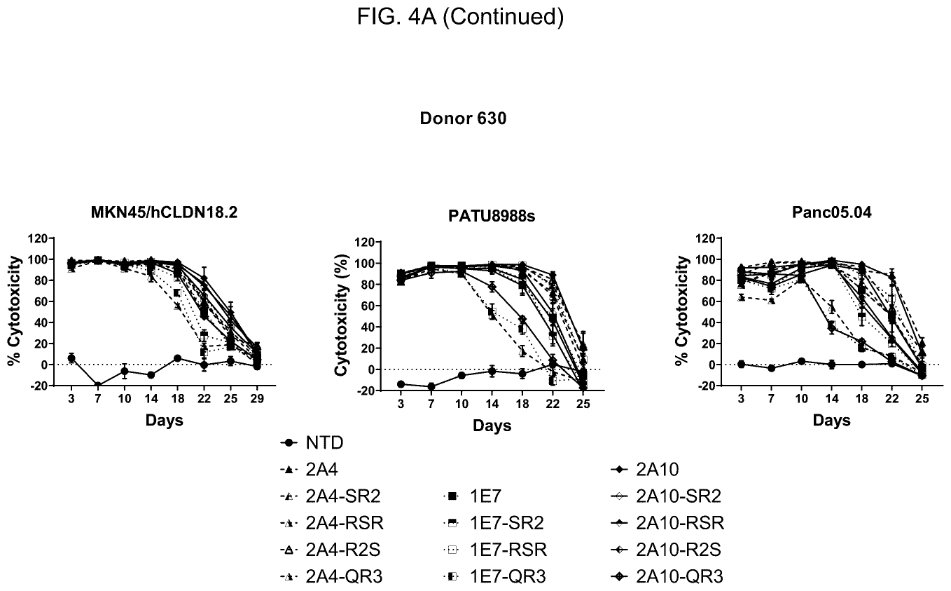

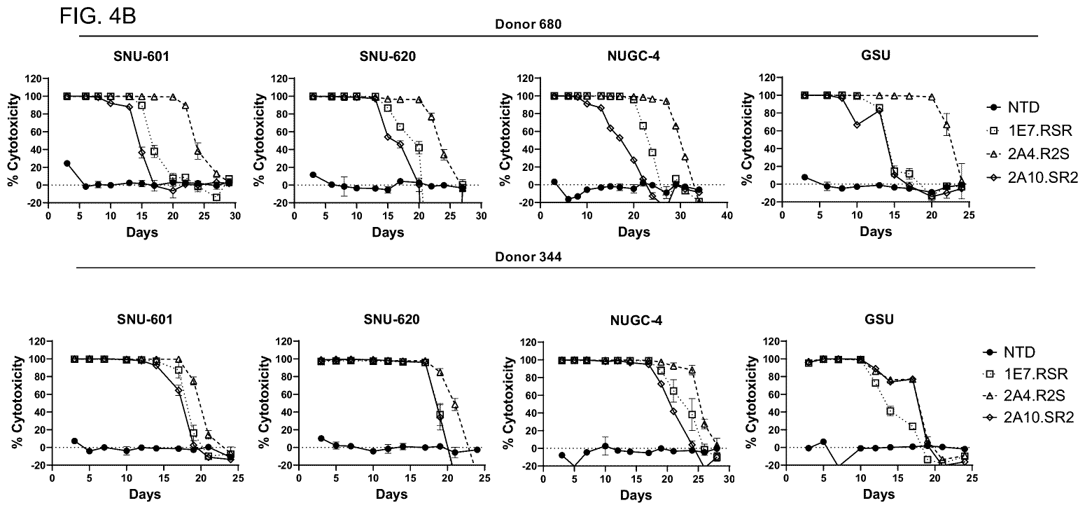

Average percentage of lysis and standard error of mean were plotted in FIGS. 4A-B. FIG. 4A shows the long-term cytotoxicity against one gastric cancer cell line overexpressing Claudin 18.2 (MKN45/hClaudin 18.2) and pancreatic cancer cell lines expressing endogenous Claudin 18.2 (PATU8988s, Panc05.04). Optimal clones with highest target cell lysis during the entire assay period were selected and further tested in another study against four gastric cancer cell lines expressing endogenous Claudin 18.2 (SNU-601, SNU-620, NUGC-4, GSU). As shown in FIG. 4B, 2A4.R2S appeared to be more potent than other two clones in two different donors.

### Example 4. In Vitro Cytokine Secretion of Claudin 18.2 CAR T Cells

Cytokines secreted from T cells produced according to the methods in Example 2 were measured using Human ProInflammatory 9-Plex Tissue Culture Kit (Meso Scale Discovery, 15007B). Briefly, 100 μL RPMI with 10% FBS were added in 96-well plates with or without 1×10⁴ target cells (SNU-601, PATU8988s). Claudin 18.2 CAR T cells were thawed and added at an effector:target (E:T) ratio of 1:1 in 100 μL RPMI medium with 10% FBS. Twenty-four hours later, medium from the co-culture was collected from each well and spun down to pellet T cells. The supernatant was stored at −80° C. and then thawed for cytokine analysis using Meso Scale Discovery analysis according to manufacturer's protocol.

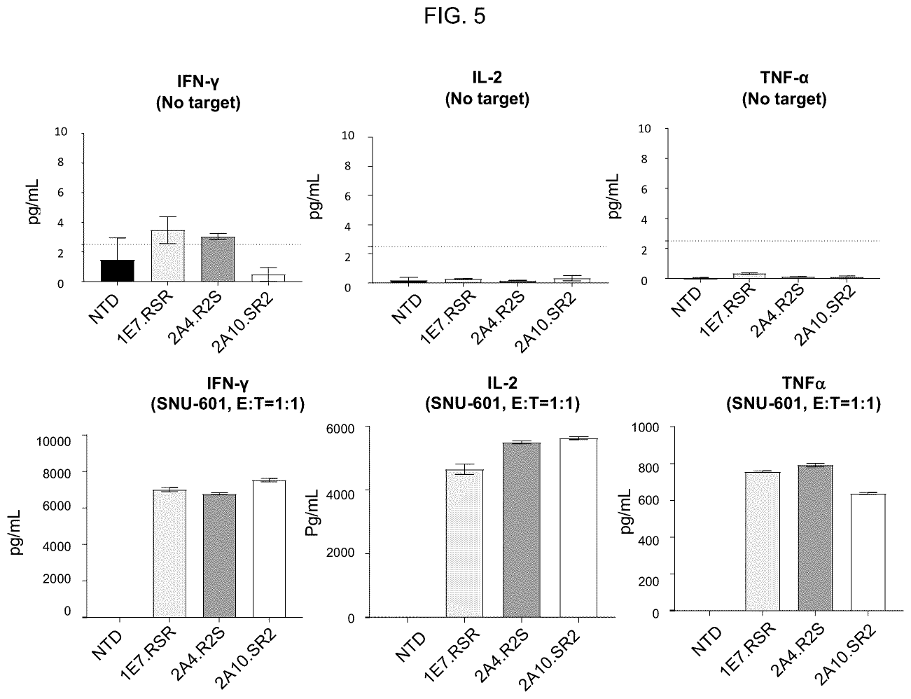

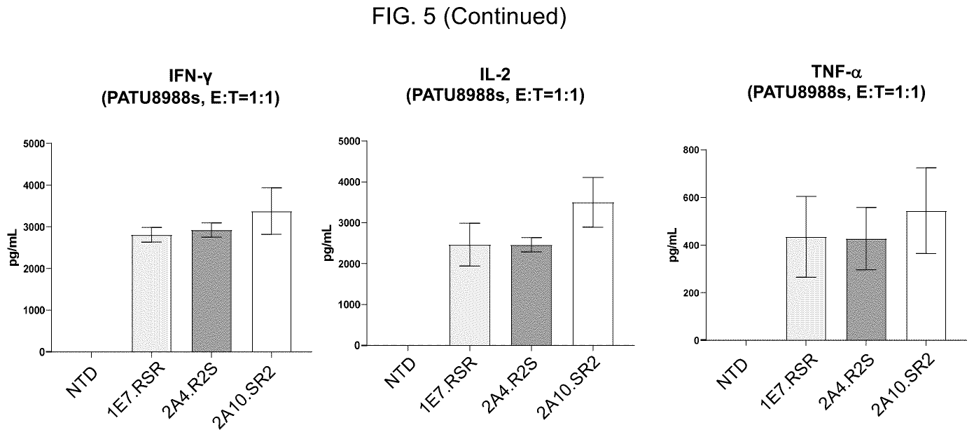

FIG. 5 presents experimental data showing Claudin 18.2 CAR T cells secreted cytokines in a Claudin 18.2 dependent manner. While low levels of spontaneous cytokines were detected when no targets were present, the co-culture with SNU-601 or PATU8988s induced significant release of IFN-γ, IL-2 and TNF-α. The dotted line in each graph indicates limit of detection for individual cytokines.

### Example 5. In Vivo Anti-Tumor Efficacy of Claudin 18.2 CAR T Cells

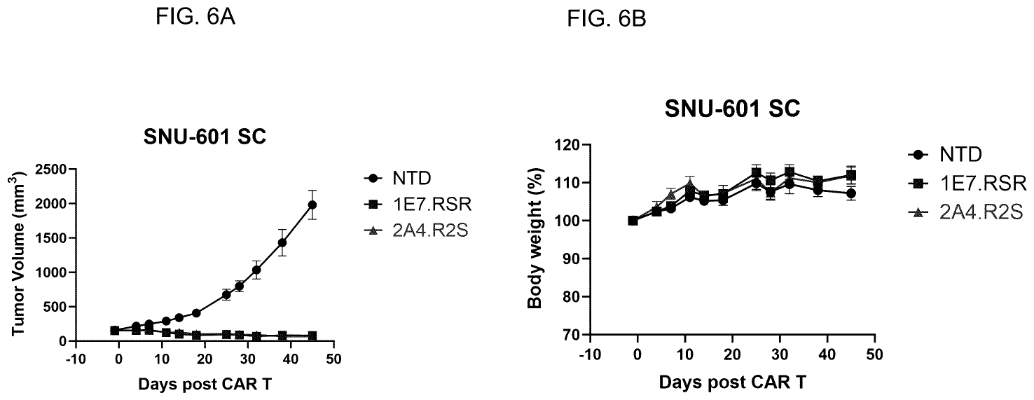

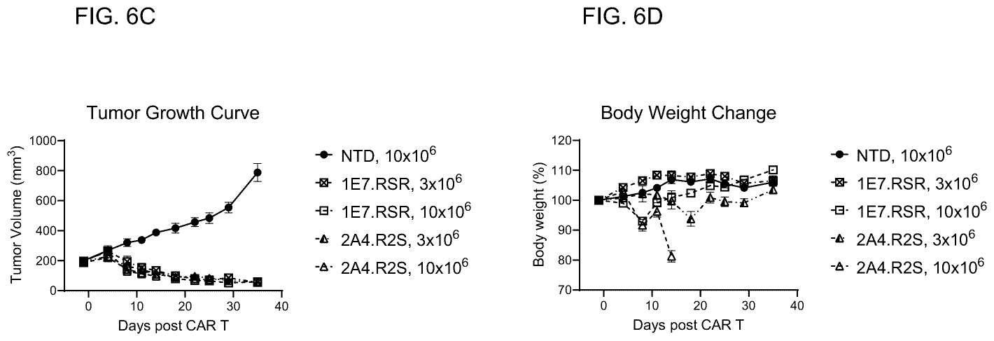

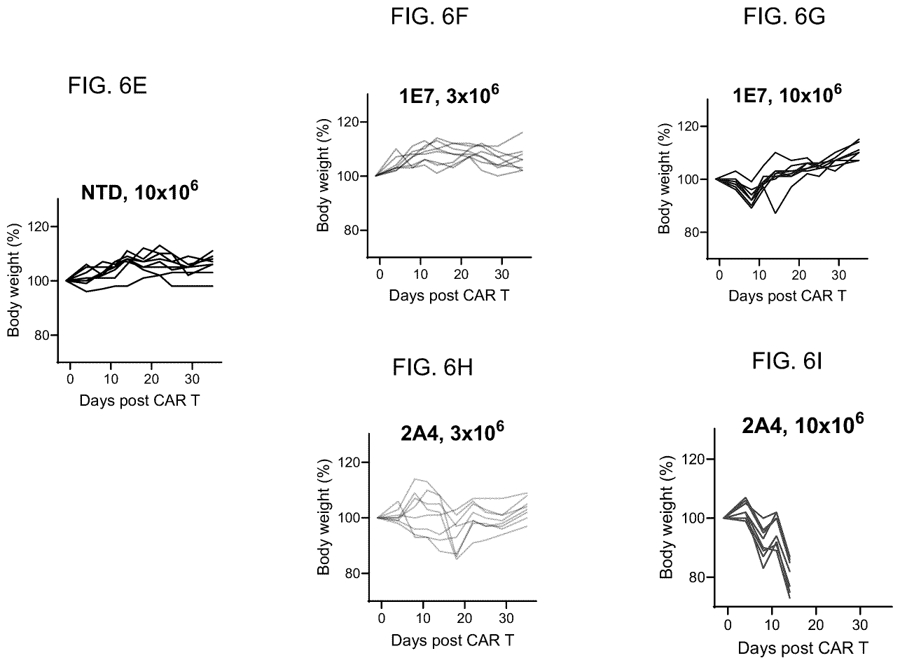

To test the in vivo anti-tumor activity of Claudin 18.2 CAR T cells, SNU-601 gastric cancer xenograft model was used. On day 0, immunodeficient NSG mice were implanted with 5×10⁶ SNU-601 cells subcutaneously and tumor growth was monitored by digital caliper. Tumor size was calculated using the formula Tumor volume=(width²×length/2). Once the tumors attained a volume of 200 mm³, the mice were randomized and treated with freshly thawed Claudin 18.2 CAR T or non-transduced (NTD) T cells intravenously. Cells were resuspended in PBS and injected by tail vein injection in a volume of 200 μL. Tumors and body weight continued to be monitored every 3-4 days until the end of the study. FIGS. 6A-6B show experimental data demonstrating that anti-Claudin 18.2 CAR T cells at the 1×10⁶ CAR+ cell dose can control and eliminate established gastric cancer tumors in the in vivo SNU-601 tumor model (FIG. 6A) without causing body weight loss (FIG. 6B). At a higher dose of 3×10⁶ CAR T cells, weight loss was observed in some of the mice receiving Claudin 18.2 clone 2A4 CAR T cells, followed by a rebound of body weight at later time points. See FIG. 6D (average) and FIG. 6H (individual plot). Mice receiving 10×10⁶ Claudin 18.2 clone 2A4 CAR T cells showed pronounced body weight loss and the studies with these mice were terminated at early time points according to the established animal care protocol. The data in FIG. 6C show that both anti-Claudin 18.2 clone 1E7 CAR and clone 2A4 CAR effective controlled tumor growth in the SNU-601 SC mouse model at all doses tested but noting that the 10×10⁶ CAR T dose data for 2A4 were available only up to day 15. Mice that received anti-Claudin 18.2 clone 1E7 CAR T cells at the high dose of 10×10⁶ CAR T cells exhibited initial weight loss but regained body weight at later time points (FIGS. 6D and 6G). Thus, a therapeutic index can be established for both the clones 2A4 and 1E7 CARs.

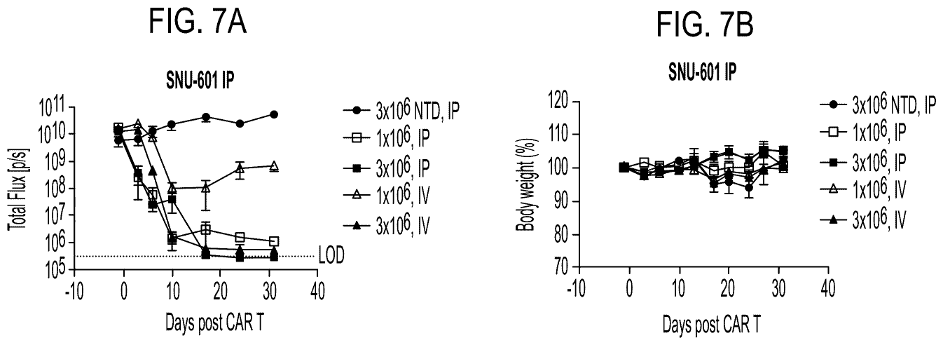

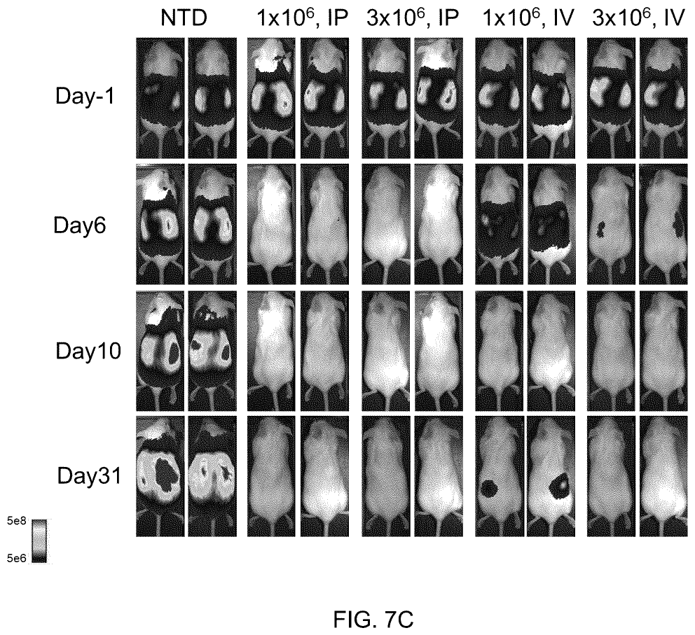

Further experiments were conducted using an intraperitoneal xenograft model which mimics the peritoneal metastases. To establish this model, mice were injected with 3×10⁶ SNU-601 cells intraperitoneally and the engraftment of tumor cells were confirmed using luminescence imaging. Mice were then treated with the Claudin 18.2 clone 1E7 CAR T cells via different injection routes (IP: intraperitoneal or IV: intravenous) at two dose levels (1×10⁶ or 3×10⁶ CAR⁺ cells). Tumor regression was measured by bioluminescence photometry. The data in FIG. 7B show that in the SNU-601 intraperitoneal xenograft model, the lower dose 1×10⁶ CAR T cells administered intravenously did not effectively eliminate tumor in the SNU-601 intraperitoneal xenograft model. The same dose administered intraperitoneally effectively eliminated tumor in the same model (FIG. 7A), suggesting that, without wishing to be bound by any particular mechanisms, local, regional delivery of CAR T cells can be superior to systemic delivery at least in this model. With local or regional delivery, lower doses may be used to further ensure safety. None of the animals showed significant loss of body weight (FIG. 7B). Results represent mean f SEM. Representative bioluminescence images of same mice as in FIGS. 7A-B on Days −1, 6, 10, and 31 post CAR T treatment are shown in FIG. 7C.

In vivo anti-tumor efficacy and safety of the Claudin 18.2 CAR T cells was tested again in a different animal model, the NUGC-4 subcutaneous model, in comparison with two Tool CARs. Tool CAR 1 contains the anti-Claudin 18.2 antibody described in Shah et al., Nat Med, 2133-2141 (2023) and Tool CAR 2 is described in Jiang et al., 2019, JNCI Natl Cancer Inst, 111(4):djy134. On day 0, immunodeficient NSG mice were implanted with 3×10⁶ NUGC-4 cells subcutaneously and tumor growth was monitored by digital caliper as before. On day 20, the tumors attained a volume of about 150-200 mm³, and the mice were randomized and treated with freshly thawed Claudin 18.2 CAR T or non-transduced (NTD) T cells intravenously. Cells were resuspended in PBS and injected at the dose of 1×10⁶ CAR+ cells or 3×10⁶ CAR+ cells per mouse by tail vein injection in a volume of 200 μL. Tumors and body weight continued to be monitored every week starting on Day 4 post CAR T dosing until the end of the study.

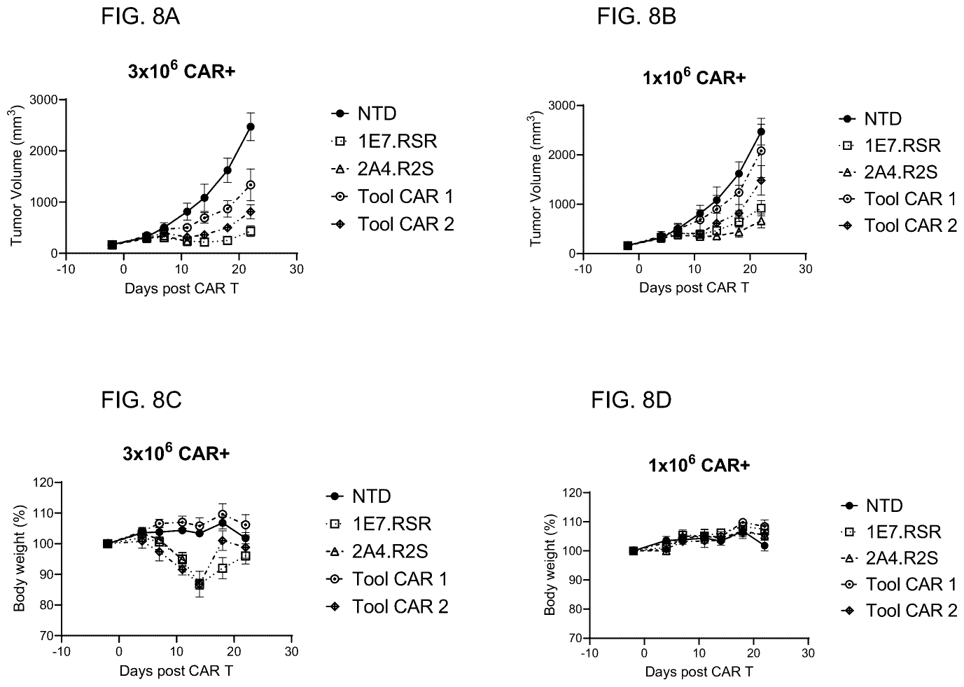

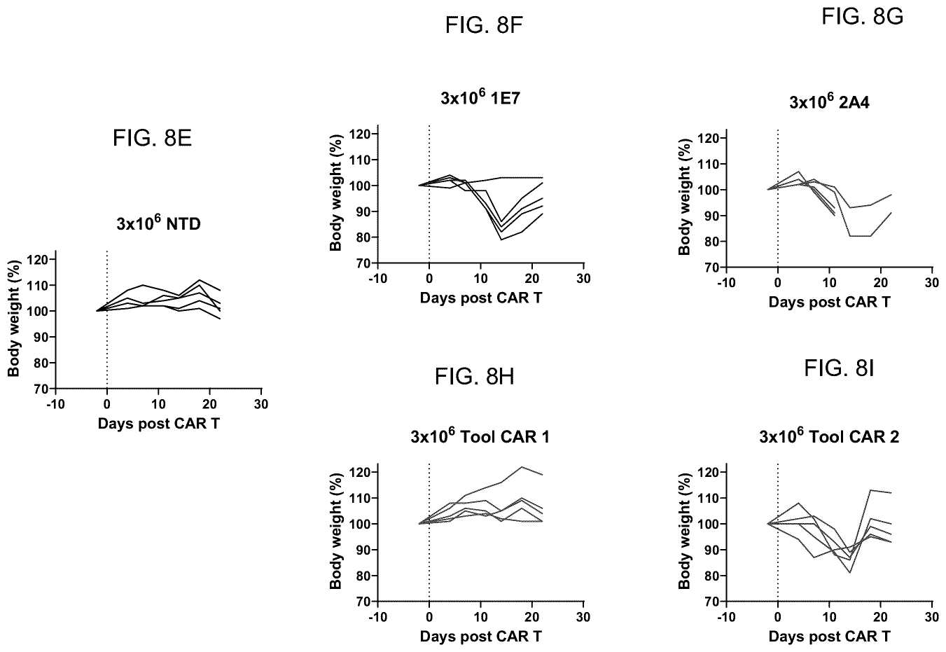

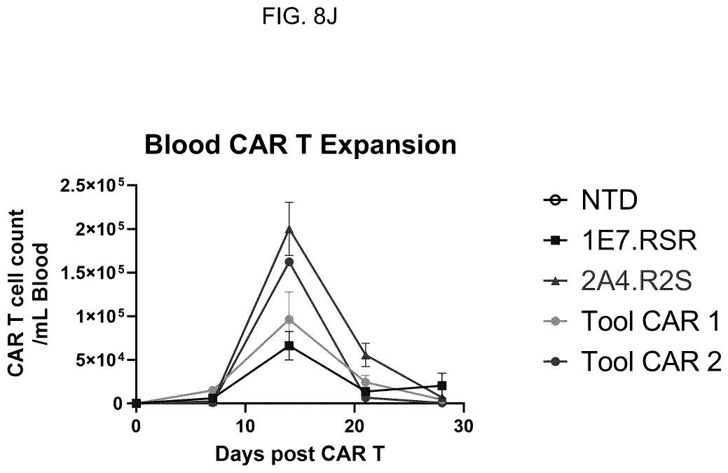

FIGS. 8A and 8B show that both anti-Claudin 18.2 clones 1E7 and 2A4 CAR T cells more effectively controlled the established gastric cancer tumors than Tool CAR 1 and Tool CAR 2 at the 1×10⁶ CAR T cell dose. Anti-Claudin 18.2 clone 2A4 CAR T cells performed even better than clone 1E7 in controlling tumor volume; but at the higher dose of 3×10⁶ CAR T cells, some of the mice that received anti-Claudin 18.2 clone 2A4 CAR T exhibited pronounced weight loss, which led to the termination of the studies of these mice according to the established animal care protocol (FIG. 8C). Individual mouse weight changes were plotted against days post CAR T treatment of the 3×10⁶ CAR T cell dose.

As shown in FIGS. 8F-8I, except for mice that received Tool CAR 1 (FIG. 8H), the least effective CAR in controlling tumor volume (FIG. 8A), initial weight losses were observed in most mice injected with anti-Claudin 18.2 CAR T cells that showed better efficacy than Tool CAR 1 (compare FIGS. 8F, 8G, with FIG. 8I). Mice that received CAR T cells of Tool CAR 2 showed similar initial weight losses, and less tumor volume reduction, as compared to mice that received anti-Claudin 18.2 clones 1E7 and 2A4 CART cells. Compare FIG. 8I with 8F and 8G and the activity data in FIG. 8A. Therefore, initial weight loss may be associated with CAR T efficacy, and the anti-Claudin 18.2 clones 1E7 and 2A4 CAR achieved better efficacy than Tool CAR 1 and Tool CAR 2, while exhibited comparable initial weight loss with the Tool CAR 2. Of the two most effective clones 1E7 and 2A4, all the mice treated with the clone 1E7 CAR T cells and all the mice treated with the clone 2A4 CAR T cells that were kept in the studies regained body weight at later time points. See FIGS. 8F and 8G. The data demonstrated that at selected doses, anti-Claudin 18.2 clones 1E7 and 2A4 CAR T cells can effectively reduce established gastric cancer tumors in mice without causing irreversible body weight loss. Finally, CAR T cells expansion in the blood was analyzed. The number of CAR T cells collected from mice treated with 3×10⁶ CAR+ cells was plotted against days post CAR T treatment (FIG. 8J). Claudin 18.2 clone 2A4 CAR T expanded the best, and while the Claudin 18.2 clone 1E7 CAR T cells did not expand as well as the Claudin 18.2 clone 2A4 CAR T cells, 1E7 exhibited comparable tumor killing activity as 2A4 in this experiment (FIG. 8A).

### Example 6: Generation of Claudin 18.2 Specific CAR T Cells

This example describes the construction of anti-Claudin 18.2 chimeric antigen receptors (CARs).

The anti-Claudin 18.2 antibody clones 1E7, 2A4, 9G2, 2A10, 12H6, 10D11, 17F11, 6B2 and 4A5 were re-formatted as CARs. The amino acid sequences of the heavy chain variable regions and light chain variable regions of these antibodies (Table 1a and Table 1b) were used to design single chain variable fragments (scFvs) (exemplary amino acid sequences set forth in Table 1c) having the following general structure: heavy chain variable region-linker-light chain variable region. The linker had the following amino acid sequences GGGGSGGGGSGGGGSGGGGS (SEQ ID NO: 135). scFv amino acid sequences were combined with hinge, transmembrane, and cytoplasmic amino acid sequence to generate CARs, as described below.

Exemplary chimeric antigen receptors were designed to comprise the following elements from 5′ to 3′ (see Tables 7a and 7b): the CD8α signal sequence (SEQ ID NO: 134), an anti-Claudin 18.2 scFv, hinge and transmembrane regions of the human CD8α molecule (SEQ ID NO: 136), the cytoplasmic portion of the 41BB molecule (SEQ ID NO: 137) and the cytoplasmic portion of the CD3ζ molecule (SEQ ID NO: 138 or 139).

TABLE 6a
Amino acid sequences of exemplary Claudin 18.2 targeting CARs
with signal peptide

| SEQ | | |
|---|---|---|
| ID | Name/ | |
| NO: | Component | Sequence |
| 13 | CD8α signal | MALPVTALLLPLALLLHAARPQITLKESGPTLVKPTQTLT |
| | sequence, 1E7 | LTCTFSGFSLSTSGVGVGWIRQPPGKAPEWLAQIYWNDE |
| | scFv, CD8α hinge | KRYSSSLKSRLTITKDTSKNQVVLKMTNMDPVDTATYY |
| | and | CAHRRGIGNWFDPWGQGTLVTVSSGGGGSGGGGSGGG |
| | transmembrane | GSGGGGSDIQMTQSPSSVSASVGDRVTITCRASQGISSWL |
| | regions, 41BB | AWYQQKPGKAPNLLIYAASGLQSGVPSRFSGSGSGTDFT |
| | cytoplasmic | LTISSLQPEDFASYYCQQANSFPFTFGPGTKVDIKTTTPAP |
| | signaling domain, | RPPTPAPTIASQPLSLRPEACRPAAGGAVHTRGLDFACDI |
| | CD3ζ cytoplasmic | YIWAPLAGTCGVLLLSLVITLYCKRGRKKLLYIFKQPFM |
| | signaling domain | RPVQTTQEEDGCSCRFPEEEEGGCELRVKFSRSADAPAY |
| | | QQGQNQLYNELNLGRREEYDVLDKRRGRDPEMGGKPR |
| | | RKNPQEGLYNELQKDKMAEAYSEIGMKGERRRGKGHD |
| | | GLYQGLSTATKDTYDALHMQALPPR |
| 28 | CD8α signal | MALPVTALLLPLALLLHAARPQITLKESGPTLVKPTQTLT |
| | sequence, 2A4 | LTCTFSGFSLSTSGVGVGWIRQTPGKALEWLTQIYWNDE |
| | scFv, CD8α hinge | KRYSPSLRNRLTITKDTSKNQVVLTMTNMDPVDTATYY |
| | and | CAHRRGVGNWFDPWGQGILVTVSSGGGGSGGGGSGGG |
| | transmembrane | GSGGGGSDIQMTQSPSSVSASVGDRVTITCRASQGISSWL |
| | regions, 41BB | AWYQQKPGKAPKLLIYAASSLQSGVSSRFSGSESGTDFT |
| | cytoplasmic | LTISSLQPEDFATYYCQQANSFPFTFGPGTKVDIKTTTPAP |
| | signaling domain, | RPPTPAPTIASQPLSLRPEACRPAAGGAVHTRGLDFACDI |
| | CD3ζ cytoplasmic | YIWAPLAGTCGVLLLSLVITLYCKRGRKKLLYIFKQPFM |
| | signaling domain | RPVQTTQEEDGCSCRFPEEEEGGCELRVKFSRSADAPAY |
| | | QQGQNQLYNELNLGRREEYDVLDKRRGRDPEMGGKPR |
| | | RKNPQEGLYNELQKDKMAEAYSEIGMKGERRRGKGHD |
| | | GLYQGLSTATKDTYDALHMQALPPR |
| 43 | CD8α signal | MALPVTALLLPLALLLHAARPEVHLLESGGGLVQPWGS |
| | sequence, 9G2 | LTLSCAASGFTFSNYAMNWVRQAPGKGLEWVSGISGSG |
| | scFv, CD8α hinge | GSTYDADSVKGRFTISRDNSKNTLFLQMNSPRAEDTAVY |
| | and | YCATQGYSFGYFESWGQGTLVTVSSGGGGSGGGGSGG |
| | transmembrane | GGSGGGGSEIVLTQSPATLSLSPGERATLSCRASQNVNR |
| | regions, 41BB | YLAWYHQKPGQAPRLLIYDAFNRATGIPARFSGSGSGTD |
| | cytoplasmic | FTLTINSLEPEDFAVYYCQQRSDWPLTFGGGTKLEIKTTT |
| | signaling domain, | PAPRPPTPAPTIASQPLSLRPEACRPAAGGAVHTRGLDFA |
| | CD3ζ cytoplasmic | CDIYIWAPLAGTCGVLLLSLVITLYCKRGRKKLLYIFKQP |
| | signaling domain | FMRPVQTTQEEDGCSCRFPEEEEGGCELRVKFSRSADAP |
| | | AYQQGQNQLYNELNLGRREEYDVLDKRRGRDPEMGGK |
| | | PRRKNPQEGLYNELQKDKMAEAYSEIGMKGERRRGKG |
| | | HDGLYQGLSTATKDTYDALHMQALPPR |
| 58 | CD8α signal | MALPVTALLLPLALLLHAARPQVQLQESGPGLVKPSETL |
| | sequence, 2A10 | SLTCTVSAGSISSYYWNWIRQPAGKGLEWIGRIYTSGST |
| | scFv, CD8α hinge | NYNPSLRSRVTMSVDTSKNQFSLKLSSVTATDTAVYYC |
| | and | ASASYTYFDSFDIWGQGTMVTVSSGGGGSGGGGSGGGG |
| | transmembrane | SGGGGSDIQLTQSPSFLSASVGDRVTITCRASQDIRNFLA |
| | regions, 41BB | WYQQKPGKAPKLLIYAASTLQSGVPSRFSGSGSGTEFAL |
| | cytoplasmic | TVSSLQPEDFATYYCQQVNSYPRTFGQGTKVEIKTTTPA |
| | signaling domain, | PRPPTPAPTIASQPLSLRPEACRPAAGGAVHTRGLDFACD |
| | CD3ζ cytoplasmic | IYIWAPLAGTCGVLLLSLVITLYCKRGRKKLLYIFKQPFM |
| | signaling domain | RPVQTTQEEDGCSCRFPEEEEGGCELRVKFSRSADAPAY |
| | | QQGQNQLYNELNLGRREEYDVLDKRRGRDPEMGGKPR |
| | | RKNPQEGLYNELQKDKMAEAYSEIGMKGERRRGKGHD |
| | | GLYQGLSTATKDTYDALHMQALPPR |
| 73 | CD8α signal | MALPVTALLLPLALLLHAARPEVOLVESGGGLVQPGGS |
| | sequence, 12H6 | LRLSCAASGFTFSRYWMSWVRQAPGKGLEWVANIKHD |
| | scFv, CD8α hinge | GSEKYYVDSVKGRFTFSRDNAKTSLYLQMNSLRVEDTA |
| | and | LYYCARYYGGPFDYWGQGTLVTVSSGGGGSGGGGSGG |
| | transmembrane | GGSGGGGSEIVLTQSPGTLSLSPGERATLSCRASQSVRSS |
| | regions, 41BB | YLAWYQQKPGQAPRLLIFGASSRATGIPDRFSGSGSGTD |
| | cytoplasmic | FTLTISRLEPEDFAVYYCQQFGSSLTFGGGTKVEIKTTTP |
| | signaling domain, | APRPPTPAPTIASQPLSLRPEACRPAAGGAVHTRGLDFAC |
| | CD3ζ cytoplasmic | DIYIWAPLAGTCGVLLLSLVITLYCKRGRKKLLYIFKQPF |
| | signaling domain | MRPVQTTQEEDGCSCRFPEEEEGGCELRVKFSRSADAPA |
| | | YQQGQNQLYNELNLGRREEYDVLDKRRGRDPEMGGKP |
| | | RRKNPQEGLYNELQKDKMAEAYSEIGMKGERRRGKGH |
| | | DGLYQGLSTATKDTYDALHMQALPPR |
| 87 | 10D11 | MALPVTALLLPLALLLHAARPEVQLLESGGGLEQPGGSL |
| | | RLSCAASGFTFSSYAMSWVRQAPGKGLEWVSAISGSGG |
| | | STHYADSVKGRFTISRDNARNTLYLQMNSLRAEDTAVY |
| | | YCAKEGYVGSWYAPFDYWGQGTLVTVSSGGGGSGGGG |
| | | SGGGGSGGGGSQLVLTQSPSASASLGASVKLTCTLSSGH |
| | | SSYAIAWHQQQPEKGPRYLMKLNSGGSHSKGDGIPDRFS |
| | | GSSSGAERYLTISSLQSEDEADYYCQTWDTGIRVFGGGT |
| | | KLTVLTTTPAPRPPTPAPTIASQPLSLRPEACRPAAGGAV |
| | | HTRGLDFACDIYIWAPLAGTCGVLLLSLVITLYCKRGRK |
| | | KLLYIFKQPFMRPVQTTQEEDGCSCRFPEEEEGGCELRV |
| | | KFSRSADAPAYQQGQNQLYNELNLGRREEYDVLDKRR |
| | | GRDPEMGGKPRRKNPQEGLYNELQKDKMAEAYSEIGM |
| | | KGERRRGKGHDGLYQGLSTATKDTYDALHMQALPPR |
| 100 | 17F11 | MALPVTALLLPLALLLHAARPQVTLRESGPALVKPTQTL |
| | | TLTCTVSGVSLSTSGMCVSWIRQPLGKALEWLGFIDWD |
| | | DDKYYNTSLKTRLTISKDTSKNQVVLTMTNMDPVDTAT |
| | | YYCARIRGYSGSYDAFDIWGQGTVVIVSSGGGGSGGGG |
| | | SGGGGSGGGGSDIQMTQSPSSLSASVGDRVTITCRASQGI |
| | | SNYLAWYQQKPGRVPKLLIYAASTLQSGVPSRFSGSGSG |
| | | TDFTLTISSLQPEDVATYYCQKYISAPFTFGPGTKVDIKTT |
| | | TPAPRPPTPAPTIASQPLSLRPEACRPAAGGAVHTRGLDF |
| | | ACDIYIWAPLAGTCGVLLLSLVITLYCKRGRKKLLYIFKQ |
| | | PFMRPVQTTQEEDGCSCRFPEEEEGGCELRVKFSRSADA |
| | | PAYQQGQNQLYNELNLGRREEYDVLDKRRGRDPEMGG |
| | | KPRRKNPQEGLYNELQKDKMAEAYSEIGMKGERRRGK |
| | | GHDGLYQGLSTATKDTYDALHMQALPPR |
| 113 | 6B2 | MALPVTALLLPLALLLHAARPQVTLKESGLTLMKPTQT |
| | | HTLTCTFSGFSLSTSGVGVGWIRQTPGKALEWLTQIYWN |
| | | DEKRYSPSLKNRLTITKDTSKNQVVLTMTNMDPVDTAT |
| | | YYCAHRRGVGNWFDPWGQGTLVTVSSGGGGSGGGGSG |
| | | GGGSGGGGSDIQMTQSPSSVSASVGDRVTITCRASQGISS |
| | | WLAWYQQKPGKAPKLLIYAASSLQSGVSSSFSGSASGTE |
| | | FTLTISNLQPEDFAIYYCQQAFSFPFTFGPGTKVDIKTTTP |
| | | APRPPTPAPTIASQPLSLRPEACRPAAGGAVHTRGLDFAC |
| | | DIYIWAPLAGTCGVLLLSLVITLYCKRGRKKLLYIFKQPF |
| | | MRPVQTTQEEDGCSCRFPEEEEGGCELRVKFSRSADAPA |
| | | YQQGQNQLYNELNLGRREEYDVLDKRRGRDPEMGGKP |
| | | RRKNPQEGLYNELQKDKMAEAYSEIGMKGERRRGKGH |
| | | DGLYQGLSTATKDTYDALHMQALPPR |
| 126 | 4A5 | MALPVTALLLPLALLLHAARPQVTLKESGGGLVQPGGS |
| | | LRLSCAASGFTFSSYAMSWVRQAPGKGLEWVSAISgSGG |
| | | STYYADSVKGRFTISRDNSKNTLYLQMNSLRAEDTAVY |
| | | YCAKDLGATDYWGQGTLVTVSSGGGGSGGGGSGGGGS |
| | | GGGGSDIQMTQSPSSVSASVGDRVTITCRASQGISSWLA |
| | | WYQQKPGKAPKLLIYAASSLQSGVPSRFSGSGSGTDFTL |
| | | TISSLQPEDFATYYCQQANSFPLTFGGGTKVEIKTTTPAP |
| | | RPPTPAPTIASQPLSLRPEACRPAAGGAVHTRGLDFACDI |
| | | YIWAPLAGTCGVLLLSLVITLYCKRGRKKLLYIFKQPFM |
| | | RPVQTTQEEDGCSCRFPEEEEGGCELRVKFSRSADAPAY |
| | | QQGQNQLYNELNLGRREEYDVLDKRRGRDPEMGGKPR |
| | | RKNPQEGLYNELQKDKMAEAYSEIGMKGERRRGKGHD |
| | | GLYQGLSTATKDTYDALHMQALPPR |

TABLE 6b
Amino acid sequences of exemplary Claudin 18.2 targeting CARs
without signal peptide

| SEQ | | |
|---|---|---|
| ID | Name/ | |
| NO: | Component | Sequence |
| 179 | 1E7 scFv, CD8α | QITLKESGPTLVKPTQTLTLTCTFSGFSLSTSGVGVGWIR |
| | hinge and | QPPGKAPEWLAQIYWNDEKRYSSSLKSRLTITKDTSKNQ |
| | transmembrane | VVLKMTNMDPVDTATYYCAHRRGIGNWFDPWGQGTL |
| | regions, 41BB | VTVSSGGGGSGGGGSGGGGSGGGGSDIQMTQSPSSVSA |
| | cytoplasmic | SVGDRVTITCRASQGISSWLAWYQQKPGKAPNLLIYAAS |
| | signaling domain, | GLQSGVPSRFSGSGSGTDFTLTISSLQPEDFASYYCQQAN |
| | CD3ζ cytoplasmic | SFPFTFGPGTKVDIKTTTPAPRPPTPAPTIASQPLSLRPEAC |
| | signaling domain | RPAAGGAVHTRGLDFACDIYIWAPLAGTCGVLLLSLVIT |
| | | LYCKRGRKKLLYIFKQPFMRPVQTTQEEDGCSCRFPEEE |
| | | EGGCELRVKFSRSADAPAYQQGQNQLYNELNLGRREEY |
| | | DVLDKRRGRDPEMGGKPRRKNPQEGLYNELQKDKMAE |
| | | AYSEIGMKGERRRGKGHDGLYQGLSTATKDTYDALHM |
| | | QALPPR |
| 180 | 2A4 scFv, CD8α | QITLKESGPTLVKPTQTLTLTCTFSGFSLSTSGVGVGWIR |
| | hinge and | QTPGKALEWLTQIYWNDEKRYSPSLRNRLTITKDTSKNQ |
| | transmembrane | VVLTMTNMDPVDTATYYCAHRRGVGNWFDPWGQGIL |
| | regions, 41BB | VTVSSGGGGSGGGGSGGGGSGGGGSDIQMTQSPSSVSA |
| | cytoplasmic | SVGDRVTITCRASQGISSWLAWYQQKPGKAPKLLIYAAS |
| | signaling domain, | SLQSGVSSRFSGSESGTDFTLTISSLQPEDFATYYCQQAN |
| | CD3ζ cytoplasmic | SFPFTFGPGTKVDIKTTTPAPRPPTPAPTIASQPLSLRPEAC |
| | signaling domain | RPAAGGAVHTRGLDFACDIYIWAPLAGTCGVLLLSLVIT |
| | | LYCKRGRKKLLYIFKQPFMRPVQTTQEEDGCSCRFPEEE |
| | | EGGCELRVKFSRSADAPAYQQGQNQLYNELNLGRREEY |
| | | DVLDKRRGRDPEMGGKPRRKNPQEGLYNELQKDKMAE |
| | | AYSEIGMKGERRRGKGHDGLYQGLSTATKDTYDALHM |
| | | QALPPR |
| 181 | 9G2 scFv, CD8α | EVHLLESGGGLVQPWGSLTLSCAASGFTFSNYAMNWVR |
| | hinge and | QAPGKGLEWVSGISGSGGSTYDADSVKGRFTISRDNSKN |
| | transmembrane | TLFLQMNSPRAEDTAVYYCATQGYSFGYFESWGQGTLV |
| | regions, 41BB | TVSSGGGGSGGGGSGGGGSGGGGSEIVLTQSPATLSLSP |
| | cytoplasmic | GERATLSCRASQNVNRYLAWYHQKPGQAPRLLIYDAFN |
| | signaling domain, | RATGIPARFSGSGSGTDFTLTINSLEPEDFAVYYCQQRSD |
| | CD3ζ cytoplasmic | WPLTFGGGTKLEIKTTTPAPRPPTPAPTIASQPLSLRPEAC |
| | signaling domain | RPAAGGAVHTRGLDFACDIYIWAPLAGTCGVLLLSLVIT |
| | | LYCKRGRKKLLYIFKQPFMRPVQTTQEEDGCSCRFPEEE |
| | | EGGCELRVKFSRSADAPAYQQGQNQLYNELNLGRREEY |
| | | DVLDKRRGRDPEMGGKPRRKNPQEGLYNELQKDKMAE |
| | | AYSEIGMKGERRRGKGHDGLYQGLSTATKDTYDALHM |
| | | QALPPR |
| 182 | 2A10 scFv, CD8α | QVQLQESGPGLVKPSETLSLTCTVSAGSISSYYWNWIRQ |
| | hinge and | PAGKGLEWIGRIYTSGSTNYNPSLRSRVTMSVDTSKNQF |
| | transmembrane | SLKLSSVTATDTAVYYCASASYTYFDSFDIWGQGTMVT |
| | regions, 41BB | VSSGGGGSGGGGSGGGGSGGGGSDIQLTQSPSFLSASVG |
| | cytoplasmic | DRVTITCRASQDIRNFLAWYQQKPGKAPKLLIYAASTLQ |
| | signaling domain, | SGVPSRFSGSGSGTEFALTVSSLQPEDFATYYCQQVNSYP |
| | CD3ζ cytoplasmic | RTFGQGTKVEIKTTTPAPRPPTPAPTIASQPLSLRPEACRP |
| | signaling domain | AAGGAVHTRGLDFACDIYIWAPLAGTCGVLLLSLVITLY |
| | | CKRGRKKLLYIFKQPFMRPVQTTQEEDGCSCRFPEEEEG |
| | | GCELRVKFSRSADAPAYQQGQNQLYNELNLGRREEYDV |
| | | LDKRRGRDPEMGGKPRRKNPQEGLYNELQKDKMAEAY |
| | | SEIGMKGERRRGKGHDGLYQGLSTATKDTYDALHMQA |
| | | LPPR |
| 183 | 12H6 scFv, CD8α | EVQLVESGGGLVQPGGSLRLSCAASGFTFSRYWMSWVR |
| | hinge and | QAPGKGLEWVANIKHDGSEKYYVDSVKGRFTFSRDNA |
| | transmembrane | KTSLYLQMNSLRVEDTALYYCARYYGGPFDYWGQGTL |
| | regions, 41BB | VTVSSGGGGSGGGGSGGGGSGGGGSEIVLTQSPGTLSLS |
| | cytoplasmic | PGERATLSCRASQSVRSSYLAWYQQKPGQAPRLLIFGAS |
| | signaling domain, | SRATGIPDRFSGSGSGTDFTLTISRLEPEDFAVYYCQQFG |
| | CD3ζ cytoplasmic | SSLTFGGGTKVEIKTTTPAPRPPTPAPTIASQPLSLRPEAC |
| | signaling domain | RPAAGGAVHTRGLDFACDIYIWAPLAGTCGVLLLSLVIT |
| | | LYCKRGRKKLLYIFKQPFMRPVQTTQEEDGCSCRFPEEE |
| | | EGGCELRVKFSRSADAPAYQQGQNQLYNELNLGRREEY |
| | | DVLDKRRGRDPEMGGKPRRKNPQEGLYNELQKDKMAE |
| | | AYSEIGMKGERRRGKGHDGLYQGLSTATKDTYDALHM |
| | | QALPPR |
| 195 | 10D11 | EVQLLESGGGLEQPGGSLRLSCAASGFTFSSYAMSWVR |
| | | QAPGKGLEWVSAISGSGGSTHYADSVKGRFTISRDNARN |
| | | TLYLQMNSLRAEDTAVYYCAKEGYVGSWYAPFDYWG |
| | | QGTLVTVSSGGGGSGGGGSGGGGSGGGGSQLVLTQSPS |
| | | ASASLGASVKLTCTLSSGHSSYAIAWHQQQPEKGPRYL |
| | | MKLNSGGSHSKGDGIPDRFSGSSSGAERYLTISSLQSEDE |
| | | ADYYCQTWDTGIRVFGGGTKLTVLTTTPAPRPPTPAPTI |
| | | ASQPLSLRPEACRPAAGGAVHTRGLDFACDIYIWAPLAG |
| | | TCGVLLLSLVITLYCKRGRKKLLYIFKQPFMRPVQTTQE |
| | | EDGCSCRFPEEEEGGCELRVKFSRSADAPAYQQGQNQL |
| | | YNELNLGRREEYDVLDKRRGRDPEMGGKPRRKNPQEG |
| | | LYNELQKDKMAEAYSEIGMKGERRRGKGHDGLYQGLS |
| | | TATKDTYDALHMQALPPR |
| 196 | 17F11 | QVTLRESGPALVKPTQTLTLTCTVSGVSLSTSGMCVSWI |
| | | RQPLGKALEWLGFIDWDDDKYYNTSLKTRLTISKDTSK |
| | | NQVVLTMTNMDPVDTATYYCARIRGYSGSYDAFDIWG |
| | | QGTVVIVSSGGGGSGGGGSGGGGSGGGGSDIQMTQSPSS |
| | | LSASVGDRVTITCRASQGISNYLAWYQQKPGRVPKLLIY |
| | | AASTLQSGVPSRFSGSGSGTDFTLTISSLQPEDVATYYCQ |
| | | KYISAPFTFGPGTKVDIKTTTPAPRPPTPAPTIASQPLSLRP |
| | | EACRPAAGGAVHTRGLDFACDIYIWAPLAGTCGVLLLSL |
| | | VITLYCKRGRKKLLYIFKQPFMRPVQTTQEEDGCSCRFP |
| | | EEEEGGCELRVKFSRSADAPAYQQGQNQLYNELNLGRR |
| | | EEYDVLDKRRGRDPEMGGKPRRKNPQEGLYNELQKDK |
| | | MAEAYSEIGMKGERRRGKGHDGLYQGLSTATKDTYDA |
| | | LHMQALPPR |
| 197 | 6B2 | QVTLKESGLTLMKPTQTHTLTCTFSGFSLSTSGVGVGWI |
| | | RQTPGKALEWLTQIYWNDEKRYSPSLKNRLTITKDTSKN |
| | | QVVLTMTNMDPVDTATYYCAHRRGVGNWFDPWGQGT |
| | | LVTVSSGGGGSGGGGSGGGGSGGGGSDIQMTQSPSSVS |
| | | ASVGDRVTITCRASQGISSWLAWYQQKPGKAPKLLIYA |
| | | ASSLQSGVSSSFSGSASGTEFTLTISNLQPEDFAIYYCQQA |
| | | FSFPFTFGPGTKVDIKTTTPAPRPPTPAPTIASQPLSLRPEA |
| | | CRPAAGGAVHTRGLDFACDIYIWAPLAGTCGVLLLSLVI |
| | | TLYCKRGRKKLLYIFKQPFMRPVQTTQEEDGCSCRFPEE |
| | | EEGGCELRVKFSRSADAPAYQQGQNQLYNELNLGRREE |
| | | YDVLDKRRGRDPEMGGKPRRKNPQEGLYNELQKDKMA |
| | | EAYSEIGMKGERRRGKGHDGLYQGLSTATKDTYDALH |
| | | MQALPPR |
| 198 | 4A5 | QVTLKESGGGLVQPGGSLRLSCAASGFTFSSYAMSWVR |
| | | QAPGKGLEWVSAISgSGGSTYYADSVKGRFTISRDNSKN |
| | | TLYLQMNSLRAEDTAVYYCAKDLGATDYWGQGTLVTV |
| | | SSGGGGSGGGGSGGGGSGGGGSDIQMTQSPSSVSASVG |
| | | DRVTITCRASQGISSWLAWYQQKPGKAPKLLIYAASSLQ |
| | | SGVPSRFSGSGSGTDFTLTISSLQPEDFATYYCQQANSFP |
| | | LTFGGGTKVEIKTTTPAPRPPTPAPTIASQPLSLRPEACRP |
| | | AAGGAVHTRGLDFACDIYIWAPLAGTCGVLLLSLVITLY |
| | | CKRGRKKLLYIFKQPFMRPVQTTQEEDGCSCRFPEEEEG |
| | | GCELRVKFSRSADAPAYQQGQNQLYNELNLGRREEYDV |
| | | LDKRRGRDPEMGGKPRRKNPQEGLYNELQKDKMAEAY |
| | | SEIGMKGERRRGKGHDGLYQGLSTATKDTYDALHMQA |
| | | LPPR |
| 200 | 1E7 scFv, CD28 | QITLKESGPTLVKPTQTLTLTCTFSGFSLSTSGVGVGWIR |
| | hinge and | QPPGKAPEWLAQIYWNDEKRYSSSLKSRLTITKDTSKNQ |
| | transmembrane | VVLKMTNMDPVDTATYYCAHRRGIGNWFDPWGQGTL |
| | regions, 41BB | VTVSSGGGGSGGGGSGGGGSGGGGSDIQMTQSPSSVSA |
| | cytoplasmic | SVGDRVTITCRASQGISSWLAWYQQKPGKAPNLLIYAAS |
| | signaling domain, | GLQSGVPSRFSGSGSGTDFTLTISSLQPEDFASYYCQQAN |
| | CD3ζ cytoplasmic | SFPFTFGPGTKVDIKIEVMYPPPYLDNEKSNGTIIHVKGK |
| | signaling domain | HLCPSPLFPGPSKPFWVLVVVGGVLACYSLLVTVAFIIFW |
| | | VKRGRKKLLYIFKQPFMRPVQTTQEEDGCSCRFPEEEEG |
| | | GCELRVKFSRSADAPAYQQGQNQLYNELNLGRREEYDV |
| | | LDKRRGRDPEMGGKPRRKNPQEGLYNELQKDKMAEAY |
| | | SEIGMKGERRRGKGHDGLYQGLSTATKDTYDALHMQA |
| | | LPPR |
| 201 | 2A4 scFv, CD28 | QITLKESGPTLVKPTQTLTLTCTFSGFSLSTSGVGVGWIR |
| | hinge and | QTPGKALEWLTQIYWNDEKRYSPSLRNRLTITKDTSKNQ |
| | transmembrane | VVLTMTNMDPVDTATYYCAHRRGVGNWFDPWGQGIL |
| | regions, 41BB | VTVSSGGGGSGGGGSGGGGSGGGGSDIQMTQSPSSVSA |
| | cytoplasmic | SVGDRVTITCRASQGISSWLAWYQQKPGKAPKLLIYAAS |
| | signaling domain, | SLQSGVSSRFSGSESGTDFTLTISSLQPEDFATYYCQQAN |
| | CD3ζ cytoplasmic | SFPFTFGPGTKVDIKIEVMYPPPYLDNEKSNGTIIHVKGK |
| | signaling domain | HLCPSPLFPGPSKPFWVLVVVGGVLACYSLLVTVAFIIFW |
| | | VKRGRKKLLYIFKQPFMRPVQTTQEEDGCSCRFPEEEEG |
| | | GCELRVKFSRSADAPAYQQGQNQLYNELNLGRREEYDV |
| | | LDKRRGRDPEMGGKPRRKNPQEGLYNELQKDKMAEAY |
| | | SEIGMKGERRRGKGHDGLYQGLSTATKDTYDALHMQA |
| | | LPPR |

TABLE 7a
Amino acid sequences of exemplary Claudin 18.2 targeting CAR components

| SEQ | | |
|---|---|---|
| ID | Name/ | |
| NO: | Component | Sequence |
| 134 | CD8α signal | MALPVTALLLPLALLLHAARP |
| | sequence | |
| 135 | linker (“(G₄S)₄”) | GGGGSGGGGSGGGGSGGGGS |
| 136 | CD8α hinge and | TTTPAPRPPTPAPTIASQPLSLRPEACRPAAGGAVHTRGL |
| | transmembrane | DFACDIYIWAPLAGTCGVLLLSLVIT |
| | regions | |
| 137 | 41BB cytoplasmic | KRGRKKLLYIFKQPFMRPVQTTQEEDGCSCRFPEEEEGG |
| | signaling domain | CEL |
| 138 | CD3ζ cytoplasmic | RVKFSRSADAPAYQQGQNQLYNELNLGRREEYDVLDK |
| | signaling domain | RRGRDPEMGGKPRRKNPQEGLYNELQKDKMAEAYSEIG |
| | | MKGERRRGKGHDGLYQGLSTATKDTYDALHMQALPPR |
| 139 | CD3ζ cytoplasmic | LRVKFSRSADAPAYKQGQNQLYNELNLGRREEYDVLDK |
| | signaling domain | RRGRDPEMGGKPRRKNPQEGLYNELQKDKMAEAYSEIG |
| | | MKGERRRGKGHDGLYQGLSTATKDTYDALHMQALPPR |
| 202 | CD3 epsilon | KNRKAKAKPVTRGAGAGGRQRGQNKERPPPVPNPDYEP |
| | cytoplasmic | IRKGQRDLYSGLNQRRI |
| | domain | |
| 203 | CD3 gamma | GQDGVRQSRASDKQTLLPNDQLYQPLKDREDDQYSHLQ |
| | cytoplasmic | GNQLRRN |
| | domain | |
| 153 | CD8α hinge | TTTPAPRPPTPAPTIASQPLSLRPEACRPAAGGAVHTRGL |
| | domain | DFACD |
| 154 | CD8α | IYIWAPLAGTCGVLLLSLVIT |
| | transmembrane | |
| | domain | |
| 155 | CD8α | IYIWAPLAGTCGVLLLSLVITLYC |
| | transmembrane | |
| | domain (alternate) | |
| 156 | CD28 hinge | IEVMYPPPYLDNEKSNGTIIHVKGKHLCPSPLFPGPSKP |
| 199 | CD28 hinge | KIEVMYPPPYLDNEKSNGTIIHVKGKHLCPSPLFPGPSKP |
| 157 | CD28-TM (CD28 | FWVLVVVGGVLACYSLLVTVAFIIFWV |
| | transmembrane | |
| | domain) | |
| 158 | CD28-IC (CD28 | RSKRSRGGHSDYMNMTPRRPGPTRKHYQPYAPPRDFAA |
| | co-stimulatory | YRS |
| | domain, LL to | |
| | GG) | |
| 159 | CD28.YMFM | RSKRSRLLHSDYMFMTPRRPGPTRKHYQPYAPPRDFAA |
| | intracellular | YRS |
| | (YMFM is | |
| | disclosed in SEQ | |
| | ID NO: 216) | |
| 160 | amino GS | GGGGSGGGGSGGGSG |
| 161 | carboxy GS | GGGSGGGGSGGGGS |
| 162 | (GGGGS)₃ linker | GGGGSGGGGSGGGGS |
| 163 | GS sequence (1) | GGGGS |
| 164 | Whitlow linker | GSTSGSGKPGSGEGSTKG |
| 167 | safety switch | CPYSNPSLCSGGGGSELPTQGTFSNVSTNVSPAKPTTTAC |
| | | PYSNPSLCSGGGGSPAPRPPTPAPTIASQPLSLRPEACRPA |
| | | AGGAVHTRGLDFACDIYIWAPLAGTCGVLLLSLVITLYC |
| | | NHRNRRRVCKCPRPVV |
| 168 | safety switch- | MGTSLLCWMALCLLGADHADACPYSNPSLCSGGGGSEL |
| | amino term. | PTQGTFSNVSTNVSPAKPTTTACPYSNPSLCSGGGGSPAP |
| | | RPPTPAPTIASQPLSLRPEACRPAAGGAVHTRGLDFACDI |
| | | YIWAPLAGTCGVLLLSLVITLYCNHRNRRRVCKCPRPVV |
| 169 | safety switch | MGTSLLCWMALCLLGADHADA |
| | signal peptide | |
| 170 | FcγRIIIα (Fc- | GLAVSTISSFFPPGYQ |
| | gamma-RIII- | |
| | alpha) binge | |
| 171 | IgG1 hinge | EPKSPDKTHTCPPCPAPPVAGPSVFLFPPKPKDTLMIART |
| | | PEVTCVVVDVSHEDPEVKFNWYVDGVEVHNAKTKPRE |
| | | EQYNSTYRVVSVLTVLHQDWLNGKEYKCKVSNKALPA |
| | | PIEKTISKAKGQPREPQVYTLPPSRDELTKNQVSLTCLVK |
| | | GFYPSDIAVEWESNGQPENNYKTTPPVLDSDGSFFLYSK |
| | | LTVDKSRWQQGNVFSCSVMHEALHNHYTQKSLSLSPGK |
| 174 | CD28 intracellular | RSKRSRLLHSDYMNMTPRRPGPTRKHYQPYAPPRDFAA |
| | (WT) | YRS |
| 177 | SG Linker | SGGGG |
| 178 | SGS linker | SGGGGS |
| 212 | WT PD1 dominant | PGWFLDSPDRPWNPPTFSPALLVVTEGDNATFTCSFSNT |
| | negative receptor | SESFVLNWYRMSPSNQTDKLAAFPEDRSQPGQDCRFRV |
| | | TQLPNGRDFHMSVVRARRNDSGTYLCGAISLAPKAQIKE |
| | | SLRAELRVTERRAEVPTAHPSPSPRPAGQFQTLVVGVVG |
| | | GLLGSLVLLVWVLAVICSRAARGTIGARRTGQ |
| 213 | High affinity PD1 | PGWFLDSPDRPWNPPTFSPALLVVTEGDNATFTCSFSNT |
| | dominant negative | SESFHVIWHRESPSGQTDTLAAFPEDRSQPGQDCRFRVT |
| | receptor | QLPNGRDFHMSVVRARRNDSGTYVCGVISLAPKIQIKES |
| | | LRAELRVTERRAEVPTAHPSPSPRPAGQFQTLVVGVVGG |
| | | LLGSLVLLVWVLAVICSRAARGTIGARRTGQ |
| 214 | TGRβR dominant | TIPPHVQKSVNNDMIVTDNNGAVKFPQLCKFCDVRFSTC |
| | negative receptor | DNQKSCMSNCSITSICEKPQEVCVAVWRKNDENITLETV |
| | | CHDPKLPYHDFILEDAASPKCIMKEKKKPGETFFMCSCS |
| | | SDECNDNIIFSEEYNTSNPDLLLVIFQVTGISLLPPLGVAIS |
| | | VILIFYCYRVNRQQKLSS |

TABLE 7b
Exemplary nucleic acid sequences encoding CAR components

| SEQ ID | | |
|---|---|---|
| NO: | Description | Sequence |
| 151 | DNA | GGCGGTGGAGGCTCCGGAGGGGGGGGCTCTGGCGGAGGGGG |
| | sequence | CTCC |
| | encoding | |
| | linker | |
| | (G₄S)₄ (SEQ | |
| | ID NO: | |
| | 135) | |
| 152 | Alternative | GGCGGCGGCGGCTCTGGAGGAGGAGGCAGCGGCGGAGGAG |
| | DNA | GCTCCGGAGGCGGCGGCTCT |
| | sequence | |
| | encoding | |
| | linker | |
| | (G₄S)₄ (SEQ | |
| | ID NO: 135 | |
| 165 | Whitlow | GGGTCTACATCCGGCTCCGGGAAGCCCGGAAGTGGCGAAGG |
| | linker | TAGTACAAAGGGG |
| | encoding | |
| | sequence | |
| 166 | Leader | ATGGCACTCCCCGTAACTGCTCTGCTGCTGCCGTTGGCATTG |
| | (signal) | CTCCTGCACGCCGCACGCCCG |
| | peptide | |
| | encoding | |
| | sequence | |
| 172 | 4-1BB | AAGCGCGGCAGGAAGAAGCTCCTCTACATTTTTAAGCAGCCT |
| | encoding | TTTATGAGGCCCGTACAGACAACACAGGAGGAAGATGGCTG |
| | sequence | TAGCTGCAGATTTCCCGAGGAGGAGGAAGGTGGGTGCGAGC |
| | | TG |
| 173 | CD28 | AGATCCAAAAGAAGCCGCCTGCTCCATAGCGATTACATGAA |
| | intracellular | TATGACTCCACGCCGCCCTGGCCCCACAAGGAAACACTACCA |
| | domain | GCCTTACGCACCACCTAGAGATTTCGCTGCCTATCGGAGC |
| | encoding | |
| | sequence | |
| 175 | CD3 zeta | AGGGTGAAGTTTTCCAGATCTGCAGATGCACCAGCGTATCAG |
| | encoding | CAGGGCCAGAACCAACTGTATAACGAGCTCAACCTGGGACG |
| | sequence | CAGGGAAGAGTATGACGTTTTGGACAAGCGCAGAGGACGGG |
| | | ACCCTGAGATGGGTGGCAAACCAAGACGAAAAAACCCCCAG |
| | | GAGGGTCTCTATAATGAGCTGCAGAAGGATAAGATGGCTGA |
| | | AGCCTATTCTGAAATAGGCATGAAAGGAGAGCGGAGAAGGG |
| | | GAAAAGGGCACGACGGTTTGTACCAGGGACTCAGCACTGCT |
| | | ACGAAGGATACTTATGACGCTCTCCACATGCAAGCCCTGCCA |
| | | CCTAGG |

### Example 7: Anti-Claudin 18.2 CAR Constructs with a Safety Switch

This example describes the construction of anti-Claudin 18.2 CARs with a safety switch. The anti-Claudin 18.2 CARs disclosed herein may be formatted to include different safety switch structures, e.g. any of the safety switch structures listed below (Table 8). In Table 8, “R” refers to a rituximab recognition site, also referred to as CD20 mimotope, “Q” refers to a QBEND-10 epitope, and “S” refers to scFv e.g. anti-Claudin scFv as disclosed herein.

TABLE 8
Schematic Structure of Exemplary Safety Switches

| Format | Structure |
|---|---|
| QR3 | CD8α signal sequence - linker- CD20 mimotope - linker - anti-Claudin 18.2 |
| | scFv - linker - CD20 mimotope - linker - QBEND-10 epitope - linker - |
| | CD20 mimotope - hinge and transmembrane regions of human CD8 α |
| | molecule - 41BB signaling domain - CD3ζ signaling domain |
| SR2 | CD8α signal sequence - anti-Claudin 18.2 scFv - linker - CD20 mimotope - |
| | linker- CD20 mimotope -linker - hinge and transmembrane regions of |
| | human CD8 α molecule - 41BB signaling domain - CD3ζ signaling domain |
| RSR | CD8α signal sequence -- linker - CD20 mimotope - linker- anti-Claudin |
| | 18.2 scFv - linker - CD20 mimotope - linker-hinge and transmembrane |
| | regions of human CD8 α molecule - 41BB signaling domain - CD3ζ |
| | signaling domain |
| R2S | CD8α signal sequence -- linker - CD20 mimotope - linker- CD20 |
| | mimotope - linker - anti-Claudin 18.2 scFv- linker-hinge and |
| | transmembrane regions of human CD8 α molecule - 41BB signaling |
| | domain - CD3ζ signaling domain |

Exemplary protein sequences ofanti-Claudin 18.2 CAR constructs including a safety switch are shown in Table 9a. Exemplary safety switch constructs may comprise the CD8 signal sequence (SEQ ID NO: 134), an anti-Claudin 18.2 scFv as described herein, CD2 mimotope (SEQ ID NO: 140), QBEND-1 epitope (SEQ ID NO: 148 or SEQ ID NO: 149), hinge and transmembrane regions of the human CD8α molecule (SEQ ID NO: 136), the cytoplasmic portion of the 4-1BB molecule (SEQ ID NO: 137) and the cytoplasmic portion of the CD3 molecule (SEQ ID NO: 138 or SEQ ID NO: 139). Exemplary safety switch constructs may comprise an anti-Claudin 18.2 scFv as described herein, CD20 mimotope (SEQ ID NO: 140), QBEND-10 epitope (SEQ ID NO: 148 or SEQ ID NO: 149), hinge and transmembrane regions of the human CD8α molecule (SEQ ID NO: 136), the cytoplasmic portion of the 4-1BB molecule (SEQ ID NO: 137) and the cytoplasmic portion of the CD3ζ molecule (SEQ ID NO: 138 or SEQ ID NO: 139) (no CD8α signal sequence).

TABLE 9a
Exemplary anti-Claudin 18.2 CAR and safety switch amino acid sequences
with signal sequence

| SEQ ID | | |
|---|---|---|
| NO: | Name / Component | Sequence |
| 128 | 1E7-RSR | MALPVTALLLPLALLLHAARPGGGGSCPYSNPSLCGGGG |
| | CD8α signal | SQITLKESGPTLVKPTQTLTLTCTFSGFSLSTSGVGVGWIR |
| | sequence, CD20 | QPPGKAPEWLAQIYWNDEKRYSSSLKSRLTITKDTSKNQ |
| | mimotope, 1E7 | VVLKMTNMDPVDTATYYCAHRRGIGNWFDPWGQGTLV |
| | scFv, CD20 | TVSSGGGGSGGGGSGGGGSGGGGSDIQMTQSPSSVSASV |
| | mimotope, hinge | GDRVTITCRASQGISSWLAWYQQKPGKAPNLLIYAASGL |
| | and transmembrane | QSGVPSRFSGSGSGTDFTLTISSLQPEDFASYYCQQANSFP |
| | regions of human | FTFGPGTKVDIKGGGGSCPYSNPSLCGGGGSTTTPAPRPP |
| | CD8α molecule, | TPAPTIASQPLSLRPEACRPAAGGAVHTRGLDFACDIYIW |
| | 41BB signaling | APLAGTCGVLLLSLVITLYCKRGRKKLLYIFKQPFMRPVQ |
| | domain, CD3ζ | TTQEEDGCSCRFPEEEEGGCELRVKFSRSADAPAYQQGQ |
| | signaling domain | NQLYNELNLGRREEYDVLDKRRGRDPEMGGKPRRKNPQ |
| | | EGLYNELQKDKMAEAYSEIGMKGERRRGKGHDGLYQGL |
| | | STATKDTYDALHMQALPPR |
| 130 | 2A4-R2S | MALPVTALLLPLALLLHAARPGGGGSCPYSNPSLCGGGG |
| | CD8α signal | SCPYSNPSLCGGGGSQITLKESGPTLVKPTQTLTLTCTFSG |
| | sequence, CD20 | FSLSTSGVGVGWIRQTPGKALEWLTQIYWNDEKRYSPSL |
| | mimotope, CD20 | RNRLTITKDTSKNQVVLTMTNMDPVDTATYYCAHRRGV |
| | mimotope, 2A4 | GNWFDPWGQGILVTVSSGGGGSGGGGSGGGGSGGGGSD |
| | scFv, hinge and | IQMTQSPSSVSASVGDRVTITCRASQGISSWLAWYQQKPG |
| | transmembrane | KAPKLLIYAASSLQSGVSSRFSGSESGTDFTLTISSLQPEDF |
| | regions of human | ATYYCQQANSFPFTFGPGTKVDIKTTTPAPRPPTPAPTIAS |
| | CD8 a molecule, | QPLSLRPEACRPAAGGAVHTRGLDFACDIYIWAPLAGTC |
| | 41BB signaling | GVLLLSLVITLYCKRGRKKLLYIFKQPFMRPVQTTQEEDG |
| | domain, CD3ζ | CSCRFPEEEEGGCELRVKFSRSADAPAYQQGQNQLYNEL |
| | signaling domain | NLGRREEYDVLDKRRGRDPEMGGKPRRKNPQEGLYNEL |
| | | QKDKMAEAYSEIGMKGERRRGKGHDGLYQGLSTATKDT |
| | | YDALHMQALPPR |
| 132 | 2A10-SR2 | MALPVTALLLPLALLLHAARPQVQLQESGPGLVKPSETLS |
| | CD8α signal | LTCTVSAGSISSYYWNWIRQPAGKGLEWIGRIYTSGSTNY |
| | sequence, 2A10 | NPSLRSRVTMSVDTSKNQFSLKLSSVTATDTAVYYCASA |
| | scFv, CD20 | SYTYFDSFDIWGQGTMVTVSSGGGGSGGGGSGGGGSGG |
| | mimotope, CD20 | GGSDIQLTQSPSFLSASVGDRVTITCRASQDIRNFLAWYQ |
| | mimotope, hinge | QKPGKAPKLLIYAASTLQSGVPSRFSGSGSGTEFALTVSSL |
| | and transmembrane | QPEDFATYYCQQVNSYPRTFGQGTKVEIKGSGGGGSCPY |
| | regions of human | SNPSLCSGGGGSCPYSNPSLCSGGGGSTTTPAPRPPTPAPT |
| | CD8 a molecule, | IASQPLSLRPEACRPAAGGAVHTRGLDFACDIYIWAPLAG |
| | 41BB signaling | TCGVLLLSLVITLYCKRGRKKLLYIFKQPFMRPVQTTQEE |
| | domain, CD3ζ | DGCSCRFPEEEEGGCELRVKFSRSADAPAYQQGQNQLYN |
| | signaling domain | ELNLGRREEYDVLDKRRGRDPEMGGKPRRKNPQEGLYN |
| | | ELQKDKMAEAYSEIGMKGERRRGKGHDGLYQGLSTATK |
| | | DTYDALHMQALPPR |

TABLE 9b
Exemplary anti-Claudin 18.2 CAR and safety switch nucleic acid sequences

| SEQ ID | | |
|---|---|---|
| NO: | Description | Sequence |
| 129 | 1E7-RSR | ATGGCCCTGCCCGTCACTGCTCTGCTGCTGCCTCTGGCTCTGC |
| | DNA | TGCTGCACGCCGCCCGCCCTGGGGGGGGAGGATCTTGTCCAT |
| | sequence | ATTCCAACCCATCTCTGTGCGGAGGAGGAGGATCCCAGATCA |
| | | CACTGAAGGAGTCTGGCCCTACCCTGGTGAAGCCAACCCAG |
| | | ACACTGACCCTGACATGTACCTTTAGCGGCTTCTCTCTGAGC |
| | | ACCTCCGGAGTGGGAGTGGGATGGATCAGGCAGCCACCTGG |
| | | CAAGGCACCTGAGTGGCTGGCCCAGATCTACTGGAACGACG |
| | | AGAAGCGGTATAGCTCCTCTCTGAAGTCTAGACTGACAATCA |
| | | CCAAGGATACATCCAAGAACCAGGTGGTGCTGAAGATGACC |
| | | AATATGGACCCAGTGGATACAGCCACCTACTATTGCGCCCAC |
| | | CGGAGAGGCATCGGCAATTGGTTTGACCCATGGGGACAGGG |
| | | CACACTGGTGACCGTGAGCTCCGGAGGAGGAGGAAGCGGCG |
| | | GAGGAGGCAGCGGCGGCGGCGGCTCTGGCGGCGGCGGCAGC |
| | | GACATCCAGATGACACAGAGCCCATCTAGCGTGTCTGCCAGC |
| | | GTGGGCGATAGGGTGACAATCACCTGCAGGGCATCCCAGGG |
| | | AATCTCCTCTTGGCTGGCCTGGTACCAGCAGAAGCCAGGCAA |
| | | GGCCCCCAACCTGCTGATCTATGCAGCAAGCGGACTGCAGTC |
| | | CGGAGTGCCTTCTAGATTTTCCGGCTCTGGCAGCGGCACCGA |
| | | CTTCACACTGACCATCAGCTCCCTGCAGCCAGAGGATTTCGC |
| | | CAGCTACTATTGTCAGCAGGCCAATTCCTTCCCCTTTACATTC |
| | | GGCCCTGGCACCAAGGTGGATATCAAGGGGGGGGGCGGAAG |
| | | TTGTCCATACTCAAATCCAAGCCTGTGCGGCGGAGGCGGCTC |
| | | TACTACCACTCCAGCACCTAGGCCACCTACACCTGCACCAAC |
| | | CATCGCCAGCCAGCCTCTGTCCCTGAGACCAGAGGCCTGTAG |
| | | GCCAGCAGCAGGAGGAGCAGTGCACACCCGGGGCCTGGACT |
| | | TCGCCTGCGATATCTACATCTGGGCACCACTGGCAGGAACAT |
| | | GTGGCGTGCTGCTGCTGTCCCTGGTCATCACCCTGTACTGCA |
| | | AGAGAGGCAGGAAGAAGCTGCTGTATATCTTCAAGCAGCCC |
| | | TTCATGAGACCCGTGCAGACAACCCAGGAGGAGGACGGCTG |
| | | CAGCTGTAGGTTCCCAGAGGAGGAGGAGGGAGGATGTGAGC |
| | | TGCGCGTGAAGTTTTCCCGGTCTGCCGATGCACCTGCATACC |
| | | AGCAGGGACAGAACCAGCTGTATAACGAGCTGAATCTGGGC |
| | | CGGAGAGAGGAGTACGACGTGCTGGATAAGAGGAGGGGAA |
| | | GGGACCCTGAGATGGGAGGCAAGCCTCGGAGAAAGAACCCA |
| | | CAGGAGGGCCTGTACAATGAGCTGCAGAAGGACAAGATGGC |
| | | CGAGGCCTATAGCGAGATCGGCATGAAGGGAGAGAGGCGCC |
| | | GGGGCAAGGGACACGATGGCCTGTATCAGGGCCTGTCAACC |
| | | GCTACAAAAGATACCTACGATGCTCTGCACATGCAGGCTCTG |
| | | CCACCAAGA |
| 131 | 2A4-R2S | ATGGCTCTGCCCGTCACTGCTCTGCTGCTGCCCCTGGCTCTGC |
| | CAR DNA | TGCTGCACGCCGCAAGACCCGGAGGAGGAGGAAGCTGTCCC |
| | sequence | TATTCCAACCCATCTCTGTGCGGCGGCGGAGGAAGCTGTCCC |
| | | TACAGCAATCCTTCCCTGTGCGGAGGAGGAGGAAGCCAGAT |
| | | CACACTGAAGGAGTCCGGCCCTACCCTGGTGAAGCCAACCC |
| | | AGACACTGACCCTGACATGTACCTTCTCCGGCTTTAGCCTGT |
| | | CCACCTCTGGAGTGGGAGTGGGATGGATCAGGCAGACACCA |
| | | GGCAAGGCCCTGGAGTGGCTGACCCAGATCTACTGGAACGA |
| | | CGAGAAGCGGTACAGCCCTTCCCTGAGGAATCGCCTGACAA |
| | | TCACCAAGGATACCAGCAAGAACCAGGTGGTGCTGACAATG |
| | | ACCAATATGGACCCAGTGGATACAGCCACCTACTATTGCGCA |
| | | CACAGGAGAGGAGTGGGAAACTGGTTCGACCCATGGGGACA |
| | | GGGCATCCTGGTGACAGTGAGCTCCGGCGGCGGCGGCTCTG |
| | | GAGGAGGAGGAAGCGGAGGAGGAGGAAGCGGGGGCGGCGG |
| | | CTCTGACATCCAGATGACCCAGTCTCCTTCTAGCGTGTCTGC |
| | | CAGCGTGGGCGATCGGGTGACAATCACCTGCAGAGCCTCCC |
| | | AGGGCATCTCCTCTTGGCTGGCCTGGTACCAGCAGAAGCCAG |
| | | GCAAGGCCCCCAAGCTGCTGATCTATGCAGCAAGCTCCCTGC |
| | | AGAGCGGCGTGTCTAGCCGGTTCTCCGGCTCTGAGAGCGGCA |
| | | CAGACTTTACACTGACCATCTCCTCTCTGCAGCCAGAGGATT |
| | | TTGCCACCTACTATTGTCAGCAGGCCAATTCCTTCCCCTTCAC |
| | | ATTCGGACCTGGCACAAAAGTGGACATCAAGACTACTACCC |
| | | CCGCCCCTAGGCCACCTACACCTGCACCAACCATCGCCAGCC |
| | | AGCCTCTGTCCCTGAGACCAGAGGCCTGTAGGCCAGCAGCA |
| | | GGAGGAGCAGTGCACACCCGGGGCCTGGACTTCGCCTGCGA |
| | | TATCTACATCTGGGCACCACTGGCAGGAACATGTGGCGTGCT |
| | | GCTGCTGTCCCTGGTCATCACCCTGTACTGCAAGAGAGGCAG |
| | | GAAGAAGCTGCTGTATATCTTCAAGCAGCCCTTCATGAGACC |
| | | CGTGCAGACAACCCAGGAGGAGGACGGCTGCAGCTGTAGGT |
| | | TCCCAGAGGAGGAGGAGGGAGGATGTGAGCTGCGCGTGAAG |
| | | TTTTCCCGGTCTGCCGATGCACCTGCATACCAGCAGGGACAG |
| | | AACCAGCTGTATAACGAGCTGAATCTGGGCCGGAGAGAGGA |
| | | GTACGACGTGCTGGATAAGAGGAGGGGAAGGGACCCTGAGA |
| | | TGGGAGGCAAGCCTCGGAGAAAGAACCCACAGGAGGGCCTG |
| | | TACAATGAGCTGCAGAAGGACAAGATGGCCGAGGCCTATAG |
| | | CGAGATCGGCATGAAGGGAGAGAGGCGCCGGGGCAAGGGA |
| | | CACGATGGCCTGTATCAGGGCCTGTCAACCGCTACAAAAGAT |
| | | ACCTACGATGCTCTGCACATGCAGGCTCTGCCACCAAGA |
| 133 | 2A10-SR2 | ATGGCTCTGCCTGTCACCGCTCTGCTGCTGCCTCTGGCTCTGC |
| | CAR DNA | TGCTGCACGCTGCTCGCCCCCAGGTCCAGCTGCAGGAATCCG |
| | Sequence | GGCCTGGCCTGGTGAAGCCATCTGAGACCCTGAGCCTGACCT |
| | | GCACAGTGTCCGCCGGCTCTATCAGCTCCTACTATTGGAACT |
| | | GGATCAGACAGCCAGCAGGCAAGGGACTGGAGTGGATCGGA |
| | | AGGATCTACACATCTGGCAGCACCAACTATAATCCCAGCCTG |
| | | CGGTCCAGAGTGACAATGTCCGTGGACACCTCTAAGAATCA |
| | | GTTCTCCCTGAAGCTGTCTAGCGTGACCGCCACAGATACCGC |
| | | CGTGTACTATTGTGCCTCCGCCTCTTACACATATTTCGACAGC |
| | | TTTGATATCTGGGGCCAGGGCACAATGGTGACCGTGTCCTCT |
| | | GGAGGAGGAGGAAGCGGAGGAGGAGGAAGCGGCGGCGGCG |
| | | GCTCTGGCGGCGGCGGCAGCGACATCCAGCTGACCCAGAGC |
| | | CCTTCCTTCCTGTCTGCCAGCGTGGGCGACAGGGTGACAATC |
| | | ACCTGCCGCGCCAGCCAGGATATCCGGAACTTTCTGGCCTGG |
| | | TACCAGCAGAAGCCCGGCAAGGCCCCTAAGCTGCTGATCTAT |
| | | GCAGCAAGCACACTGCAGTCCGGAGTGCCATCTCGCTTCTCC |
| | | GGATCTGGAAGCGGCACAGAGTTTGCACTGACCGTGAGCTC |
| | | CCTGCAGCCAGAGGATTTCGCCACCTACTATTGTCAGCAGGT |
| | | GAACTCCTACCCTCGGACATTTGGCCAGGGCACCAAGGTGG |
| | | AGATCAAGGGATCCGGAGGAGGAGGATCTTGCCCATATTCT |
| | | AATCCCAGCCTGTGCTCCGGCGGCGGCGGCAGCTGTCCATAC |
| | | TCCAATCCATCACTGTGCAGCGGGGGGGGGGGGTCAACCAC |
| | | TACACCAGCACCTAGGCCACCTACACCTGCACCAACCATCGC |
| | | CAGCCAGCCTCTGTCCCTGAGACCAGAGGCCTGTAGGCCAGC |
| | | AGCAGGAGGAGCAGTGCACACCCGGGGCCTGGACTTCGCCT |
| | | GCGATATCTACATCTGGGCACCACTGGCAGGAACATGTGGC |
| | | GTGCTGCTGCTGTCCCTGGTCATCACCCTGTACTGCAAGAGA |
| | | GGCAGGAAGAAGCTGCTGTATATCTTCAAGCAGCCCTTCATG |
| | | AGACCCGTGCAGACAACCCAGGAGGAGGACGGCTGCAGCTG |
| | | TAGGTTCCCAGAGGAGGAGGAGGGAGGATGTGAGCTGCGCG |
| | | TGAAGTTTTCCCGGTCTGCCGATGCACCTGCATACCAGCAGG |
| | | GACAGAACCAGCTGTATAACGAGCTGAATCTGGGCCGGAGA |
| | | GAGGAGTACGACGTGCTGGATAAGAGGAGGGGAAGGGACC |
| | | CTGAGATGGGAGGCAAGCCTCGGAGAAAGAACCCACAGGAG |
| | | GGCCTGTACAATGAGCTGCAGAAGGACAAGATGGCCGAGGC |
| | | CTATAGCGAGATCGGCATGAAGGGAGAGAGGCGCCGGGGCA |
| | | AGGGACACGATGGCCTGTATCAGGGCCTGTCAACCGCTACA |
| | | AAAGATACCTACGATGCTCTGCACATGCAGGCTCTGCCACCA |
| | | AGA |

TABLE 9c
Exemplary anti-Claudin 18.2 CAR and safety switch amino acid sequences
without signal sequence

| SEQ ID | | |
|---|---|---|
| NO: | Name / Component | Sequence |
| 184 | 1E7-RSR | GGGGSCPYSNPSLCGGGGSQITLKESGPTLVKPTQTLTLT |
| | CD20 mimotope, | CTFSGFSLSTSGVGVGWIRQPPGKAPEWLAQIYWNDEKR |
| | 1E7 scFv, CD20 | YSSSLKSRLTITKDTSKNQVVLKMTNMDPVDTATYYCAH |
| | mimotope, hinge | RRGIGNWFDPWGQGTLVTVSSGGGGSGGGGSGGGGSGG |
| | and transmembrane | GGSDIQMTQSPSSVSASVGDRVTITCRASQGISSWLAWYQ |
| | regions of human | QKPGKAPNLLIYAASGLQSGVPSRFSGSGSGTDFTLTISSL |
| | CD8α molecule, | QPEDFASYYCQQANSFPFTFGPGTKVDIKGGGGSCPYSNP |
| | 41BB signaling | SLCGGGGSTTTPAPRPPTPAPTIASQPLSLRPEACRPAAGG |
| | domain, CD3ζ | AVHTRGLDFACDIYIWAPLAGTCGVLLLSLVITLYCKRGR |
| | signaling domain | KKLLYIFKQPFMRPVQTTQEEDGCSCRFPEEEEGGCELRV |
| | | KFSRSADAPAYQQGQNQLYNELNLGRREEYDVLDKRRG |
| | | RDPEMGGKPRRKNPQEGLYNELQKDKMAEAYSEIGMKG |
| | | ERRRGKGHDGLYQGLSTATKDTYDALHMQALPPR |
| 185 | 2A4-R2S | GGGGSCPYSNPSLCGGGGSCPYSNPSLCGGGGSQITLKES |
| | CD20 mimotope, | GPTLVKPTQTLTLTCTFSGFSLSTSGVGVGWIRQTPGKAL |
| | CD20 mimotope, | EWLTQIYWNDEKRYSPSLRNRLTITKDTSKNQVVLTMTN |
| | 2A4 scFv, hinge | MDPVDTATYYCAHRRGVGNWFDPWGQGILVTVSSGGG |
| | and transmembrane | GSGGGGSGGGGSGGGGSDIQMTQSPSSVSASVGDRVTIT |
| | regions of human | CRASQGISSWLAWYQQKPGKAPKLLIYAASSLQSGVSSR |
| | CD8 α molecule, | FSGSESGTDFTLTISSLQPEDFATYYCQQANSFPFTFGPGT |
| | 41BB signaling | KVDIKTTTPAPRPPTPAPTIASQPLSLRPEACRPAAGGAVH |
| | domain, CD3ζ | TRGLDFACDIYIWAPLAGTCGVLLLSLVITLYCKRGRKKL |
| | signaling domain | LYIFKQPFMRPVQTTQEEDGCSCRFPEEEEGGCELRVKFS |
| | | RSADAPAYQQGQNQLYNELNLGRREEYDVLDKRRGRDP |
| | | EMGGKPRRKNPQEGLYNELQKDKMAEAYSEIGMKGERR |
| | | RGKGHDGLYQGLSTATKDTYDALHMQALPPR |
| 186 | 2A10-SR2 | QVQLQESGPGLVKPSETLSLTCTVSAGSISSYYWNWIRQP |
| | 2A10 scFv, CD20 | AGKGLEWIGRIYTSGSTNYNPSLRSRVTMSVDTSKNQFSL |
| | mimotope, CD20 | KLSSVTATDTAVYYCASASYTYFDSFDIWGQGTMVTVSS |
| | mimotope, hinge | GGGGSGGGGSGGGGSGGGGSDIQLTQSPSFLSASVGDRV |
| | and transmembrane | TITCRASQDIRNFLAWYQQKPGKAPKLLIYAASTLQSGVP |
| | regions of human | SRFSGSGSGTEFALTVSSLQPEDFATYYCQQVNSYPRTFG |
| | CD8 α molecule, | QGTKVEIKGSGGGGSCPYSNPSLCSGGGGSCPYSNPSLCS |
| | 41BB signaling | GGGGSTTTPAPRPPTPAPTIASQPLSLRPEACRPAAGGAV |
| | domain, CD3ζ | HTRGLDFACDIYIWAPLAGTCGVLLLSLVITLYCKRGRKK |
| | signaling domain | LLYIFKQPFMRPVQTTQEEDGCSCRFPEEEEGGCELRVKF |
| | | SRSADAPAYQQGQNQLYNELNLGRREEYDVLDKRRGRD |
| | | PEMGGKPRRKNPQEGLYNELQKDKMAEAYSEIGMKGER |
| | | RRGKGHDGLYQGLSTATKDTYDALHMQALPPR |
| 208 | CD20 mimotope, | GGGGSCPYSNPSLCGGGGSQITLKESGPTLVKPTQTLTLT |
| | 1E7 scFv, CD20 | CTFSGFSLSTSGVGVGWIRQPPGKAPEWLAQIYWNDEKR |
| | mimotope, hinge | YSSSLKSRLTITKDTSKNQVVLKMTNMDPVDTATYYCAH |
| | and transmembrane | RRGIGNWFDPWGQGTLVTVSSGGGGSGGGGSGGGGSGG |
| | regions of human | GGSDIQMTQSPSSVSASVGDRVTITCRASQGISSWLAWYQ |
| | CD28 molecule, | QKPGKAPNLLIYAASGLQSGVPSRFSGSGSGTDFTLTISSL |
| | 41BB signaling | QPEDFASYYCQQANSFPFTFGPGTKVDIKGGGGSCPYSNP |
| | domain, CD3ζ | SLCGGGGSIEVMYPPPYLDNEKSNGTIIHVKGKHLCPSPLF |
| | signaling domain | PGPSKPFWVLVVVGGVLACYSLLVTVAFIIFWVKRGRKK |
| | | LLYIFKQPFMRPVQTTQEEDGCSCRFPEEEEGGCELRVKF |
| | | SRSADAPAYQQGQNQLYNELNLGRREEYDVLDKRRGRD |
| | | PEMGGKPRRKNPQEGLYNELQKDKMAEAYSEIGMKGER |
| | | RRGKGHDGLYQGLSTATKDTYDALHMQALPPR |
| 209 | CD20 mimotope, | GGGGSCPYSNPSLCGGGGSCPYSNPSLCGGGGSQITLKES |
| | CD20 mimotope, | GPTLVKPTQTLTLTCTFSGFSLSTSGVGVGWIRQTPGKAL |
| | 2A4 scFv, hinge | EWLTQIYWNDEKRYSPSLRNRLTITKDTSKNQVVLTMTN |
| | and transmembrane | MDPVDTATYYCAHRRGVGNWFDPWGQGILVTVSSGGG |
| | regions of human | GSGGGGSGGGGSGGGGSDIQMTQSPSSVSASVGDRVTIT |
| | CD28 molecule, | CRASQGISSWLAWYQQKPGKAPKLLIYAASSLQSGVSSR |
| | 41BB signaling | FSGSESGTDFTLTISSLQPEDFATYYCQQANSFPFTFGPGT |
| | domain, CD3ζ | KVDIKIEVMYPPPYLDNEKSNGTIIHVKGKHLCPSPLFPGP |
| | signaling domain | SKPFWVLVVVGGVLACYSLLVTVAFIIFWVKRGRKKLLY |
| | | IFKQPFMRPVQTTQEEDGCSCRFPEEEEGGCELRVKFSRS |
| | | ADAPAYQQGQNQLYNELNLGRREEYDVLDKRRGRDPE |
| | | MGGKPRRKNPQEGLYNELQKDKMAEAYSEIGMKGERRR |
| | | GKGHDGLYQGLSTATKDTYDALHMQALPPR |
| 210 | 1E7/CD70 tandem | GGGGSCPYSNPSLCGGGGSDIQMTQSPSAMSASVGDRVT |
| | CAR aa sequence | ITCRASQDISNYLAWFQQKPGKVPKRLIYAASSLQSGVPS |
| | | RFSGSGSGTEFTLTISSLLPEDFATYYCLQLNSFPFTFGGGT |
| | | KVEINGGGGSGGGGSGGGGSGGGGSQVTLKESGPVLVKP |
| | | TETLTLTCTVSGFSLSNARMGVTWIRQPPGKALEWLAHIF |
| | | SNDEKSYSTSLKSRLTISKDTSKTQVVLTMTNMDPVDTA |
| | | TYYCARIRDYYDISSYYDYWGQGTLVSVSSGGGGSGGG |
| | | GSGGGGSGGGGSQITLKESGPTLVKPTQTLTLTCTFSGFSL |
| | | STSGVGVGWIRQPPGKAPEWLAQIYWNDEKRYSSSLKSR |
| | | LTITKDTSKNQVVLKMTNMDPVDTATYYCAHRRGIGNW |
| | | FDPWGQGTLVTVSSGGGGSGGGGSGGGGSGGGGSDIQM |
| | | TQSPSSVSASVGDRVTITCRASQGISSWLAWYQQKPGKA |
| | | PNLLIYAASGLQSGVPSRFSGSGSGTDFTLTISSLQPEDFAS |
| | | YYCQQANSFPFTFGPGTKVDIKGGGGSCPYSNPSLCGGG |
| | | GSTTTPAPRPPTPAPTIASQPLSLRPEACRPAAGGAVHTRG |
| | | LDFACDIYIWAPLAGTCGVLLLSLVITLYCKRGRKKLLYI |
| | | FKQPFMRPVQTTQEEDGCSCRFPEEEEGGCELRVKFSRSA |
| | | DAPAYQQGQNQLYNELNLGRREEYDVLDKRRGRDPEM |
| | | GGKPRRKNPQEGLYNELQKDKMAEAYSEIGMKGERRRG |
| | | KGHDGLYQGLSTATKDTYDALHMQALPPR |
| 211 | 2A4/CD70 tandem | GGGGSCPYSNPSLCGGGGSCPYSNPSLCGGGGSDIQMTQ |
| | CAR aa sequence | SPSAMSASVGDRVTITCRASQDISNYLAWFQQKPGKVPK |
| | | RLIYAASSLQSGVPSRFSGSGSGTEFTLTISSLLPEDFATYY |
| | | CLQLNSFPFTFGGGTKVEINGGGGSGGGGSGGGGSGGGG |
| | | SQVTLKESGPVLVKPTETLTLTCTVSGFSLSNARMGVTWI |
| | | RQPPGKALEWLAHIFSNDEKSYSTSLKSRLTISKDTSKTQ |
| | | VVLTMTNMDPVDTATYYCARIRDYYDISSYYDYWGQGT |
| | | LVSVSSGGGGSGGGGGGGGSGGGGSQITLKESGPTLVK |
| | | PTQTLTLTCTFSGFSLSTSGVGVGWIRQTPGKALEWLTQI |
| | | YWNDEKRYSPSLRNRLTITKDTSKNQVVLTMTNMDPVD |
| | | TATYYCAHRRGVGNWFDPWGQGILVTVSSGGGGSGGGG |
| | | SGGGGSGGGGSDIQMTQSPSSVSASVGDRVTITCRASQGI |
| | | SSWLAWYQQKPGKAPKLLIYAASSLQSGVSSRFSGSESGT |
| | | DFTLTISSLQPEDFATYYCQQANSFPFTFGPGTKVDIKTTT |
| | | PAPRPPTPAPTIASQPLSLRPEACRPAAGGAVHTRGLDFA |
| | | CDIYIWAPLAGTCGVLLLSLVITLYCKRGRKKLLYIFKQPF |
| | | MRPVQTTQEEDGCSCRFPEEEEGGCELRVKFSRSADAPA |
| | | YQQGQNQLYNELNLGRREEYDVLDKRRGRDPEMGGKPR |
| | | RKNPQEGLYNELQKDKMAEAYSEIGMKGERRRGKGHDG |
| | | LYQGLSTATKDTYDALHMQALPPR |

### Example 8: In Vitro Detection and Depletion of Claudin 18.2 CAR T Cells Using Rituximab-Based Safety-Switch

In order to deplete or turn off CAR T cells in the event of unwanted activity, a rituximab off-switch is developed by insertion of rituximab mimotopes at one or more varying locations in the extracellular region of the CARs as described herein. A complement-dependent cytotoxicity assay is used to evaluate rituximab-dependent in vitro depletion of Claudin 18.2 CAR-T cells. In this assay, frozen CAR-T cells are thawed and 1×10⁵ cells are incubated in RPMI 1640 medium supplemented with 10% FBS in 96-well plates. Cells are incubated for 3 hours in the absence or presence of 25% baby rabbit complement (Cedarlane, CL3441-S) and rituximab antibodies (produced in-house; 100 mg/mL). Cells are stained with recombinant Claudin 18.2 (Adipogen) and cytotoxicity is analyzed by flow cytometry. Anti-Claudin 18.2 CAR-T cells can be detected by both recombinant Claudin 18.2 and rituximab staining. Claudin 18.2 CAR-T cells are depleted in vitro in a rituximab-dependent and complement-dependent manner.

### Example 9: Analysis of Potential Off- and On-Target Risks for the Claudin 18.2 CAR T Cells

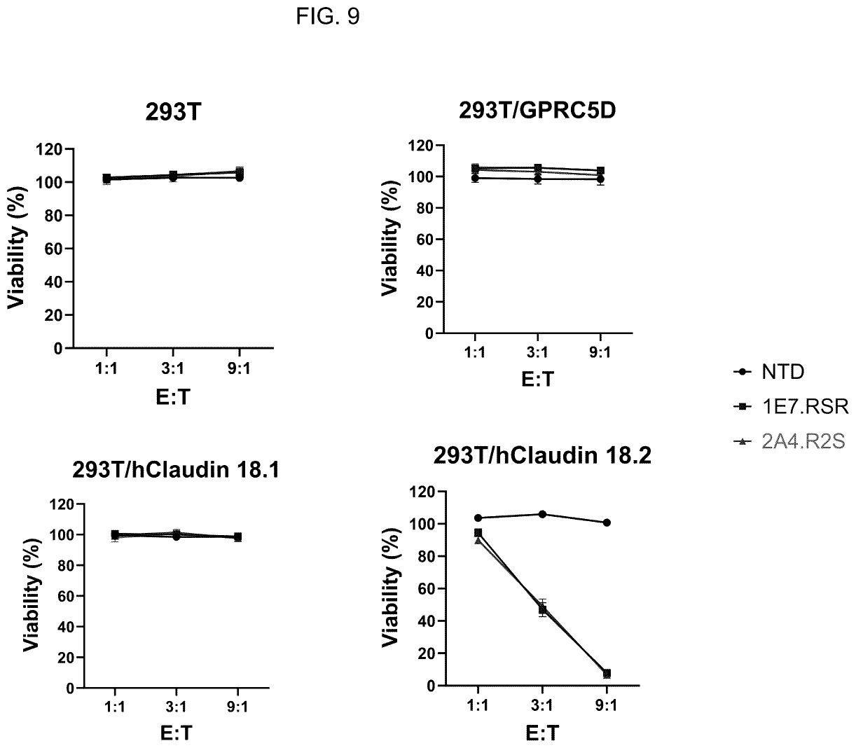

We next examined whether the anti-Claudin 18.2 antibodies may cross react with any other membrane proteins. A membrane proteome array was used to assess the binding profile of Claudin18.2 CARs to identify potential off-target hits for both clones 1E7 and 2A4. The membrane protein GPRC5D was identified as a potential off-target hit for clone 1E7, and none was identified for clone 2A4. Any off-target toxicity of the anti-Claudin 8.2 clones 1E7 and A4 CAR T was tested in the short-term killing assay. 293T cells or 293T cells expressing GPRC5D, Claudin18.1, or Claudin18.2 were co-cultured with the Claudin18.2 CARs for 72 hours in the short-term killing assay as described herein at different E:T ratios. The data in FIG. 9 demonstrate that the Claudin 18.2 CAR T cells exhibit an acceptable safety profile because the cytotoxicity was specific for Claudin 18.2. Results represent mean f SEM, n=3 technical replicates. Experiments were performed three times with CAR T cells from two different donors, and representative data are shown in FIG. 9.

Claudin 18.2 has been shown to express on normal stomach tissues. We next conducted histopathology analysis to evaluate any potential on-target toxicity of the Claudin 18.2 CAR T cells. An in vivo NUGC4 subcutaneous tumor model was used for the experiment. Briefly, mice were inoculated with tumor cells subcutaneously on Day −20, and the CAR T cells were administered intravenously on Day 0. The tissues were harvested on Day 40 or were sooner if rapid body weight loss was observed. Histopathology analysis showed infiltration and tissue damage in the stomach, which was expected and matches the normal tissue expression of Claudin18.2. The histopathological scores of both the 1E7 and 2A4 Claudin 18.2 CARs are comparable to the scores of the two Tool CARs that have been in clinical development either as a CAR or an antibody therapeutic. The findings are summarized below in Table 11.

TABLE 11

| | | Treatment | | | | |
|---|---|---|---|---|---|---|
| | | | Claudin 18.2 | Claudin 18.2 | Tool | Tool |
| | | NTD | CAR (1E7), | CAR (2A4), | CAR 1, | CAR 2, |
| | | 1 × 10⁷ | 1 × 10⁷ | 1 × 10⁷ | 1 × 10⁷ | 1 × 10⁷ |
| Tissue | Findings | (n = 3) | (n = 3) | (n = 3) | (n = 3) | (n = 3) |
| Stomach | Infiltrate, | 0 | Moderate | Moderate | Moderate | Moderate |
| (Claudin 18.2+/ | mononuclear/ | | (n = 2) | (n = 2) | (n = 3) | (n = 3) |
| Claudin 18.1−) | mixed cell | | | Marked | | |
| | | | | (n = 1) | | |
| | Ulcer/Erosion | 0 | Mild | Moderate | 0 | Marked |
| | | | (n = 1) | (n = 1) | | (n = 3) |
| | Hyperplasia | 0 | Mild | Minimal | Mild | Mild |
| | | | (n = 2) | (n = 1) | (n = 1) | (n = 1) |
| | | | | | | Moderate |
| | | | | | | (n = 2) |
| Lung | Infiltrate, | 0 | Moderate | Moderate | Mild | Mild |
| (Claudin 18.2−/ | mononuclear/ | | (n = 2) | (n = 1) | (n = 1) | (n = 3) |
| Claudin 18.1+) | mixed cell | | Marked | Marked | Moderate | |
| | | | (n = 1) | (n = 2) | (n = 2) | |

### Example 10: Claudin 18.2 CAR T Cells with Enhanced Resistance to Host Cell Rejection

We have previously demonstrated that co-expressing in the CAR T cells a rejection avoidance protein, such as a CD70 binding protein, or a CD70 CAR can improve resistance of the CAR T cells to alloreactive immune cells. See WO2022/266203, incorporated herein in its entirety for all purposes. An expression cassette that encodes a Claudin 18.2 CAR and a CD70 CAR (anti-CD70 scFv-CD8 hinge and transmembrane domains-CD3z) linked by P2A was constructed (the Claudin 18.2/CD70 dual CAR). Alternatively, the anti-CD70 scFv coding sequence was added to the 5′ end of the coding sequence of the Claudin 18.2 scFv to generate the CD70/Claudin 18.2 tandem CAR construct. Clone 2A4 was tested in this experiment. CAR T cells expressing either the Claudin 18.2 CAR alone, the dual CAR or the tandem CAR were produced and the expression of the CD70 scFv and Claudin 18.2 scFv on the cell surface from respective CAR T cells was verified by flow cytometry (data not shown).

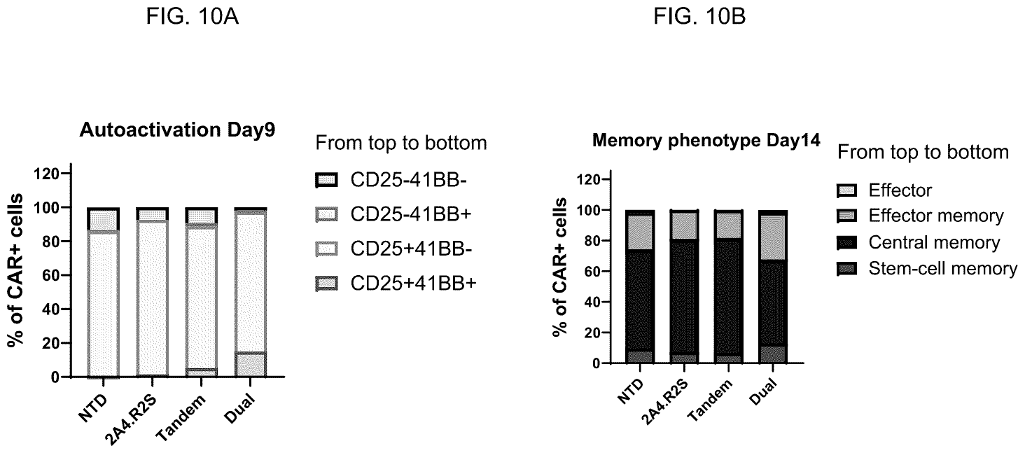

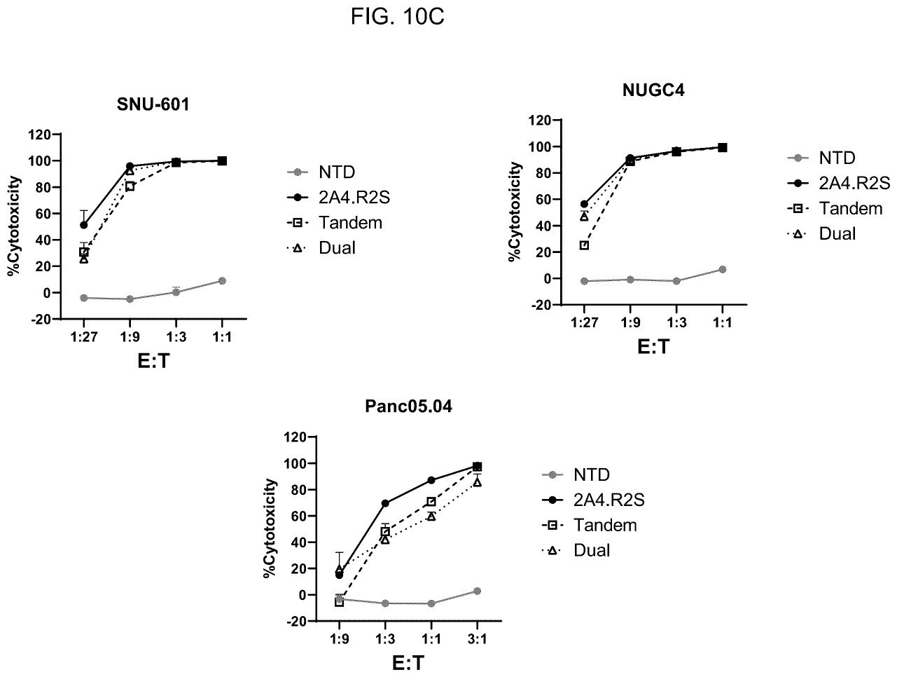

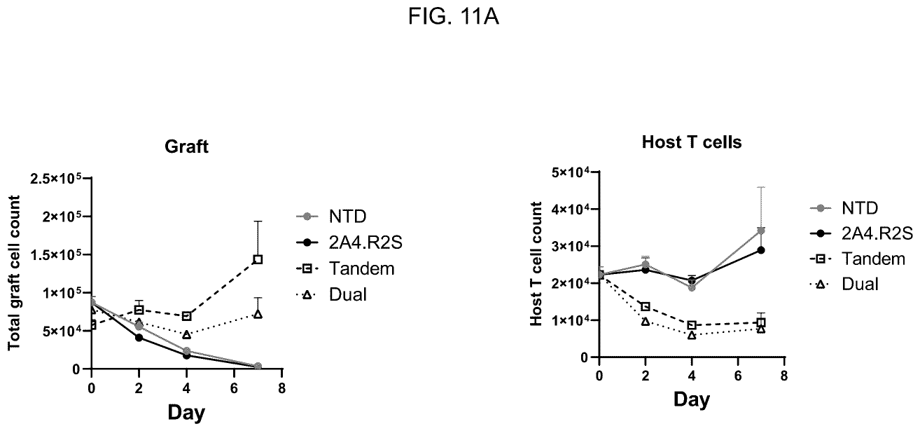

The properties of the Claudin 18.2/CD70 dual and tandem CAR T cells were analyzed as compared to the Claudin 18.2 CAR T cells, including the levels of activation markers (FIG. 10A), and T cell phenotype (FIG. 10B). The expression of a CD70 CAR did not significantly affect the cytotoxicity activity of the Claudin 18.2 CAR T cells (FIG. 10C).

To evaluate the activities of the Claudin 18.2 CAR T cells co-expressing an allo-rejection avoidance protein, such as a CD70 binding protein or a CD70 CAR, we performed alloreactive T cell mixed lymphocyte reaction (MLR) assays. To prime alloreactive T cells, unedited graft donor T cells were irradiated at 30 Gy and co-cultured with host PBMCs at a ratio of 1:1 in RPMI supplemented with 10% FBS and 20 IU/mL of recombinant human IL-2, IL-7, and IL-15. At day 4 of the co-culture, half the media was replaced with fresh RPMI supplemented with 10% FBS. At day 7, T cells were isolated using human Pan T cell isolation kit (Miltenyi Biotech) as instructed by the manufacturer protocol. Primed alloreactive T cells were then co-cultured with graft T cells at 1:1 ratio in 200 uL RPMI medium supplemented with 10% FBS and 20 U/mL of recombinant human IL-2 in round-bottomed 96-well plates. If MLR co-cultures exceeded 4 days, half the medium was replaced on day 4. Cells were analyzed by flow cytometry at the indicated time points.

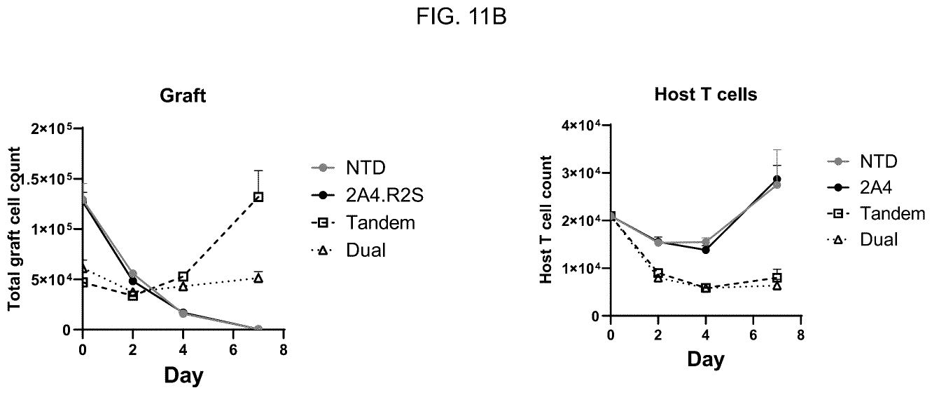

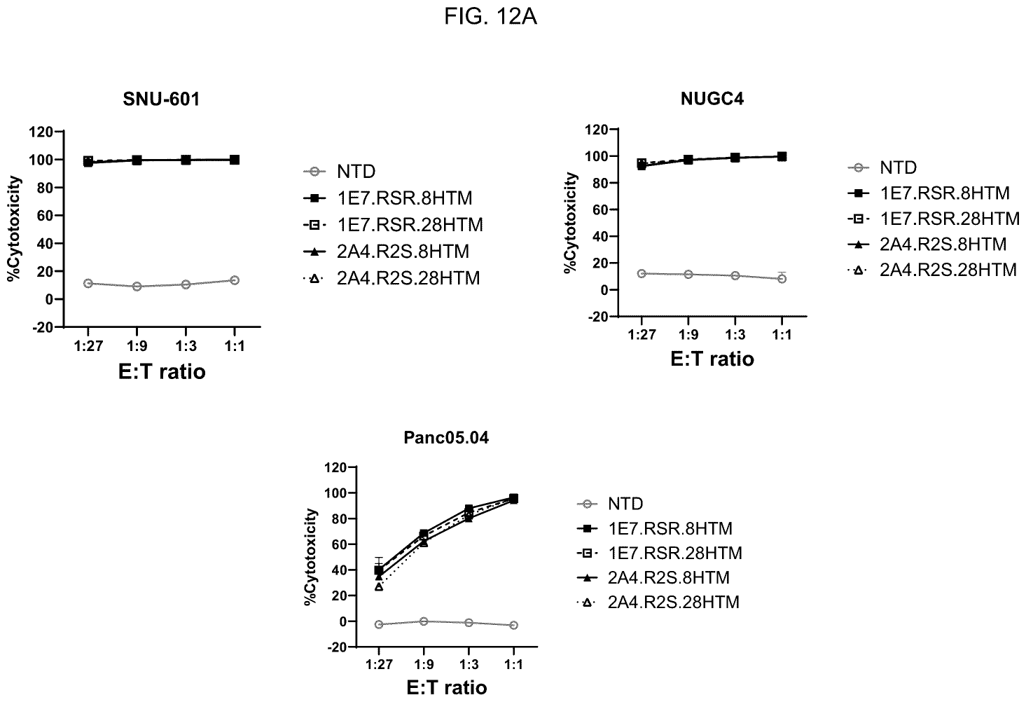

As shown in FIG. 11A, Claudin 18.2 CAR T cells expressing the CD70 CAR resisted alloreactive T cell-mediated rejection while NTD as well as CAR T cells that did not express the CD70 CAR were completely rejected (left panel). Both Tandem and Dual CARs showed improved survival to varying degrees and significantly reduced the absolute numbers of the host T cells, evidence of effective immune rejection avoidance activity. Conversely, host T cells expanded when co-cultured with NTD and Claudin 18.2 CAR T cells without expressing the CD70 CAR (right panel). The same experiment was repeated with a different graft-donor pair (FIG. 11B).

### Example 11: Claudin 18.2 CAR with CD28 Hinge and Transmembrane Domains

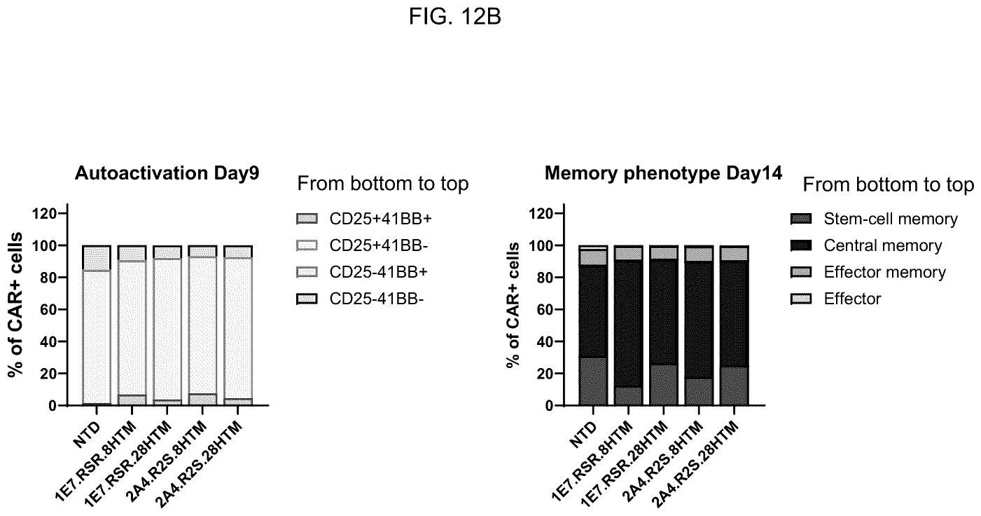

We next constructed anti-Claudin 18.2 CAR constructs with CD28 hinge and transmembrane domains (CD28 HTM) and compared the CAR T cells with anti-Claudin 18.2 CAR T cells with the CD8α hinge and transmembrane domains (8HTM). The results in FIG. 12A show that the CAR T cells exhibited similar cytotoxicity in the short term-killing assay, and the CAR T cells expressing the CD28 HTM show slightly less activation markers and more stem central memory cells (FIG. 12B).

TABLE 12
Other Exemplary Sequences

| SEQ ID NO | Description | Sequence |
|---|---|---|
| 204 | Anti-CD70 antibody | QVTLKESGPVLVKPTETLTLTCTVSGFSLSNARMG |
| | scFv | VTWIRQPPGKALEWLAHIFSNDEKSYSTSLKSRLTI |
| | | SKDTSKTQVVLTMTNMDPVDTATYYCARIRDYY |
| | | DISSYYDYWGQGTLVSVSSGGGGSGGGGSGGGGS |
| | | DIQMTQSPSAMSASVGDRVTITCRASQDISNYLAW |
| | | FQQKPGKVPKRLIYAASSLQSGVPSRFSGSGSGTEF |
| | | TLTISSLLPEDFATYYCLQLNSFPFTFGGGTKVEIN |
| 205 | Anti-CD70 antibody | DIQMTQSPSAMSASVGDRVTITCRASQDISNYLA |
| | VL | WFQQKPGKVPKRLIYAASSLQSGVPSRFSGSGS |
| | | GTEFTLTISSLLPEDFATYYCLQLNSFPFTFGGGT |
| | | KVEIN |
| 206 | Anti-CD70 antibody | QVTLKESGPVLVKPTETLTLTCTVSGFSLSNARM |
| | VL | GVTWIRQPPGKALEWLAHIFSNDEKSYSTSLKSR |
| | | LTISKDTSKTQVVLTMTNMDPVDTATYYCARIR |
| | | DYYDISSYYDYWGQGTLVSVSS |
| 207 | CD70 binding protein | MALPVTALLLPLALLLHAARPQVTLKESGPVLVKP |
| | (underlined signal | TETLTLTCTVSGFSLSNARMGVTWIRQPPGKALEW |
| | peptide) | LAHIFSNDEKSYSTSLKSRLTISKDTSKTQVVLTMT |
| | | NMDPVDTATYYCARIRDYYDISSYYDYWGQGTL |
| | | VSVSSGGGGSGGGGSGGGGSDIQMTQSPSAMSAS |
| | | VGDRVTITCRASQDISNYLAWFQQKPGKVPKRLIY |
| | | AASSLQSGVPSRFSGSGSGTEFTLTISSLLPEDFATY |
| | | YCLQLNSFPFTFGGGTKVEINTTTPAPRPPTPAPTIA |
| | | SQPLSLRPEACRPAAGGAVHTRGLDFACDIYIWAP |
| | | LAGTCGVLLLSLVITLYCRVKFSRSADAPAYQQGQ |
| | | NQLYNELNLGRREEYDVLDKRRGRDPEMGGKPR |
| | | RKNPQEGLYNELQKDKMAEAYSEIGMKGERRRG |
| | | KGHDGLYQGLSTATKDTYDALHMQALPPR |

All references cited herein, including patents, patent applications, papers, text books, and the like, and the references cited therein, to the extent that they are not already, are hereby incorporated by reference in their entirety.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

1. (canceled)

2. (canceled)

3. (canceled)

4. A chimeric antigen receptor comprising an extracellular domain, a transmembrane domain, and an intracellular domain, wherein the extracellular domain comprises a Claudin 18.2 antigen binding domain that specifically binds to Claudin 18.2, and wherein the antigen binding domain comprises at least one of:
(a) a variable heavy chain CDR1 (CDRH1) comprising an amino acid sequence selected from SEQ ID NOs: 1-3; a CDRH2 comprising an amino acid sequence selected from SEQ ID NOs: 4-5; a CDRH3 comprising the amino acid sequence of SEQ ID NO: 6; a variable light chain CDR1 (CDRL1) comprising the amino acid sequence of SEQ ID NO: 7; a CDRL2 comprising the amino acid sequence of SEQ ID NO: 8; and a CDRL3 comprising the amino acid sequence of SEQ ID NO: 9; or
(b) a CDRH1 comprising an amino acid sequence selected from SEQ ID NOs: 16-18; a CDRH2 comprising an amino acid sequence selected from SEQ ID NOs: 19-20; a CDRH3 comprising the amino acid sequence of SEQ ID NO: 21; a CDRL1 comprising the amino acid sequence of SEQ ID NO: 22; a CDRL2 comprising the amino acid sequence of SEQ ID NO: 23; and a CDRL3 comprising the amino acid sequence of SEQ ID NO: 24; or
(c) a CDRH1 comprising an amino acid sequence selected from SEQ ID NOs: 31-33; a CDRH2 comprising an amino acid sequence selected from SEQ ID NOs: 34-35; a CDRH3 comprising the amino acid sequence of SEQ ID NO: 36; a CDRL1 comprising the amino acid sequence of SEQ ID NO: 37; a CDRL2 comprising the amino acid sequence of SEQ ID NO: 38; and a CDRL3 comprising the amino acid sequence of SEQ ID NO: 39; or
(d) a CDRH1 comprising an amino acid sequence selected from SEQ ID NOs: 46-48; a CDRH2 comprising an amino acid sequence selected from SEQ ID NOs: 49-50; a CDRH3 comprising the amino acid sequence of SEQ ID NO: 51; a CDRL1 comprising the amino acid sequence of SEQ ID NO: 52; a CDRL2 comprising the amino acid sequence of SEQ ID NO: 53; and a CDRL3 comprising the amino acid sequence of SEQ ID NO: 54; or
(e) a CDRH1 comprising an amino acid sequence selected from SEQ ID NOs: 61-63; a CDRH2 comprising an amino acid sequence selected from SEQ ID NOs: 64-65; a CDRH3 comprising the amino acid sequence of SEQ ID NO: 66; a CDRL1 comprising the amino acid sequence of SEQ ID NO: 67; a CDRL2 comprising the amino acid sequence of SEQ ID NO: 68; and a CDRL3 comprising the amino acid sequence of SEQ ID NO: 69; or
(f) a CDRH1 comprising an amino acid sequence selected from SEQ ID NOs: 76-78; a CDRH2 comprising an amino acid sequence selected from SEQ ID NOs: 79-80; a CDRH3 comprising the amino acid sequence of SEQ ID NO: 81; a CDRL1 comprising the amino acid sequence of SEQ ID NO: 82; a CDRL2 comprising the amino acid sequence of SEQ ID NO: 83; and a CDRL3 comprising the amino acid sequence of SEQ ID NO: 84; or
(g) a CDRH1 comprising an amino acid sequence selected from SEQ ID NOs: 89-91; a CDRH2 comprising an amino acid sequence selected from SEQ ID NOs: 92-93; a CDRH3 comprising the amino acid sequence of SEQ ID NO: 94; a CDRL1 comprising the amino acid sequence of SEQ ID NO: 95; a CDRL2 comprising the amino acid sequence of SEQ ID NO: 96; and a CDRL3 comprising the amino acid sequence of SEQ ID NO: 97; or
(h) a CDRH1 comprising an amino acid sequence selected from SEQ ID NOs: 102-104; a CDRH2 comprising an amino acid sequence selected from SEQ ID NOs: 105-106; a CDRH3 comprising the amino acid sequence of SEQ ID NO: 107; a CDRL1 comprising the amino acid sequence of SEQ ID NO: 108; a CDRL2 comprising the amino acid sequence of SEQ ID NO: 109; and a CDRL3 comprising the amino acid sequence of SEQ ID NO: 110; or
(i) a CDRH1 comprising an amino acid sequence selected from SEQ ID NOs: 115-117; a CDRH2 comprising an amino acid sequence selected from SEQ ID NOs: 118-119; a CDRH3 comprising the amino acid sequence of SEQ ID NO: 120; a CDRL1 comprising the amino acid sequence of SEQ ID NO: 121; a CDRL2 comprising the amino acid sequence of SEQ ID NO: 122; and a CDRL3 comprising the amino acid sequence of SEQ ID NO: 123.

5. (canceled)

6. The chimeric antigen receptor of claim 4 wherein the antigen binding domain comprises:
(a) a variable heavy chain (VH) comprising an amino acid sequence that is at least 80%, at least 85%, at least 90%, at least 95%, at least 96%, at least 97%, at least 98%, at least 99% or 100% identical to the amino acid sequence of SEQ ID NO: 10, and
a variable light chain (VL) comprising an amino acid sequence that is at least 80%, at least 85%, at least 90%, at least 95%, at least 96%, at least 97%, at least 98%, at least 99% or 100% identical to the amino acid sequence of SEQ ID NO: 11; or
(b) a VH comprising an amino acid sequence that is at least 80%, at least 85%, at least 90%, at least 95%, at least 96%, at least 97%, at least 98%, at least 99% or 100% identical to the amino acid sequence of SEQ ID NO: 25; and a VL comprising an amino acid sequence that is at least 80%, at least 85%, at least 90%, at least 95%, at least 96%, at least 97%, at least 98%, at least 99% or 100% identical to the amino acid sequence of SEQ ID NO: 26; or
(c) a VH comprising an amino acid sequence that is at least 80%, at least 85%, at least 90%, at least 95%, at least 96%, at least 97%, at least 98%, at least 99% or 100% identical to the amino acid sequence of SEQ ID NO: 40; and a VL comprising an amino acid sequence that is at least 80%, at least 85%, at least 90%, at least 95%, at least 96%, at least 97%, at least 98%, at least 99% or 100% identical to the amino acid sequence of SEQ ID NO: 41; or
(d) a VH comprising an amino acid sequence that is at least 80%, at least 85%, at least 90%, at least 95%, at least 96%, at least 97%, at least 98%, at least 99% or 100% identical to the amino acid sequence of SEQ ID NO: 55; and a VL comprising an amino acid sequence that is at least 80%, at least 85%, at least 90%, at least 95%, at least 96%, at least 97%, at least 98%, at least 99% or 100% identical to the amino acid sequence of SEQ ID NO: 56; or
(e) a VH comprising an amino acid sequence that is at least 80%, at least 85%, at least 90%, at least 95%, at least 96%, at least 97%, at least 98%, at least 99% or 100% identical to the amino acid sequence of SEQ ID NO: 70; and a VL comprising an amino acid sequence that is at least 80%, at least 85%, at least 90%, at least 95%, at least 96%, at least 97%, at least 98%, at least 99% or 100% identical to the amino acid sequence of SEQ ID NO: 71; or
(f) a VH comprising an amino acid sequence that is at least 80%, at least 85%, at least 90%, at least 95%, at least 96%, at least 97%, at least 98%, at least 99% or 100% identical to the amino acid sequence of SEQ ID NO: 85; and a VL comprising an amino acid sequence that is at least 80%, at least 85%, at least 90%, at least 95%, at least 96%, at least 97%, at least 98%, at least 99% or 100% identical to the amino acid sequence of SEQ ID NO: 86; or
(g) a VH comprising an amino acid sequence that is at least 80%, at least 85%, at least 90%, at least 95%, at least 96%, at least 97%, at least 98%, at least 99% or 100% identical to the amino acid sequence of SEQ ID NO: 98; and a VL comprising an amino acid sequence that is at least 80%, at least 85%, at least 90%, at least 95%, at least 96%, at least 97%, at least 98%, at least 99% or 100% identical to the amino acid sequence of SEQ ID NO: 99; or
(h) a VH comprising an amino acid sequence that is at least 80%, at least 85%, at least 90%, at least 95%, at least 96%, at least 97%, at least 98%, at least 99% or 100% identical to the amino acid sequence of SEQ ID NO: 111; and a VL comprising an amino acid sequence that is at least 80%, at least 85%, at least 90%, at least 95%, at least 96%, at least 97%, at least 98%, at least 99% or 100% identical to the amino acid sequence of SEQ ID NO: 112; or
(i) a VH comprising an amino acid sequence that is at least 80%, at least 85%, at least 90%, at least 95%, at least 96%, at least 97%, at least 98%, at least 99% or 100% identical to the amino acid sequence of SEQ ID NO: 124; and a VL comprising an amino acid sequence that is at least 80%, at least 85%, at least 90%, at least 95%, at least 96%, at least 97%, at least 98%, at least 99% or 100% identical to the amino acid sequence of SEQ ID NO: 125.

7. The chimeric antigen receptor of claim 4, wherein the antigen binding domain comprises:
(a) a heavy chain variable domain (VH) comprising the amino acid sequence of SEQ ID NO: 10; and a light chain variable domain (VL) comprising the amino acid sequence of SEQ ID NO: 11; or
(b) a VH comprising the amino acid sequence of SEQ ID NO: 25; and a VL comprising the amino acid sequence of SEQ ID NO: 26; or
(c) a VH comprising the amino acid sequence of SEQ ID NO: 40; and a VL comprising the amino acid sequence of SEQ ID NO: 41; or
(d) a VH comprising the amino acid sequence of SEQ ID NO: 55; and a VL comprising the amino acid sequence of SEQ ID NO: 56; or
(c) a VH comprising the amino acid sequence of SEQ ID NO: 70; and a VL comprising the amino acid sequence of SEQ ID NO: 71; or
(f) a VH comprising the amino acid sequence of SEQ ID NO: 85; and a VL comprising the amino acid sequence of SEQ ID NO: 86; or
(g) a VH comprising the amino acid sequence of SEQ ID NO: 98; and a VL comprising the amino acid sequence of SEQ ID NO: 99; or
(h) a VH comprising the amino acid sequence of SEQ ID NO: 111; and a VL comprising the amino acid sequence of SEQ ID NO: 112; or
(i) a VH comprising the amino acid sequence of SEQ ID NO: 124; and a VL comprising the amino acid sequence of SEQ ID NO: 125.

8. The chimeric antigen receptor of claim 4, wherein the antigen binding domain comprises an scFv comprising an amino acid sequence selected from the group consisting of SEQ ID NOS: 12, 27, 42, 57, 72, 187, 189, 191 and 193.

9. The chimeric antigen receptor of claim 4, wherein the chimeric antigen receptor comprises an amino acid sequence that is about 80%, 85%, 90%, 95%, 96%, 98%, 99% or 100% identical to any one of SEQ ID NOs: 13, 28, 43, 58, 73, 87, 100, 113, 126, 128, 130, 132, 179-186, 195-198, 200-201 and 208-211, with or without a signal sequence.

10. The chimeric antigen receptor of claim 4, wherein the transmembrane domain comprises a transmembrane domain of human CD8a, CD28, or CD2.

11. (canceled)

12. The chimeric antigen receptor of claim 10, wherein the chimeric antigen receptor further comprises a hinge domain that comprises a hinge domain of human CD8a, CD28 or CD2.

13. The chimeric antigen receptor of claim 11, wherein the hinge and transmembrane domains are the hinge and transmembrane domains of human CD8a, CD28 or CD2.

14. (canceled)

15. The chimeric antigen receptor of claim 13, wherein the intracellular domain comprises at least one costimulatory domain, wherein the costimulatory domain is a signaling region of CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1 (CD1 1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), activating NK cell receptors, BTLA, a Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, CD49D, ITGA6, VLA-6, CD49f, ITGAD, CD1 1d, ITGAE, CD103, ITGAL, CD1 1a, LFA-1, ITGAM, CD1 1b, ITGAX, CD1 1c, ITGB1, CD29, ITGB2, CD18, LFA-1, ITGB7, NKG2D, TNFR2, TRANCE/RANKL, DNAMI (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

16. The chimeric antigen receptor of claim 15, wherein the costimulatory domain comprises one or more signaling regions selected from the group consisting of a 4-1BB/CD137 signaling region, a CD28 signaling region, and a variant thereof.

17. The chimeric antigen receptor of claim 16, wherein the costimulatory domain comprises a signaling region comprising the amino acid sequence of one or more of SEQ ID NOS: 137, 158, 159, and 174.

18. The chimeric antigen receptor of claim 4, wherein the intracellular domain comprises a CD3 signalling domain.

19. (canceled)

20. (canceled)

21. (canceled)

22. The chimeric antigen receptor of claim 4, wherein the chimeric antigen receptor is encoded by a polynucleotide sequence selected from the group consisting of SEQ ID NOs: 15, 30, 45, 60, 75, 88, 101, 114 and 127.

23. The chimeric antigen receptor of claim 4, wherein the extracellular domain further comprises an anti-CD70 scFv that specifically binds to CD70.

24. The chimeric antigen receptor of claim 23, wherein the anti-CD70 scFv comprises the amino acid sequence of SEQ ID NO: 204, 205, and/or 206.

25. The chimeric antigen receptor of claim 4, further comprising a safety switch.

26. The chimeric antigen receptor of claim 25, wherein the safety switch comprises a CD20 mimotope or a QBEND-10 epitope.

27. (canceled)

28. The chimeric antigen receptor of claim 26, wherein the chimeric antigen receptor comprises one or more safety switch in the format of QR3, SR2, RSR, or R2S.

29. (canceled)

30. An isolated polynucleotide encoding the chimeric antigen receptor of claim 4.

31. A vector comprising the polynucleotide of claim 30.

32. (canceled)

33. An engineered immune cell comprising or expressing the chimeric antigen receptor of claim 4.

34. (canceled)

35. (canceled)

36. (canceled)

37. (canceled)

38. The engineered immune cell of claim 33, wherein the engineered immune cell further comprises or expresses a CD70 binding protein.

39. The engineered immune cell of claim 38, wherein the CD70 binding protein comprises an anti-CD70 antibody or an antigen binding fragment thereof, and a transmembrane domain.

40. (canceled)

41. (canceled)

42. (canceled)

43. (canceled)

44. (canceled)

45. A pharmaceutical composition comprising the engineered immune cell of claim 33 and at least one pharmaceutical acceptable excipient.

46. A method of treating a disease or disorder in a subject in need thereof comprising administering to the subject an effective amount of the engineered immune cell of claim 33.

47. The method of claim 46, wherein the engineered immune cell is administered to the subject intravenously, subcutaneously, or intraperitoneally.

48. (canceled)

49. (canceled)

50. (canceled)

51. (canceled)

52. (canceled)

53. An anti-Claudin 18.2 antibody comprising:
(a) a variable heavy chain CDR1 (CDRH1) comprising an amino acid sequence selected from SEQ ID NOs: 1-3; a CDRH2 comprising an amino acid sequence selected from SEQ ID NOs: 4-5; a CDRH3 comprising the amino acid sequence of SEQ ID NO: 6; a variable light chain CDR1 (CDRL1) comprising the amino acid sequence of SEQ ID NO: 7; a CDRL2 comprising the amino acid sequence of SEQ ID NO: 8; and a CDRL3 comprising the amino acid sequence of SEQ ID NO: 9; or
(b) a CDRH1 comprising an amino acid sequence selected from SEQ ID NOs: 16-18; a CDRH2 comprising an amino acid sequence selected from SEQ ID NOs: 19-20; a CDRH3 comprising the amino acid sequence of SEQ ID NO: 21; a CDRL1 comprising the amino acid sequence of SEQ ID NO: 22; a CDRL2 comprising the amino acid sequence of SEQ ID NO: 23; and a CDRL3 comprising the amino acid sequence of SEQ ID NO: 24; or
(c) a CDRH1 comprising an amino acid sequence selected from SEQ ID NOs: 31-33; a CDRH2 comprising an amino acid sequence selected from SEQ ID NOs: 34-35; a CDRH3 comprising the amino acid sequence of SEQ ID NO: 36; a CDRL1 comprising the amino acid sequence of SEQ ID NO: 37; a CDRL2 comprising the amino acid sequence of SEQ ID NO: 38; and a CDRL3 comprising the amino acid sequence of SEQ ID NO: 39; or
(d) a CDRH1 comprising an amino acid sequence selected from SEQ ID NOs: 46-48; a CDRH2 comprising an amino acid sequence selected from SEQ ID NOs: 49-50; a CDRH3 comprising the amino acid sequence of SEQ ID NO: 51; a CDRL1 comprising the amino acid sequence of SEQ ID NO: 52; a CDRL2 comprising the amino acid sequence of SEQ ID NO: 53; and a CDRL3 comprising the amino acid sequence of SEQ ID NO: 54; or
(e) a CDRH1 comprising an amino acid sequence selected from SEQ ID NOs: 61-63; a CDRH2 comprising an amino acid sequence selected from SEQ ID NOs: 64-65; a CDRH3 comprising the amino acid sequence of SEQ ID NO: 66; a CDRL1 comprising the amino acid sequence of SEQ ID NO: 67; a CDRL2 comprising the amino acid sequence of SEQ ID NO: 68; and a CDRL3 comprising the amino acid sequence of SEQ ID NO: 69; or
(f) a CDRH1 comprising an amino acid sequence selected from SEQ ID NOs: 76-78; a CDRH2 comprising an amino acid sequence selected from SEQ ID NOs: 79-80; a CDRH3 comprising the amino acid sequence of SEQ ID NO: 81; a CDRL1 comprising the amino acid sequence of SEQ ID NO: 82; a CDRL2 comprising the amino acid sequence of SEQ ID NO: 83; and a CDRL3 comprising the amino acid sequence of SEQ ID NO: 84; or
(g) a CDRH1 comprising an amino acid sequence selected from SEQ ID NOs: 89-91; a CDRH2 comprising an amino acid sequence selected from SEQ ID NOs: 92-93; a CDRH3 comprising the amino acid sequence of SEQ ID NO: 94; a CDRL1 comprising the amino acid sequence of SEQ ID NO: 95; a CDRL2 comprising the amino acid sequence of SEQ ID NO: 96; and a CDRL3 comprising the amino acid sequence of SEQ ID NO: 97; or
(h) a CDRH1 comprising an amino acid sequence selected from SEQ ID NOs: 102-104; a CDRH2 comprising an amino acid sequence selected from SEQ ID NOs: 105-106; a CDRH3 comprising the amino acid sequence of SEQ ID NO: 107; a CDRL1 comprising the amino acid sequence of SEQ ID NO: 108; a CDRL2 comprising the amino acid sequence of SEQ ID NO: 109; and a CDRL3 comprising the amino acid sequence of SEQ ID NO: 110; or
(i) a CDRH1 comprising an amino acid sequence selected from SEQ ID NOs: 115-117; a CDRH2 comprising an amino acid sequence selected from SEQ ID NOs: 118-119; a CDRH3 comprising the amino acid sequence of SEQ ID NO: 120; a CDRL1 comprising the amino acid sequence of SEQ ID NO: 121; a CDRL2 comprising the amino acid sequence of SEQ ID NO: 122; and a CDRL3 comprising the amino acid sequence of SEQ ID NO: 123.

54. (canceled)

55. (canceled)

56. The anti-Claudin 18.2 antibody of claim 53 comprising
(a) a variable heavy chain (VH) comprising an amino acid sequence that is at least 80%, 85%, 90%, 95%, 96%, 98%, 99% or 100% identical to the amino acid sequence of SEQ ID NO: 10, and a variable light (VL) chain (VL) comprising an amino acid-sequence that is at least 80%, 85%, 90%, 95%, 96%, 98%, 99% or 100% identical to the amino acid sequence of SEQ ID NO: 11; or
(b) a VH comprising an amino acid sequence that is at least 80%, 85%, 90%, 95%, 96%, 98%, 99% or 100% identical to the amino acid sequence of SEQ ID NO: 25, and a VL comprising an amino acid sequence that is at least 80%, 85%, 90%, 95%, 96%, 98%, 99% or 100% identical to the amino acid sequence of SEQ ID NO: 26; or
(c) a VH comprising an amino acid sequence that is at least 80%, 85%, 90%, 95%, 96%, 98%, 99% or 100% identical to the amino acid sequence of SEQ ID NO: 40, and a VL comprising an amino acid sequence that is at least 80%, 85%, 90%, 95%, 96%, 98%, 99% or 100% identical to the amino acid sequence of SEQ ID NO: 41; or
(d) a VH comprising an amino acid sequence that is at least 80%, 85%, 90%, 95%, 96%, 98%, 99% or 100% identical to the amino acid sequence of SEQ ID NO: 55, and a VL comprising an amino acid sequence that is at least 80%, 85%, 90%, 95%, 96%, 98%, 99% or 100% identical to the amino acid sequence of SEQ ID NO: 56; or
(e) a VH comprising an amino acid sequence that is at least 0%, 85%, 90%, 95%, 96%, 98%, 99% or 100% identical to the amino acid sequence of SEQ ID NO: 70, and a VL comprising an amino acid sequence that is at least 80%, 85%, 90%, 95%, 96%, 98%, 99% or 100% identical to the amino acid sequence of SEQ ID NO: 71; or
(f) a VH comprising an amino acid sequence that is at least 80%, 85%, 90%, 95%, 96%, 98%, 99% or 100% identical to the amino acid sequence of SEQ ID NO: 85, and a VL comprising an amino acid sequence that is at least 80%, 85%, 90%, 95%, 96%, 98%, 99% or 100% identical to the amino acid sequence of SEQ ID NO: 86; or
(g) a VH comprising an amino acid sequence that is at least 80%, 85%, 90%, 95%, 96%, 98%, 99% or 100% identical to the amino acid sequence of SEQ ID NO: 98, and a VL comprising an amino acid sequence that is at least 80%, 85%, 90%, 95%, 96%, 98%, 99% or 100% identical to the amino acid sequence of SEQ ID NO: 99; or
(h) a VH comprising an amino acid sequence that is at least 80%, 85%, 90%, 95%, 96%, 98%, 99% or 100% identical to the amino acid sequence of SEQ ID NO: 111, and a VL comprising an amino acid sequence that is at least 80%, 85%, 90%, 95%, 96%, 98%, 99% or 100% identical to the amino acid sequence of SEQ ID NO: 112; or
(i) a VH comprising an amino acid sequence that is at least 80%, 85%, 90%, 95%, 96%, 98%, 99% or 100% identical to the amino acid sequence of SEQ ID NO: 124, and a VL comprising an amino acid sequence that is at least 80%, 85%, 90%, 95%, 96%, 98%, 99% or 100% identical to the amino acid sequence of SEQ ID NO: 125.

57. (canceled)

58. (canceled)

59. (canceled)

60. (canceled)

61. (canceled)

62. A pharmaceutical composition comprising the anti-Claudin 18.2 antibody of claim 53 and a pharmaceutically acceptable excipient.

63. A method of treating a disease or disorder in a subject in need thereof comprising administering to the subject the anti-Claudin 18.2 antibody of claim 53.

64. (canceled)

65. (canceled)
